(19) United States
(12) Patent Application Publication
(10) Pub. No.: US 2005/0123912 A1
(43) Pub. Date: Jun. 9, 2005

(54) Nucleic-acid associated proteins

(76) Inventors: Ines Barroso, Cambridge (GB); Mariah R Baughn, Los Angeles, CA (US); Shanya D Becha, San Francisco, CA (US); Julie J Blake, San Francisco, CA (US); Mark L Borowsky, Needham, MA (US); Neil Burford, Durham, CT (US); Brendan M Duggan, Sunnyvale, CA (US); Vicki S Elliott, San Jose, CA (US); Brooke M Emerling, Chicago, IL (US); Ian J Forsythe, Edmonton (CA); Kimberly J Gietzen, San Jose, CA (US); Ann E Gorvad, Bellingham, WA (US); Jennifer A Griffin, Fremont, CA (US); April JA Hafalia, Daly City, CA (US); Cynthia D Honchell, San Francisco, CA (US); Craig H Ison, San Jose, CA (US); Farrah A Khan, Canton, MI (US); Preeti G Lal, Santa Clara, CA (US); Ernestine A Lee, Kensington, CA (US); Sally Lee, San Jose, CA (US); Soo Yeun Lee, Mountain View, CA (US); Joana X Li, Millbrae, CA (US); Dyung Aina M Lu, San Jose, CA (US); Yan Lu, Mountain View, CA (US); Patricia M Lehr-Mason, Morgan Hill, CA (US); Danniel B Nguyen, San Jose, CA (US); Jayalaxmi Ramkumar, Fremont, CA (US); William W Sprague, Sacramento, CA (US); Y Tom Tang, San Jose, CA (US); Kavitha Thangavelu, Sunnyvale, CA (US); Michael B Thornton, Oakland, CA (US); Uyen K Tran, San Jose, CA (US); Narinder K Chawla, Union City, CA (US); Bridget A Warren, San Marcos, CA (US); Yuming Xu, Mountain View, CA (US); Monique G Yao, Mountain View, CA (US); Henry Yue, Sunnyvale, CA (US); Huibin Yue, Cupertino, CA (US); Yeganeh Zebarjadian, San Francisco, CA (US)

Correspondence Address: INCYTE CORPORATION;EXPERIMENTAL STATION, ROUTE 141 & HENRY CLAY ROAD, BLDG. E336, WILMINGTON, DE, 19880, US

(21) Appl. No.: 10/486,977

(22) PCT Filed: Aug. 14, 2002
(86) PCT No.: PCT/US02/25829

(51) Int. Cl. C12Q 1/68; C07H 21/04; C12N 9/22

(52) U.S. Cl. 435/6; 435/69.1; 435/199; 435/320.1; 435/325; 536/23.2

(57) ABSTRACT

Various embodiments of the invention provide human nucleic acid-associated proteins (NAAP) and polynucleotides which identify and encode NAAP. Embodiments of the invention also provide expression vectors, host cells, antibodies, agonists, and antagonists. Other embodiments provide methods for diagnosing, treating, or preventing disorders associated with aberrant expression of NAAP.

### TECHNICAL FIELD

The invention relates to novel nucleic acids, nucleic acid-associated proteins encoded by these nucleic acids, and to the use of these nucleic acids and proteins in the diagnosis, treatment, and prevention of cell proliferative, DNA repair, neurological reproductive, developmental, and autoimmune/inflammatory disorders, and infections. The invention also relates to the assessment of the effects of exogenous compounds on the expression of nucleic acids and nucleic acid-associated proteins.

### BACKGROUND OF THE INVENTION

Multicellular organisms are comprised of diverse cell types that differ dramatically both in structure and function. The identity of a cell is determined by its characteristic pattern of gene expression, and different cell types express overlapping but distinctive sets of genes throughout development. Spatial and temporal regulation of gene expression is critical for the control of cell proliferation, cell differentiation, apoptosis, and other processes that contribute to organismal development. Furthermore, gene expression is regulated in response to extracellular signals that mediate cell-cell communication and coordinate the activities of different cell types. Appropriate gene regulation also ensures that cells function efficiently by expressing only those genes whose functions are required at a given time.

The cell nucleus contains all of the genetic information of the cell in the form of DNA, and the components and machinery necessary for replication of DNA and for transcription of DNA into RNA. (See Alberts, B. et al. (1994) Molecular Biology of the Cell, Garland Publishing Inc., New York N.Y., pp. 335-399.) DNA is organized into compact structures in the nucleus by interactions with various DNA-binding proteins such as histones and non-histone chromosomal proteins. DNA-specific nucleases, DNAses, partially degrade these compacted structures prior to DNA replication or transcription. DNA replication takes place with the aid of DNA helicases which unwind the double-stranded DNA helix, and DNA polymerases that duplicate the separated DNA strands.

Transcription Factors

Transcriptional regulatory proteins are essential for the control of gene expression. Some of these proteins function as transcription factors that initiate, activate, repress, or terminate gene transcription. Transcription factors generally bind to the promoter, enhancer, and upstream regulatory regions of a gene in a sequence-specific manner, although some factors bind regulatory elements within or downstream of a gene coding region. Transcription factors may bind to a specific region of DNA singly or as a complex with other accessory factors. (Reviewed in Lewin, B. (1990) Genes IV, Oxford University Press, New York, N.Y., and Cell Press, Cambridge, Mass., pp. 554-570.)

The double helix structure and repeated sequences of DNA create topological and chemical features which can be recognized by transcription factors. These features are hydrogen bond donor and acceptor groups, hydrophobic patches, major and minor grooves, and regular, repeated stretches of sequence which induce distinct bends in the helix. Typically, transcription factors recognize specific DNA sequence motifs of about 20 nucleotides in length. Multiple, adjacent transcription factor-binding motifs may be required for gene regulation.

Many transcription factors incorporate DNA-binding structural motifs which comprise either a helices or β sheets that bind to the major groove of DNA. Four well-characterized structural motifs are helix-turn-helix, zinc finger, leucine zipper, and helix-loop-helix. Proteins containing these motifs may act alone as monomers, or they may form homo- or heterodimers that interact with DNA.

The helix-turn-helix motif consists of two α helices connected at a fixed angle by a short chain of amino acids. One of the helices binds to the major groove. Helix-turn-helix motifs are exemplified by the homeobox motif which is present in homeodomain proteins. These proteins are critical for specifying the anterior-posterior body axis during development and are conserved throughout the animal kingdom. The Antennapedia and Ultrabithorax proteins of Drosophila melanogaster are prototypical homeodomain proteins. (Pabo, C. O. and R. T. Sauer (1992) Annu. Rev. Biochem. 61:1053-1095.)

Homeobox genes are a family of highly conserved regulatory genes that encode transcription factors. They are essential during embryonic development. They are important in limb formation and reproductive tract development. They function in uterine receptivity and implantation in mice and probably serve a similar role in humans (Daftary, G. S. and H. S. Taylor (2000) Semin. Reprod. Med. 18:311-320). Homeobox gene mutations play a role in susceptibility to autism (Ingram, J. L. et al. (2000) Teratology 62:393-405) and are implicated in human diseases, such as diabetes to cancer (Cillo, C. et al. (2001) J. Cell Physiol. 188:161-169).

The helix-loop-helix motif (HLH) consists of a short a helix connected by a loop to a longer a helix. The loop is flexible and allows the two helices to fold back against each other and to bind to DNA. The protooncogene Myc, a transcription factor that activates genes required for cellular proliferation, contains a prototypical HLH motif.

A zinc finger is a cysteine-rich, compactly folded protein motif in which specifically placed cysteines, and in some cases histidines, coordinate Zn⁺². Several types of zinc finger motifs have been identified. Though originally identified in DNA-binding proteins as regions that interact directly with DNA, zinc fingers occur in a variety of proteins that do not bind DNA (Lodish, H. et al. (1995) Molecular Cell Biology, Scientific American Books, New York, N.Y., pp. 447-451). For example, Galcheva-Gargova, Z. et al. ((1996) Science 272:1797-1802) have identified zinc finger proteins that interact with various cytokine receptors.

The zinc finger motif, which binds zinc ions, generally contains tandem repeats of about 30 amino acids consisting of periodically spaced cysteine and histidine residues. Examples of this sequence pattern, designated C2H2 and C3HC4 (“RING” finger), have been described (Lewin, supra). Zinc finger proteins each contain an α helix and an antiparallel β sheet whose proximity and conformation are maintained by the zinc ion. Contact with DNA is made by the arginine preceding the α helix and by the second, third, and sixth residues of the α helix. Variants of the zinc finger motif include poorly defined cysteine-rich motifs which bind zinc or other metal ions. These motifs may not contain histidine residues and are generally nonrepetitive. The zinc finger motif may be repeated in a tandem array within a protein, such that the a helix of each zinc finger in the protein makes contact with the major groove of the DNA double helix. This repeated contact between the protein and the DNA produces a strong and specific DNA-protein interaction. The strength and specificity of the interaction can be regulated by the number of zinc finger motifs within the protein. Though originally identified in DNA-binding proteins as regions that interact directly with DNA, zinc fingers occur in a variety of proteins that do not bind DNA (Lodish, H. et al. (1995) Molecular Cell Biology, Scientific American Books, New York N.Y., pp. 447-451). For example, Galcheva-Gargova, Z. et al. (1996; Science 272:1797-1802) have identified zinc finger proteins that interact with various cytokine receptors.

The C2H2-type zinc finger signature motif contains a 28 amino acid sequence, including 2 conserved Cys and 2 conserved His residues in a C-2-C-12-H-3-H type motif. The motif generally occurs in multiple tandem repeats. A cysteine-rich domain including the motif Asp-His-His-Cys (DHHC-CRD) has been identified as a distinct subgroup of zinc finger proteins. The DHHC-CRD region has been implicated in growth and development. One DHHC CRD mutant shows defective function of Ras, a small membrane-associated GTP-binding protein that regulates cell growth and differentiation, while other DHHC-CRD proteins probably function in pathways not involving Ras (Bartels, D. J. et al. (1999) Mol. Cell Biol. 19:6775-6787).

Zinc-finger transcription factors are often accompanied by modular sequence motifs such as the Kruppel-associated box (KRAB) and the SCAN domain. For example, the hypoalphalipoproteinemia susceptibility gene ZNF202 encodes a SCAN box and a KRAB domain followed by eight C2H zinc-finger motifs (Honer, C. et al. (2001) Biochim. Biophys. Acta 1517:441-448). The SCAN domain is a highly conserved, leucine-rich motif of approximately 60 amino acids found at the amino-terminal end of zinc finger transcription factors. SCAN domains are most often linked to C2H2 zinc finger motifs through their carboxyl-terminal end. Biochemical binding studies have established the SCAN domain as a selective hetero- and homotypic oligomerization domain. SCAN domain-mediated protein complexes may function to modulate the biological function of transcription factors (Schumacher, C. et al. (2000) J. Biol. Chem. 275:17173-17179).

The KRAB (Kruppel-associated box) domain is a conserved amino acid sequence spanning approximately 75 amino acids and is found in almost one-third of the 300 to 700 genes encoding C2H2 zinc fingers. The KRAB domain is found N-terminally with respect to the finger repeats. The KRAB domain is generally encoded by two exons; the KRAB-A region or box is encoded by one exon and the KRAB-B region or box is encoded by a second exon. The function of the KRAB domain is the repression of transcription. Transcription repression is accomplished by recruitment of either the KRAB-associated protein-1, a transcriptional corepressor, or the KRAB-A interacting protein. Proteins containing the KRAB domain are likely to play a regulatory role during development (Williams, A. J. et al. (1999) Mol. Cell Biol. 19:8526-8535). A subgroup of highly related human KRAB zinc finger proteins detectable in all human tissues is highly expressed in human T lymphoid cells (Bellefroid, E. J. et al. (1993) EMBO J. 12:1363-1374). The 2NF85 KRAB zinc finger gene, a member of the human ZNF91 family, is highly expressed in normal adult testis, in seminomas, and in the NT2/D1 teratocarcinoma cell line (Poncelet, D. A. et al. (1998) DNA Cell Biol. 17:931-943).

The C4 motif is found in hormone-regulated proteins. The C4 motif generally includes only 2 repeats. A number of eukaryotic and viral proteins contain a conserved cysteine-rich domain of 40 to 60 residues (called C3HC4 zinc-finger or RING finger) that binds two atoms of zinc, and is probably involved in mediating protein-protein interactions. The 3D “cross-brace” structure of the zinc ligation system is unique to the RING domain. The spacing of the cysteines in such a domain is C-x(2)-C-x(9 to 39)C-x(1 to 3)-H-x(2 to 3)-C-x(2)-C-x(4 to 48)-C-x(2)-C. The PHD finger is a C4HC3 zinc-finger-like motif found in nuclear proteins thought to be involved in chromatin-mediated transcriptional regulation.

GATA-type transcription factors contain one or two zinc finger domains which bind specifically to a region of DNA that contains the consecutive nucleotide sequence GATA. NMR studies indicate that the zinc finger comprises two irregular anti-parallel β sheets and an α helix, followed by a long loop to the C-terminal end of the finger (Ominchinski, J. G. (1993) Science 261:438-446). The helix and the loop connecting the two β-sheets contact the major groove of the DNA, while the C-terminal part, which determines the specificity of binding, wraps around into the minor groove.

The LIM motif consists of about 60 amino acid residues and contains seven conserved cysteine residues and a histidine within a consensus sequence (Schmeichel, K. L. and M. C. Beckerle (1994) Cell 79:211-219). The LIM family includes transcription factors and cytoskeletal proteins which may be involved in development, differentiation, and cell growth. One example is actin-binding LIM protein, which may play roles in regulation of the cytoskeleton and cellular morphogenesis (Roof, D. J. et al. (1997) J. Cell Biol. 138:575-588). The N-terminal domain of actin-binding LIM protein has four double zinc finger motifs with the LIM consensus sequence. The C-terminal domain of actin-binding LIM protein shows sequence similarity to known actin-binding proteins such as dematin and villin. Actin-binding LIM protein binds to F-actin through its dematin-like C-terminal domain. The LIM domain may mediate protein-protein interactions with other LIM-binding proteins.

Myeloid cell development is controlled by tissue-specific transcription factors. Myeloid zinc finger proteins (MZF) include MZF-1 and MZF-2. MZF-1 functions in regulation of the development of neutrophilic granulocytes. A murine homolog MZF-2 is expressed in myeloid cells, particularly in the cells committed to the neutrophilic lineage. MZF-2 is down-regulated by G-CSF and appears to have a unique function in neutrophil development (Murai, K. et al. (1997) Genes Cells 2:581-591).

The leucine zipper motif comprises a stretch of amino acids rich in leucine which can form an amphipathic α helix. This structure provides the basis for dimerization of two leucine zipper proteins. The region adjacent to the leucine zipper is usually basic, and upon protein dimerization, is optimally positioned for binding to the major groove. Proteins containing such motifs are generally referred to as bZIP transcription factors. The leucine zipper motif is found in the proto-oncogenes Fos and Jun, which comprise the heterodimeric transcription factor AP1 involved in cell growth and the determination of cell lineage (Papavassiliou, A. G. (1995) N. Engl. J. Med. 332:45-47).

The NF-kappa-B/Rel signature defines a family of eukaryotic transcription factors involved in oncogenesis, embryonic development, differentiation and immune response. Most transcription factors containing the Rel homology domain (RHD) bind as dimers to a consensus DNA sequence motif termed kappa-B. Members of the Rel family share a highly conserved 300 amino acid domain termed the Rel homology domain. The characteristic Rel C-terminal domain is involved in gene activation and cytoplasmic anchoring functions. Proteins known to contain the RHD domain include vertebrate nuclear factor NF-kappa-B, which is a heterodimer of a DNA-binding subunit and the transcription factor p65, mammalian transcription factor RelB, and vertebrate proto-oncogene c-rel, a protein associated with differentiation and lymphopoiesis (Kabrun, N. and P. J. Enrietto (1994) Semin. Cancer Biol. 5:103-112).

A DNA binding motif termed ARID (AT-rich interactive domain) distinguishes an evolutionarily conserved family of proteins. The approximately 100-residue ARID sequence is present in a series of proteins strongly implicated in the regulation of cell growth, development, and tissue-specific gene expression. ARID proteins include Bright (a regulator of B-cell-specific gene expression), dead ringer (involved in development), and MRF-2 (which represses expression from the cytomegaloviris enhancer) (Dallas, P. B. et al. (2000) Mol. Cell Biol. 20:3137-3146).

The ELM2 (Eg1-27 and MTA1 homology 2) domain is found in metastasis-associated protein MTA1 and protein ER1. The Caenorhabditis elegans gene eg1-27 is required for embryonic patterning MTA1, a human gene with elevated expression in metastatic carcinomas, is a component of a protein complex with histone deacetylase and nucleosome remodelling activities (Solari, F. et al. (1999) Development 126:2483-2494). The ELM2 domain is usually found to the N terminus of a myb-like DNA binding domain. ELM2 is also found associated with an ARID DNA.

The Iroquois (Irx) family of genes are found in nematodes, insects and vertebrates. Irx genes usually occur in one or two genomic clusters of three genes each and encode transcriptional controllers that possess a characteristic homeodomain. The Irx genes function early in development to specify the identity of diverse territories of the body. Later in development in both Drosophila and vertebrates, the Irx genes function again to subdivide those territories into smaller domains. (For a review of Iroquois genes, see Cavodeassi, F. et al. (2001) Development 128:2847-2855.) For example, mouse and human Irx4 proteins are 83% conserved and their 63-aa homeodomain is more than 93% identical to that of the Drosophila Iroquois patterning genes. Irx4 transcripts are predominantly expressed in the cardiac ventricles. The homeobox gene Irx4 mediates ventricular differentiation during cardiac development (Bruneau, B. G. et al. (2000) Dev. Biol. 217:266-77).

Histidine triad (HIT) proteins share residues in distinctive dimeric, 10-stranded half-barrel structures that form two identical purine nucleotide-binding sites. Hint (histidine triad nucleotide-binding protein)-related proteins, found in all forms of life, and fragile histidine triad (Fhit)-related proteins, found in animals and fungi, represent the two main branches of the HIT superfamily. Fhit homologs bind and cleave diadenosine polyphosphates. Fhit-Ap(n)A complexes appear to function in a proapoptotic tumor suppression pathway in epithelial tissues (Brenner C. et al. (1999) J. Cell Physiol.181:179-187).

Most transcription factors contain characteristic DNA binding motifs, and variations on the above motifs and new motifs have been and are currently being characterized (Faisst, S. and S. Meyer (1992) Nucleic Acids Res. 20:3-26). These include the forkhead motif, found in transcription factors involved in development and oncogenesis (Hacker, U. et al. (1995) EMBO J 14:5306-5317), and the T-box protein T-domain, which forms a novel major and minor groove DNA contact T-box genes such as Brachyury (T) are essential for tissue specification in development (Muller, C. W. and B. G. Herrmann (1997) Nature 389:884-888). Mga is a novel protein which interacts with Max, a small bHLHZip protein required by Myc, Mad, and Mnt proteins to function as transcription factors. Max is required of these proteins for specific DNA binding to E-box sequences. Mga, like Myc, contains the basic-helix-loop-helix-leucine zipper motif (bHLHZip) and requires heterodimerization with Max for binding to the preferred Myc-Max-binding site CACGTG, but otherwise shows no relationship with Myc, Mad, or Mnt proteins. Mga also contains a DNA-binding domain called a T-box or T-domain. The T-domain, a highly conserved DNA-binding motif originally defined in the gastrulation-associated gene, Brachyury, is characteristic of the Tbx family of transcription factors. Mga binds the preferred Brachyury-binding sequence and represses transcription of reporter genes containing promoter-proximal Brachyury-binding sites. Mga is converted to a transcription activator of both Myc-Max and Brachyury site-containing reporters in a Max-dependent manner. Mga apparently functions as a dual-specificity transcription factor that regulates the expression of both Max-network and T-box family target genes (Hurlin, P. J. et al. (1999) EMBO J. 18:7019-7028).

PGC-1 stands for thermogenic peroxisome proliferator-activated receptor gamma (PPAR-gamma) coactivator 1. It activates mitochondrial biogenesis in part through a direct interaction with nuclear respiratory factor 1 (NRF-1). A functional relative, PRC (PGC-1-related coactivator) is ubiquitously expressed in murine and human tissues and cell lines; but unlike PGC-1, PRC is not dramatically up-regulated during thermogenesis in brown fat. Its expression is down-regulated in quiescent BALB/3T3 cells and is rapidly induced by reintroduction of serum, conditions where PGC-1 is not detected. Similar to PGC-1, PRC activates NRF-1-dependent promoters. PRC interacts in vitro with the NRF-1 DNA binding domain through two distinct recognition motifs that are separated by an unstructured proline-rich region. PRC also contains a potent transcriptional activation domain in its amino terminus adjacent to an LXXLL motif. The spatial arrangement of these functional domains coincides with those found in PGC-1 (Andersson, U. and Scarpulla, R. C. (2001) Mol. Cell. Biol. 21:3738-3749).

Chromatin Associated Proteins

In the nucleus, DNA is packaged into chromatin, the compact organization of which limits the accessibility of DNA to transcription factors and plays a key role in gene regulation (Lewin, supra, pp. 409-410). The compact structure of chromatin is determined and influenced by chromatin-associated proteins such as the histones, the high mobility group (HMG) proteins, and the chromodomain proteins. There are five classes of histones, H1, H2A, H2B, H3, and H4, all of which are highly basic, low molecular weight proteins. The fundamental unit of chromatin, the nucleosome, consists of 200 base pairs of DNA associated with two copies each of H2A, H2B, H3, and H4. H1 links adjacent nucleosomes. HMG proteins are low molecular weight, non-histone proteins that may play a role in unwinding DNA and stabilizing single-stranded DNA. Chromodomain proteins play a key role in the formation of highly compacted heterochromatin, which is transcriptionally silent. Protamines are small, highly basic proteins that substitute for histones in sperm chromatin during the haploid phase of spermatogenesis. They pack sperm DNA into a highly condensed, stable, and inactive complex (Prosite PDOC00047 Protamine P1 signature).

Higher-order structures of chromosomes involve the interaction of histones and chromosomal DNA with a series of nonhistone proteins. For example, HIRA is a histone binding protein that is a major candidate for causing developmental disorders associated with deletions in chromosome 22, including DiGeorge syndrome and velocardiofacial syndrome. HIRA interacts with core histones as well as the HIRA interacting protein HIRIP3 to form a complex that may have a role in regulating chromatin structure during development (Lorain, S. et al. (1998) Mol. Cell. Biol. 18:5546-5556).

Diseases and Disorders Related to Gene Regulation

Mutations in transcription factors contribute to oncogenesis. This is likely due to the role of transcription factors in the expression of genes involved in cell proliferation. For example, mutations in transcription factors encoded by proto-oncogenes, such as Fos, Jun, Myc, Rel, and Spi1, may be oncogenic due to increased stimulation of cell proliferation. Conversely, mutations in transcription factors encoded by tumor suppressor genes, such as p53, RB1, and WT1, may be oncogenic due to decreased inhibition of cell proliferation (Latchman, D. (1995) Gene Regulation: A Eukaryotic Perspective, Chapman and Hall, London, UK, pp. 242-255).

Many neoplastic disorders in humans can be attributed to inappropriate gene expression. Malignant cell growth may result from either excessive expression of tumor promoting genes or insufficient expression of tumor suppressor genes (Cleary, M. L. (1992) Cancer Surv. 15:89-104). The zinc finger-type transcriptional regulator WT1 is a tumor-suppressor protein that is inactivated in children with Wilm's tumor. Deletions of the WT1 gene, or point mutations which destroy the DNA-binding activity of the protein, are associated with development of the pediatric nephroblastoma, Wilms tumor, and Denys-Drash syndrome (Rauscher, F. J. (1993) FASEB J. 7:896-903). The oncogene bcl-6, which plays an important role in large-cell lymphoma, is also a zinc-finger protein (Papavassiliou, A. G. (1995) N. Engl. J. Med. 332:45-47).

Chromosomal translocations may also produce chimeric loci that fuse the coding sequence of one gene with the regulatory regions of a second unrelated gene. Such an arrangement likely results in inappropriate gene transcription, potentially contributing to malignancy. In Burkitt's lymphoma, for example, the transcription factor Myc is translocated to the immunoglobulin heavy chain locus, greatly enhancing Myc expression and resulting in rapid cell growth leading to leukemia (Latchman, D. S. (1996) N. Engl. J. Med. 334:28-33). Human acute leukemias involve reciprocal chromosome translocations that fuse the ALL-1 gene located at chromosome region 11q23 to a series of partner genes positioned on a variety of human chromosomes. The fused genes encode chimeric proteins. The AF17 gene encodes a protein of 1093 amino acids, containing a leucine-zipper dimerization motif located 3′ of the fusion point and a cysteine-rich domain at the N terminus that shows homology to a domain within the protein Br140 (peregrin) (Prasad R. et al. (1994) Proc. Natl. Acad. Sci. USA 91:8107-8111).

Certain proteins enriched in glutamine are associated with various neurological disorders including spinocerebellar ataxia, bipolar effective disorder, schizophrenia, and autism (Margolis, R. L. et al. (1997) Human Genetics 100:114-122). These proteins contain regions with as many as 15 or more consecutive glutamine residues and may function as transcription factors with a potential role in regulation of neurodevelopment or neuroplasticity.

Impaired transcriptional regulation may lead to Alzheimer's disease, a progressive neurodegenerative disorder that is characterized by the formation of senile plaques and neurofibrillary tangles containing amyloid beta peptide. These plaques are found in limbic and association cortices of the brain, including hippocampus, temporal cortices, cingulate cortex, amygdala, nucleus basalis and locus caeruleus. Early in Alzheimer's pathology, physiological changes are visible in the cingulate cortex (Minoshima, S. et al. (1997) Ann. Neurol. 42:85-94). In subjects with advanced Alzheimer's disease, accumulating plaques damage the neuronal architecture in limbic areas and eventually cripple the memory process.

In addition, the immune system responds to infection or trauma by activating a cascade of events that coordinate the progressive selection, amplification, and mobilization of cellular defense mechanisms. A complex and balanced program of gene activation and repression is involved in this process. However, hyperactivity of the immune system as a result of improper or insufficient regulation of gene expression may result in considerable tissue or organ damage. This damage is well-documented in immunological responses associated with arthritis, allergens, heart attack, stroke, and infections. (Isselbacher, K. J. et al. Harrison's Principles of Internal Medicine, 13/e, McGraw Hill, Inc. and Teton Data Systems Software, 1996.) In particular, a zinc finger protein termed Staf50 (for Stimulated trans-acting factor of 50 kDa) is a transcriptional regulator and is induced in various cell lines by interferon-I and -II. Staf50 appears to mediate the antiviral activity of interferon by down-regulating the viral transcription directed by the long terminal repeat promoter region of human immunodeficiency virus type-1 in transfected cells (Tissot, C. (1995) J. Biol. Chem. 270:14891-14898). Also, the causative gene for autoimmune polyendocrinopathy-candidiasis-ectodermal dystrophy (APECED) was recently isolated and found to encode a protein with two PHD-type zinc finger motifs (Bjorses, P. et al. (1998) Hum. Mol. Genet. 7:1547-1553).

Furthermore, the generation of multicellular organisms is based upon the induction and coordination of cell differentiation at the appropriate stages of development. Central to this process is differential gene expression, which confers the distinct identities of cells and tissues throughout the body. Failure to regulate gene expression during development could result in developmental disorders. Human developmental disorders caused by mutations in zinc finger-type transcriptional regulators include: urogenital developmental abnormalities associated with WT1; Greig cephalopolysyndactyly, Pallister-Hall syndrome, and postaxial polydactyly type A (GLI3), and Townes-Brocks syndrome, characterized by anal, renal, limb, and ear abnormalities (SALL1) (Engelkamp, D. and V. van Heyningen (1996) Curr. Opin. Genet. Dev. 6:334-342; Kohlhase, J. et al. (1999) Am. J. Hum. Genet. 64:435-445).

Synthesis of Nucleic Acids

Polymerases

DNA and RNA replication are critical processes for cell replication and function. DNA and RNA replication are mediated by the enzymes DNA and RNA polymerase, respectively, by a “templating” process in which the nucleotide sequence of a DNA or RNA strand is copied by complementary base-pairing into a complementary nucleic acid sequence of either DNA or RNA. However, there are fundamental differences between the two processes.

DNA polymerase catalyzes the stepwise addition of a deoxyribonucleotide to the 3′-OH end of a polynucleotide strand (the primer strand) that is paired to a second (template) strand. The new DNA strand therefore grows in the 5′ to 3′ direction (Alberts, et al.,supra, pp. 251-254). The substrates for the polymerization reaction are the corresponding deoxynucleotide triphosphates which must base-pair with the correct nucleotide on the template strand in order to be recognized by the polymerase. Because DNA exists as a double-stranded helix, each of the two strands may serve as a template for the formation of a new complementary strand. Each of the two daughter cells of a dividing cell therefore inherits a new DNA double helix containing one old and one new strand. Thus, DNA is said to be replicated “semiconservatively” by DNA polymerase. In addition to the synthesis of new DNA, DNA polymerase is also involved in the repair of damaged DNA as discussed below under “Ligases.”

In contrast to DNA polymerase, RNA polymerase uses a DNA template strand to “transcribe” DNA into RNA using ribonucleotide triphosphates as substrates. Like DNA polymerization, RNA polymerization proceeds in a 5′ to 3′ direction by addition of a ribonucleoside monophosphate to the 3′-OH end of a growing RNA chain. DNA transcription generates messenger RNAs (mRNA) that carry information for protein synthesis, as well as the transfer, ribosomal, and other RNAs that have structural or catalytic functions. In eukaryotes, three discrete RNA polymerases synthesize the three different types of RNA (Alberts et al., supra, pp. 367-368). RNA polymerase I makes the large ribosomal RNAs, RNA polymerase II makes the mRNAs that will be translated into proteins, and RNA polymerase III makes a variety of small, stable RNAs, including 5S ribosomal RNA and the transfer RNAs (tRNA). In all cases, RNA synthesis is initiated by binding of the RNA polymerase to a promoter region on the DNA and synthesis begins at a start site within the promoter. Synthesis is completed at a stop (termination) signal in the DNA whereupon both the polymerase and the completed RNA chain are released.

Ligases

DNA repair is the process by which accidental base changes, such as those produced by oxidative damage, hydrolytic attack, or uncontrolled methylation of DNA, are corrected before replication or transcription of the DNA can occur. Because of the efficiency of the DNA repair process, fewer than one in a thousand accidental base changes causes a mutation (Alberts et al., supra, pp. 245-249). The three steps common to most types of DNA repair are (1) excision of the damaged or altered base or nucleotide by DNA nucleases, (2) insertion of the correct nucleotide in the gap left by the excised nucleotide by DNA polymerase using the complementary strand as the template and, (3) sealing the break left between the inserted nucleotide(s) and the existing DNA strand by DNA ligase. In the last reaction, DNA ligase uses the energy from ATP hydrolysis to activate the 5′ end of the broken phosphodiester bond before forming the new bond with the 3′-OH of the DNA strand. In Bloom's syndrome, an inherited human disease, individuals are partially deficient in DNA ligation and consequently have an increased incidence of cancer (Alberts et al., supra, p. 247).

Nucleases

Nucleases comprise enzymes that hydrolyze both DNA (DNase) and RNA (Rnase). They serve different purposes in nucleic acid metabolism. Nucleases hydrolyze the phosphodiester bonds between adjacent nucleotides either at internal positions (endonucleases) or at the terminal 3′ or 5′ nucleotide positions (exonucleases). A DNA exonuclease activity in DNA polymerase, for example, serves to remove improperly paired nucleotides attached to the 3′-OH end of the growing DNA strand by the polymerase and thereby serves a “proofreading” function. As mentioned above, DNA endonuclease activity is involved in the excision step of the DNA repair process.

RNases also serve a variety of functions. For example, RNase P is a ribonucleoprotein enzyme which cleaves the 5′ end of pre-tRNAs as part of their maturation process. RNase H digests the RNA strand of an RNA/DNA hybrid. Such hybrids occur in cells invaded by retroviruses, and RNase H is an important enzyme in the retroviral replication cycle. Pancreatic RNase secreted by the pancreas into the intestine hydrolyzes RNA present in ingested foods. RNase activity in serum and cell extracts is elevated in a variety of cancers and infectious diseases (Schein, C. H. (1997) Nat. Biotechnol. 15:529-536). Regulation of RNase activity is being investigated as a means to control tumor angiogenesis, allergic reactions, viral infection and replication, and fungal infections.

Modification of Nucleic Acids

DNA Repair

Cells are constantly faced with replication errors and environmental assault (such as ultraviolet irradiation) that can produce DNA damage. Damage to DNA consists of any change that modifies the structure of the molecule. Changes to DNA can be divided into two general classes, single base changes and structural distortions. Single base changes affect the sequence but not the overall structure of the DNA. Since single base changes do not affect transcription or replication, they exert their effect on future generations. Structural distortions affect the structure of the DNA. A single strand nick or removal of a base may prevent a strand from acting as a viable template for synthesis of DNA or RNA. Intrastrand or interstrand covalent linkage between bases, or the addition of a bulky adduct to a base, may distort the structure of the double helix and interfere with transcription and replication. Any damage to DNA can produce a mutation, and the mutation may produce a disorder, such as cancer.

Changes in DNA are recognized by repair systems within the cell. These repair systems act to correct the damage and thus prevent any deleterious affects of a mutational event. Repair systems can be divided into three general types, direct repair, excision repair, and retrieval systems. When the repair systems are eliminated, cells become exceedingly sensitive to environmental mutagens, such as ultraviolet irradiation. Disorders associated with a loss in DNA repair systems often exhibit a high sensitivity to environmental mutagens. Examples of such disorders include xeroderma pigmentosum, Bloom's syndrome, and Werner's syndrome. Xeroderma pigmentosum results in a hypersensitivity to sunlight, especially ultraviolet, and produces skin defects. Bloom's syndrome results in an increased frequency of chromosomal aberrations, including sister chromosome exchanges (Yamagata, K. et al. (1998) Proc. Natl. Acad. Sci. USA 95:8733-8738).

Direct repair involves the reversal or simple removal of the damaged region of DNA. Mismatches involving normal bases are repaired based on certain biases within the repair system. For example, mismatched GT base pairs are frequently caused by deamination of 5-methyl-cytosine to form thymine. Therefore, repair systems convert mismatched GT pairs to GC, instead of AT. Repair also favors the non-methylated strand in hemimethylated DNA, since this strand represents the newly synthesized daughter strand. The recognition of hemimethylated DNA and repair of mismatches on the non-methylated strand involve the products of the genes mutH, mutL, mutS (which specifically recognizes mismatched base pairs), the helicase encoded by the uvrD gene, and the methylase encoded by the dam gene. C-5 cytosine-specific DNA methylases are enzymes that specifically methylate the C-5 carbon of cytosines in DNA (Kumar, S. et al. (1994) Nucleic Acids Res. 22:1-10).

Excision repair is a system in which mispaired or damaged bases are removed from DNA and a new stretch of DNA is synthesized to replace them In the incision step, the damaged structure is recognized by an endonuclease that cleaves the DNA strand on both sides of the damage. In the excision step, a 5′-3′exonuclease removes a stretch of the damaged DNA strand. In the synthesis step, the resulting single-stranded region serves as a template for a DNA polymerase to synthesize a replacement for the excised sequence. Finally, DNA ligase covalently links the 3′ end of the new material to the old material. In mammals, DNA polymerase beta serves as the DNA repair polymerase. Mutations in the human DNA polymerase beta gene are associated with several types of cancer (Bhattacharyya, N. et al. (1999) DNA Cell Biol. 18:549-554; Matsuzaki, J. et al. (1996) Mol. Carcinog. 15:38-43).

Methylases

Methylation of specific nucleotides occurs in both DNA and RNA, and serves different functions in the two macromolecules. Methylation of cytosine residues to form 5-methyl cytosine in DNA occurs specifically in CG sequences which are base-paired with one another in the DNA double-helix. The pattern of methylation is passed from generation to generation during DNA replication by an enzyme called “maintenance methylase” that acts preferentially on those CG sequences that are base-paired with a CG sequence that is already methylated. Such methylation appears to distinguish active from inactive genes by preventing the binding of regulatory proteins that “turn on” the gene, but permiting the binding of proteins that inactivate the gene (Alberts et al., supra, pp. 448-451). N-6 adenine-specific methylases are enzymes that specifically methylate the amino group at the C-6 position of adenines in DNA. These enzymes are found in the three known types of bacterial restriction-modification, systems (Prosite PDOC00087 N-6 Adenine-specific DNA methylases signature). In RNA metabolism, “tRNA methylase” produces one of several nucleotide modifications in tRNA that affect the conformation and base-pairing of the molecule and facilitate the recognition of the appropriate mRNA codons by specific tRNAs. The primary methylation pattern is the dimethylation of guanine residues to form N,N-dimethyl guanine.

Helicases and Single-stranded Binding Proteins

Helicases are enzymes that destabilize and unwind double helix structures in both DNA and RNA. Since DNA replication occurs more or less simultaneously on both strands, the two strands must first separate to generate a replication “fork” for DNA polymerase to act on. Two types of replication proteins contribute to this process, DNA helicases and single-stranded binding proteins. DNA helicases hydrolyze ATP and use the energy of hydrolysis to separate the DNA strands. Single-stranded binding proteins (SSBs) then bind to the exposed DNA strands, without covering the bases, thereby temporarily stabilizing them for templating by the DNA polymerase (Alberts et al., supra, pp. 255-256).

RNA helicases also alter and regulate RNA conformation and secondary structure. Like the DNA helicases, RNA helicases utilize energy derived from ATP hydrolysis to destabilize and unwind RNA duplexes. The most well-characterized and ubiquitous family of RNA helicases is the DEAD-box family, so named for the conserved B-type ATP-binding motif which is diagnostic of proteins in this family. Over 40 DEAD-box helicases have been identified in organisms as diverse as bacteria, insects, yeast, amphibians, mammals, and plants. DEAD-box helicases function in diverse processes such as translation initiation, splicing, ribosome assembly, and RNA editing, transport, and stability. Examples of these RNA helicases include yeast Drs1 protein, which is involved in ribosomal RNA processing; yeast TIF1 and TIF2 and mammalian eIF-4A, which are essential to the initiation of RNA translation; and human p68 antigen, which regulates cell growth and division (Ripmaster, T. L. et al. (1992) Proc. Natl. Acad. Sci. USA 89:11131-11135; Chang, T.-H. et al. (1990) Proc. Natl. Acad. Sci. USA 87:1571-1575). These RNA helicases demonstrate strong sequence homology over a stretch of some 420 amino acids. Included among these conserved sequences are the consensus sequence for the A motif of an ATP binding protein; the “DEAD box” sequence, associated with ATPase activity; the sequence SAT, associated with the actual helicase unwinding region; and an octapeptide consensus sequence, required for RNA binding and ATP hydrolysis (Pause, A. et al. (1993) Mol. Cell Biol. 13:6789-6798). Differences outside of these conserved regions are believed to reflect differences in the functional roles of individual proteins (Chang et al., supra).

Some DEAD-box helicases play tissue- and stage-specific roles in spermatogenesis and embryogenesis. Overexpression of the DEAD-box 1 protein (DDX1) may play a role in the progression of neuroblastoma (Nb) and retinoblastoma (Rb) tumors (Godbout, R. et al. (1998) J. Biol. Chem. 273:21161-21168). These observations suggest that DDX1 may promote or enhance tumor progression by altering the normal secondary structure and expression levels of RNA in cancer cells. Other DEAD-box helicases have been implicated either directly or indirectly in tumorigenesis (Godbout et al., supra). For example, murine p68 is mutated in ultraviolet light-induced tumors, and human DDX6 is located at a chromosomal breakpoint associated with B-cell lymphoma. Similarly, a chimeric protein comprised of DDX10 and NUP98, a nucleoporin protein, may be involved in the pathogenesis of certain myeloid malignancies.

Topoisomerases

Besides the need to separate DNA strands prior to replication, the two strands must be “unwound” from one another prior to their separation by DNA helicases. This function is performed by proteins known as DNA topoisomerases. DNA topoisomerase effectively acts as a reversible nuclease that hydrolyzes a phosphodiesterase bond in a DNA strand, permits the two strands to rotate freely about one another to remove the strain of the helix, and then rejoins the original phosphodiester bond between the two strands. Topoisomerases are essential enzymes responsible for the topological rearrangement of DNA brought about by transcription, replication, chromatin formation, recombination, and chromosome segregation. Superhelical coils are introduced into DNA by the passage of processive enzymes such as RNA polymerase, or by the separation of DNA strands by a helicase prior to replication. Knotting and concatenation can occur in the process of DNA synthesis, storage, and repair. All topoisomerases work by breaking a phosphodiester bond in the ribose-phosphate backbone of DNA. A catalytic tyrosine residue on the enzyme makes a nucleophilic attack on the scissile phosphodiester bond, resulting in a reaction intermediate in which a covalent bond is formed between the enzyme and one end of the broken strand. A tyrosine-DNA phosphodiesterase functions in DNA repair by hydrolyzing this bond in occasional dead-end topoisomerase I-DNA intermediates (Pouliot, J. J. et al. (1999) Science 286:552-555).

Two types of DNA topoisomerase exist, types I and II. Type I topoisomerases work as monomers, making a break in a single strand of DNA while type II topoisomerases, working as homodimers, cleave both strands. DNA Topoisomerase I causes a single-strand break in a DNA helix to allow the rotation of the two strands of the helix about the remaining phosphodiester bond in the opposite strand. DNA topoisomerase II causes a transient break in both strands of a DNA helix where two double helices cross over one another. This type of topoisomerase can efficiently separate two interlocked DNA circles (Alberts et al., supra, pp. 260-262). Type II topoisomerases are largely confined to proliferating cells in eukaryotes, such as cancer cells. For this reason they are targets for anticancer drugs. Topoisomerase II has been implicated in multi-drug resistance (MDR) as it appears to aid in the repair of DNA damage inflicted by DNA binding agents such as doxorubicin and vincristine. The type II topoisomerases are specific targets of drug classes that comprise complex-stabilizing (epipodophyllotoxins, anthracyclines) and catalytic (merbarone, bisdioxopiperazines) inhibitors (Beck, W. T. et al. (1999) Drug Resist. Update 2:382-389). Topoisomerases include topo IIalpha-1 and topo IIbeta-1; topo IIalpha-2 and topo IIbeta-2, are novel variants that appear to be conserved between chicken and human. Topo IIalpha-2 encodes a protein with an additional 35 amino acids inserted after K321 of the chicken topo IIalpha-1 protein sequence. Topo IIbeta-2 encodes a protein missing 86 amino acids following V27 in the topo IIbeta-1 protein sequence. Alternatively spliced forms of human topo IIalpha are also observed (Petruti-Mot, A. S. and Earnshaw, W. C. (2000) Gene 258:183-192).

The topoisomerase I family includes topoisomerases I and III (topo I and topo III). The crystal structure of human topoisomerase I suggests that rotation about the intact DNA strand is partially controlled by the enzyme. In this “controlled rotation” model, protein-DNA interactions limit the rotation, which is driven by torsional strain in the DNA (Stewart, L. et al. (1998) Science 379:1534-1541). Structurally, topo I can be recognized by its catalytic tyrosine residue and a number of other conserved residues in the active site region. Topo I is thought to function during transcription. Two topo IIIs are known in humans, and they are homologous to prokaryotic topoisomerase I, with a conserved tyrosine and active site signature specific to this family. Topo III has been suggested to play a role in meiotic recombination. A mouse topo III is highly expressed in testis tissue and its expression increases with the increase in the number of cells in pachytene (Seki, T. et al. (1998) J. Biol. Chem. 273:28553-28556).

The topoisomerase II family includes two isozymes (IIα and IIβ) encoded by different genes. Topo II cleaves double stranded DNA in a reproducible, nonrandom fashion, preferentially in an AT rich region, but the basis of cleavage site selectivity is not known. Structurally, topo II is made up of four domains, the first two of which are structurally similar and probably distantly homologous to similar domains in eukaryotic topo I. The second domain bears the catalytic tyrosine, as well as a highly conserved pentapeptide. The IIα isoform appears to be responsible for unlinking DNA during chromosome segregation. Cell lines expressing IIα but not IIβ suggest that IIβ is dispensable in cellular processes; however, IIβ knockout mice died perinatally due to a failure in neural development. That the major abnormalities occurred in predominantly late developmental events (neurogenesis) suggests that IIβ is needed not at mitosis, but rather during DNA repair (Yang, X. et al. (2000) Science 287:131-134).

Topoisomerases have been implicated in a number of disease states, and topoisomerase poisons have proven to be effective anti-tumor drugs for some human malignancies. Topo I is mislocalized in Fanconi's anemia, and may be involved in the chromosomal breakage seen in this disorder (Wunder, E. (1984) Hum. Genet. 68:276-281). Overexpression of a truncated topo III in ataxia-telangiectasia (A-T) cells partially suppresses the A-T phenotype, probably through a dominant negative mechanism. This suggests that topo III is deregulated in A-T (Fritz, E. et al. (1997) Proc. Natl. Acad. Sci. USA 94:4538-4542). Topo III also interacts with the Bloom's Syndrome gene product, and has been suggested to have a role as a tumor suppressor (Wu, L. et al. (2000) J. Biol. Chem 275:9636-9644). Aberrant topo II activity is often associated with cancer or increased cancer risk Greatly lowered topo II activity has been found in some, but not all A-T cell lines (Mohamed, R. et al. (1987) Biochem. Biophys. Res. Commun. 149:233-238). On the other hand, topo II can break DNA in the region of the A-T gene (ATM), which controls all DNA damage-responsive cell cycle checkpoints (Kaufmann, W. K. (1998) Proc. Soc. Exp. Biol. Med. 217:327-334). The ability of topoisomerases to break DNA has been used as the basis of antitumor drugs. Topoisomerase poisons act by increasing the number of dead-end covalent DNA-enzyme complexes in the cell, ultimately triggering cell death pathways (Fortune, J. M. and N. Osheroff (2000) Prog. Nucleic Acid Res. Mol. Biol. 64:221-253; Guichard, S. M. and M. K. Danks (1999) Curr. Opin. Oncol. 11:482-489). Antibodies against topo I are found in the serum of systemic sclerosis patients, and the levels of the antibody may be used as a marker of pulmonary involvement in the disease (Diot, E. et al. (1999) Chest 116:715-720). Finally, the DNA binding region of human topo I has been used as a DNA delivery vehicle for gene therapy (Chen, T. Y. et al. (2000) Appl. Microbiol. Biotechnol. 53:558-567).

Recombinases

Genetic recombination is the process of rearranging DNA sequences within an organism's genome to provide genetic variation for the organism in response to changes in the environment. DNA recombination allows variation in the particular combination of genes present in an individual's genome, as well as the timing and level of expression of these genes (Alberts et al., supra, pp. 263-273). Two broad classes of genetic recombination are commonly recognized, general recombination and site-specific recombination. General recombination involves genetic exchange between any homologous pair of DNA sequences usually located on two copies of the same chromosome. The process is aided by enzymes, recombinases, that “nick” one strand of a DNA duplex more or less randomly and permit exchange with a complementary strand on another duplex. The process does not normally change the arrangement of genes in a chromosome. In site-specific recombination, the recombinase recognizes specific nucleotide sequences present in one or both of the recombining molecules. Base-pairing is not involved in this form of recombination and therefore it does not require DNA homology between the recombining molecules. Unlike general recombination, this form of recombination can alter the relative positions of nucleotide sequences in chromosomes.

RNA Metabolism

Much of the regulation of gene expression in eucaryotic cells occurs at the posttranscriptional level. Messenger RNAs (mRNA), which are produced in the cell nucleus from primary transcripts of protein-encoding genes, are processed and transported to the cytoplasm where the protein synthesis machinery is located. RNA-binding proteins are a group of proteins that participate in the processing, editing, transport, localization, and posttranscriptional regulation of mRNAs, and comprise the protein component of ribosomes as well. The RNA-binding activity of many of these proteins is mediated by a series of RNA-binding motifs identified within them. These domains include the RNP motif, the arginine-rich motif, the RGG box, and the KH motif (Burd, C. G. and G. Dreyfuss (1994) Science 265:615-621). The RNP motif is the most widely found and best characterized of these motifs. The RNP motif is composed of 90-100 amino acids which form an RNA-binding domain and is found in one or more copies in proteins that bind pre-mRNA, mRNA, pre-ribosomal RNA, and small nuclear RNAs. The RNP motif is composed of two short sequences (RNP-1 and RNP-2) and a number of other mostly hydrophobic, conserved amino acids interspersed throughout the motif (Burd and Dreyfuss, supra; ExPASy PROSITE document PDOC0030).

Ribonucleic acid (RNA) is a linear single-stranded polymer of four nucleotides, ATP, CTP, UTP, and GTP. In most organisms, RNA is transcribed as a copy of deoxyribonucleic acid (DNA), the genetic material of the organism. In retroviruses RNA rather than DNA serves as the genetic material. RNA copies of the genetic material encode proteins or serve various structural, catalytic, or regulatory roles in organisms. RNA is classified according to its cellular localization and function. Messenger RNAs (mRNAs) encode polypeptides. Ribosomal RNAs (rRNAs) are assembled, along with ribosomal proteins, into ribosomes, which are cytoplasmic particles that translate mRNA into polypeptides. Transfer RNAs (tRNAs) are cytosolic adaptor molecules that function in mRNA translation by recognizing both an mRNA codon and the amino acid that matches that codon. Heterogeneous nuclear RNAs (hnRNAs) include mRNA precursors and other nuclear RNAs of various sizes. Small nuclear RNAs (snRNAs) are a part of the nuclear spliceosome complex that removes intervening, non-coding sequences (introns) and rejoins exons in pre-mRNAs.

Proteins are associated with RNA during its transcription from DNA, RNA processing, and translation of mRNA into protein. Proteins are also associated with RNA as it is used for structural, catalytic, and regulatory purposes.

Transcription

Transcription in eukaryotes is catalyzed by three species of RNA polymerase: RNA polymerase I for rRNA synthesis, RNA polymerase II for mRNA synthesis and RNA polymerase III for tRNA and 5S rRNA synthesis. Each RNA polymerase is composed of more than 10 different polypeptides. The RNA polymerase III enzymes are the most complex of the nuclear polymerases. They contain the largest number of subunits; their basal transcription machinery includes the core transcription factors (TF) IIIA, IIIB and IIIC; and they have promoters that are mostly located within transcribed DNA (Akira Ishihama et al. (1998) Curr. Opin. Microbiol. 1:190-196). cDNA and genomic clones have been isolated for the second-largest subunit of RNA polymerase III in Drosophila melanogaster. The deduced polypeptide, named DmRP128, consists of 1135 amino acids with a calculated molecular weight of 128 kDa. The protein sequence shares conserved regions of homology with other cloned the second-largest subunits of RNA polymerases (Seifarth, W. et al. (1991) Mol. Gen. Genet. 228:424-432).

RNA Processing

Ribosomal RNAs (rRNAs) are assembled, along with ribosomal proteins, into ribosomes, which are cytoplasmic particles that translate messenger RNA (mRNA) into polypeptides. The eukaryotic ribosome is composed of a 60S (large) subunit and a 40S (small) subunit, which together form the 80S ribosome. In addition to the 18S, 28S, 5S, and 5.8S rRNAs, ribosomes contain from 50 to over 80 different ribosomal proteins, depending on the organism. Ribosomal proteins are classified according to which subunit they belong (i.e., L, if associated with the large 60S large subunit or S if associated with the small 40S subunit). E. coli ribosomes have been the most thoroughly studied and contain 50 proteins, many of which are conserved in all life forms. The structures of nine ribosomal proteins have been solved to less than 3.0D resolution (i.e., S5, S6, S17, L1, L6, L9, L12, L14, L30), revealing common motifs, such as b-a-b protein folds in addition to acidic and basic RNA-binding motifs positioned between b-strands. Most ribosomal proteins are believed to contact rRNA directly (reviewed in Liljas, A. and M. Garber (1995) Curr. Opin. Struct. Biol. 5:721-727; see also Woodson, S. A. and N. B. Leontis (1998) Curr. Opin. Struct. Biol. 8:294-300; Ramakrishnan, V. and S. W. White (1998) Trends Biochem. Sci. 23:208-212).

Ribosomal proteins may undergo post-translational modifications or interact with other ribosome-associated proteins to regulate translation. For example, the highly homologous 40S ribosomal protein S6 kinases (S6K1 and S6K2) play a key role in the regulation of cell growth by controlling the biosynthesis of translational components which make up the protein synthetic apparatus (including the ribosomal proteins). In the case of S6K1, at least eight phosphorylation sites are believed to mediate kinase activation in a hierarchical fashion (Dufner, A and G. Thomas (1999) Exp. Cell. Res. 253:100-109). Some of the ribosomal proteins, including L1, also function as translational repressors by binding to polycistronic mRNAs encoding ribosomal proteins (Liljas, supra and Garber, supra).

Recent evidence suggests that a number of ribosomal proteins have secondary functions independent of their involvement in protein biosynthesis. These proteins functions as regulators of cell proliferation and, in some instances, as inducers of cell death. For example, the expression of human ribosomal protein L13a has been shown to induce apoptosis by arresting cell growth in the G2/M phase of the cell cycle. Inhibition of expression of L13a induces apoptosis in target cells, which suggests that this protein is necessary, in the appropriate amount, for cell survival. Similar results have been obtained in yeast where inactivation of yeast homologues of L13a, rp22 and rp23, results in severe growth retardation and death. A closely related ribosomal protein, L7, arrests cells in G1 and also induces apoptosis. Thus, it appears that a subset of ribosomal proteins may function as cell cycle checkpoints and compose a new family of cell proliferation regulators.

Mapping of individual ribosomal proteins on the surface of intact ribosomes is accomplished using 3D immunocryoelectronmicroscopy, whereby antibodies raised against specific ribosomal proteins are visualized. Progress has been made toward the mapping of L1, L7, and L12 while the structure of the intact ribosome has been solved to only 20-25D resolution and inconsistencies exist among different crude structures (Frank, J. (1997) Curr. Opin. Struct. Biol. 7:266-272).

Three distinct sites have been identified on the ribosome. The aminoacyl-tRNA acceptor site (A site) receives charged tRNAs (with the exception of the initiator-tRNA). The peptidyl-tRNA site (P site) binds the nascent polypeptide as the amino acid from the A site is added to the elongating chain. Deacylated tRNAs bind in the exit site (E site) prior to their release from the ribosome. (The structure of the ribosome is reviewed in Stryer, L. (1995) Biochemistry, W. H. Freeman and Company, New York N.Y., pp. 888-908; Lodish, supra, pp. 119-138; and Lewin, B. (1997) Genes VI, Oxford University Press, Inc. New York N.Y.).

Various proteins are necessary for processing of transcribed RNAs in the nucleus. Pre-mRNA processing steps include capping at the 5′ end with methylguanosine, polyadenylating the 3′ end, and splicing to remove introns. The primary RNA transript from DNA is a faithful copy of the gene containing both exon and intron sequences, and the latter sequences must be cut out of the RNA transcript to produce a mRNA that codes for a protein. This “splicing” of the mRNA sequence takes place in the nucleus with the aid of a large, multicomponent ribonucleoprotein complex known as a spliceosome. The spliceosomal complex is comprised of five small nuclear ribonucleoprotein particles (snRNPs) designated U1, U2, U4, U5, and U6. Each snRNP contains a single species of snRNA and about ten proteins. The RNA components of some snRNPs recognize and base-pair with intron consensus sequences. The protein components mediate spliceosome assembly and the splicing reaction. Autoantibodies to snRNP proteins are found in the blood of patients with systemic lupus erythematosus (Stryer, supra, p. 863).

Heterogeneous nuclear ribonucleoproteins (hnRNPs) have been identified that have roles in splicing, exporting of the mature RNAs to the cytoplasm, and mRNA translation (Biamonti, G. et al. (1998) Clin. Exp. Rheumatol. 16:317-326). Some examples of hnRNPs include the yeast proteins Hrp1p, involved in cleavage and polyadenylation at the 3′ end of the RNA; Cbp80p, involved in capping the 5′ end of the RNA; and Np13p, a homolog of mammalian hnRNP A1, involved in export of mRNA from the nucleus (Shen, E. C. et al. (1998) Genes Dev. 12:679-691). HnRNPs have been shown to be important targets of the autoimmune response in rheumatic diseases (Biamonti et al., supra).

Many snRNP and hnRNP proteins are characterized by an RNA recognition motif (RRM) (reviewed in Birney, E. et al. (1993) Nucleic Acids Res. 21:5803-5816). The RRM is about 80 amino acids in length and forms four β-strands and two α-helices arranged in an α/β sandwich. The RRM contains a core RNP-1 octapeptide motif along with surrounding conserved sequences. In addition to snRNP proteins, examples of RNA-binding proteins which contain the above motifs include heteronuclear ribonucleoproteins which stabilize nascent RNA and factors which regulate alternative splicing. Alternative splicing factors include developmentally regulated proteins, specific examples of which have been identified in lower eukaryotes such as Drosophila melanogaster and Caenorhabditis elegans. These proteins play key roles in developmental processes such as pattern formation and sex determination, respectively (Hodgkin, J. et al. (1994) Development 120:3681-3689).

The 3′ ends of most eukaryote mRNAs are also posttranscriptionally modified by polyadenylation. Polyadenylation proceeds through two enzymatically distinct steps: (i) the endonucleolytic cleavage of nascent mRNAs at cis-acting polyadenylation signals in the 3′-untranslated (non-coding) region and (ii) the addition of a poly(A) tract to the 5′ mRNA fragment. The presence of cis-acting RNA sequences is necessary for both steps. These sequences include 5′-AAUAAA-3′ located 10-30 nucleotides upstream of the cleavage site and a less well-conserved GU- or U-rich sequence element located 10-30 nucleotides downstream of the cleavage site. Cleavage stimulation factor (CstF), cleavage factor I (CF I), and cleavage factor II (CF II) are involved in the cleavage reaction while cleavage and polyadenylation specificity factor (CPSF) and poly(A) polymerase (PAP) are necessary for both cleavage and polyadenylation. An additional enzyme, poly(A)-binding protein II (PAB II), promotes poly(A) tract elongation (Rüegsegger, U. et al. (1996) J. Biol. Chem. 271:6107-6113; and references within).

Translation

Correct translation of the genetic code depends upon each amino acid forming a linkage with the appropriate transfer RNA (tRNA). The aminoacyl-tRNA synthetases (aaRSs) are essential proteins found in all living organisms. The aaRSs are responsible for the activation and correct attachment of an amino acid with its cognate tRNA, as the first step in protein biosynthesis. Prokaryotic organisms have at least twenty different types of aaRSs, one for each different amino acid, while eukaryotes usually have two aaRSs, a cytosolic form and a mitochondrial form, for each different amino acid. The 20 aaRS enzymes can be divided into two structural classes. Class I enzymes add amino acids to the 2′ hydroxyl at the 3′ end of tRNAs while Class II enzymes add amino acids to the 3′ hydroxyl at the 3′ end of tRNAs. Each class is characterized by a distinctive topology of the catalytic domain. Class I enzymes contain a catalytic domain based on the nucleotide-binding ‘Rossman fold’. In particular, a consensus tetrapeptide motif is highly conserved (Prosite Document PDOC00161, Aminoacyl-transfer RNA synthetases class-I signature). Class I enzymes are specific for arginine, cysteine, glutamic acid, glutamine, isoleucine, leucine, methionine, tyrosine, tryptophan, and valine. Class II enzymes contain a central catalytic domain, which consists of a seven-stranded antiparallel β-sheet domain, as well as N— and C-terminal regulatory domains. Class II enzymes are separated into two groups based on the heterodimeric or homodimeric structure of the enzyme; the latter group is further subdivided by the structure of the N— and C-terminal regulatory domains (Hartlein, M. and S. Cusack (1995) J. Mol. Evol. 40:519-530). Class II enzymes are specific for alanine, asparagine, aspartic acid, glycine, histidine, lysine, phenylalanine, proline, serine, and threonine.

Certain aaRSs also have editing functions. IleRS, for example, can misactivate valine to form Val-tRNA^{Ile}, but this product is cleared by a hydrolytic activity that destroys the mischarged product. This editing activity is located within a second catalytic site found in the connective polypeptide 1 region (CP1), a long insertion sequence within the Rossman fold domain of Class I enzymes (Schimmel, P. et al. (1998) FASEB J. 12:1599-1609). AaRSs also play a role in tRNA processing. It has been shown that mature tRNAs are charged with their respective amino acids in the nucleus before export to the cytoplasm, and charging may serve as a quality control mechanism to insure the tRNAs are functional (Martinis, S. A. et al. (1999) EMBO J. 18:4591-4596).

Under optimal conditions, polypeptide synthesis proceeds at a rate of approximately 40 amino acid residues per second. The rate of misincorporation during translation in on the order of 10⁻⁴ and is primarily the result of aminoacyl-t-RNAs being charged with the incorrect amino acid. Incorrectly charged tRNA are toxic to cells as they result in the incorporation of incorrect amino acid residues into an elongating polypeptide. The rate of translation is presumed to be a compromise between the optimal rate of elongation and the need for translational fidelity. Mathematical calculations predict that 10⁻⁴ is indeed the maximum acceptable error rate for protein synthesis in a biological system (reviewed in Stryer, supra; and Watson, J. et al. (1987) The Benjamin/Cummings Publishing Co., Inc. Menlo Park, Calif.). A particularly error prone aminoacyl-tRNA charging event is the charging of tRNA^{Gln} with Gln. A mechanism exits for the correction of this mischarging event which likely has its origins in evolution. Gln was among the last of the 20 naturally occurring amino acids used in polypeptide synthesis to appear in nature. Gram positive eubacteria, cyanobacteria, Archeae, and eukaryotic organelles possess a noncanonical pathway for the synthesis of Gln-tRNA^{Gln} based on the transformation of Glu-tRNA^{Gln} (synthesized by Glu-tRNA synthetase, GluRS) using the enzyme Glu-tRNA^{Gln} amidotransferase (Glu-AdT). The reactions involved in the transamidation pathway are as follows (Curnow, A. W. et al. (1997) Nucleic Acids Symposium 36:24):

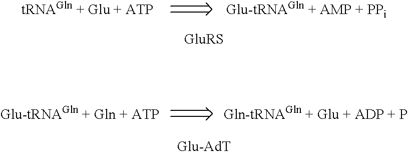

A similar enzyme, Asp-tRNA^{Asn} amidotransferase, exists in Archaea, which transforms Asp-tRNA^{Asn} to Asn-tRNA^{Asn}. Formylase, the enzyme that transforms Met-tRNA^{fMet} to fMet-tRNA^{fMet} in eubacteria, is likely to be a related enzyme. A hydrolytic activity has also been identified that destroys mischarged Val-tRNA^{Ile} (Schimmel et al., supra). One likely scenario for the evolution of Glu-AdT in primitive life forms is the absence of a specific glutaminyl-tRNA synthetase (GlnRS), requiring an alternative pathway for the synthesis of Gln-tRNA^{Gln}. In fact, deletion of the Glu-AdT operon in Gram positive bacteria is lethal (Curnow, A. W. et al. (1997) Proc. Natl. Acad. Sci. USA 94:11819-11826). The existence of GluRS activity in other organisms has been inferred by the high degree of conservation in translation machinery in nature; however, GluRS has not been identified in all organisms, including Homo sapiens. Such an enzyme would be responsible for ensuring translational fidelity and reducing the synthesis of defective polypeptides.

In addition to their function in protein synthesis, specific aminoacyl tRNA synthetases also play roles in cellular fidelity, RNA splicing, RNA trafficking, apoptosis, and transcriptional and translational regulation. For example, human tyrosyl-tRNA synthetase can be proteolytically cleaved into two fragments with distinct cytokine activities. The carboxy-terminal domain exhibits monocyte and leukocyte chemotaxis activity as well as stimulating production of myeloperoxidase, tumor necrosis factor-α, and tissue factor. The N-terminal domain binds to the interleukin-8 type A receptor and functions as an interleukin-8-like cytokine. Human tyrosyl-tRNA synthetase is secreted from apoptotic tumor cells and may accelerate apoptosis (Wakasugi, K., and P. Schimmel (1999) Science 284:147-151). Mitochondrial Neurospora crassa TyrRS and S. cerevisiae LeuRS are essential factors for certain group I intron splicing activities, and human mitochondrial LeuRS can substitute for the yeast LeuRS in a yeast null strain. Certain bacterial aaRSs are involved in regulating their own transcription or translation (Martini et al., supra). Several aaRSs are able to synthesize diadenosine oligophosphates, a class of signalling molecules with roles in cell proliferation, differentiation, and apoptosis (Kisselev, L. L. et al. (1998) FEBS Lett. 427:157-163; Vartanian, A. et al. (1999) FEBS Lett. 456:175-180).

Autoantibodies against aminoacyl-tRNAs are generated by patients with autoimmune diseases such as rheumatic arthritis, dermatomyositis and polymyositis, and correlate strongly with complicating interstitial lung disease (ILD) (Freist, W. et al. (1999) Biol. Chem. 380:623-646; Freist, W. et al. (1996) Biol. Chem. Hoppe Seyler 377:343-356). These antibodies appear to be generated in response to viral infection, and coxsackie virus has been used to induce experimental viral myositis in animals.

Comparison of aaRS structures between humans and pathogens has been useful in the design of novel antibiotics (Schimmel et al., supra). Genetically engineered aaRSs have been utilized to allow site-specific incorporation of unnatural amino acids into proteins in vivo (Liu, D. R. et al. (1997) Proc. Natl. Acad. Sci. USA 94:10092-10097).

tRNA Modifications

The modified ribonucleoside, pseudouridine (Ψ), is present ubiquitously in the anticodon regions of transfer RNAs (tRNAs), large and small ribosomal RNAs (rRNAs), and small nuclear RNAs (snRNAs). Ψ is the most common of the modified nucleosides (i.e., other than G, A, U, and C) present in tRNAs. Only a few yeast tRNAs that are not involved in protein synthesis do not contain Ψ (Cortese, R. et al. (1974) J. Biol. Chem, 249:1103-1108). The enzyme responsible for the conversion of uridine to Ψ, pseudouridine synthase (pseudouridylate synthase), was first isolated from Salmonella typhimurium (Arena, F. et al. (1978) Nucleic Acids Res. 5:4523-4536). The enzyme has since been isolated from a number of mammals, including steer and mice (Green, C. J. et al. (1982) J. Biol. Chen 257:3045-52; and Chen, J. and J. R. Patton (1999) RNA 5:409-419). tRNA pseudouridine synthases have been the most extensively studied members of the family. They require a thiol donor (e.g., cysteine) and a monovalent cation (e.g., ammonia or potassium) for optimal activity. Additional cofactors or high energy molecules (e.g., ATP or GTP) are not required (Green et al., supra). Other eukaryotic pseudouridine synthases have been identified that appear to be specific for rRNA (reviewed in Smith, C. M. and J. A. Steitz (1997) Cell 89:669-672) and a dual-specificity enzyme has been identified that uses both tRNA and rRNA substrates (Wrzesinski, J. et al. (1995) RNA 1: 437-448). The absence of Ψ in the anticodon loop of tRNAs results in reduced growth in both bacteria (Singer, C. E. et al. (1972) Nature New Biol. 238:72-74) and yeast (Lecointe, F. (1998) J. Biol. Chem. 273:1316-1323), although the genetic defect is not lethal.

Another ribonucleoside modification that occurs primarily in eukaryotic cells is the conversion of guanosine to N²,N²-dimethylguanosine (m²₂G) at position 26 or 10 at the base of the D-stem of cytosolic and mitochondrial tRNAs. This posttranscriptional modification is believed to stabilize tRNA structure by preventing the formation of alternative tRNA secondary and tertiary structures. Yeast tRNA^{Asp} is unusual in that it does not contain this modification. The modification does not occur in eubacteria, presumably because the structure of tRNAs in these cells and organelles is sequence constrained and does not require posttranscriptional modification to prevent the formation of alternative structures (Steinberg, S. and R. Cedergren (1995) RNA 1:886-891, and references within). The enzyme responsible for the conversion of guanosine to m²₂G is a 63 kDa S-adenosylmethionine (SAM)-dependent tRNA N²,N²-dimethyl-guanosine methyltransferase (also referred to as the TRM1 gene product and herein referred to as TRM) (Edqvist, J. (1995) Biochimie 77:54-61). The enzyme localizes to both the nucleus and the mitochondria (Li, J.-M. et al. (1989) J. Cell Biol. 109:1411-1419). Based on studies with TRM from Xenopus laevis, there appears to be a requirement for base pairing at positions C11-G24 and G10-C25 immediately preceding the G26 to be modified, with other structural features of the tRNA also being required for the proper presentation of the G26 substrate (Edqvist. J. et al. (1992) Nucleic Acids Res. 20:6575-6581). Studies in yeast suggest that cells carrying a weak ochre tRNA suppressor (sup3-i) are unable to suppress translation termination in the absence of TRM activity, suggesting a role for TRM in modifying the frequency of suppression in eukaryotic cells (Niederberger, C. et al. (1999) FEBS Lett. 464:67-70), in addition to the more general function of ensuring the proper three-dimensional structures for tRNA.

Translation Initiation

Initiation of translation can be divided into three stages. The first stage brings an initiator transfer RNA (Met-tRNA_{f}) together with the 40S ribosomal subunit to form the 43S preinitiation complex. The second stage binds the 43S preinitiation complex to the mRNA, followed by migration of the complex to the correct AUG initiation codon. The third stage brings the 60S ribosomal subunit to the 40S subunit to generate an 80S ribosome at the inititation codon. Regulation of translation primarily involves the first and second stage in the initiation process (Pain, V. M. (1996) Eur. J. Biochem. 236:747-771).

Several initiation factors, many of which contain multiple subunits, are involved in bringing an initiator tRNA and the 40S ribosomal subunit together. eIF2, a guanine nucleotide binding protein, recruits the initiator tRNA to the 40S ribosomal subunit. Only when eIF2 is bound to GTP does it associate with the initiator tRNA. eIF2B, a guanine nucleotide exchange protein, is responsible for converting eIF2 from the GDP-bound inactive form to the GTP-bound active form. Two other factors, eIF1A and eIF3 bind and stabilize the 40S subunit by interacting with the 18S ribosomal RNA and specific ribosomal structural proteins. eIF3 is also involved in association of the 40S ribosomal subunit with mRNA. The Met-tRNA_{f}, eFI1A, eIF3, and 40S ribosomal subunit together make up the 43S preinitiation complex (Pain, supra).

Additional factors are required for binding of the 43S preinitiation complex to an mRNA molecule, and the process is regulated at several levels. eIF4F is a complex consisting of three proteins: eIF4E, eIF4A, and eIF4G. eIF4E recognizes and binds to the mRNA 5′-terminal m⁷GTP cap, eIF4A is a bidirectional RNA-dependent helicase, and eIF4G is a scaffolding polypeptide. eIF4G has three binding domains. The N-terminal third of eIF4G interacts with eIF4E, the central third interacts with eIF4A, and the C-terminal third interacts with eIF3 bound to the 43S preinitiation complex. Thus, eIF4G acts as a bridge between the 40S ribosomal subunit and the mRNA (Hentze, M. W. (1997) Science 275:500-501).

The ability of eIF4F to initiate binding of the 43S preinitiation complex is regulated by structural features of the mRNA. The mRNA molecule has an untranslated region (UTR) between the 5′ cap and the AUG start codon. In some mRNAs this region forms secondary structures that impede binding of the 43S preinitiation complex. The helicase activity of eIF4A is thought to function in removing this secondary structure to facilitate binding of the 43S preinitiation complex (Pain, supra).

Translation Elongation

Elongation is the process whereby additional amino acids are joined to the initiator methionine to form the complete polypeptide chain. The elongation factors EF1α, EF1βγ, and EF2 are involved in elongating the polypeptide chain following initiation. EF1α is a GTP-binding protein. In EF1α's GTP-bound form, it brings an aminoacyl-tRNA to the ribosome's A site. The amino acid attached to the newly arrived aminoacyl-tRNA forms a peptide bond with the initiatior methionine. The GTP on EF1α is hydrolyzed to GDP, and EF1α-GDP dissociates from the ribosome. EF1βγ binds EF1α-GDP and induces the dissociation of GDP from EF1α, allowing EF1α to bind GTP and a new cycle to begin.

As subsequent aminoacyl-tRNAs are brought to the ribosome, EF-G, another GTP-binding protein, catalyzes the translocation of tRNAs from the A site to the P site and finally to the E site of the ribosome. This allows the ribosome and the mRNA to remain attached during translation.

Translation Termination

The release factor eRF carries out termination of translation. eRF recognizes stop codons in the mRNA, leading to the release of the polypeptide chain from the ribosome.

Expression Profiling

Microarrays are analytical tools used in bioanalysis. A microarray has a plurality of molecules spatially distributed over, and stably associated with, the surface of a solid support. Microarrays of polypeptides, polynucleotides, and/or antibodies have been developed and find use in a variety of applications, such as gene sequencing, monitoring gene expression, gene mapping, bacterial identification, drug discovery, and combinatorial chemistry. The potential application of gene expression profiling is particularly relevant to improving diagnosis, prognosis, and treatment of disease.

One area in particular in which microarrays find use is in gene expression analysis. Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for identifying genes that are tissue specific, are affected by a substance being tested in a toxicology assay, are part of a signaling cascade, carry out housekeeping functions, or are specifically related to a particular genetic predisposition, condition, disease, or disorder.

Lung cancer is the leading cause of cancer death in the United States, affecting more than 100,000 men and 50,000 women each year. Nearly 90% of the patients diagnosed with lung cancer are cigarette smokers. Tobacco smoke contains thousands of noxious substances that induce carcinogen metabolizing enzymes and covalent DNA adduct formation in the exposed bronchial epithelium In nearly 80% of patients diagnosed with lung cancer, metastasis has already occurred. Most commonly lung cancers metastasize to pleura, brain, bone, pericardium, and liver. The decision to treat with surgery, radiation therapy, or chemotherapy is made on the basis of tumor histology, response to growth factors or hormones, and sensitivity to inhibitors or drugs. With current treatments, most patients die within one year of diagnosis. Earlier diagnosis and a systematic approach to identification, staging, and treatment of lung cancer could positively affect patient outcome.

Lung cancers progress through a series of morphologically distinct stages from hyperplasia to invasive carcinoma. Malignant lung cancers are divided into two groups comprising four histopathological classes. The Non Small Cell Lung Carcinoma (NSCLC) group includes squamous cell carcinomas, adenocarcinomas, and large cell carcinomas and accounts for about 70% of all lung cancer cases. Adenocarcinomas typically arise in the peripheral airways and often form mucin secreting glands. Squamous cell carcinomas typically arise in proximal airways. The histogenesis of squamous cell carcinomas may be related to chronic inflammation and injury to the bronchial epithelium, leading to squamous metaplasia. The Small Cell Lung Carcinoma (SCLC) group accounts for about 20% of lung cancer cases. SCLCs typically arise in proximal airways and exhibit a number of paraneoplastic syndromes including inappropriate production of adrenocorticotropin and anti-diuretic hormone.

Lung cancer cells accumulate numerous genetic lesions, many of which are associated with cytologically visible chromosomal aberrations. The high frequency of chromosomal deletions associated with lung cancer may reflect the role of multiple tumor suppressor loci in the etiology of this disease. Deletion of the short arm of chromosome 3 is found in over 90% of cases and represents one of the earliest genetic lesions leading to lung cancer. Deletions at chromosome arms 9p and 17p are also common. Other frequently observed genetic lesions include overexpression of telomerase, activation of oncogenes such as K-ras and c-myc, and inactivation of tumor suppressor genes such as RB, p53 and CDKN2.

Genes differentially regulated in lung cancer have been identified by a variety of methods. Using mRNA differential display technology, Manda et al. (1999; Genomics 51:5-14) identified five genes differentially expressed in lung cancer cell lines compared to normal bronchial epithelial cells. Among the known genes, pulmonary surfactant apoprotein A and alpha 2 macroglobulin were down regulated whereas nm23H1 was upregulated. Petersen et al. (2000; Int J. Cancer, 86:512-517) used suppression subtractive hybridization to identify 552 clones differentially expressed in lung tumor derived cell lines, 205 of which represented known genes. Among the known genes, thrombospondin-1, fibronectin, intercellular adhesion molecule 1, and cytokeratins 6 and 18 were previously observed to be differentially expressed in lung cancers. Wang et al. (2000; Oncogene 19:1519-1528) used a combination of microarray analysis and subtractive hybridization to identify 17 genes differentially overexpresssed in squamous cell carcinoma compared with normal lung epithelium Among the known genes they identified were keratin isoform 6, KOC, SPRC, IGFb2, connexin 26, plakofillin 1 and cytokeratin 13.

There are more than 180,000 new cases of breast cancer diagnosed each year, and the mortality rate for breast cancer approaches 10% of all deaths in females between the ages of 45-54 (K. Gish (1999) AWIS Magazine 28:7-10). However the survival rate based on early diagnosis of localized breast cancer is extremely high (97%), compared with the advanced stage of the disease in which the tumor has spread beyond the breast (22%). Current procedures for clinical breast examination are lacking in sensitivity and specificity, and efforts are underway to develop comprehensive gene expression profiles for breast cancer that may be used in conjunction with conventional screening methods to improve diagnosis and prognosis of this disease (Perou C. M. et al. (2000) Nature 406:747-752).

Breast cancer is a genetic disease commonly caused by mutations in breast epithelial cells. Mutations in two genes, BRCA1 and BRCA2, are known to greatly predispose a woman to breast cancer and may be passed on from parents to children (Gish, supra). However, this type of hereditary breast cancer accounts for only about 5% to 9% of breast cancers, while the vast majority of breast cancer is due to nohinherited mutations that occur in breast epithelial cells.

A good deal is already known about the expression of specific genes associated with breast cancer. For example, the relationship between expression of epidermal growth factor (EGF) and its receptor, EGFR, to human mammary carcinoma has been particularly well studied. (See Khazaie, K. et al. (1993) Cancer and Metastasis Rev. 12:255-274), and references cited therein for a review of this area.) Overexpression of EGFR, particularly coupled with down-regulation of the estrogen receptor, is a marker of poor prognosis in breast cancer patients. In addition, EGFR expression in breast tumor metastases is frequently elevated relative to the primary tumor, suggesting that EGFR is involved in tumor progression and metastasis. This is supported by accumulating evidence that EGF has effects on cell functions related to metastatic potential, such as cell motility, chemotaxis, secretion and differentiation. Changes in expression of other members of the erbB receptor family, of which EGFR is one, have also been implicated in breast cancer. The abundance of erbB receptors, such as HER-2/neu, HER-3, and HER-4, and their ligands in breast cancer points to their functional importance in the pathogenesis of the disease, and may therefore provide targets for therapy of the disease (Bacus, S. S. et al. (1994) Am. J. Clin. Pathol. 102:S13-S24). Other known markers of breast cancer include a human secreted frizzled protein mRNA that is downregulated in breast tumors; the matrix G1a protein which is overexpressed is human breast carcinoma cells; Drg1 or RTP, a gene whose expression is diminished in colon, breast, and prostate tumors; maspin, a tumor suppressor gene downregulated in invasive breast carcinomas; and CaN19, a member of the S100 protein family, all of which are down regulated in mammary carcinoma cells relative to normal mammary epithelial cells (Zhou Z. et al. (1998) Int. J. Cancer 78:95-99; Chen, L. et al. (1990) Oncogene 5:1391-1395; Ulrix W. et al (1999) FEBS Lett. 455:23-26; Sager, R. et al. (1996) Curr. Top. Microbiol. Immunol. 213:51-64; and Lee, S. W. et al. (1992) Proc. Natl. Acad. Sci. USA 89:2504-2508).

The potential application of gene expression profiling is particularly relevant to measuring the toxic response to potential therapeutic compounds and of the metabolic response to therapeutic agents. Diseases treated with steroids and disorders caused by the metabolic response to treatment with steroids include adenomatosis, cholestasis, cirrhosis, hemangioma, Henoch-Schonlein purpura, hepatitis, hepatocellular and metastatic carcinomas, idiopathic thrombocytopenic purpura, porphyria, sarcoidosis, and Wilson disease. Response may be measured by comparing both the levels and sequences expressed in tissues from subjects exposed to or treated with steroid compounds such as mifepristone, progesterone, beclomethasone, medroxyprogesterone, budesonide, prednisone, dexamethasone, betamethasone, or danazol with the levels and sequences expressed in normal untreated tissue.

Steroids are a class of lipid-soluble molecules, including cholesterol, bile acids, vitamin D, and hormones, that share a common four-ring structure based on cyclopentanoperhydrophenanthrene and that carrry out a wide variety of functions. Cholesterol, for example, is a component of cell membranes that controls membrane fluidity. It is also a precursor for bile acids which solubilize lipids and facilitate absorption in the small intestine during digestion. Vitamin D regulates the absorption of calcium in the small intestine and controls the concentration of calcium in plasma. Steroid hormones, produced by the adrenal cortex, ovaries, and testes, include glucocorticoids, mineralocorticoids, androgens, and estrogens. They control various biological processes by binding to intracellular receptors that regulate transcription of specific genes in the nucleus. Glucocorticoids, for example, increase blood glucose concentrations by regulation of gluconeogenesis in the liver, increase blood concentrations of fatty acids by promoting lipolysis in adipose tissues, modulate sensitivity to catcholamines in the central nervous system, and reduce inflammation. The principal mineralocorticoid, aldosterone, is produced by the adrenal cortex and acts on cells of the distal tubules of the kidney to enhance sodium ion reabsorption. Androgens, produced by the interstitial cells of Leydig in the testis, include the male sex hormone testosterone, which triggers changes at puberty, the production of sperm and maintenance of secondary sexual characteristics. Female sex hormones, estrogen and progesterone, are produced by the ovaries and also by the placenta and adrenal cortex of the fetus during pregnancy. Estrogen regulates female reproductive processes and secondary sexual characteristics. Progesterone regulates changes in the endometrium during the menstrual cycle and pregnancy.

Steroid hormones are widely used for fertility control and in anti-inflammatory treatments for physical injuries and diseases such as arthritis, asthma, and auto-immune disorders. Progesterone, a naturally occurring progestin, is primarily used to treat amenorrhea, abnormal uterine bleeding, or as a contraceptive. Endogenous progesterone is responsible for inducing secretory activity in the endometrium of the estrogen-primed uterus in preparation for the implantation of a fertilized egg and for the maintenance of pregnancy. It is secreted from the corpus luteum in response to luteinizing hormone (LH). The primary contraceptive effect of exogenous progestins involves the suppression of the midcycle surge of LH. At the cellular level, progestins diffuse freely into target cells and bind to the progesterone receptor. Target cells include the female reproductive tract, the mammary gland, the hypothalamus, and the pituitary. Once bound to the receptor, progestins slow the frequency of release of gonadotropin releasing hormone from the hypothalamus and blunt the pre-ovulatory LH surge, thereby preventing follicular maturation and ovulation. Progesterone has minimal estrogenic and androgenic activity. Progesterone is metabolized hepatically to pregnanediol and conjugated with glucuronic acid.

Medroxyprogesterone (MAH), also known as 6α-methyl-17-hydroxyprogesterone, is a synthetic progestin with a pharmacological activity about 15 times greater than progesterone. MAH is used for the treatment of renal and endometrial carcinomas, amenorrhea, abnormal uterine bleeding, and endometriosis associated with hormonal imbalance. MAH has a stimulatory effect on respiratory centers and has been used in cases of low blood oxygenation caused by sleep apnea, chronic obstructive pulmonary disease, or hypercapnia.

Mifepristone, also known as RU-486, is an antiprogesterone drug that blocks receptors of progesterone. It counteracts the effects of progesterone, which is needed to sustain pregnancy. Mifepristone induces spontaneous abortion when administered in early pregnancy followed by treatment with the prostaglandin, misoprostol. Further, studies show that mifepristone at a substantially lower dose can be highly effective as a postcoital contraceptive when administered within five days after unprotected intercourse, thus providing women with a “morning-after pill” in case of contraceptive failure or sexual assault. Mifepristone also has potential uses in the treatment of breast and ovarian cancers in cases in which tumors are progesterone-dependent It interferes with steroid-dependent growth of brain meningiomas, and may be useful in treatment of endometriosis where it blocks the estrogen-dependent growth of endometrial tissues. It may also be useful in treatment of uterine fibroid tumors and Cushing's Syndrome. Mifepristone binds to glucocorticoid receptors and interferes with cortisol binding. Mifepristone also may act as an anti-glucocorticoid and be effective for treating conditions where cortisol levels are elevated such as AIDS, anorexia nervosa, ulcers, diabetes, Parkinson's disease, multiple sclerosis, and Alzheimer's disease.

Danazol is a synthetic steroid derived from ethinyl testosterone. Danazol indirectly reduces estrogen production by lowering pituitary synthesis of follicle-stimulating hormone and LH. Danazol also binds to sex hormone receptors in target tissues, thereby exhibiting anabolic, antiestrognic, and weakly androgenic activity. Danazol does not possess any progestogenic activity, and does not suppress normal pituitary release of corticotropin or release of cortisol by the adrenal glands. Danazol is used in the treatment of endometriosis to relieve pain and inhibit endometrial cell growth. It is also used to treat fibrocystic breast disease and hereditary angioedema.

Corticosteroids are used to relieve inflammation and to suppress the immune response. They inhibit eosinophil, basophil, and airway epithelial cell function by regulation of cytokines that mediate the inflammatory response. They inhibit leukocyte infiltration at the site of inflammation, interfere in the function of mediators of the inflammatory response, and suppress the humoral immune response. Corticosteroids are used to treat allergies, asthma, arthritis, and skin conditions. Beclomethasone is a synthetic glucocorticoid that is used to treat steroid-dependent asthma, to relieve symptoms associated with allergic or nonallergic (vasomotor) rhinitis, or to prevent recurrent nasal polyps following surgical removal. The anti-inflammatory and vasoconstrictive effects of intranasal beclomethasone are 5000 times greater than those produced by hydrocortisone. Budesonide is a corticosteroid used to control symptoms associated with allergic rhinitis or asthma Budesonide has high topical anti-inflammatory activity but low systemic activity. Dexamethasone is a synthetic glucocorticoid used in anti-inflammatory or immunosuppressive compositions. It is also used in inhalants to prevent symptoms of asthma. Due to its greater ability to reach the central nervous system, dexamethasone is usually the treatment of choice to control cerebral edema. Dexamethasone is approximately 20-30 times more potent than hydrocortisone and 5-7 times more potent than prednisone. Prednisone is metabolized in the liver to its active form, prednisolone, a glucocorticoid with anti-inflammatory properties. Prednisone is approximately 4 times more potent than hydrocortisone and the duration of action of prednisone is intermediate between hydrocortisone and dexamethasone. Prednisone is used to treat allograft rejection, asthma, systemic lupus erythematosus, arthritis, ulcerative colitis, and other inflammatory conditions. Betamethasone is a synthetic glucocorticoid with antiinflammatory and immunosuppressive activity and is used to treat psoriasis and fungal infections, such as athlete's foot and ringworm.

The anti-inflammatory actions of corticosteroids are thought to involve phospholipase A₂ inhibitory proteins, collectively called lipocortins. Lipocortins, in turn, control the biosynthesis of potent mediators of inflammation such as prostaglandins and leukotrienes by inhibiting the release of the precursor molecule arachidonic acid. Proposed mechanisms of action include decreased IgE synthesis, increased number of β-adrenergic receptors on leukocytes, and decreased arachidonic acid metabolism During an immediate allergic reaction, such as in chronic bronchial asthma, allergens bridge the IgE antibodies on the surface of mast cells, which triggers these cells to release chemotactic substances. Mast cell influx and activation, therefore, is partially responsible for the inflammation and hyperirritability of the oral mucosa in asthmatic patients. This inflammation can be retarded by administration of corticosteroids.

The effects upon liver metabolism and hormone clearance mechanisms are important to understand the pharmacodynamics of a drug. The human C3A cell line is a clonal derivative of HepG2/C3 (hepatoma cell line, isolated from a 15-year-old male with liver tumor), which was selected for strong contact inhibition of growth. The use of a clonal population enhances the reproducibility of the cells. C3A cells have many characteristics of primary human hepatocytes in culture: i) expression of insulin receptor and insulin-like growth factor II receptor; ii) secretion of a high ratio of serum albumin compared with α-fetoprotein iii) conversion of ammonia to urea and glutamine; iv) metabolize aromatic amino acids; and v) proliferate in glucose-free and insulin-free medium The C3A cell line is now well established as an in vitro model of the mature human liver (Mickelson et al. (1995) Hepatology 22:866-875; Nagendra et al. (1997) Am J Physiol 272:G408-G416).

There is a need in the art for new compositions, including nucleic acids and proteins, for the diagnosis, prevention, and treatment of cell proliferative, DNA repair, neurological, reproductive, developmental, and autoimmune/inflammatory disorders, and infections.

### SUMMARY OF THE INVENTION

Various embodiments of the invention provide purified polypeptides, nucleic acid-associated proteins, referred to collectively as “NAAP” and individually as “NAAP-1,” “NAAP-2,” “NAAP-3,” “NAAP4,” “NAAP-5,” “NAAP-6,” “NAAP-7,” “NAAP-8,” “NAAP-9,” “NAAP-10,” “NAAP-11,” “NAAP-12,” “NAAP-13,” “NAAP-14,” “NAAP-15,” “NAAP-16,” “NAAP-17,” “NAAP-18,” “NAAP-19,” “NAAP-20,” “NAAP-21,” “NAAP-22,” “NAAP-23,” ”NAAP-24,” “NAAP-25,” “NAAP-26,” “NAAP-27,” “NAAP-28,” “NAAP-29,” “NAAP-30,” “NAAP-31,” “NAAP-32,” “NAAP-33,” and methods for using these proteins and their encoding polynucleotides for the detection, diagnosis, and treatment of diseases and medical conditions. Embodiments also provide methods for utilizing the purified nucleic acid-associated proteins and/or their encoding polynucleotides for facilitating the drug discovery process, including determination of efficacy, dosage, toxicity, and pharmacology. Related embodiments provide methods for utilizing the purified nucleic acid-associated proteins and/or their encoding polynucleotides for investigating the pathogenesis of diseases and medical conditions.

An embodiment provides an isolated polypeptide selected from the group consisting of a) a polypeptide comprising an amino acid sequence selected from the group consisting of SEQ ID NO:1-33, b) a polypeptide comprising a naturally occurring amino acid sequence at least 90% identical or at least about 90% identical to an amino acid sequence selected from the group consisting of SEQ ID NO:1-33, c) a biologically active fragment of a polypeptide having an amino acid sequence selected from the group consisting of SEQ ID NO:1-33, and d) an immunogenic fragment of a polypeptide having an amino acid sequence selected from the group consisting of SEQ ID NO:1-33. Another embodiment provides an isolated polypeptide comprising an amino acid sequence of SEQ ID NO:1-33.

Still another embodiment provides an isolated polynucleotide encoding a polypeptide selected from the group consisting of a) a polypeptide comprising an amino acid sequence selected from the group consisting of SEQ ID NO:1-33, b) a polypeptide comprising a naturally occurring amino acid sequence at least 90% identical or at least about 90% identical to an amino acid sequence selected from the group consisting of SEQ ID NO:1-33, c) a biologically active fragment of a polypeptide having an amino acid sequence selected from the group consisting of SEQ ID NO:1-33, and d) an immunogenic fragment of a polypeptide having an amino acid sequence selected from the group consisting of SEQ ID NO:1-33. In another embodiment, the polynucleotide encodes a polypeptide selected from the group consisting of SEQ ID NO:1-33. In an alternative embodiment, the polynucleotide is selected from the group consisting of SEQ ID NO:34-66.

Still another embodiment provides a recombinant polynucleotide comprising a promoter sequence operably linked to a polynucleotide encoding a polypeptide selected from the group consisting of a) a polypeptide comprising an amino acid sequence selected from the group consisting of SEQ ID NO:1-33, b) a polypeptide comprising a naturally occurring amino acid sequence at least 90% identical or at least about 90% identical to an amino acid sequence selected from the group consisting of SEQ ID NO:1-33, c) a biologically active fragment of a polypeptide having an amino acid sequence selected from the group consisting of SEQ ID NO:1-33, and d) an immunogenic fragment of a polypeptide having an amino acid sequence selected from the group consisting of SEQ ID NO:1-33. Another embodiment provides a cell transformed with the recombinant polynucleotide. Yet another embodiment provides a transgenic organism comprising the recombinant polynucleotide.

Another embodiment provides a method for producing a polypeptide selected from the group consisting of a) a polypeptide comprising an amino acid sequence selected from the group consisting of SEQ ID NO:1-33, b) a polypeptide comprising a naturally occurring amino acid sequence at least 90% identical or at least about 90% identical to an amino acid sequence selected from the group consisting of SEQ ID NO:1-33, c) a biologically active fragment of a polypeptide having an amino acid sequence selected from the group consisting of SEQ ID NO:1-33, and d) an immunogenic fragment of a polypeptide having an amino acid sequence selected from the group consisting of SEQ ID NO:1-33. The method comprises a) culturing a cell under conditions suitable for expression of the polypeptide, wherein said cell is transformed with a recombinant polynucleotide comprising a promoter sequence operably linked to a polynucleotide encoding the polypeptide, and b) recovering the polypeptide so expressed.

Yet another embodiment provides an isolated antibody which specifically binds to a polypeptide selected from the group consisting of a) a polypeptide comprising an amino acid sequence selected from the group consisting of SEQ ID NO:1-33, b) a polypeptide comprising a naturally occurring amino acid sequence at least 90% identical or at least about 90% identical to an amino acid sequence selected from the group consisting of SEQ ID NO:1-33, c) a biologically active fragment of a polypeptide having an amino acid sequence selected from the group consisting of SEQ ID NO:1-33, and d) an immunogenic fragment of a polypeptide having an amino acid sequence selected from the group consisting of SEQ ID NO:1-33.

Still yet another embodiment provides an isolated polynucleotide selected from the group consisting of a) a polynucleotide comprising a polynucleotide sequence selected from the group consisting of SEQ ID NO:34-66, b) a polynucleotide comprising a naturally occurring polynucleotide sequence at least 90% identical or at least about 90% identical to a polynucleotide sequence selected from the group consisting of SEQ ID NO:34-66, c) a polynucleotide complementary to the polynucleotide of a), d) a polynucleotide complementary to the polynucleotide of b), and e) an RNA equivalent of a)-d). In other embodiments, the polynucleotide can comprise at least about 20, 30, 40, 60, 80, or 100 contiguous nucleotides.

Yet another embodiment provides a method for detecting a target polynucleotide in a sample, said target polynucleotide being selected from the group consisting of a) a polynucleotide comprising a polynucleotide sequence selected from the group consisting of SEQ ID NO:34-66, b) a polynucleotide comprising a naturally occurring polynucleotide sequence at least 90% identical or at least about 90% identical to a polynucleotide sequence selected from the group consisting of SEQ ID NO:34-66, c) a polynucleotide complementary to the polynucleotide of a), d) a polynucleotide complementary to the polynucleotide of b), and e) an RNA equivalent of a)-d). The method comprises a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and b) detecting the presence or absence of said hybridization complex. In a related embodiment, the method can include detecting the amount of the hybridization complex. In still other embodiments, the probe can comprise at least about 20, 30, 40, 60, 80, or 100 contiguous nucleotides.

Still yet another embodiment provides a method for detecting a target polynucleotide in a sample, said target polynucleotide being selected from the group consisting of a) a polynucleotide comprising a polynucleotide sequence selected from the group consisting of SEQ ID NO:34-66, b) a polynucleotide comprising a naturally occurring polynucleotide sequence at least 90% identical or at least about 90% identical to a polynucleotide sequence selected from the group consisting of SEQ ID NO:34-66, c) a polynucleotide complementary to the polynucleotide of a), d) a polynucleotide complementary to the polynucleotide of b), and e) an RNA equivalent of a)-d). The method comprises a) amplifying said target polynucleotide or fragment thereof using polymerase chain reaction amplification, and b) detecting the presence or absence of said amplified target polynucleotide or fragment thereof. In a related embodiment, the method can include detecting the amount of the amplified target polynucleotide or fragment thereof.

Another embodiment provides a composition comprising an effective amount of a polypeptide selected from the group consisting of a) a polypeptide comprising an amino acid sequence selected from the group consisting of SEQ ID NO:1-33, b) a polypeptide comprising a naturally occurring amino acid sequence at least 90% identical or at least about 90% identical to an amino acid sequence selected from the group consisting of SEQ ID NO:1-33, c) a biologically active fragment of a polypeptide having an amino acid sequence selected from the group consisting of SEQ ID NO:1-33, and d) an immunogenic fragment of a polypeptide having an amino acid sequence selected from the group consisting of SEQ ID NO:1-33, and a pharmaceutically acceptable excipient. In one embodiment, the composition can comprise an amino acid sequence selected from the group consisting of SEQ ID NO:1-33. Other embodiments provide a method of treating a disease or condition associated with decreased or abnormal expression of functional NAAP, comprising administering to a patient in need of such treatment the composition.

Yet another embodiment provides a method for screening a compound for effectiveness as an agonist of a polypeptide selected from the group consisting of a) a polypeptide comprising an amino acid sequence selected from the group consisting of SEQ ID NO:1-33, b) a polypeptide comprising a naturally occurring amino acid sequence at least 90% identical or at least about 90% identical to an amino acid sequence selected from the group consisting of SEQ ID NO:1-33, c) a biologically active fragment of a polypeptide having an amino acid sequence selected from the group consisting of SEQ ID NO:1-33, and d) an immunogenic fragment of a polypeptide having an amino acid sequence selected from the group consisting of SEQ ID NO:1-33. The method comprises a) exposing a sample comprising the polypeptide to a compound, and b) detecting agonist activity in the sample. Another embodiment provides a composition comprising an agonist compound identified by the method and a pharmaceutically acceptable excipient. Yet another embodiment provides a method of treating a disease or condition associated with decreased expression of functional NAAP, comprising administering to a patient in need of such treatment the composition.

Still yet another embodiment provides a method for screening a compound for effectiveness as an antagonist of a polypeptide selected from the group consisting of a) a polypeptide comprising an amino acid sequence selected from the group consisting of SEQ ID NO:1-33, b) a polypeptide comprising a naturally occurring amino acid sequence at least 90% identical or at least about 90% identical to an amino acid sequence selected from the group consisting of SEQ ID NO:1-33, c) a biologically active fragment of a polypeptide having an amino acid sequence selected from the group consisting of SEQ ID NO:1-33, and d) an immunogenic fragment of a polypeptide having an amino acid sequence selected from the group consisting of SEQ ID NO:1-33. The method comprises a) exposing a sample comprising the polypeptide to a compound, and b) detecting antagonist activity in the sample. Another embodiment provides a composition comprising an antagonist compound identified by the method and a pharmaceutically acceptable excipient. Yet another embodiment provides a method of treating a disease or condition associated with overexpression of functional NAAP, comprising administering to a patient in need of such treatment the composition.

Another embodiment provides a method of screening for a compound that specifically binds to a polypeptide selected from the group consisting of a) a polypeptide comprising an amino acid sequence selected from the group consisting of SEQ ID NO:1-33, b) a polypeptide comprising a naturally occurring amino acid sequence at least 90% identical or at least about 90% identical to an amino acid sequence selected from the group consisting of SEQ ID NO:1-33, c) a biologically active fragment of a polypeptide having an amino acid sequence selected from the group consisting of SEQ ID NO:1-33, and d) an immunogenic fragment of a polypeptide having an amino acid sequence selected from the group consisting of SEQ ID NO:1-33. The method comprises a) combining the polypeptide with at least one test compound under suitable conditions, and b) detecting binding of the polypeptide to the test compound, thereby identifying a compound that specifically binds to the polypeptide.

Yet another embodiment provides a method of screening for a compound that modulates the activity of a polypeptide selected from the group consisting of a) a polypeptide comprising an amino acid sequence selected from the group consisting of SEQ ID NO:1-33, b) a polypeptide comprising a naturally occurring amino acid sequence at least 90% identical or at least about 90% identical to an amino acid sequence selected from the group consisting of SEQ ID NO:1-33, c) a biologically active fragment of a polypeptide having an amino acid sequence selected from the group consisting of SEQ ID NO:1-33, and d) an immunogenic fragment of a polypeptide having an amino acid sequence selected from the group consisting of SEQ ID NO:1-33. The method comprises a) combining the polypeptide with at least one test compound under conditions permissive for the activity of the polypeptide, b) assessing the activity of the polypeptide in the presence of the test compound, and c) comparing the activity of the polypeptide in the presence of the test compound with the activity of the polypeptide in the absence of the test compound, wherein a change in the activity of the polypeptide in the presence of the test compound is indicative of a compound that modulates the activity of the polypeptide.

Still yet another embodiment provides a method for screening a compound for effectiveness in altering expression of a target polynucleotide, wherein said target polynucleotide comprises a polynucleotide sequence selected from the group consisting of SEQ ID NO:34-66, the method comprising a) exposing a sample comprising the target polynucleotide to a compound, b) detecting altered expression of the target polynucleotide, and c) comparing the expression of the target polynucleotide in the presence of varying amounts of the compound and in the absence of the compound.

Another embodiment provides a method for assessing toxicity of a test compound, said method comprising a) treating a biological sample containing nucleic acids with the test compound; b) hybridizing the nucleic acids of the treated biological sample with a probe comprising at least 20 contiguous nucleotides of a polynucleotide selected from the group consisting of i) a polynucleotide comprising a polynucleotide sequence selected from the group consisting of SEQ ID NO:34-66, ii) a polynucleotide comprising a naturally occurring polynucleotide sequence at least 90% identical or at least about 90% identical to a polynucleotide sequence selected from the group consisting of SEQ ID NO:34-66, iii) a polynucleotide having a sequence complementary to i), iv) a polynucleotide complementary to the polynucleotide of ii), and v) an RNA equivalent of i)-iv). Hybridization occurs under conditions whereby a specific hybridization complex is formed between said probe and a target polynucleotide in the biological sample, said target polynucleotide selected from the group consisting of i) a polynucleotide comprising a polynucleotide sequence selected from the group consisting of SEQ ID NO:34-66, ii) a polynucleotide comprising a naturally occurring polynucleotide sequence at least 90% identical or at least about 90% identical to a polynucleotide sequence selected from the group consisting of SEQ ID NO:34-66, iii) a polynucleotide complementary to the polynucleotide of i), iv) a polynucleotide complementary to the polynucleotide of ii), and v) an RNA equivalent of i)-iv). Alternatively, the target polynucleotide can comprise a fragment of a polynucleotide selected from the group consisting of i)-v) above; c) quantifying the amount of hybridization complex; and d) comparing the amount of hybridization complex in the treated biological sample with the amount of hybridization complex in an untreated biological sample, wherein a difference in the amount of hybridization complex in the treated biological sample is indicative of toxicity of the test compound.

### BRIEF DESCRIPTION OF THE TABLES

Table 1 summarizes the nomenclature for full length polynucleotide and polypeptide embodiments of the invention.

Table 2 shows the GenBank identification number and annotation of the nearest GenBank homolog for polypeptide embodiments of the invention. The probability scores for the matches between each polypeptide and its homolog(s) are also shown.

Table 3 shows structural features of polypeptide embodiments, including predicted motifs and domains, along with the methods, algorithms, and searchable databases used for analysis of the polypeptides.

Table 4 lists the cDNA and/or genomic DNA fragments which were used to assemble polynucleotide embodiments, along with selected fragments of the polynucleotides.

Table 5 shows representative cDNA libraries for polynucleotide embodiments.

Table 6 provides an appendix which describes the tissues and vectors used for construction of the cDNA libraries shown in Table 5.

Table 7 shows the tools, programs, and algorithms used to analyze polynucleotides and polypeptides, along with applicable descriptions, references, and threshold parameters.

### DESCRIPTION OF THE INVENTION

Before the present proteins, nucleic acids, and methods are described, it is understood that embodiments of the invention are not limited to the particular machines, instruments, materials, and methods described, as these may vary. It is also to be understood that the terminology used herein is for the purpose of describing particular embodiments only, and is not intended to limit the scope of the invention.

As used herein and in the appended claims, the singular forms “a,” “an,” and “the” include plural reference unless the context clearly dictates otherwise. Thus, for example, a reference to “a host cell” includes a plurality of such host cells, and a reference to “an antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any machines, materials, and methods similar or equivalent to those described herein can be used to practice or test the present invention, the preferred machines, materials and methods are now described. All publications mentioned herein are cited for the purpose of describing and disclosing the cell lines, protocols, reagents and vectors which are reported in the publications and which might be used in connection with various embodiments of the invention. Nothing herein is to be construed as an admission that the invention is not entitled to antedate such disclosure by virtue of prior invention.

Definitions

“NAAP” refers to the amino acid sequences of substantially purified NAAP obtained from any species, particularly a mammalian species, including bovine, ovine, porcine, murine, equine, and human, and from any source, whether natural, synthetic, semi-synthetic, or recombinant.

The term “agonist” refers to a molecule which intensifies or mimics the biological activity of NAAP. Agonists may include proteins, nucleic acids, carbohydrates, small molecules, or any other compound or composition which modulates the activity of NAAP either by directly interacting with NAAP or by acting on components of the biological pathway in which NAAP participates.

An “allelic variant” is an alternative form of the gene encoding NAAP. Allelic variants may result from at least one mutation in the nucleic acid sequence and may result in altered mRNAs or in polypeptides whose structure or function may or may not be altered. A gene may have none, one, or many allelic variants of its naturally occurring form Common mutational changes which give rise to allelic variants are generally ascribed to natural deletions, additions, or substitutions of nucleotides. Each of these types of changes may occur alone, or in combination with the others, one or more times in a given sequence.

“Altered” nucleic acid sequences encoding NAAP include those sequences with deletions, insertions, or substitutions of different nucleotides, resulting in a polypeptide the same as NAAP or a polypeptide with at least one functional characteristic of NAAP. Included within this definition are polymorphisms which may or may not be readily detectable using a particular oligonucleotide probe of the polynucleotide encoding NAAP, and improper or unexpected hybridization to allelic variants, with a locus other than the normal chromosomal locus for the polynucleotide encoding NAAP. The encoded protein may also be “altered,” and may contain deletions, insertions, or substitutions of amino acid residues which produce a silent change and result in a functionally equivalent NAAP. Deliberate amino acid substitutions may be made on the basis of one or more similarities in polarity, charge, solubility, hydrophobicity, hydrophilicity, and/or the amphipathic nature of the residues, as long as the biological or immunological activity of NAAP is retained. For example, negatively charged amino acids may include aspartic acid and glutamic acid, and positively charged amino acids may include lysine and arginine. Amino acids with uncharged polar side chains having similar hydrophilicity values may include: asparagine and glutamine; and serine and threonine. Amino acids with uncharged side chains having similar hydrophilicity values may include: leucine, isoleucine, and valine; glycine and alanine; and phenylalanine and tyrosine.

The terms “amino acid” and “amino acid sequence” can refer to an oligopeptide, a peptide, a polypeptide, or a protein sequence, or a fragment of any of these, and to naturally occurring or synthetic molecules. Where “amino acid sequence” is recited to refer to a sequence of a naturally occurring protein molecule, “amino acid sequence” and like terms are not meant to limit the amino acid sequence to the complete native amino acid sequence associated with the recited protein molecule.

“Amplification” relates to the production of additional copies of a nucleic acid. Amplification may be carried out using polymerase chain reaction (PCR) technologies or other nucleic acid amplification technologies well known in the art.

The term “antagonist” refers to a molecule which inhibits or attenuates the biological activity of NAAP. Antagonists may include proteins such as antibodies, anticalins, nucleic acids, carbohydrates, small molecules, or any other compound or composition which modulates the activity of NAAP either by directly interacting with NAAP or by acting on components of the biological pathway in which NAAP participates.

The term “antibody” refers to intact immunoglobulin molecules as well as to fragments thereof, such as Fab, F(ab′)₂, and Fv fragments, which are capable of binding an epitopic determinant. Antibodies that bind NAAP polypeptides can be prepared using intact polypeptides or using fragments containing small peptides of interest as the immunizing antigen. The polypeptide or oligopeptide used to immunize an animal (e.g., a mouse, a rat, or a rabbit) can be derived from the translation of RNA, or synthesized chemically, and can be conjugated to a carrier protein if desired. Commonly used carriers that are chemically coupled to peptides include bovine serum albumin, thyroglobulin, and keyhole limpet hemocyanin (KLH). The coupled peptide is then used to immunize the animal.

The term “antigenic determinant” refers to that region of a molecule (i.e., an epitope) that makes contact with a particular antibody. When a protein or a fragment of a protein is used to immunize a host animal, numerous regions of the protein may induce the production of antibodies which bind specifically to antigenic determinants (particular regions or three-dimensional structures on the protein). An antigenic determinant may compete with the intact antigen (i.e., the immunogen used to elicit the immune response) for binding to an antibody.

The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment), described in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′-OH group of a ribonucleotide may be replaced by 2′-F or 2′-NH₂), which may improve a desired property, e.g., resistance to nucleases or longer lifetime in blood. Aptamers may be conjugated to other molecules, e.g., a high molecular weight carrier to slow clearance of the aptamer from the circulatory system. Aptamers may be specifically cross-linked to their cognate ligands, e.g., by photo-activation of a cross-linker (Brody, E. N. and L. Gold (2000) J. Biotechnol. 74:5-13).

The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl. Acad. Sci. USA 96:3606-3610).

The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

The term “antisense” refers to any composition capable of base-pairing with the “sense” (coding) strand of a polynucleotide having a specific nucleic acid sequence. Antisense compositions may include DNA; RNA; peptide nucleic acid (PNA); oligonucleotides having modified backbone linkages such as phosphorothioates, methylphosphonates, or benzylphosphonates; oligonucleotides having modified sugar groups such as 2′-methoxyethyl sugars or 2′-methoxyethoxy sugars; or oligonucleotides having modified bases such as 5-methyl cytosine, 2′-deoxyuracil, or 7-deaza-2′-deoxyguanosine. Antisense molecules may be produced by any method including chemical synthesis or transcription. Once introduced into a cell, the complementary antisense molecule base-pairs with a naturally occurring nucleic acid sequence produced by the cell to form duplexes which block either transcription or translation. The designation “negative” or “minus” can refer to the antisense strand, and the designation “positive” or “plus” can refer to the sense strand of a reference DNA molecule.

The term “biologically active” refers to a protein having structural, regulatory, or biochemical functions of a naturally occurring molecule. Likewise, “immunologically active” or “immunogenic” refers to the capability of the natural, recombinant, or synthetic NAAP, or of any oligopeptide thereof, to induce a specific immune response in appropriate animals or cells and to bind with specific antibodies.

“Complementary” describes the relationship between two single-stranded nucleic acid sequences that anneal by base-pairing. For example, 5′-AGT-3′ pairs with its complement, 3′-TCA-5′.

A “composition comprising a given polynucleotide” and a “composition comprising a given polypeptide” can refer to any composition containing the given polynucleotide or polypeptide. The composition may comprise a dry formulation or an aqueous solution. Compositions comprising polynucleotides encoding NAAP or fragments of NAAP may be employed as hybridization probes. The probes may be stored in freeze-dried form and may be associated with a stabilizing agent such as a carbohydrate. In hybridizations, the probe may be deployed in an aqueous solution containing salts (e.g., NaCl), detergents (e.g., sodium dodecyl sulfate; SDS), and other components (e.g., Denhardt's solution, dry milk, salmon sperm DNA, etc.).

“Consensus sequence” refers to a nucleic acid sequence which has been subjected to repeated DNA sequence analysis to resolve uncalled bases, extended using the XL-PCR kit (Applied Biosystems, Foster City Calif.) in the 5′ and/or the 3′ direction, and resequenced, or which has been assembled from one or more overlapping cDNA, EST, or genomic DNA fragments using a computer program for fragment assembly, such as the GELVIEW fragment assembly system (GCG, Madison Wis.) or Phrap (University of Washington, Seattle Wash.). Some sequences have been both extended and assembled to produce the consensus sequence.

“Conservative amino acid substitutions” are those substitutions that are predicted to least interfere with the properties of the original protein, i.e., the structure and especially the function of the protein is conserved and not significantly changed by such substitutions. The table below shows amino acids which may be substituted for an original amino acid in a protein and which are regarded as conservative amino acid substitutions.

| Original Residue | Conservative Substitution |
|---|---|
| Ala | Gly, Ser |
| Arg | His, Lys |
| Asn | Asp, Gln, His |
| Asp | Asn, Glu |
| Cys | Ala, Ser |
| Gln | Asn, Glu, His |
| Glu | Asp, Gln, His |
| Gly | Ala |
| His | Asn, Arg, Gln, Glu |
| Ile | Leu, Val |
| Leu | Ile, Val |
| Lys | Arg, Gln, Glu |
| Met | Leu, Ile |
| Phe | His, Met, Leu, Trp, Tyr |
| Ser | Cys, Thr |
| Thr | Ser, Val |
| Trp | Phe, Tyr |
| Tyr | His, Phe, Trp |
| Val | Ile, Leu, Thr |

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

A “deletion” refers to a change in the amino acid or nucleotide sequence that results in the absence of one or more amino acid residues or nucleotides.

The term “derivative” refers to a chemically modified polynucleotide or polypeptide. Chemical modifications of a polynucleotide can include, for example, replacement of hydrogen by an alkyl, acyl, hydroxyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “detectable label” refers to a reporter molecule or enzyme that is capable of generating a measurable signal and is covalently or noncovalently joined to a polynucleotide or polypeptide.

“Differential expression” refers to increased or upregulated; or decreased, downregulated, or absent gene or protein expression, determined by comparing at least two different samples. Such comparisons may be carried out between, for example, a treated and an untreated sample, or a diseased and a normal sample.

“Exon shuffling” refers to the recombination of different coding regions (exons). Since an exon may represent a structural or functional domain of the encoded protein, new proteins may be assembled through the novel reassortment of stable substructures, thus allowing acceleration of the evolution of new protein functions.

A “fragment” is a unique portion of NAAP or a polynucleotide encoding NAAP which can be identical in sequence to, but shorter in length than, the parent sequence. A fragment may comprise up to the entire length of the defined sequence, minus one nucleotide/amino acid residue. For example, a fragment may comprise from about 5 to about 1000 contiguous nucleotides or amino acid residues. A fragment used as a probe, primer, antigen, therapeutic molecule, or for other purposes, may be at least 5, 10, 15, 16, 20, 25, 30, 40, 50, 60, 75, 100, 150, 250 or at least 500 contiguous nucleotides or amino acid residues in length. Fragments may be preferentially selected from certain regions of a molecule. For example, a polypeptide fragment may comprise a certain length of contiguous amino acids selected from the first 250 or 500 amino acids (or first 25% or 50%) of a polypeptide as shown in a certain defined sequence. Clearly these lengths are exemplary, and any length that is supported by the specification, including the Sequence Listing, tables, and figures, may be encompassed by the present embodiments.

A fragment of SEQ ID NO:34-66 can comprise a region of unique polynucleotide sequence that specifically identifies SEQ ID NO:34-66, for example, as distinct from any other sequence in the genome from which the fragment was obtained. A fragment of SEQ ID NO:34-66 can be employed in one or more embodiments of methods of the invention, for example, in hybridization and amplification technologies and in analogous methods that distinguish SEQ ID NO:34-66 from related polynucleotides. The precise length of a fragment of SEQ ID NO:34-66 and the region of SEQ ID NO:34-66 to which the fragment corresponds are routinely determinable by one of ordinary skill in the art based on the intended purpose for the fragment.

A fragment of SEQ ID NO:1-33 is encoded by a fragment of SEQ ID NO:34-66. A fragment of SEQ ID NO:1-33 can comprise a region of unique amino acid sequence that specifically identifies SEQ ID NO:1-33. For example, a fragment of SEQ ID NO:1-33 can be used as an immunogenic peptide for the development of antibodies that specifically recognize SEQ ID NO:1-33. The precise length of a fragment of SEQ ID NO:1-33 and the region of SEQ ID NO:1-33 to which the fragment corresponds can be determined based on the intended purpose for the fragment using one or more analytical methods described herein or otherwise known in the art.

A “full length” polynucleotide is one containing at least a translation initiation codon (e.g., methionine) followed by an open reading frame and a translation termination codon. A “full length” polynucleotide sequence encodes a “full length” polypeptide sequence.

“Homology” refers to sequence similarity or, interchangeably, sequence identity, between two or more polynucleotide sequences or two or more polypeptide sequences.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between polynucleotide sequences may be determined using one or more computer algorithms or programs known in the art or described herein. For example, percent identity can be determined using the default parameters of the CLUSTAL V algorithm as incorporated into the MEGALIGN version 3.12e sequence alignment program. This program is part of the LASERGENE software package, a suite of molecular biological analysis programs (DNASTAR, Madison Wis.). CLUSTAL V is described in Higgins, D. G. and P. M. Sharp (1989; CABIOS 5:151-153) and in Higgins, D. G. et al. (1992; CABIOS 8:189-191). For pairwise alignments of polynucleotide sequences, the default parameters are set as follows: Ktuple=2, gap penalty=5, window=4, and “diagonals saved”=4. The “weighted” residue weight table is selected as the default. Percent identity is reported by CLUSTAL V as the “percent similarity” between aligned polynucleotide sequences.

Alternatively, a suite of commonly used and freely available sequence comparison algorithms which can be used is provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nih.gov/BLAST/. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at http://www.ncbi.nlm.nih.gov/gorf/bl2.html. The “BLAST 2 Sequences” tool can be used for both blastn and blastp (discussed below). BLAST programs are commonly used with gap and other parameters set to default settings. For example, to compare two nucleotide sequences, one may use blastn with the “BLAST 2 Sequences” tool Version 2.0.12 (Apr. 21, 2000) set at default parameters. Such default parameters may be, for example:
Matrix: BLOSUM62
Reward for match: 1
Penalty for mismatch: −2
Open Gap: 5 and Extension Gap: 2 penalties
Gap×drop-off. 50
Expect: 10
Word Size: 11
Filter: on

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide.

Percent identity between polypeptide sequences may be determined using the default parameters of the CLUSTAL V algorithm as incorporated into the MEGALIGN version 3.12e sequence alignment program (described and referenced above). For pairwise alignments of polypeptide sequences using CLUSTAL V, the default parameters are set as follows: Ktuple=1, gap penalty=3, window=5, and “diagonals saved”=5. The PAM250 matrix is selected as the default residue weight table. As with polynucleotide alignments, the percent identity is reported by CLUSTAL V as the “percent similarity” between aligned polypeptide sequence pairs.

Alternatively the NCBI BLAST software suite may be used. For example, for a pairwise comparison of two polypeptide sequences, one may use the “BLAST 2 Sequences” tool Version 2.0.12 (Apr. 21, 2000) with blastp set at default parameters. Such default parameters may be, for example:
Matrix: BLOSUM62
Open Gap: 11 and Extension Gap: 1 penalties
Gap×drop-off. 50
Expect: 10
Word Size: 3
Filter: on

Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

“Human artificial chromosomes” (HACs) are linear microchromosomes which may contain DNA sequences of about 6 kb to 10 Mb in size and which contain all of the elements required for chromosome replication, segregation and maintenance.

The term “humanized antibody” refers to an antibody molecule in which the amino acid sequence in the non-antigen binding regions has been altered so that the antibody more closely resembles a human antibody, and still retains its original binding ability.

“Hybridization” refers to the process by which a polynucleotide strand anneals with a complementary strand through base pairing under defined hybridization conditions. Specific hybridization is an indication that two nucleic acid sequences share a high degree of complementarity. Specific hybridization complexes form under permissive annealing conditions and remain hybridized after the “washing” step(s). The washing step(s) is particularly important in determining the stringency of the hybridization process, with more stringent conditions allowing less non-specific binding, i.e., binding between pairs of nucleic acid strands that are not perfectly matched. Permissive conditions for annealing of nucleic acid sequences are routinely determinable by one of ordinary skill in the art and may be consistent among hybridization experiments, whereas wash conditions may be varied among experiments to achieve the desired stringency, and therefore hybridization specificity. Permissive annealing conditions occur, for example, at 68° C. in the presence of about 6×SSC, about 1% (w/v) SDS, and about 100 μg/ml sheared, denatured salmon sperm DNA.

Generally, stringency of hybridization is expressed, in part, with reference to the temperature under which the wash step is carried out. Such wash temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. An equation for calculating Tₘ and conditions for nucleic acid hybridization are well known and can be found in Sambrook, J. et al. (1989) Molecular Cloning: A Laboratory Manual, 2^{nd} ed., vol. 1-3, Cold Spring Harbor Press, Plainview N.Y.; specifically see volume 2, chapter 9.

High stringency conditions for hybridization between polynucleotides of the present invention include wash conditions of 68° C. in the presence of about 0.2 x SSC and about 0.1% SDS, for 1 hour. Alternatively, temperatures of about 65° C., 60° C., 55° C., or 42° C. may be used. SSC concentration may be varied from about 0.1 to 2×SSC, with SDS being present at about 0.1%. Typically, blocking reagents are used to block non-specific hybridization. Such blocking reagents include, for instance, sheared and denatured salmon sperm DNA at about 100-200 μg/ml. Organic solvent, such as formamide at a concentration of about 35-50% v/v, may also be used under particular circumstances, such as for RNA:DNA hybridizations. Useful variations on these wash conditions will be readily apparent to those of ordinary skill in the art. Hybridization, particularly under high stringency conditions, may be suggestive of evolutionary similarity between the nucleotides. Such similarity is strongly indicative of a similar role for the nucleotides and their encoded polypeptides.

The term “hybridization complex” refers to a complex formed between two nucleic acids by virtue of the formation of hydrogen bonds between complementary bases. A hybridization complex may be formed in solution (e.g., C₀t or R₀t analysis) or formed between one nucleic acid present in solution and another nucleic acid immobilized on a solid support (e.g., paper, membranes, filters, chips, pins or glass slides, or any other appropriate substrate to which cells or their nucleic acids have been fixed).

The words “insertion” and “addition” refer to changes in an amino acid or polynucleotide sequence resulting in the addition of one or more amino acid residues or nucleotides, respectively.

“Immune response” can refer to conditions associated with inflammation, trauma, immune disorders, or infectious or genetic disease, etc. These conditions can be characterized by expression of various factors, e.g., cytokines, chemokines, and other signaling molecules, which may affect cellular and systemic defense systems.

An “immunogenic fragment” is a polypeptide or oligopeptide fragment of NAAP which is capable of eliciting an immune response when introduced into a living organism, for example, a mammal. The term “immunogenic fragment” also includes any polypeptide or oligopeptide fragment of NAAP which is useful in any of the antibody production methods disclosed herein or known in the art.

The term “microarray” refers to an arrangement of a plurality of polynucleotides, polypeptides, antibodies, or other chemical compounds on a substrate.

The terms “element” and “array element” refer to a polynucleotide, polypeptide, antibody, or other chemical compound having a unique and defined position on a microarray.

The term “modulate” refers to a change in the activity of NAAP. For example, modulation may cause an increase or a decrease in protein activity, binding characteristics, or any other biological, functional, or immunological properties of NAAP.

The phrases “nucleic acid” and “nucleic acid sequence” refer to a nucleotide, oligonucleotide, polynucleotide, or any fragment thereof. These phrases also refer to DNA or RNA of genomic or synthetic origin which may be single-stranded or double-stranded and may represent the sense or the antisense strand, to peptide nucleic acid (PNA), or to any DNA-like or RNA-like material.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

“Peptide nucleic acid” (PNA) refers to an antisense molecule or anti-gene agent which comprises an oligonucleotide of at least about 5 nucleotides in length linked to a peptide backbone of amino acid residues ending in lysine. The terminal lysine confers solubility to the composition. PNAs preferentially bind complementary single stranded DNA or RNA and stop transcript elongation, and may be pegylated to extend their lifespan in the cell.

“Post-translational modification” of an NAAP may involve lipidation, glycosylation, phosphorylation, acetylation, racemization, proteolytic cleavage, and other modifications known in the art. These processes may occur synthetically or biochemically. Biochemical modifications will vary by cell type depending on the enzymatic milieu of NAAP.

“Probe” refers to nucleic acids encoding NAAP, their complements, or fragments thereof, which are used to detect identical, allelic or related nucleic acids. Probes are isolated oligonucleotides or polynucleotides attached to a detectable label or reporter molecule. Typical labels include radioactive isotopes, ligands, chemiluminescent agents, and enzymes. “Priers” are short nucleic acids, usually DNA oligonucleotides, which may be annealed to a target polynucleotide by complementary base-pairing. The primer may then be extended along the target DNA strand by a DNA polymerase enzyme. Primer pairs can be used for amplification (and identification) of a nucleic acid, e.g., by the polymerase chain reaction (PCR).

Probes and primers as used in the present invention typically comprise at least 15 contiguous nucleotides of a known sequence. In order to enhance specificity, longer probes and primers may also be employed, such as probes and primers that comprise at least 20, 25, 30, 40, 50, 60, 70, 80, 90, 100, or at least 150 consecutive nucleotides of the disclosed nucleic acid sequences. Probes and primers may be considerably longer than these examples, and it is understood that any length supported by the specification, including the tables, figures, and Sequence Listing, may be used.

Methods for preparing and using probes and primers are described in the references, for example Sambrook, J. et al. (1989; Molecular Cloning: A Laboratory Manual, 2^{nd} ed., vol. 1-3, Cold Spring Harbor Press, Plainview N.Y.), Ausubel, F. M. et al. (1999) Short Protocols in Molecular Biology, 4^{th} ed., John Wiley & Sons, New York N.Y.), and Innis, M. et al. (1990; PCR Protocols, A Guide to Methods and Applications, Academic Press, San Diego Calif.). PCR primer pairs can be derived from a known sequence, for example, by using computer programs intended for that purpose such as Primer (Version 0.5, 1991, Whitehead Institute for Biomedical Research, Cambridge Mass.).

Oligonucleotides for use as primers are selected using software known in the art for such purpose. For example, OLIGO 4.06 software is useful for the selection of PCR primer pairs of up to 100 nucleotides each, and for the analysis of oligonucleotides and larger polynucleotides of up to 5,000 nucleotides from an input polynucleotide sequence of up to 32 kilobases. Similar primer selection programs have incorporated additional features for expanded capabilities. For example, the PrimOU primer selection program (available to the public from the Genome Center at University of Texas South West Medical Center, Dallas Tex.) is capable of choosing specific primers from megabase sequences and is thus useful for designing primers on a genome-wide scope. The Primer3 primer selection program (available to the public from the Whitehead Institute/MIT Center for Genome Research, Cambridge Mass.) allows the user to input a “mispriming library,” in which sequences to avoid as primer binding sites are user-specified. Primer3 is useful, in particular, for the selection of oligonucleotides for microarrays. (The source code for the latter two primer selection programs may also be obtained from their respective sources and modified to meet the user's specific needs.) The PrimeGen program (available to the public from the UK Human Genome Mapping Project Resource Centre, Cambridge UK) designs primers based on multiple sequence alignments, thereby allowing selection of primers that hybridize to either the most conserved or least conserved regions of aligned nucleic acid sequences. Hence, this program is useful for identification of both unique and conserved oligonucleotides and polynucleotide fragments. The oligonucleotides and polynucleotide fragments identified by any of the above selection methods are useful in hybridization technologies, for example, as PCR or sequencing primers, microarray elements, or specific probes to identify fully or partially complementary polynucleotides in a sample of nucleic acids. Methods of oligonucleotide selection are not limited to those described above.

A “recombinant nucleic acid” is a nucleic acid that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, supra. The term recombinant includes nucleic acids that have been altered'solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

Alternatively, such recombinant nucleic acids may be part of a viral vector, e.g., based on a vaccinia virus, that could be use to vaccinate a mammal wherein the recombinant nucleic acid is expressed, inducing a protective immunological response in the mammal.

A “regulatory element” refers to a nucleic acid sequence usually derived from untranslated regions of a gene and includes enhancers, promoters, introns, and 5′ and 3′ untranslated regions (UTRs). Regulatory elements interact with host or viral proteins which control transcription, translation, or RNA stability.

“Reporter molecules” are chemical or biochemical moieties used for labeling a nucleic acid, amino acid, or antibody. Reporter molecules include radionuclides; enzymes; fluorescent, chemiluminescent, or chromogenic agents; substrates; cofactors; inhibitors; magnetic particles; and other moieties known in the art.

An “RNA equivalent,” in reference to a DNA molecule, is composed of the same linear sequence of nucleotides as the reference DNA molecule with the exception that all occurrences of the nitrogenous base thymine are replaced with uracil, and the sugar backbone is composed of ribose instead of deoxyribose.

The term “sample” is used in its broadest sense. A sample suspected of containing NAAP, nucleic acids encoding NAAP, or fragments thereof may comprise a bodily fluid; an extract from a cell, chromosome, organelle, or membrane isolated from a cell; a cell; genomic DNA, RNA, or cDNA, in solution or bound to a substrate; a tissue; a tissue print; etc.

The terms “specific binding” and “specifically binding” refer to that interaction between a protein or peptide and an agonist, an antibody, an antagonist, a small molecule, or any natural or synthetic binding composition. The interaction is dependent upon the presence of a particular structure of the protein, e.g., the antigenic determinant or epitope, recognized by the binding molecule. For example, if an antibody is specific for epitope “A,” the presence of a polypeptide comprising the epitope A, or the presence of free unlabeled A, in a reaction containing free labeled A and the antibody will reduce the amount of labeled A that binds to the antibody.

The term “substantially purified” refers to nucleic acid or amino acid sequences that are removed from their natural environment and are isolated or separated, and are at least about 60% free, preferably at least about 75% free, and most preferably at least about 90% free from other components with which they are naturally associated.

A “substitution” refers to the replacement of one or more amino acid residues or nucleotides by different amino acid residues or nucleotides, respectively.

“Substrate” refers to any suitable rigid or semi-rigid support including membranes, filters, chips, slides, wafers, fibers, magnetic or nonmagnetic beads, gels, tubing, plates, polymers, microparticles and capillaries. The substrate can have a variety of surface forms, such as wells, trenches, pins, channels and pores, to which polynucleotides or polypeptides are bound.

A “transcript image” or “expression profile” refers to the collective pattern of gene expression by a particular cell type or tissue under given conditions at a given time.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

A “transgenic organism,” as used herein, is any organism, including but not limited to animals and plants, in which one or more of the cells of the organism contains heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. In another embodiment, the nucleic acid can be introduced by infection with a recombinant viral vector, such as a lentiviral vector (Lois, C. et al. (2002) Science 295:868-872). The term genetic manipulation does not include classical cross-breeding, or in vitro fertilization, but rather is directed to the introduction of a recombinant DNA molecule. The transgenic organisms contemplated in accordance with the present invention include bacteria, cyanobacteria, fungi, plants and animals. The isolated DNA of the present invention can be introduced into the host by methods known in the art, for example infection, transfection, transformation or transconjugation. Techniques for transferring the DNA of the present invention into such organisms are widely known and provided in references such as Sambrook et al. (1989), supra.

A “variant” of a particular nucleic acid sequence is defined as a nucleic acid sequence having at least 40% sequence identity to the particular nucleic acid sequence over a certain length of one of the nucleic acid sequences using blastn with the “BLAST 2 Sequences” tool Version 2.0.9 (May 07, 1999) set at default parameters. Such a pair of nucleic acids may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length. A variant may be described as, for example, an “alielic” (as defined above), “splice,” “species,” or “polymorphic” variant. A splice variant may have significant identity to a reference molecule, but will generally have a greater or lesser number of polynucleotides due to alternate splicing of exons during mRNA processing. The corresponding polypeptide may possess additional functional domains or lack domains that are present in the reference molecule. Species variants are polynucleotides that vary from one species to another. The resulting polypeptides will generally have significant amino acid identity relative to each other. A polymorphic variant is a variation in the polynucleotide sequence of a particular gene between individuals of a given species. Polymorphic variants also may encompass “single nucleotide polymorphisms” (SNPs) in which the polynucleotide sequence varies by one nucleotide base. The presence of SNPs may be indicative of, for example, a certain population, a disease state, or a propensity for a disease state.

A “variant” of a particular polypeptide sequence is defined as a polypeptide sequence having at least 40% sequence identity to the particular polypeptide sequence over a certain length of one of the polypeptide sequences using blastp with the “BLAST 2 Sequences” tool Version 2.0.9 (May 07, 1999) set at default parameters. Such a pair of polypeptides may show, for example, at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length of one of the polypeptides.

### THE INVENTION

Various embodiments of the invention include new human nucleic acid-associated proteins (NAAP), the polynucleotides encoding NAAP, and the use of these compositions for the diagnosis, treatment, or prevention of cell proliferative, DNA repair, neurological, reproductive, developmental, and autoimmune/inflammatory disorders, and infections.

Table 1 summarizes the nomenclature for the full length polynucleotide and polypeptide embodiments of the invention. Each polynucleotide and its corresponding polypeptide are correlated to a single Incyte project identification number (Incyte Project ID). Each polypeptide sequence is denoted by both a polypeptide sequence identification number (Polypeptide SEQ ID NO:) and an Incyte polypeptide sequence number (Incyte Polypeptide ID) as shown. Each polynucleotide sequence is denoted by both a polynucleotide sequence identification number (Polynucleotide SEQ ID NO:) and an Incyte polynucleotide consensus sequence number (Incyte Polynucleotide ID) as shown. Column 6 shows the Incyte ID numbers of physical, full length clones corresponding to polypeptide and polynucleotide embodiments. The full length clones encode polypeptides which have at least 95% sequence identity to the polypeptides shown in column 3.

Table 2 shows sequences with homology to the polypeptides of the invention as identified by BLAST analysis against the GenBank protein (genpept) database. Columns 1 and 2 show the polypeptide sequence identification number (Polypeptide SEQ ID NO:) and the corresponding Incyte polypeptide sequence number (Incyte Polypeptide ID) for polypeptides of the invention. Column 3 shows the GenBank identification number (GenBank ID NO:) of the nearest GenBank homolog. Column 4 shows the probability scores for the matches between each polypeptide and its homolog(s). Column 5 shows the annotation of the GenBank homolog(s) along with relevant citations where applicable, all of which are expressly incorporated by reference herein.

Table 3 shows various structural features of the polypeptides of the invention. Columns 1 and 2 show the polypeptide sequence identification number (SEQ ID NO:) and the corresponding Incyte polypeptide sequence number (Incyte Polypeptide ID) for each polypeptide of the invention. Column 3 shows the number of amino acid residues in each polypeptide. Column 4 shows potential phosphorylation sites, and column 5 shows potential glycosylation sites, as determined by the MOTIFS program of the GCG sequence analysis software package (Genetics Computer Group, Madison Wis.). Column 6 shows amino acid residues comprising signature sequences, domains, and motifs. Column 7 shows analytical methods for protein structure/function analysis and in some cases, searchable databases to which the analytical methods were applied.

Together, Tables 2 and 3 summarize the properties of polypeptides of the invention, and these properties establish that the claimed polypeptides are nucleic acid-associated proteins. For example, SEQ ID NO:3 is 94% identical, from residue Ml to residue G1023, to Mus musculus 5′-3′ exonuclease (GenBank ID g1894791) as determined by the Basic Local Alignment Search Tool (BLAST). (See Table 2.) The BLAST probability score is 0, which indicates the probability of obtaining the observed polypeptide sequence alignment by chance. (See Table 3.) Data from further BLAST and MOTIFS analyses provide further corroborative evidence that SEQ ID NO:3 is an exonuclease.

In another example, SEQ ID NO:7 is 85% identical, from residue S205 to residue S900, to Rattus norvegicus zinc finger protein RIN ZF (GenBank ID g4557143) as determined by the Basic Local Alignment Search Tool (BLAST). (See Table 2.) The BLAST probability score is 0.0, which indicates the probability of obtaining the observed polypeptide sequence alignment by chance. SEQ ID NO:7 also contains a BTB/POZ domain, and a C2H2-type zinc finger domain as determined by searching for statistically significant matches in the hidden Markov model (H)-based PFAM database of conserved protein family domains. (See Table 3.) Data from BUMPS, BLAST-PRODOM, BLAST-DOMO, and MOTIFS analyses provide further corroborative evidence that SEQ ID NO:7 is a zinc finger protein.

In another example, SEQ ID NO:16 is 81% identical, from residue Ml to residue D104, to human acidic ribosomal phosphoprotein (P1) (GenBank ID g190234) as determined by the Basic Local Alignment Search Tool (BLAST). (See Table 2.) The BLAST probability score is 2.7e-40, which indicates the probability of obtaining the observed polypeptide sequence alignment by chance. SEQ ID NO:16 also contains a 60s acidic ribosomal protein domain as determined by searching for statistically significant matches in the hidden Markov model (HMM)-based PFAM database of conserved protein family domains. (See Table 3.) Data from further BLAST analyses provide corroborative evidence that SEQ ID NO:16 is an acidic ribosomal phosphoprotein.

In another example, SEQ ID NO:18 is 68% identical, from residue A17 to residue Y1131, to Drosophila melanogaster RNA polymerase III second-largest subunit (GenBank ID g10963) as determined by the Basic Local Alignment Search Tool (BLAST). (See Table 2.) The BLAST probability score is 0.0, which indicates the probability of obtaining the observed polypeptide sequence alignment by chance. SEQ ID NO:18 also contains an RNA polymerase beta subunit domain as determined by searching for statistically significant matches in the hidden Markov model (HMM)-based PFAM database of conserved protein family domains. (See Table 3.) Data from BLIMPS, MOTIFS, and other BLAST analyses provide further corroborative evidence that SEQ ID NO:18 is an RNA polymerase beta subunit.

In another example, SEQ ID NO:19 is 72% identical, from residue MI to residue D2170, to mouse MGA protein (GenBank ID g6692607) as determined by the Basic Local Alignment Search Tool (BLAST). (See Table 2.) The BLAST probability score is 0.0, which indicates the probability of obtaining the observed polypeptide sequence alignment by chance. SEQ ID NO:19 also contains a helix-loop-helix DNA-binding domain and a T-box domain as determined by searching for statistically significant matches in the hidden Markov model (HMM)-based PFAM database of conserved protein family domains. (See Table 3.) Data from BLIMPS, MOTIFS, and other BLAST analyses provide further corroborative evidence that SEQ ID NO:19 is a MGA protein.

For example, SEQ ID NO:21 is 37% identical, from residue R514 to residue N1368, to Oryza sativa putative ATP-dependent RNA helicase A (GenBank ID g14090215) as determined by the Basic Local Alignment Search Tool (BLAST). (See Table 2.) The BLAST probability score is 2.4e-137, which indicates the probability of obtaining the observed polypeptide sequence alignment by chance. SEQ ID NO:21 also contains a DEAD/DEAH box helicase domain, a helicase conserved C-terminal domain, and a signal peptide as determined by searching for statistically significant matches in the hidden Markov model (HMM)-based PFAM database of conserved protein family domains. (See Table 3.) Data from BLIMPS, MOTIFS, and PROFILESCAN analyses and BLAST analyses of the PRODOM and DOMO databases provide further corroborative evidence that SEQ ID NO:21 is a helicase.

In a further example, SEQ ID NO:22 is 99% identical, from residue Q243 to residue E589 and 38% identical, from residue S151 to residue E589, to human zinc finger protein ZNF131 (GenBank ID g493572) as determined by the Basic Local Alignment Search Tool (BLAST). (See Table 2.) The BLAST probability score is 7.5e-191, which indicates the probability of obtaining the observed polypeptide sequence alignment by chance. SEQ ID NO:22 also contains BTB/POZ and C2H2 type zinc finger domains as determined by searching for statistically significant matches in the hidden Markov model (HMM)-based PFAM database of conserved protein family domains. (See Table 3.) Data from MOTIFS analysis and BLAST analysis of the DOMO database provide further corroborative evidence that SEQ ID NO:22 is a zinc finger protein. SEQ ID NO:1-2, SEQ ID NO:4-6, SEQ ID NO:8-15, SEQ ID NO:17, SEQ ID NO:20 and SEQ ID NO:23-33 were analyzed and annotated in a similar manner. The algorithms and parameters for the analysis of SEQ ID NO:1-33 are described in Table 7.

As shown in Table 4, the full length polynucleotide embodiments were assembled using cDNA sequences or coding (exon) sequences derived from genomic DNA, or any combination of these two types of sequences. Column 1 lists the polynucleotide sequence identification number (Polynucleotide SEQ ID NO:), the corresponding Incyte polynucleotide consensus sequence number (Incyte ID) for each polynucleotide of the invention, and the length of each polynucleotide sequence in basepairs. Column 2 shows the nucleotide start (5′) and stop (3′) positions of the cDNA and/or genomic sequences used to assemble the full length polynucleotide embodiments, and of fragments of the polynucleotides which are useful, for example, in hybridization or amplification technologies that identify SEQ ID NO:34-66 or that distinguish between SEQ ID NO:34-66 and related polynucleotides.

The polynucleotide fragments described in Column 2 of Table 4 may refer specifically, for example, to Incyte cDNAs derived from tissue-specific cDNA libraries or from pooled cDNA libraries. Alternatively, the polynucleotide fragments described in column 2 may refer to GenBank cDNAs or ESTs which contributed to the assembly of the full length polynucleotides. In addition, the polynucleotide fragments described in column 2 may identify sequences derived from the ENSEMBL (The Sanger Centre, Cambridge, UK) database (i.e., those sequences including the designation “ENST”). Alternatively, the polynucleotide fragments described in column 2 may be derived from the NCBI RefSeq Nucleotide Sequence Records Database (i.e., those sequences including the designation “NM” or “NT”) or the NCBI RefSeq Protein Sequence Records (i.e., those sequences including the designation “NP”). Alternatively, the polynucleotide fragments described in column 2 may refer to assemblages of both cDNA and Genscan-predicted exons brought together by an “exon stitching” algorithm For example, a polynucleotide sequence identified as FL_XXXXX_N₁_{^{—}}N₂_{^{—}}YYYYY_N₃_{^{—}}N₄_{^{—}} represents a “stitched” sequence in which XXXXXX is the identification number of the cluster of sequences to which the algorithm was applied, and YYYYY is the number of the prediction generated by the algorithm, and N_{1,2,3 . . .} , if present, represent specific exons that may have been manually edited during analysis (See Example V). Alternatively, the polynucleotide fragments in column 2 may refer to assemblages of exons brought together by an “exon-stretching” algorithm. For example, a polynucleotide sequence identified as FLXXXXXX_gAAAAA_gBBBBB_{—}1_N is a “stretched” sequence, with XXXX being the Incyte project identification number, gAAAAA being the GenBank identification number of the human genomic sequence to which the “exon-stretching” algorithm was applied, GBBBBB being the GenBank identification number or NCBI RefSeq identification number of the nearest GenBank protein homolog, and N referring to specific exons (See Example V). In instances where a RefSeq sequence was used as a protein homolog for the “exon-stretching” algorithm, a RefSeq identifier (denoted by “NM,” “NP,” or “NT”) may be used in place of the GenBank identifier (i.e., gBBBBB).

Alternatively, a prefix identifies component sequences that were hand-edited, predicted from genomic DNA sequences, or derived from a combination of sequence analysis methods. The following Table lists examples of component sequence prefixes and corresponding sequence analysis methods associated with the prefixes (see Example IV and Example V).

| Prefix | Type of analysis and/or examples of programs |
|---|---|
| GNN, | Exon prediction from genomic sequences using, for example, |
| GFG, | GENSCAN (Stanford University, CA, USA) or FGENES |
| ENST | (Computer Genomics Group, The Sanger Centre, |
| | Cambridge, UK). |
| GBI | Hand-edited analysis of genomic sequences. |
| FL | Stitched or stretched genomic sequences (see Example V). |
| INCY | Full length transcript and exon prediction from mapping of EST |
| | sequences to the genome. Genomic location and EST |
| | composition data are combined to predict the exons and |
| | resulting transcript. |

In some cases, Incyte cDNA coverage redundant with the sequence coverage shown in Table 4 was obtained to confirm the final consensus polynucleotide sequence, but the relevant Incyte cDNA identification numbers are not shown.

Table 5 shows the representative cDNA libraries for those full length polynucleotides which were assembled using Incyte cDNA sequences. The representative cDNA library is the Incyte cDNA library which is most frequently represented by the Incyte cDNA sequences which were used to assemble and confirm the above polynucleotides. The tissues and vectors which were used to construct the cDNA libraries shown in Table 5 are described in Table 6.

The invention also encompasses NAAP variants. A preferred NAAP variant is one which has at least about 80%, or alternatively at least about 90%, or even at least about 95% amino acid sequence identity to the NAAP amino acid sequence, and which contains at least one functional or structural characteristic of NAAP.

Various embodiments also encompass polynucleotides which encode NAAP. In a particular embodiment, the invention encompasses a polynucleotide sequence comprising a sequence selected from the group consisting of SEQ ID NO:34-66, which encodes NAAP. The polynucleotide sequences of SEQ ID NO:34-66, as presented in the Sequence Listing, embrace the equivalent RNA sequences, wherein occurrences of the nitrogenous base thymine are replaced with uracil, and the sugar backbone is composed of ribose instead of deoxyribose.

The invention also encompasses variants of a polynucleotide encoding NAAP. In particular, such a variant polynucleotide will have at least about 70%, or alternatively at least about 85%, or even at least about 95% polynucleotide sequence identity to a polynucleotide encoding NAAP. A particular aspect of the invention encompasses a variant of a polynucleotide comprising a sequence selected from the group consisting of SEQ ID NO:34-66 which has at least about 70%, or alternatively at least about 85%, or even at least about 95% polynucleotide sequence identity to a nucleic acid sequence selected from the group consisting of SEQ ID NO:34-66. Any one of the polynucleotide variants described above can encode a polypeptide which contains at least one functional or structural characteristic of NAAP.

In addition, or in the alternative, a polynucleotide variant of the invention is a splice variant of a polynucleotide encoding NAAP. A splice variant may have portions which have significant sequence identity to a polynucleotide encoding NAAP, but will generally have a greater or lesser number of polynucleotides due to additions or deletions of blocks of sequence arising from alternate splicing of exons during mRNA processing. A splice variant may have less than about 70%, or alternatively less than about 60%, or alternatively less than about 50% polynucleotide sequence identity to a polynucleotide encoding NAAP over its entire length; however, portions of the splice variant will have at least about 70%, or alternatively at least about 85%, or alternatively at least about 95%, or alternatively 100% polynucleotide sequence identity to portions of the polynucleotide encoding NAAP. For example, a polynucleotide comprising a sequence of SEQ ID NO:36 and a polynucleotide comprising a sequence of SEQ ID NO:61 are splice variants of each other. Any one of the splice variants described above can encode a polypeptide which contains at least one functional or structural characteristic of NAAP.

It will be appreciated by those skilled in the art that as a result of the degeneracy of the genetic code, a multitude of polynucleotide sequences encoding NAAP, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring NAAP, and all such variations are to be considered as being specifically disclosed.

Although polynucleotides which encode NAAP and its variants are generally capable of hybridizing to polynucleotides encoding naturally occurring NAAP under appropriately selected conditions of stringency, it may be advantageous to produce polynucleotides encoding NAAP or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding NAAP and its derivatives without altering the encoded amino acid sequences include the production of RNA transcripts having more desirable properties, such as a greater half-life, than transcripts produced from the naturally occurring sequence.

The invention also encompasses production of polynucleotides which encode NAAP and NAAP derivatives, or fragments thereof, entirely by synthetic chemistry. After production, the synthetic polynucleotide may be inserted into any of the many available expression vectors and cell systems using reagents well known in the art. Moreover, synthetic chemistry may be used to introduce mutations into a polynucleotide encoding NAAP or any fragment thereof.

Embodiments of the invention can also include polynucleotides that are capable of hybridizing to the claimed polynucleotides, and, in particular, to those having the sequences shown in SEQ ID NO:34-66 and fragments thereof, under various conditions of stringency (Wahl, G. M. and S. L. Berger (1987) Methods Enzymol. 152:399-407; Kimmel, A. R. (1987) Methods Enzymol. 152:507-511). Hybridization conditions, including annealing and wash conditions, are described in “Definitions.”

Methods for DNA sequencing are well known in the art and may be used to practice any of the embodiments of the invention. The methods may employ such enzymes as the Klenow fragment of DNA polymerase I, SEQUENASE (US Biochemical, Cleveland Ohio), Taq polymerase (Applied Biosystems), thermostable T7 polymerase (Amersham Biosciences, Piscataway N.J.), or combinations of polymerases and proofreading exonucleases such as those found in the ELONGASE amplification system (Invitrogen, Carlsbad Calif.). Preferably, sequence preparation is automated with machines such as the MICROLAB 2200 liquid transfer system (Hamilton, Reno Nev.), PTC200 thermal cycler (M J Research, Watertown Mass.) and ABI CATALYST 800 thermal cycler (Applied Biosystems). Sequencing is then carried out using either the ABI 373 or 377 DNA sequencing system (Applied Biosystems), the MEGABACE 1000 DNA sequencing system (Amersham Biosciences), or other systems known in the art. The resulting sequences are analyzed using a variety of algorithms which are well known in the art (Ausubel et al., supra, ch. 7; Meyers, R. A. (1995) Molecular Biology and Biotechnology, Wiley VCH, New York N.Y., pp. 856-853).

The nucleic acids encoding NAAP may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector (Sarkar, G. (1993) PCR Methods Applic. 2:318-322). Another method, inverse PCR, uses primers that extend in divergent directions to amplify unknown sequence from a circularized template. The template is derived from restriction fragments comprising a known genomic locus and surrounding sequences (Triglia, T. et al. (1988) Nucleic Acids Res. 16:8186). A third method, capture PCR, involves PCR amplification of DNA fragments adjacent to known sequences in human and yeast artificial chromosome DNA (Lagerstrom, M. et al. (1991) PCR Methods Applic. 1:111-119). In this method, multiple restriction enzyme digestions and ligations may be used to insert an engineered double-stranded sequence into a region of unknown sequence before performing PCR. Other methods which may be used to retrieve unknown sequences are known in the art (Parker, J. D. et al. (1991) Nucleic Acids Res. 19:3055-3060). Additionally, one may use PCR, nested primers, and PROMOTERFINDER libraries (Clontech, Palo Alto Calif.) to walk genomic DNA. This procedure avoids the need to screen libraries and is useful in finding intron/exon junctions. For all PCR-based methods, primers may be designed using commercially available software, such as OLIGO 4.06 primer analysis software (National Biosciences, Plymouth Minn.) or another appropriate program, to be about 22 to 30 nucleotides in length, to have a GC content of about 50% or more, and to anneal to the template at temperatures of about 68° C. to 72° C.

When screening for full length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems which are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., GENOTYPER and SEQUENCE NAVIGATOR, Applied Biosystems), and the entire process from loading of samples to computer analysis and electronic data display may be computer controlled Capillary electrophoresis is especially preferable for sequencing small DNA fragments which may be present in limited amounts in a particular sample.

In another embodiment of the invention, polynucleotides or fragments thereof which encode NAAP may be cloned in recombinant DNA molecules that direct expression of NAAP, or fragments or functional equivalents thereof, in appropriate host cells. Due to the inherent degeneracy of the genetic code, other polynucleotides which encode substantially the same or a functionally equivalent polypeptides may be produced and used to express NAAP.

The polynucleotides of the invention can be engineered using methods generally known in the art in order to alter NAAP-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

The nucleotides of the present invention may be subjected to DNA shuffling techniques such as MOLECULARBREEDING (Maxygen Inc., Santa Clara Calif.; described in U.S. Pat. No. 5,837,458; Chang, C.-C. et al. (1999) Nat. Biotechnol. 17:793-797; Christians, F. C. et al. (1999) Nat. Biotechnol. 17:259-264; and Crameri, A. et al. (1996) Nat. Biotechnol. 14:315-319) to alter or improve the biological properties of NAAP, such as its biological or enzymatic activity or its ability to bind to other molecules or compounds. DNA shuffling is a process by which a library of gene variants is produced using PCR-mediated recombination of gene fragments. The library is then subjected to selection or screening procedures that identify those gene variants with the desired properties. These preferred variants may then be pooled and further subjected to recursive rounds of DNA shuffling and selection/screening. Thus, genetic diversity is created through “artificial” breeding and rapid molecular evolution. For example, fragments of a single gene containing random point mutations may be recombined, screened, and then reshuffled until the desired properties are optimized. Alternatively, fragments of a given gene may be recombined with fragments of homologous genes in the same gene family, either from the same or different species, thereby maximizing the genetic diversity of multiple naturally occurring genes in a directed and controllable manner.

In another embodiment, polynucleotides encoding NAAP may be synthesized, in whole or in part, using one or more chemical methods well known in the art (Caruthers, M. H. et al. (1980) Nucleic Acids Symp. Ser. 7:215-223; Horn, T. et al. (1980) Nucleic Acids Symp. Ser. 7:225-232). Alternatively, NAAP itself or a fragment thereof may be synthesized using chemical methods known in the art. For example, peptide synthesis can be performed using various solution-phase or solid-phase techniques (Creighton, T. (1984) Proteins, Structures and Molecular Properties, W H Freeman, New York N.Y., pp. 55-60; Roberge, J. Y. et al. (1995) Science 269:202-204). Automated synthesis may be achieved using the ABI 431A peptide synthesizer (Applied Biosystems). Additionally, the amino acid sequence of NAAP, or any part thereof, may be altered during direct synthesis and/or combined with sequences from other proteins, or any part thereof, to produce a variant polypeptide or a polypeptide having a sequence of a naturally occurring polypeptide.

The peptide may be substantially purified by preparative high performance liquid chromatography (Chiez, R. M. and F. Z. Regnier (1990) Methods Enzymol. 182:392-421). The composition of the synthetic peptides may be confirmed by amino acid analysis or by sequencing. (Creighton, supra, pp. 28-53).

In order to express a biologically active NAAP, the polynucleotides encoding NAAP or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotides encoding NAAP. Such elements may vary in their strength and specificity. Specific initiation signals may also be used to achieve more efficient translation of polynucleotides encoding NAAP. Such signals include the ATG initiation codon and adjacent sequences, e.g. the Kozak sequence. In cases where a polynucleotide sequence encoding NAAP and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used (Scharf, D. et al. (1994) Results Probl. Cell Differ. 20:125-162).

Methods which are well known to those skilled in the art may be used to construct expression vectors containing polynucleotides encoding NAAP and appropriate transcriptional and translation control elements. These methods include in vitro recombinant DNA techniques, synthetic techniques, and in vivo genetic recombination (Sambrook, J. et al. (1989) Molecular Cloning, A Laboratory Manual, Cold Spring Harbor Press, Plainview N.Y., ch. 4, 8, and 16-17; Ausubel et al., supra, ch. 1, 3, and 15).

A variety of expression vector/host systems may be utilized to contain and express polynucleotides encoding NAAP. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); plant cell systems transformed with viral expression vectors (e.g., cauliflower mosaic virus, CaMV, or tobacco mosaic virus, TMV) or with bacterial expression vectors (e.g., Ti or pBR322 plasmids); or animal cell systems (Sambrook, supra; Ausubel et al., supra; Van Heeke, G. and S. M. Schuster (1989) J. Biol. Chem. 264:5503-5509; Engelhard, E. K. et al. (1994) Proc. Natl. Acad. Sci. USA 91:3224-3227; Sandig, V. et al. (1996) Hum. Gene Ther. 7:1937-1945; Takamatsu, N. (1987) EMBO J. 6:307-311; The McGraw Hill Yearbook of Science and Technology (1992) McGraw Hill, New York N.Y., pp. 191-196; Logan, J. and T. Shenk (1984) Proc. Natl. Acad. Sci. USA 81:3655-3659; Harrington, J. J. et al. (1997) Nat. Genet. 15:345-355). Expression vectors derived from retroviruses, adenoviruses, or herpes or vaccinia viruses, or from various bacterial plasmids, may be used for delivery of polynucleotides to the targeted organ, tissue, or cell population (Di Nicola, M. et al. (1998) Cancer Gen. Ther. 5:350-356; Yu, M. et al. (1993) Proc. Natl. Acad. Sci. USA 90:6340-6344; Buller, R. M. et al. (1985) Nature 317:813-815; McGregor, D. P. et al. (1994) Mol. Immunol. 31:219-226; Verma, I. M. and N. Somia (1997) Nature 389:239-242). The invention is not limited by the host cell employed.

In bacterial systems, a number of cloning and expression vectors may be selected depending upon the use intended for polynucleotides encoding NAAP. For example, routine cloning, subcloning, and propagation of polynucleotides encoding NAAP can be achieved using a multifunctional E. coli vector such as PBLUESCRIPT (Stratagene, La Jolla Calif.) or PSPORT1 plasmid (Invitrogen). Ligation of polynucleotides encoding NAAP into the vector's multiple cloning site disrupts the lacZ gene, allowing a colorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence (Van Heeke, G. and S. M. Schuster (1989) J. Biol. Chem. 264:5503-5509). When large quantities of NAAP are needed, e.g. for the production of antibodies, vectors which direct high level expression of NAAP may be used. For example, vectors containing the strong, inducible SP6 or T7 bacteriophage promoter may be used.

Yeast expression systems may be used for production of NAAP. A number of vectors containing constitutive or inducible promoters, such as alpha factor, alcohol oxidase, and PGH promoters, may be used in the yeast Saccharomyces cerevisiae or Pichia pastoris. In addition, such vectors direct either the secretion or intracellular retention of expressed proteins and enable integration of foreign polynucleotide sequences into the host genome for stable propagation (Ausubel et al., supra; Bitter, G. A. et al. (1987) Methods Enzymol. 153:516-544; Scorer, C. A. et al. (1994) Bio/Technology 12:181-184).

Plant systems may also be used for expression of NAAP. Transcription of polynucleotides encoding NAAP may be driven by viral promoters, e.g., the 35S and 19S promoters of CaMV used alone or in combination with the omega leader sequence from TMV (Takamatsu, N. (1987) EMBO J. 6:307-311). Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; Winter, J. et al. (1991) Results Probl. Cell Differ. 17:85-105). These constructs can be introduced into plant cells by direct DNA transformation or pathogen-mediated transfection (The McGraw Hill Yearbook of Science and Technology (1992) McGraw Hill, New York N.Y., pp. 191-196).

In mammalian cells, a number of viral-based expression systems may be utilized. In cases where an adenovirus is used as an expression vector, polynucleotides encoding NAAP may be ligated into an adenovirus transcription/translation complex consisting of the late promoter and tripartite leader sequence. Insertion in a non-essential E1 or E3 region of the viral genome may be used to obtain infective virus which expresses NAAP in host cells (Logan, J. and T. Shenk (1984) Proc. Natl. Acad. Sci. USA 81:3655-3659). In addition, transcription enhancers, such as the Rous sarcoma virus (RSV) enhancer, may be used to increase expression in mammalian host cells. SV40 or EBV-based vectors may also be used for high-level protein expression.

Human artificial chromosomes (HACs) may also be employed to deliver larger fragments of DNA than can be contained in and expressed from a plasmid. HACs of about 6 kb to 10 Mb are constructed and delivered via conventional delivery methods (liposomes, polycationic amino polymers, or vesicles) for therapeutic purposes (Harrington, J. J. et al. (1997) Nat. Genet. 15:345-355).

For long term production of recombinant proteins in mammalian systems, stable expression of NAAP in cell lines is preferred. For example, polynucleotides encoding NAAP can be transformed into cell lines using expression vectors which may contain viral origins of replication and/or endogenous expression elements and a selectable marker gene on the same or on a separate vector. Following the introduction of the vector, cells may be allowed to grow for about 1 to 2 days in enriched media before being switched to selective media. The purpose of the selectable marker is to confer resistance to a selective agent, and its presence allows growth and recovery of cells which successfully express the introduced sequences. Resistant clones of stably transformed cells may be propagated using tissue culture techniques appropriate to the cell type.

Any number of selection systems may be used to recover transformed cell lines. These include, but are not limited to, the herpes simplex virus thymidine kinase and adenine phosphoribosyltransferase genes, for use in tk⁻ and apr⁻ cells, respectively (Wigler, M. et al. (1977) Cell 11:223-232; Lowy, L. et al. (1980) Cell 22:817-823). Also, antimetabolite, antibiotic, or herbicide resistance can be used as the basis for selection. For example, dhfr confers resistance to methotrexate; neo confers resistance to the aminoglycosides neomycin and G-418; and als and pat confer resistance to chlorsulfuron and phosphinotricin acetyltransferase, respectively (Wigler, M. et al. (1980) Proc. Natl. Acad. Sci. USA 77:3567-3570; Colbere-Garapin, F. et al. (1981) J. Mol. Biol. 150:1-14). Additional selectable genes have been described, e.g., trpB and hisD, which alter cellular requirements for metabolites (Hartman, S. C. and R. C. Mulligan (1988) Proc. Natl. Acad. Sci. USA 85:8047-8051). Visible markers, e.g., anthocyanins, green fluorescent proteins (GFP; Clontech), β-glucuronidase and its substrate β-glucuronide, or luciferase and its substrate luciferin may be used. These markers can be used not only to identify transformants, but also to quantify the amount of transient or stable protein expression attributable to a specific vector system (Rhodes, C. A. (1995) Methods Mol. Biol. 55:121-131).

Although the presence/absence of marker gene expression suggests that the gene of interest is also present, the presence and expression of the gene may need to be confirmed. For example, if the sequence encoding NAAP is inserted within a marker gene sequence, transformed cells containing polynucleotides encoding NAAP can be identified by the absence of marker gene function. Alternatively, a marker gene can be placed in tandem with a sequence encoding NAAP under the control of a single promoter. Expression of the marker gene in response to induction or selection usually indicates expression of the tandem gene as well.

In general, host cells that contain the polynucleotide encoding NAAP and that express NAAP may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of NAAP using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on NAAP is preferred, but a competitive binding assay may be employed. These and other assays are well known in the art (Hampton, R. et al. (1990) Serological Methods, a Laboratory Manual, APS Press, St. Paul Minn., Sect. IV; Coligan, J. E. et al. (1997) Current Protocols in Immunology, Greene Pub. Associates and Wiley-Interscience, New York N.Y.; Pound, J. D. (1998) Immunochemical Protocols, Humana Press, Totowa N.J.).

A wide variety of labels and conjugation techniques are known by those skilled in the art and may be used in various nucleic acid and amino acid assays. Means for producing labeled hybridization or PCR probes for detecting sequences related to polynucleotides encoding NAAP include oligolabeling, nick translation, end-labeling, or PCR amplification using a labeled nucleotide. Alternatively, polynucleotides encoding NAAP, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Biosciences, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Host cells transformed with polynucleotides encoding NAAP may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode NAAP may be designed to contain signal sequences which direct secretion of NAAP through a prokaryotic or eukaryotic cell membrane.

In addition, a host cell strain may be chosen for its ability to modulate expression of the inserted polynucleotides or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” or “pro” form of the protein may also be used to specify protein targeting, folding, and/or activity. Different host cells which have specific cellular machinery and characteristic mechanisms for post-translational activities (e.g., CHO, HeLa, MDCK, HEK293, and WI38) are available from the American Type Culture Collection (ATCC, Manassas Va.) and may be chosen to ensure the correct modification and processing of the foreign protein.

In another embodiment of the invention, natural, modified, or recombinant polynucleotides encoding NAAP may be ligated to a heterologous sequence resulting in translation of a fusion protein in any of the aforementioned host systems. For example, a chimeric NAAP protein containing a heterologous moiety that can be recognized by a commercially available antibody may facilitate the screening of peptide libraries for inhibitors of NAAP activity. Heterologous protein and peptide moieties may also facilitate purification of fusion proteins using commercially available affinity matrices. Such moieties include, but are not limited to, glutathione S-transferase (GST), maltose binding protein (MBP), thioredoxin (Trx), calmodulin binding peptide (CBP), 6-His, FLAG, c-myc, and hemagglutinin (HA). GST, MBP, Trx, CBP, and 6-His enable purification of their cognate fusion proteins on immobilized glutathione, maltose, phenylarsine oxide, calmodulin, and metal-chelate resins, respectively. FLAG, c-myc, and hemagglutinin (HA) enable immunoaffinity purification of fusion proteins using commercially available monoclonal and polyclonal antibodies that specifically recognize these epitope tags. A fusion protein may also be engineered to contain a proteolytic cleavage site located between the NAAP encoding sequence and the heterologous protein sequence, so that NAAP may be cleaved away from the heterologous moiety following purification. Methods for fusion protein expression and purification are discussed in Ausubel et al. (supra, ch. 10 and 16). A variety of commercially available kits may also be used to facilitate expression and purification of fusion proteins.

In another embodiment, synthesis of radiolabeled NAAP may be achieved in vitro using the TNT rabbit reticulocyte lysate or wheat germ extract system (Promega). These systems couple transcription and translation of protein-coding sequences operably associated with the 17, T3, or SP6 promoters. Translation takes place in the presence of a radiolabeled amino acid precursor, for example, ³⁵S-methionine.

NAAP, fragments of NAAP, or variants of NAAP may be used to screen for compounds that specifically bind to NAAP. One or more test compounds may be screened for specific binding to NAAP. In various embodiments, 1, 2, 3, 4, 5, 10, 20, 50, 100, or 200 test compounds can be screened for specific binding to NAAP. Examples of test compounds can include antibodies, anticalins, oligonucleotides, proteins (e.g., ligands or receptors), or small molecules.

In related embodiments, variants of NAAP can be used to screen for binding of test compounds, such as antibodies, to NAAP, a variant of NAAP, or a combination of NAAP and/or one or more variants NAAP. In an embodiment, a variant of NAAP can be used to screen for compounds that bind to a variant of NAAP, but not to NAAP having the exact sequence of a sequence of SEQ ID NO:1-33. NAAP variants used to perform such screening can have a range of about 50% to about 99% sequence identity to NAAP, with various embodiments having 60%, 70%, 75%, 80%, 85%, 90%, and 95% sequence identity.

In an embodiment, a compound identified in a screen for specific binding to NAAP can be closely related to the natural ligand of NAAP, e.g., a ligand or fragment thereof, a natural substrate, a structural or functional mimetic, or a natural binding partner (Coligan, J. E. et al. (1991) Current Protocols in Immunology 1(2):Chapter 5). In another embodiment, the compound thus identified can be a natural ligand of a receptor NAAP (Howard, A. D. et al. (2001) Trends Pharmacol. Sci.22: 132-140; Wise, A. et al. (2002) Drug Discovery Today 7:235-246).

In other embodiments, a compound identified in a screen for specific binding to NAAP can be closely related to the natural receptor to which NAAP binds, at least a fragment of the receptor, or a fragment of the receptor including all or a portion of the ligand binding site or binding pocket. For example, the compound may be a receptor for NAAP which is capable of propagating a signal, or a decoy receptor for NAAP which is not capable of propagating a signal (Ashkenazi, A. and V. M. Divit (1999) Curr. Opin. Cell Biol. 11:255-260; Mantovani, A. et al. (2001) Trends Immunol. 22:328-336). The compound can be rationally designed using known techniques. Examples of such techniques include those used to construct the compound etanercept (ENBREL; Amgen Inc., Thousand Oaks Calif.), which is efficacious for treating rheumatoid arthritis in humans. Etanercept is an engineered p75 tumor necrosis factor (TNF) receptor dimer linked to the Fc portion of human IgG₁ (Taylor, P. C. et al. (2001) Curr. Opin. Immunol. 13:611-616).

In one embodiment, two or more antibodies having similar or, alternatively, different specificities can be screened for specific binding to NAAP, fragments of NAAP, or variants of NAAP. The binding specificity of the antibodies thus screened can thereby be selected to identify particular fragments or variants of NAAP. In one embodiment, an antibody can be selected such that its binding specificity allows for preferential identification of specific fragments or variants of NAAP. In another embodiment, an antibody can be selected such that its binding specificity allows for preferential diagnosis of a specific disease or condition having increased, decreased, or otherwise abnormal production of NAAP.

In an embodiment, anticalins can be screened for specific binding to NAAP, fragments of NAAP, or variants of NAAP. Anticalins are ligand-binding proteins that have been constructed based on a lipocalin scaffold (Weiss, G. A. and H. B. Lowman (2000) Chem. Biol. 7:R177-R184; Skerra, A. (2001) J. Biotechnol. 74:257-275). The protein architecture of lipocalins can include a beta-barrel having eight antiparallel beta-strands, which supports four loops at its open end. These loops form the natural ligand-binding site of the lipocalins, a site which can be re-engineered in vitro by amino acid substitutions to impart novel binding specificities. The amino acid substitutions can be made using methods known in the art or described herein, and can include conservative substitutions (e.g., substitutions that do not alter binding specificity) or substitutions that modestly, moderately, or significantly alter binding specificity.

In one embodiment, screening for compounds which specifically bind to, stimulate, or inhibit NAAP involves producing appropriate cells which express NAAP, either as a secreted protein or on the cell membrane. Preferred cells include cells from mammals, yeast, Drosophila, or E. coli. Cells expressing NAAP or cell membrane fractions which contain NAAP are then contacted with a test compound and binding, stimulation, or inhibition of activity of either NAAP or the compound is analyzed.

An assay may simply test binding of a test compound to the polypeptide, wherein binding is detected by a fluorophore, radioisotope, enzyme conjugate, or other detectable label. For example, the assay may comprise the steps of combining at least one test compound with NAAP, either in solution or affixed to a solid support, and detecting the binding of NAAP to the compound. Alternatively, the assay may detect or measure binding of a test compound in the presence of a labeled competitor. Additionally, the assay may be carried out using cell-free preparations, chemical libraries, or natural product mixtures, and the test compound(s) may be free in solution or affixed to a solid support.

An assay can be used to assess the ability of a compound to bind to its natural ligand and/or to inhibit the binding of its natural ligand to its natural receptors. Examples of such assays include radio-labeling assays such as those described in U.S. Pat. No. 5,914,236 and U.S. Pat. No. 6,372,724. In a related embodiment, one or more amino acid substitutions can be introduced into a polypeptide compound (such as a receptor) to improve or alter its ability to bind to its natural ligands (Matthews, D. J. and J. A. Wells. (1994) Chem. Biol. 1:25-30). In another related embodiment, one or more amino acid substitutions can be introduced into a polypeptide compound (such as a ligand) to improve or alter its ability to bind to its natural receptors (Cunningham, B. C. and J. A. Wells (1991) Proc. Natl. Acad. Sci. USA 88:3407-3411; Lowman, H. B. et al. (1991) J. Biol. Chem. 266:10982-10988).

NAAP, fragments of NAAP, or variants of NAAP may be used to screen for compounds that modulate the activity of NAAP. Such compounds may include agonists, antagonists, or partial or inverse agonists. In one embodiment, an assay is performed under conditions permissive for NAAP activity, wherein NAAP is combined with at least one test compound, and the activity of NAAP in the presence of a test compound is compared with the activity of NAAP in the absence of the test compound. A change in the activity of NAAP in the presence of the test compound is indicative of a compound that modulates the activity of NAAP. Alternatively, a test compound is combined with an in vitro or cell-free system comprising NAAP under conditions suitable for NAAP activity, and the assay is performed. In either of these assays, a test compound which modulates the activity of NAAP may do so indirectly and need not come in direct contact with the test compound. At least one and up to a plurality of test compounds may be screened.

In another embodiment, polynucleotides encoding NAAP or their mammalian homologs may be “knocked out” in an animal model system using homologous recombination in embryonic stem (ES) cells. Such techniques are well known in the art and are useful for the generation of animal models of human disease (see, e.g., U.S. Pat. No. 5,175,383 and U.S. Pat. No. 5,767,337). For example, mouse ES cells, such as the mouse 129/SvJ cell line, are derived from the early mouse embryo and grown in culture. The ES cells are transformed with a vector containing the gene of interest disrupted by a marker gene, e.g., the neomycin phosphotransferase gene (neo; Capecchi, M. R. (1989) Science 244:1288-1292). The vector integrates into the corresponding region of the host genome by homologous recombination. Alternatively, homologous recombination takes place using the Cre-loxP system to knockout a gene of interest in a tissue- or developmental stage-specific manner (Marth, J. D. (1996) Clin. Invest. 97:1999-2002; Wagner, K. U. et al. (1997) Nucleic Acids Res. 25:4323-4330). Transformed ES cells are identified and microinjected into mouse cell blastocysts such as those from the C57BL/6 mouse strain. The blastocysts are surgically transferred to pseudopregnant dams, and the resulting chimeric progeny are genotyped and bred to produce heterozygous or homozygous strains. Transgenic animals thus generated may be tested with potential therapeutic or toxic agents.

Polynucleotides encoding NAAP may also be manipulated in vitro in ES cells derived from human blastocysts. Human ES cells have the potential to differentiate into at least eight separate cell lineages including endoderm, mesoderm, and ectodermal cell types. These cell lineages differentiate into, for example, neural cells, hematopoietic lineages, and cardiomyocytes (Thomson, J. A. et al. (1998) Science 282:1145-1147).

Polynucleotides encoding NAAP can also be used to create “knockin” humanized animals (pigs) or transgenic animals (mice or rats) to model human disease. With knockin technology, a region of a polynucleotide encoding NAAP is injected into animal ES cells, and the injected sequence integrates into the animal cell genome. Transformed cells are injected into blastulae, and the blastulae are implanted as described above. Transgenic progeny or inbred lines are studied and treated with potential pharmaceutical agents to obtain information on treatment of a human disease. Alternatively, a mammal inbred to overexpress NAAP, e.g., by secreting NAAP in its milk, may also serve as a convenient source of that protein (Janne, J. et al. (1998) Biotechnol. Annu. Rev. 4:55-74).

Therapeutics

Chemical and structural similarity, e.g., in the context of sequences and motifs, exists between regions of NAAP and nucleic acid-associated proteins. In addition, examples of tissues expressing NAAP can be found in Table 6 and can also be found in Example XI. Therefore, NAAP appears to play a role in cell proliferative, DNA repair, neurological, reproductive, developmental, and autoimmune/inflammatory disorders, and infections. In the treatment of disorders associated with increased NAAP expression or activity, it is desirable to decrease the expression or activity of NAAP. In the treatment of disorders associated with decreased NAAP expression or activity, it is desirable to increase the expression or activity of NAAP.

Therefore, in one embodiment, NAAP or a fragment or derivative thereof may be administered to a subject to treat or prevent a disorder associated with decreased expression or activity of NAAP. Examples of such disorders include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, a cancer of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; a DNA repair disorder such as xeroderma pigmentosum, Bloom's syndrome, and Werner's syndrome; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorder of the central nervous system, cerebral palsy, a neuroskeletal disorder, an autonomic nervous system disorder, a cranial nerve disorder, a spinal cord disease, muscular dystrophy and other neuromuscular disorder, a peripheral nervous system disorder, dermatomyositis and polymyositis, inherited, metabolic, endocrine, and toxic myopathy, myasthenia gravis, periodic paralysis, a mental disorder including mood, anxiety, and schizophrenic disorder, seasonal affective disorder (SAD), akathesia, amnesia, catatonia, diabetic neuropathy, tardive dyskinesia, dystonias, paranoid psychoses, postherpetic neuralgia, and Tourette's disorder, a reproductive disorder such as a disorder of prolactin production, infertility, including tubal disease, ovulatory defects, and endometriosis, a disruption of the estrous cycle, a disruption of the menstrual cycle, polycystic ovary syndrome, ovarian hyperstimulation syndrome, endometrial and ovarian tumors, uterine fibroids, autoimmune disorders, ectopic pregnancies, and teratogenesis; cancer of the breast, fibrocystic breast disease, and galactorrhea; a disruption of spermatogenesis, abnormal sperm physiology, cancer of the testis, cancer of the prostate, benign prostatic hyperplasia, prostatitis, Peyronie's disease, impotence, carcinoma of the male breast, and gynecomastia; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; an infection, such as those caused by a viral agent classified as adenovirus, arenavirus, bunyavirus, calicivirus, coronavirus, filovirus, hepadnavirus, herpesvirus, flavivirus, orthomyxovirus, parvovirus, papovavirus, paramyxovirus, picornavirus, poxvirus, reovirus, retrovirus, rhabdovirus, or togavirus; an infection caused by a bacterial agent classified as pneumococcus, staphylococcus, streptococcus, bacillus, corynebacterium, clostridium, meningococcus, gonococcus, listeria, moraxella, kingella, haemophilus, legionella, bordetella, gram-negative enterobacterium including shigella, salmonella, or campylobacter, pseudomonas, vibrio, brucella, francisella, yersinia, bartonella, norcardium, actinomyces, mycobacterium, spirochaetale, rickettsia, chlamydia, or mycoplasma; an infection caused by a fungal agent classified as aspergillus, blastomyces, dermatophytes, cryptococcus, coccidioides, malasezzia, histoplasma, or other mycosis-causing fungal agent; and an infection caused by a parasite classified as plasmodium or malaria-causing, parasitic entamoeba, leishmania, trypanosoma, toxoplasma, pneumocystis carinii, intestinal protozoa such as giardia, trichomonas, tissue nematode such as trichinella, intestinal nematode such as ascaris, lymphatic filarial nematode, trematode such as schistosoma, and cestode such as tapeworm.

In another embodiment, a vector capable of expressing NAAP or a fragment or derivative thereof may be administered to a subject to treat or prevent a disorder associated with decreased expression or activity of NAAP including, but not limited to, those described above.

In a further embodiment, a composition comprising a substantially purified NAAP in conjunction with a suitable pharmaceutical carrier may be administered to a subject to treat or prevent a disorder associated with decreased expression or activity of NAAP including, but not limited to, those provided above.

In still another embodiment, an agonist which modulates the activity of NAAP may be administered to a subject to treat or prevent a disorder associated with decreased expression or activity of NAAP including, but not limited to, those listed above.

In a further embodiment, an antagonist of NAAP may be administered to a subject to treat or prevent a disorder associated with increased expression or activity of NAAP. Examples of such disorders include, but are not limited to, those cell proliferative, DNA repair, neurological, reproductive, developmental, and autoimmune/inflammatory disorders, and infections described above. In one aspect, an antibody which specifically binds NAAP may be used directly as an antagonist or indirectly as a targeting or delivery mechanism for bringing a pharmaceutical agent to cells or tissues which express NAAP.

In an additional embodiment, a vector expressing the complement of the polynucleotide encoding NAAP may be administered to a subject to treat or prevent a disorder associated with increased expression or activity of NAAP including, but not limited to, those described above.

In other embodiments, any protein, agonist, antagonist, antibody, complementary sequence, or vector embodiments may be administered in combination with other appropriate therapeutic agents. Selection of the appropriate agents for use in combination therapy may be made by one of ordinary skill in the art, according to conventional pharmaceutical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects.

An antagonist of NAAP may be produced using methods which are generally known in the art. In particular, purified NAAP may be used to produce antibodies or to screen libraries of pharmaceutical agents to identify those which specifically bind NAAP. Antibodies to NAAP may also be generated using methods that are well known in the art. Such antibodies may include, but are not limited to, polyclonal, monoclonal, chimeric, and single chain antibodies, Fab fragments, and fragments produced by a Fab expression library. Neutralizing antibodies (i.e., those which inhibit dimer formation) are generally preferred for therapeutic use. Single chain antibodies (e.g., from camels or llamas) may be potent enzyme inhibitors and may have advantages in the design of peptide mimetics, and in the development of immuno-adsorbents and biosensors (Muyldermans, S. (2001) J. Biotechnol. 74:277-302).

For the production of antibodies, various hosts including goats, rabbits, rats, mice, camels, dromedaries, llamas, humans, and others may be immunized by injection with NAAP or with any fragment or oligopeptide thereof which has immunogenic properties. Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KLH, and dinitrophenol. Among adjuvants used in humans, BCG (bacilli Calmette-Guerin) and Corynebacterium parvum are especially preferable.

It is preferred that the oligopeptides, peptides, or fragments used to induce antibodies to NAAP have an amino acid sequence consisting of at least about 5 amino acids, and generally will consist of at least about 10 amino acids. It is also preferable that these oligopeptides, peptides, or fragments are identical to a portion of the amino acid sequence of the natural protein. Short stretches of NAAP amino acids may be fused with those of another protein, such as KLH, and antibodies to the chimeric molecule may be produced.

Monoclonal antibodies to NAAP may be prepared using any technique which provides for the production of antibody molecules by continuous cell lines in culture. These include, but are not limited to, the hybridoma technique, the human B-cell hybridoma technique, and the EBV-hybridoma technique (Kohler, G. et al. (1975) Nature 256:495-497; Kozbor, D. et al. (1985) J. Immunol. Methods 81:3142; Cote, R. J. et al. (1983) Proc. Natl. Acad. Sci. USA 80:2026-2030; Cole, S. P. et al. (1984) Mol. Cell Biol. 62:109-120).

In addition, techniques developed for the production of “chimeric antibodies,” such as the splicing of mouse antibody genes to human antibody genes to obtain a molecule with appropriate antigen specificity and biological activity, can be used (Morrison, S. L. et al. (1984) Proc. Natl. Acad. Sci. USA 81:6851-6855; Neuberger, M. S. et al. (1984) Nature 312:604-608; Takeda, S. et al. (1985) Nature 314:452-454). Alternatively, techniques described for the production of single chain antibodies may be adapted, using methods known in the art, to produce NAAP-specific single chain antibodies. Antibodies with related specificity, but of distinct idiotypic composition, may be generated by chain shuffling from random combinatorial immunoglobulin libraries (Burton, D. R. (1991) Proc. Natl. Acad. Sci. USA 88:10134-10137).

Antibodies may also be produced by inducing in vivo production in the lymphocyte population or by screening immunoglobulin libraries or panels of highly specific binding reagents as disclosed in the literature (Orlandi, R. et al. (1989) Proc. Natl. Acad. Sci. USA 86:3833-3837; Winter, G. et al. (1991) Nature 349:293-299).

Antibody fragments which contain specific binding sites for NAAP may also be generated. For example, such fragments include, but are not limited to, F(ab′)₂ fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity (Huse, W. D. et al. (1989) Science 246:1275-1281).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between NAAP and its specific antibody. A two-site, monoclonal-based immunoassay utilizing monoclonal antibodies reactive to two non-interfering NAAP epitopes is generally used, but a competitive binding assay may also be employed (Pound, supra).

Various methods such as Scatchard analysis in conjunction with radioimmunoassay techniques may be used to assess the affinity of antibodies for NAAP. Affinity is expressed as an association constant, Kₐ, which is defined as the molar concentration of NAAP-antibody complex divided by the molar concentrations of free antigen and free antibody under equilibrium conditions. The Kₐ determined for a preparation of polyclonal antibodies, which are heterogeneous in their affinities for multiple NAAP epitopes, represents the average affinity, or avidity, of the antibodies for NAAP. The Kₐ determined for a preparation of monoclonal antibodies, which are monospecific for a particular NAAP epitope, represents a true measure of affinity. High-affinity antibody preparations with Kₐ ranging from about 10⁹ to 10¹² L/mole are preferred for use in immunoassays in which the NAAP-antibody complex must withstand rigorous manipulations. Low-affinity antibody preparations with Kₐ ranging from about 10⁶ to 10⁷ L/mole are preferred for use in immunopurification and similar procedures which ultimately require dissociation of NAAP, preferably in active form, from the antibody (Catty, D. (1988) Antibodies, Volume I: A Practical Approach, IRL Press, Washington D.C.; Liddell, J. E. and A. Cryer (1991) A Practical Guide to Monoclonal Antibodies, John Wiley & Sons, New York N.Y.).

The titer and avidity of polyclonal antibody preparations may be further evaluated to determine the quality and suitability of such preparations for certain downstream applications. For example, a polyclonal antibody preparation containing at least 1-2 mg specific antibody/ml, preferably 5-10 mg specific antibody/ml, is generally employed in procedures requiring precipitation of NAAP-antibody complexes. Procedures for evaluating antibody specificity, titer, and avidity, and guidelines for antibody quality and usage in various applications, are generally available (Catty, supra; Coligan et al., supra).

In another embodiment of the invention, polynucleotides encoding NAAP, or any fragment or complement thereof, may be used for therapeutic purposes. In one aspect, modifications of gene expression can be achieved by designing complementary sequences or antisense molecules (DNA, RNA, PNA, or modified oligonucleotides) to the coding or regulatory regions of the gene encoding NAAP. Such technology is well known in the art, and antisense oligonucleotides or larger fragments can be designed from various locations along the coding or control regions of sequences encoding NAAP (Agrawal, S., ed. (1996) Antisense Therapeutics, Humana Press, Totawa N.J.).

In therapeutic use, any gene delivery system suitable for introduction of the antisense sequences into appropriate target cells can be used. Antisense sequences can be delivered intracellularly in the form of an expression plasmid which, upon transcription, produces a sequence complementary to at least a portion of the cellular sequence encoding the target protein (Slater, J. E. et al. (1998) J. Allergy Clin. Immunol. 102:469-475; Scanlon, K. J. et al. (1995) 9:1288-1296). Antisense sequences can also be introduced intracellularly through the use of viral vectors, such as retrovirus and adeno-associated virus vectors (Miller, A. D. (1990) Blood 76:271; Ausubel et al., supra; Uckert, W. and W. Walther (1994) Pharmacol. Ther. 63:323-347). Other gene delivery mechanisms include liposome-derived systems, artificial viral envelopes, and other systems known in the art (Rossi, J. J. (1995) Br. Med. Bull. 51:217-225; Boado, R. J. et al. (1998) J. Pharm. Sci. 87:1308-1315; Morris, M. C. et al. (1997) Nucleic Acids Res. 25:2730-2736).

In another embodiment of the invention, polynucleotides encoding NAAP may be used for somatic or germline gene therapy. Gene therapy may be performed to (i) correct a genetic deficiency (e.g., in the cases of severe combined immunodeficiency (SCID)-X1 disease characterized by X-linked inheritance (Cavazzana-Calvo, M. et al. (2000) Science 288:669-672), severe combined immunodeficiency syndrome associated with an inherited adenosine deaminase (ADA) deficiency (Blaese, R. M. et al. (1995) Science 270:475-480; Bordignon, C. et al. (1995) Science 270:470-475), cystic fibrosis (Zabner, J. et al. (1993) Cell 75:207-216; Crystal, R. G. et al. (1995) Hum. Gene Therapy 6:643-666; Crystal, R. G. et al. (1995) Hum. Gene Therapy 6:667-703), thalassamias, familial hypercholesterolemia, and hemophilia resulting from Factor VIII or Factor IX deficiencies (Crystal, R. G. (1995) Science 270:404-410; Verma, I. M. and N. Somia (1997) Nature 389:239-242)), (ii) express a conditionally lethal gene product (e.g., in the case of cancers which result from unregulated cell proliferation), or (iii) express a protein which affords protection against intracellular parasites (e.g., against human retroviruses, such as human immunodeficiency virus (HIV) (Baltimore, D. (1988) Nature 335:395-396; Poeschla, E. et al. (1996) Proc. Natl. Acad. Sci. USA 93:11395-11399), hepatitis B or C virus (HBV, HCV); fungal parasites, such as Candida albicans and Paracoccidioides brasiliensis; and protozoan parasites such as Plasmodium falciparum and Trypanosoma cruzi). In the case where a genetic deficiency in NAAP expression or regulation causes disease, the expression of NAAP from an appropriate population of transduced cells may alleviate the clinical manifestations caused by the genetic deficiency.

In a further embodiment of the invention, diseases or disorders caused by deficiencies in NAAP are treated by constructing mammalian expression vectors encoding NAAP and introducing these vectors by mechanical means into NAAP-deficient cells. Mechanical transfer technologies for use with cells in vivo or ex vitro include (i) direct DNA microinjection into individual cells, (ii) ballistic gold particle delivery, (iii) liposome-mediated transfection, (iv) receptor-mediated gene transfer, and (v) the use of DNA transposons (Morgan, R. A. and W. F. Anderson (1993) Annu. Rev. Biochem. 62:191-217; Ivics, Z. (1997) Cell 91:501-510; Boulay, J.-L. and H. Récipon (1998) Curr. Opin. Biotechnol. 9:445-450).

Expression vectors that may be effective for the expression of NAAP include, but are not limited to, the PCDNA 3.1, EPITAG, PRCCMV2, PREP, PVAX, PCR2-TOPOTA vectors (Invitrogen, Carlsbad Calif.), PCMV-SCRIPT, PCMV-TAG, PEGSH/PERV (Stratagene, La Jolla Calif.), and PTET-OFF, PTET-ON, PTRE2, PTRE2-LUC, PTK-HYG (Clontech, Palo Alto Calif.). NAAP may be expressed using (i) a constitutively active promoter, (e.g., from cytomegalovirus (CMV), Rous sarcoma virus (RSV), SV40 virus, thymidine kinase (TK), or β-actin genes), (ii) an inducible promoter (e.g., the tetracycline-regulated promoter (Gossen, M. and H. Bujard (1992) Proc. Natl. Acad. Sci. USA 89:5547-5551; Gossen, M. et al. (1995) Science 268:1766-1769; Rossi, F. M. V. and H. M. Blau (1998) Curr. Opin. Biotechnol. 9:451-456), commercially available in the T-REX plasmid (Invitrogen)); the ecdysone-inducible promoter (available in the plasmids PVGRXR and PIND; Invitrogen); the FK506/rapamycin inducible promoter; or the RU486/mifepristone inducible promoter (Rossi, F. M. V. and H. M. Blau, supra)), or (iii) a tissue-specific promoter or the native promoter of the endogenous gene encoding NAAP from a normal individual.

Commercially available liposome transformation kits (e.g., the PERFECT LIPID TRANSFECTION KIT, available from Invitrogen) allow one with ordinary skill in the art to deliver polynucleotides to target cells in culture and require minimal effort to optimize experimental parameters. In the alternative, transformation is performed using the calcium phosphate method (Graham, F. L. and A. J. Eb (1973) Virology 52:456-467), or by electroporation (Neumann, E. et al. (1982) EMBO J. 1:841-845). The introduction of DNA to primary cells requires modification of these standardized mammalian transfection protocols.

In another embodiment of the invention, diseases or disorders caused by genetic defects with respect to NAAP expression are treated by constructing a retrovirus vector consisting of (i) the polynucleotide encoding NAAP under the control of an independent promoter or the retrovirus long terminal repeat (LTR) promoter, (ii) appropriate RNA packaging signals, and (iii) a Rev-responsive element (RRE) along with additional retrovirus cis-acting RNA sequences and coding sequences required for efficient vector propagation. Retrovirus vectors (e.g., PFB and PFBNEO) are commercially available (Stratagene) and are based on published data (Riviere, I. et al. (1995) Proc. Natl. Acad. Sci. USA 92:6733-6737), incorporated by reference herein. The vector is propagated in an appropriate vector producing cell line (VPCL) that expresses an envelope gene with a tropism for receptors on the target cells or a promiscuous envelope protein such as VSVg (Armentano, D. et al. (1987) J. Virol. 61:1647-1650; Bender, M. A. et al. (1987) J. Virol. 61:1639-1646; Adam, M. A. and A. D. Miller (1988) J. Virol. 62:3802-3806; Dull, T. et al. (1998) J. Virol. 72:8463-8471; Zufferey, R. et al. (1998) J. Virol. 72:9873-9880). U.S. Pat. No. 5,910,434 to Rigg (“Method for obtaining retrovirus packaging cell lines producing high transducing efficiency retroviral supernatant”) discloses a method for obtaining retrovirus packaging cell lines and is hereby incorporated by reference. Propagation of retrovirus vectors, transduction of a population of cells (e.g., CD4⁺ T-cells), and the return of transduced cells to a patient are procedures well known to persons skilled in the art of gene therapy and have been well documented (Ranga, U. et al. (1997) J. Virol. 71:7020-7029; Bauer, G. et al. (1997) Blood 89:2259-2267; Bonyhadi, M. L. (1997) J. Virol. 71:4707-4716; Ranga, U. et al. (1998) Proc. Natl. Acad. Sci. USA 95:1201-1206; Su, L. (1997) Blood 89:2283-2290).

In an embodiment, an adenovirus-based gene therapy delivery system is used to deliver polynucleotides encoding NAAP to cells which have one or more genetic abnormalities with respect to the expression of NAAP. The construction and packaging of adenovirus-based vectors are well known to those with ordinary skill in the art. Replication defective adenovirus vectors have proven to be versatile for importing genes encoding immunoregulatory proteins into intact islets in the pancreas (Csete, M. E. et al. (1995) Transplantation 27:263-268). Potentially useful adenoviral vectors are described in U.S. Pat. No. 5,707,618 to Armentano (“Adenovirus vectors for gene therapy”), hereby incorporated by reference. For adenoviral vectors, see also Antinozzi, P. A. et al. (1999; Annu. Rev. Nutr. 19:511-544) and Verma, I. M. and N. Somia (1997; Nature 18:389:239-242).

In another embodiment, a herpes-based, gene therapy delivery system is used to deliver polynucleotides encoding NAAP to target cells which have one or more genetic abnormalities with respect to the expression of NAAP. The use of herpes simplex virus (HSV)-based vectors may be especially valuable for introducing NAAP to cells of the central nervous system, for which HSV has a tropism. The construction and packaging of herpes-based vectors are well known to those with ordinary skill in the art. A replication-competent herpes simplex virus (HSV) type 1-based vector has been used to deliver a reporter gene to the eyes of primates (Liu, X. et al. (1999) Exp. Eye Res. 169:385-395). The construction of a HSV-1 virus vector has also been disclosed in detail in U.S. Pat. No. 5,804,413 to DeLuca (“Herpes simplex virus strains for gene transfer”), which is hereby incorporated by reference. U.S. Pat. No. 5,804,413 teaches the use of recombinant HSV d92 which consists of a genome containing at least one exogenous gene to be transferred to a cell under the control of the appropriate promoter for purposes including human gene therapy. Also taught by this patent are the construction and use of recombinant HSV strains deleted for ICP4, ICP27 and ICP22. For HSV vectors, see also Goins, W. F. et al. (1999; J. Virol. 73:519-532) and Xu, H. et al. (1994; Dev. Biol. 163:152-161). The manipulation of cloned herpesvirus sequences, the generation of recombinant virus following the transfection of multiple plasmids containing different segments of the large herpesvirus genomes, the growth and propagation of herpesvirus, and the infection of cells with herpesvirus are techniques well known to those of ordinary skill in the art.

In another embodiment, an alphavirus (positive, single-stranded RNA virus) vector is used to deliver polynucleotides encoding NAAP to target cells. The biology of the prototypic alphavirus, Semliki Forest Virus (SFV), has been studied extensively and gene transfer vectors have been based on the SFV genome (Garoff, H. and K.-J. Li (1998) Curr. Opin. Biotechnol. 9:464-469). During alphavirus RNA replication, a subgenomic RNA is generated that normally encodes the viral capsid proteins. This subgenomic RNA replicates to higher levels than the full length genomic RNA, resulting in the overproduction of capsid proteins relative to the viral proteins with enzymatic activity (e.g., protease and polymerase). Similarly, inserting the coding sequence for NAAP into the alphavirus genome in place of the capsid-coding region results in the production of a large number of NAAP-coding RNAs and the synthesis of high levels of NAAP in vector transduced cells. While alphavirus infection is typically associated with cell lysis within a few days, the ability to establish a persistent infection in hamster normal kidney cells (BHK-21) with a variant of Sindbis virus (SIN) indicates that the lytic replication of alphaviruses can be altered to suit the needs of the gene therapy application (Dryga, S. A. et al. (1997) Virology 228:74-83). The wide host range of alphaviruses will allow the introduction of NAAP into a variety of cell types. The specific transduction of a subset of cells in a population may require the sorting of cells prior to transduction. The methods of manipulating infectious cDNA clones of alphaviruses, performing alphavirus cDNA and RNA transfections, and performing alphavirus infections, are well known to those with ordinary skill in the art.

Oligonucleotides derived from the transcription initiation site, e.g., between about positions −10 and +10 from the start site, may also be employed to inhibit gene expression. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) in Huber, B. E. and B. I Carr, Molecular and Immunologic Approaches, Futura Publishing, Mt. Kisco N.Y., pp. 163-177). A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of RNA molecules encoding NAAP.

Specific ribozyme cleavage sites within any potential RNA target are initially identified by scanning the target molecule for ribozyme cleavage sites, including the following sequences: GUA, GUL, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary oligonucleotides using ribonuclease protection assays.

Complementary ribonucleic acid molecules and ribozymes may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA molecules encoding NAAP. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

An additional embodiment of the invention encompasses a method for screening for a compound which is effective in altering expression of a polynucleotide encoding NAAP. Compounds which may be effective in altering expression of a specific polynucleotide may include, but are not limited to, oligonucleotides, antisense oligonucleotides, triple helix-forming oligonucleotides, transcription factors and other polypeptide transcriptional regulators, and non-macromolecular chemical entities which are capable of interacting with specific polynucleotide sequences. Effective compounds may alter polynucleotide expression by acting as either inhibitors or promoters of polynucleotide expression. Thus, in the treatment of disorders associated with increased NAAP expression or activity, a compound which specifically inhibits expression of the polynucleotide encoding NAAP may be therapeutically useful, and in the treatment of disorders associated with decreased NAAP expression or activity, a compound which specifically promotes expression of the polynucleotide encoding NAAP may be therapeutically useful.

At least one, and up to a plurality, of test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding NAAP is exposed to at least one test compound thus obtained. The sample may comprise, for example, an intact or permeabilized cell, or an in vitro cell-free or reconstituted biochemical system. Alterations in the expression of a polynucleotide encoding NAAP are assayed by any method commonly known in the art. Typically, the expression of a specific nucleotide is detected by hybridization with a probe having a nucleotide sequence complementary to the sequence of the polynucleotide encoding NAAP. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide. A screen for a compound effective in altering expression of a specific polynucleotide can be carried out, for example, using a Schizosaccharomyces pombe gene expression system (Atkins, D. et al. (1999) U.S. Pat. No. 5,932,435; Arndt, G. M. et al. (2000) Nucleic Acids Res. 28:E15) or a human cell line such as HeLa cell (Clarke, M. L. et al. (2000) Biochem. Biophys. Res. Commun. 268:8-13). A particular embodiment of the present invention involves screening a combinatorial library of oligonucleotides (such as deoxyribonucleotides, ribonucleotides, peptide nucleic acids, and modified oligonucleotides) for antisense activity against a specific polynucleotide sequence (Bruice, T. W. et al. (1997) U.S. Pat. No. 5,686,242; Bruice, T. W. et al. (2000) U.S. Pat. No. 6,022,691).

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art (Goldman, C. K. et al. (1997) Nat. Biotechnol. 15:462-466).

Any of the therapeutic methods described above may be applied to any subject in need of such therapy, including, for example, mammals such as humans, dogs, cats, cows, horses, rabbits, and monkeys.

An additional embodiment of the invention relates to the administration of a composition which generally comprises an active ingredient formulated with a pharmaceutically acceptable excipient. Excipients may include, for example, sugars, starches, celluloses, gums, and proteins. Various formulations are commonly known and are thoroughly discussed in the latest edition of Remington's Pharmaceutical Sciences (Maack Publishing, Easton Pa.). Such compositions may consist of NAAP, antibodies to NAAP, and mimetics, agonists, antagonists, or inhibitors of NAAP.

The compositions utilized in this invention may be administered by any number of routes including, but not limited to, oral, intravenous, intramuscular, intra-arterial, intramedullary, intrathecal, intraventricular, pulmonary, transdermal, subcutaneous, intraperitoneal, intranasal, enteral, topical, sublingual, or rectal means.

Compositions for pulmonary administration may be prepared in liquid or dry powder form. These compositions are generally aerosolized immediately prior to inhalation by the patient. In the case of small molecules (e.g. traditional low molecular weight organic drugs), aerosol delivery of fast-acting formulations is well-known in the art In the case of macromolecules (e.g. larger peptides and proteins), recent developments in the field of pulmonary delivery via the alveolar region of the lung have enabled the practical delivery of drugs such as insulin to blood circulation (see, e.g., Patton, J. S. et al., U.S. Pat. No. 5,997,848). Pulmonary delivery has the advantage of administration without needle injection, and obviates the need for potentially toxic penetration enhancers.

Compositions suitable for use in the invention include compositions wherein the active ingredients are contained in an effective amount to achieve the intended purpose. The determination of an effective dose is well within the capability of those skilled in the art.

Specialized forms of compositions may be prepared for direct intracellular delivery of macromolecules comprising NAAP or fragments thereof. For example, liposome preparations containing a cell-impermeable macromolecule may promote cell fusion and intracellular delivery of the macromolecule. Alternatively, NAAP or a fragment thereof may be joined to a short cationic N-terminal portion from the HIV Tat-1 protein. Fusion proteins thus generated have been found to transduce into the cells of all tissues, including the brain, in a mouse model system (Schwarze, S. R. et al. (1999) Science 285:1569-1572).

For any compound, the therapeutically effective dose can be estimated initially either in cell culture assays, e.g., of neoplastic cells, or in animal models such as mice, rats, rabbits, dogs, monkeys, or pigs. An animal model may also be used to determine the appropriate concentration range and route of administration. Such information can then be used to determine useful doses and routes for administration in humans.

A therapeutically effective dose refers to that amount of active ingredient, for example NAAP or fragments thereof, antibodies of NAAP, and agonists, antagonists or inhibitors of NAAP, which ameliorates the symptoms or condition. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The dose ratio of toxic to therapeutic effects is the therapeutic index, which can be expressed as the LD₅₀/ED₅₀ ratio. Compositions which exhibit large therapeutic indices are preferred. The data obtained from cell culture assays and animal studies are used to formulate a range of dosage for human use. The dosage contained in such compositions is preferably within a range of circulating concentrations that includes the ED₅₀ with little or no toxicity. The dosage varies within this range depending upon the dosage form employed, the sensitivity of the patient, and the route of administration.

The exact dosage will be determined by the practitioner, in light of factors related to the subject requiring treatment. Dosage and administration are adjusted to provide sufficient levels of the active moiety or to maintain the desired effect. Factors which may be taken into account include the severity of the disease state, the general health of the subject, the age, weight, and gender of the subject, time and frequency of administration, drug combination(s), reaction sensitivities, and response to therapy. Long-acting compositions may be administered every 3 to 4 days, every week, or biweekly depending on the half-life and clearance rate of the particular formulation.

Normal dosage amounts may vary from about 0.1 μg to 100,000 μg, up to a total dose of about 1 gram, depending upon the route of administration. Guidance as to particular dosages and methods of delivery is provided in the literature and generally available to practitioners in the art. Those skilled in the art will employ different formulations for nucleotides than for proteins or their inhibitors. Similarly, delivery of polynucleotides or polypeptides will be specific to particular cells, conditions, locations, etc.

Diagnostics

In another embodiment, antibodies which specifically bind NAAP may be used for the diagnosis of disorders characterized by expression of NAAP, or in assays to monitor patients being treated with NAAP or agonists, antagonists, or inhibitors of NAAP. Antibodies useful for diagnostic purposes may be prepared in the same manner as described above for therapeutics. Diagnostic assays for NAAP include methods which utilize the antibody and a label to detect NAAP in human body fluids or in extracts of cells or tissues. The antibodies may be used with or without modification, and may be labeled by covalent or non-covalent attachment of a reporter molecule. A wide variety of reporter molecules, several of which are described above, are known in the art and may be used.

A variety of protocols for measuring NAAP, including ELISAs, RIAs, and FACS, are known in the art and provide a basis for diagnosing altered or abnormal levels of NAAP expression. Normal or standard values for NAAP expression are established by combining body fluids or cell extracts taken from normal mammalian subjects, for example, human subjects, with antibodies to NAAP under conditions suitable for complex formation. The amount of standard complex formation may be quantitated by various methods, such as photometric means. Quantities of NAAP expressed in subject, control, and disease samples from biopsied tissues are compared with the standard values. Deviation between standard and subject values establishes the parameters for diagnosing disease.

In another embodiment of the invention, polynucleotides encoding NAAP may be used for diagnostic purposes. The polynucleotides which may be used include oligonucleotides, complementary RNA and DNA molecules, and PNAs. The polynucleotides may be used to detect and quantify gene expression in biopsied tissues in which expression of NAAP may be correlated with disease. The diagnostic assay may be used to determine absence, presence, and excess expression of NAAP, and to monitor regulation of NAAP levels during therapeutic intervention.

In one aspect, hybridization with PCR probes which are capable of detecting polynucleotides, including genomic sequences, encoding NAAP or closely related molecules may be used to identify nucleic acid sequences which encode NAAP. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding NAAP, allelic variants, or related sequences.

Probes may also be used for the detection of related sequences, and may have at least 50% sequence identity to any of the NAAP encoding sequences. The hybridization probes of the subject invention may be DNA or RNA and may be derived from the sequence of SEQ ID NO:34-66 or from genomic sequences including promoters, enhancers, and introns of the NAAP gene.

Means for producing specific hybridization probes for polynucleotides encoding NAAP include the cloning of polynucleotides encoding NAAP or NAAP derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, and the like.

Polynucleotides encoding NAAP may be used for the diagnosis of disorders associated with expression of NAAP. Examples of such disorders include, but are not limited to, a cell proliferative disorder such as actinic keratosis, arteriosclerosis, atherosclerosis, bursitis, cirrhosis, hepatitis, mixed connective tissue disease (MCTD), myelofibrosis, paroxysmal nocturnal hemoglobinuria, polycythemia vera, psoriasis, primary thrombocythemia, and cancers including adenocarcinoma, leukemia, lymphoma, melanoma, myeloma, sarcoma, teratocarcinoma, and, in particular, a cancer of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus; a DNA repair disorder such as xeroderma pigmentosum, Bloom's syndrome, and Werner's syndrome; a neurological disorder such as epilepsy, ischemic cerebrovascular disease, stroke, cerebral neoplasms, Alzheimer's disease, Pick's disease, Huntington's disease, dementia, Parkinson's disease and other extrapyramidal disorders, amyotrophic lateral sclerosis and other motor neuron disorders, progressive neural muscular atrophy, retinitis pigmentosa, hereditary ataxias, multiple sclerosis and other demyelinating diseases, bacterial and viral meningitis, brain abscess, subdural empyema, epidural abscess, suppurative intracranial thrombophlebitis, myelitis and radiculitis, viral central nervous system disease, prion diseases including kuru, Creutzfeldt-Jakob disease, and Gerstmann-Straussler-Scheinker syndrome, fatal familial insomnia, nutritional and metabolic diseases of the nervous system, neurofibromatosis, tuberous sclerosis, cerebelloretinal hemangioblastomatosis, encephalotrigeminal syndrome, mental retardation and other developmental disorder of the central nervous system, cerebral palsy, a neuroskeletal disorder, an autonomic nervous system disorder, a cranial nerve disorder, a spinal cord disease, muscular dystrophy and other neuromuscular disorder, a peripheral nervous system disorder, dermatomyositis and polymyositis, inherited, metabolic, endocrine, and toxic myopathy, myasthenia gravis, periodic paralysis, a mental disorder including mood, anxiety, and schizophrenic disorder, seasonal affective disorder (SAD), akathesia, amnesia, catatonia, diabetic neuropathy, tardive dyskinesia, dystonias, paranoid psychoses, postherpetic neuralgia, and Tourette's disorder; a reproductive disorder such as a disorder of prolactin production, infertility, including tubal disease, ovulatory defects, and endometriosis, a disruption of the estrous cycle, a disruption of the menstrual cycle, polycystic ovary syndrome, ovarian hyperstimulation syndrome, endometrial and ovarian tumors, uterine fibroids, autoimmune disorders, ectopic pregnancies, and teratogenesis; cancer of the breast, fibrocystic breast disease, and galactorrhea; a disruption of spermatogenesis, abnormal sperm physiology, cancer of the testis, cancer of the prostate, benign prostatic hyperplasia, prostatitis, Peyronie's disease, impotence, carcinoma of the male breast, and gynecomastia; a developmental disorder such as renal tubular acidosis, anemia, Cushing's syndrome, achondroplastic dwarfism, Duchenne and Becker muscular dystrophy, epilepsy, gonadal dysgenesis, WAGR syndrome (Wilms' tumor, aniridia, genitourinary abnormalities, and mental retardation), Smith-Magenis syndrome, myelodysplastic syndrome, hereditary mucoepithelial dysplasia, hereditary keratodermas, hereditary neuropathies such as Charcot-Marie-Tooth disease and neurofibromatosis, hypothyroidism, hydrocephalus, seizure disorders such as Syndenham's chorea and cerebral palsy, spina bifida, anencephaly, craniorachischisis, congenital glaucoma, cataract, and sensorineural hearing loss; an autoimmune/inflammatory disorder such as acquired immunodeficiency syndrome (AIDS), Addison's disease, adult respiratory distress syndrome, allergies, ankylosing spondylitis, amyloidosis, anemia, asthma, atherosclerosis, autoimmune hemolytic anemia, autoimmune thyroiditis, autoimmune polyendocrinopathy-candidiasis-ectodermal dystrophy (APECED), bronchitis, cholecystitis, contact dermatitis, Crohn's disease, atopic dermatitis, dermatomyositis, diabetes mellitus, emphysema, episodic lymphopenia with lymphocytotoxins, erythroblastosis fetalis, erythema nodosum, atrophic gastritis, glomerulonephritis, Goodpasture's syndrome, gout, Graves' disease, Hashimoto's thyroiditis, hypereosinophilia, irritable bowel syndrome, multiple sclerosis, myasthenia gravis, myocardial or pericardial inflammation, osteoarthritis, osteoporosis, pancreatitis, polymyositis, psoriasis, Reiter's syndrome, rheumatoid arthritis, scleroderma, Sjögren's syndrome, systemic anaphylaxis, systemic lupus erythematosus, systemic sclerosis, thrombocytopenic purpura, ulcerative colitis, uveitis, Werner syndrome, complications of cancer, hemodialysis, and extracorporeal circulation, viral, bacterial, fungal, parasitic, protozoal, and helminthic infections, and trauma; an infection, such as those caused by a viral agent classified as adenovirus, arenavirus, bunyavirus, calicivirus, coronavirus, filovirus, hepadnavirus, herpesvirus, flavivirus, orthomyxovirus, parvovirus, papovavirus, paramyxovirus, picornavirus, poxvirus, reovirus, retrovirus, rhabdovirus, or togavirus; an infection caused by a bacterial agent classified as pneumococcus, staphylococcus, streptococcus, bacillus, corynebacterium, clostridium, meningococcus, gonococcus, listeria, moraxella, kingella, haemophilus, legionella, bordetella, gram-negative enterobacterium including shigella, salmonella, or campylobacter, pseudomonas, vibrio, brucella, francisella, yersinia, bartonella, norcardium, actinomyces, mycobacterium, spirochaetale, rickettsia, chlamydia, or mycoplasma; an infection caused by a fungal agent classified as aspergillus, blastomyces, dermatophytes, cryptococcus, coccidioides, malasezzia, histoplasma, or other mycosis-causing fungal agent; and an infection caused by a parasite classified as plasmodium or malaria-causing, parasitic entamoeba, leishmania, trypanosoma, toxoplasma, pneumocystis carinii, intestinal protozoa such as giardia, trichomonas, tissue nematode such as trichinella, intestinal nematode such as ascaris, lymphatic filarial nematode, trematode such as schistosoma, and cestode such as tapeworm. Polynucleotides encoding NAAP may be used in Southern or northern analysis, dot blot, or other membrane-based technologies; in PCR technologies; in dipstick, pin, and multiformat ELISA-like assays; and in microarrays utilizing fluids or tissues from patients to detect altered NAAP expression. Such qualitative or quantitative methods are well known in the art.

In a particular aspect, polynucleotides encoding NAAP may be used in assays that detect the presence of associated disorders, particularly those mentioned above. Polynucleotides complementary to sequences encoding NAAP may be labeled by standard methods and added to a fluid or tissue sample from a patient under conditions suitable for the formation of hybridization complexes. After a suitable incubation period, the sample is washed and the signal is quantified and compared with a standard value. If the amount of signal in the patient sample is significantly altered in comparison to a control sample then the presence of altered levels of polynucleotides encoding NAAP in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with expression of NAAP, a normal or standard profile for expression is established. This may be accomplished by combining body fluids or cell extracts taken from normal subjects, either animal or human, with a sequence, or a fragment thereof, encoding NAAP, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

With respect to cancer, the presence of an abnormal amount of transcript (either under- or overexpressed) in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier, thereby preventing the development or further progression of the cancer.

Additional diagnostic uses for oligonucleotides designed from the sequences encoding NAAP may involve the use of PCR. These oligomers may be chemically synthesized, generated enzymatically, or produced in vitro. Oligomers will preferably contain a fragment of a polynucleotide encoding NAAP, or a fragment of a polynucleotide complementary to the polynucleotide encoding NAAP, and will be employed under optimized conditions for identification of a specific gene or condition. Oligomers may also be employed under less stringent conditions for detection or quantification of closely related DNA or RNA sequences.

In a particular aspect, oligonucleotide primers derived from polynucleotides encoding NAAP may be used to detect single nucleotide polymorphisms (SNPs). SNPs are substitutions, insertions and deletions that are a frequent cause of inherited or acquired genetic disease in humans. Methods of SNP detection include, but are not limited to, single-stranded conformation polymorphism (SSCP) and fluorescent SSCP (fSSCP) methods. In SSCP, oligonucleotide primers derived from polynucleotides encoding NAAP are used to amplify DNA using the polymerase chain reaction (PCR). The DNA may be derived, for example, from diseased or normal tissue, biopsy samples, bodily fluids, and the like. SNPs in the DNA cause differences in the secondary and tertiary structures of PCR products in single-stranded form, and these differences are detectable using gel electrophoresis in non-denaturing gels. In fSCCP, the oligonucleotide primers are fluorescently labeled, which allows detection of the amplimers in high-throughput equipment such as DNA sequencing machines. Additionally, sequence database analysis methods, termed in silico SNP (isSNP), are capable of identifying polymorphisms by comparing the sequence of individual overlapping DNA fragments which assemble into a common consensus sequence. These computer-based methods filter out sequence variations due to laboratory preparation of DNA and sequencing errors using statistical models and automated analyses of DNA sequence chromatograms. In the alternative, SNPs may be detected and characterized by mass spectrometry using, for example, the high throughput MASSARRAY system (Sequenom, Inc., San Diego Calif.).

SNPs may be used to study the genetic basis of human disease. For example, at least 16 common SNPs have been associated with non-insulin-dependent diabetes mellitus. SNPs are also useful for examining differences in disease outcomes in monogenic disorders, such as cystic fibrosis, sickle cell anemia, or chronic granulomatous disease. For example, variants in the mannose-binding lectin, MBL2, have been shown to be correlated with deleterious pulmonary outcomes in cystic fibrosis. SNPs also have utility in pharmacogenomics, the identification of genetic variants that influence a patient's response to a drug, such as life-threatening toxicity. For example, a variation in N-acetyl transferase is associated with a high incidence of peripheral neuropathy in response to the anti-tuberculosis drug isoniazid, while a variation in the core promoter of the ALOX5 gene results in diminished clinical response to treatment with an anti-asthma drug that targets the 5-lipoxygenase pathway. Analysis of the distribution of SNPs in different populations is useful for investigating genetic drift, mutation, recombination, and selection, as well as for tracing the origins of populations and their migrations (Taylor, J. G. et al. (2001) Trends Mol. Med. 7:507-512; Kwok, P.-Y. and Z. Gu (1999) Mol. Med. Today 5:538-543; Nowotny, P. et al. (2001) Curr. Opin. Neurobiol. 11:637-641).

Methods which may also be used to quantify the expression of NAAP include radiolabeling or biotinylating nucleotides, coamplification of a control nucleic acid, and interpolating results from standard curves (Melby, P. C. et al. (1993) J. Immunol. Methods 159:235-244; Duplaa, C. et al. (1993) Anal. Biochem. 212:229-236). The speed of quantitation of multiple samples may be accelerated by running the assay in a high-throughput format where the oligomer or polynucleotide of interest is presented in various dilutions and a spectrophotometric or colorimetric response gives rapid quantitation.

In further embodiments, oligonucleotides or longer fragments derived from any of the polynucleotides described herein may be used as elements on a microarray. The microarray can be used in transcript imaging techniques which monitor the relative expression levels of large numbers of genes simultaneously as described below. The microarray may also be used to identify genetic variants, mutations, and polymorphisms. This information may be used to determine gene function, to understand the genetic basis of a disorder, to diagnose a disorder, to monitor progression/regression of disease as a function of gene expression, and to develop and monitor the activities of therapeutic agents in the treatment of disease. In particular, this information may be used to develop a pharmacogenomic profile of a patient in order to select the most appropriate and effective treatment regimen for that patient. For example, therapeutic agents which are highly effective and display the fewest side effects may be selected for a patient based on his/her pharmacogenomic profile.

In another embodiment, NAAP, fragments of NAAP, or antibodies specific for NAAP may be used as elements on a microarray. The microarray may be used to monitor or measure protein-protein interactions, drug-target interactions, and gene expression profiles, as described above.

A particular embodiment relates to the use of the polynucleotides of the present invention to generate a transcript image of a tissue or cell type. A transcript image represents the global pattern of gene expression by a particular tissue or cell type. Global gene expression patterns are analyzed by quantifying the number of expressed genes and their relative abundance under given conditions and at a given time (Seilhamer et al., “Comparative Gene Transcript Analysis,” U.S. Pat. No. 5,840,484; hereby expressly incorporated by reference herein). Thus a transcript image may be generated by hybridizing the polynucleotides of the present invention or their complements to the totality of transcripts or reverse transcripts of a particular tissue or cell type. In one embodiment, the hybridization takes place in high-throughput format, wherein the polynucleotides of the present invention or their complements comprise a subset of a plurality of elements on a microarray. The resultant transcript image would provide a profile of gene activity.

Transcript images may be generated using transcripts isolated from tissues, cell lines, biopsies, or other biological samples. The transcript image may thus reflect gene expression in vivo, as in the case of a tissue or biopsy sample, or in vitro, as in the case of a cell line.

Transcript images which profile the expression of the polynucleotides of the present invention may also be used in conjunction with in vitro model systems and preclinical evaluation of pharmaceuticals, as well as toxicological testing of industrial and naturally-occurring environmental compounds. All compounds induce characteristic gene expression patterns, frequently termed molecular fingerprints or toxicant signatures, which are indicative of mechanisms of action and toxicity (Nuwaysir, E. F. et al. (1999) Mol. Carcinog. 24:153-159; Steiner, S. and N. L. Anderson (2000) Toxicol. Lett. 112-113:467-471). If a test compound has a signature similar to that of a compound with known toxicity, it is likely to share those toxic properties. These fingerprints or signatures are most useful and refined when they contain expression information from a large number of genes and gene families. Ideally, a genome-wide measurement of expression provides the highest quality signature. Even genes whose expression is not altered by any tested compounds are important as well, as the levels of expression of these genes are used to normalize the rest of the expression data. The normalization procedure is useful for comparison of expression data after treatment with different compounds. While the assignment of gene function to elements of a toxicant signature aids in interpretation of toxicity mechanisms, knowledge of gene function is not necessary for the statistical matching of signatures which leads to prediction of toxicity (see, for example, Press Release 00-02 from the National Institute of Environmental Health Sciences, released Feb. 29, 2000, available at http://www.niehs.nih.gov/oc/news/toxchip.htm). Therefore, it is important and desirable in toxicological screening using toxicant signatures to include all expressed gene sequences.

In an embodiment, the toxicity of a test compound can be assessed by treating a biological sample containing nucleic acids with the test compound. Nucleic acids that are expressed in the treated biological sample are hybridized with one or more probes specific to the polynucleotides of the present invention, so that transcript levels corresponding to the polynucleotides of the present invention may be quantified. The transcript levels in the treated biological sample are compared with levels in an untreated biological sample. Differences in the transcript levels between the two samples are indicative of a toxic response caused by the test compound in the treated sample.

Another embodiment relates to the use of the polypeptides disclosed herein to analyze the proteome of a tissue or cell type. The term proteome refers to the global pattern of protein expression in a particular tissue or cell type. Each protein component of a proteome can be subjected individually to further analysis. Proteome expression patterns, or profiles, are analyzed by quantifying the number of expressed proteins and their relative abundance under given conditions and at a given time. A profile of a cell's proteome may thus be generated by separating and analyzing the polypeptides of a particular tissue or cell type. In one embodiment, the separation is achieved using two-dimensional gel electrophoresis, in which proteins from a sample are separated by isoelectric focusing in the first dimension, and then according to molecular weight by sodium dodecyl sulfate slab gel electrophoresis in the second dimension (Steiner and Anderson, supra). The proteins are visualized in the gel as discrete and uniquely positioned spots, typically by staining the gel with an agent such as Coomassie Blue or silver or fluorescent stains. The optical density of each protein spot is generally proportional to the level of the protein in the sample. The optical densities of equivalently positioned protein spots from different samples, for example, from biological samples either treated or untreated with a test compound or therapeutic agent, are compared to identify any changes in protein spot density related to the treatment. The proteins in the spots are partially sequenced using, for example, standard methods employing chemical or enzymatic cleavage followed by mass spectrometry. The identity of the protein in a spot may be determined by comparing its partial sequence, preferably of at least 5 contiguous amino acid residues, to the polypeptide sequences of interest. In some cases, further sequence data may be obtained for definitive protein identification.

A proteomic profile may also be generated using antibodies specific for NAAP to quantify the levels of NAAP expression. In one embodiment, the antibodies are used as elements on a microarray, and protein expression levels are quantified by exposing the microarray to the sample and detecting the levels of protein bound to each array element (Lueking, A. et al. (1999) Anal. Biochem. 270:103-111; Mendoze, L. G. et al. (1999) Biotechniques 27:778-788). Detection may be performed by a variety of methods known in the art, for example, by reacting the proteins in the sample with a thiol- or amino-reactive fluorescent compound and detecting the amount of fluorescence bound at each array element.

Toxicant signatures at the proteome level are also useful for toxicological screening, and should be analyzed in parallel with toxicant signatures at the transcript level. There is a poor correlation between transcript and protein abundances for some proteins in some tissues (Anderson, N. L. and J. Seilhamer (1997) Electrophoresis 18:533-537), so proteome toxicant signatures may be useful in the analysis of compounds which do not significantly affect the transcript image, but which alter the proteomic profile. In addition, the analysis of transcripts in body fluids is difficult, due to rapid degradation of mRNA, so proteomic profiling may be more reliable and informative in such cases.

In another embodiment, the toxicity of a test compound is assessed by treating a biological sample containing proteins with the test compound. Proteins that are expressed in the treated biological sample are separated so that the amount of each protein can be quantified. The amount of each protein is compared to the amount of the corresponding protein in an untreated biological sample. A difference in the amount of protein between the two samples is indicative of a toxic response to the test compound in the treated sample. Individual proteins are identified by sequencing the amino acid residues of the individual proteins and comparing these partial sequences to the polypeptides of the present invention.

In another embodiment, the toxicity of a test compound is assessed by treating a biological sample containing proteins with the test compound. Proteins from the biological sample are incubated with antibodies specific to the polypeptides of the present invention. The amount of protein recognized by the antibodies is quantified. The amount of protein in the treated biological sample is compared with the amount in an untreated biological sample. A difference in the amount of protein between the two samples is indicative of a toxic response to the test compound in the treated sample.

Microarrays may be prepared, used, and analyzed using methods known in the art (Brennan, T. M. et al. (1995) U.S. Pat. No. 5,474,796; Schena, M. et al. (1996) Proc. Natl. Acad. Sci. USA 93:10614-10619; Baldeschweiler et al. (1995) PCT application WO95/251116; Shalon, D. et al. (1995) PCT application WO95/35505; Heller, R. A. et al. (1997) Proc. Natl. Acad. Sci. USA 94:2150-2155; Heller, M. J. et al. (1997) U.S. Pat. No. 5,605,662). Various types of microarrays are well known and thoroughly described in Schena, M., ed. (1999; DNA Microarrays: A Practical Approach, Oxford University Press, London).

In another embodiment of the invention, nucleic acid sequences encoding NAAP may be used to generate hybridization probes useful in mapping the naturally occurring genomic sequence. Either coding or noncoding sequences may be used, and in some instances, noncoding sequences may be preferable over coding sequences. For example, conservation of a coding sequence among members of a multi-gene family may potentially cause undesired cross hybridization during chromosomal mapping. The sequences may be mapped to a particular chromosome, to a specific region of a chromosome, or to artificial chromosome constructions, e.g., human artificial chromosomes (HACs), yeast artificial chromosomes (YACs), bacterial artificial chromosomes (BACs), bacterial P1 constructions, or single chromosome cDNA libraries (Harrington, J. J. et al. (1997) Nat. Genet. 15:345-355; Price, C. M. (1993) Blood Rev. 7:127-134; Trask, B. J. (1991) Trends Genet. 7:149-154). Once mapped, the nucleic acid sequences may be used to develop genetic linkage maps, for example, which correlate the inheritance of a disease state with the inheritance of a particular chromosome region or restriction fragment length polymorphism (RFLP) (Lander, E. S. and D. Botstein (1986) Proc. Natl. Acad. Sci. USA 83:7353-7357).

Fluorescent in situ hybridization (FISH) may be correlated with other physical and genetic map data (Heinz-Ulrich, et al. (1995) in Meyers, supra, pp. 965-968). Examples of genetic map data can be found in various scientific journals or at the Online Mendelian Inheritance in Man (OMIM) World Wide Web site. Correlation between the location of the gene encoding NAAP on a physical map and a specific disorder, or a predisposition to a specific disorder, may help define the region of DNA associated with that disorder and thus may further positional cloning efforts.

In situ hybridization of chromosomal preparations and physical mapping techniques, such as linkage analysis using established chromosomal markers, may be used for extending genetic maps. Often the placement of a gene on the chromosome of another mammalian species, such as mouse, may reveal associated markers even if the exact chromosomal locus is not known. This information is valuable to investigators searching for disease genes using positional cloning or other gene discovery techniques. Once the gene or genes responsible for a disease or syndrome have been crudely localized by genetic linkage to a particular genomic region, e.g., ataxia-telangiectasia to 11q22-23, any sequences mapping to that area may represent associated or regulatory genes for further investigation (Gatti, R. A. et al. (1988) Nature 336:577-580). The nucleotide sequence of the instant invention may also be used to detect differences in the chromosomal location due to translocation, inversion, etc., among normal, carrier, or affected individuals.

In another embodiment of the invention, NAAP, its catalytic or immunogenic fragments, or oligopeptides thereof can be used for screening libraries of compounds in any of a variety of drug screening techniques. The fragment employed in such screening may be free in solution, affixed to a solid support, borne on a cell surface, or located intracellularly. The formation of binding complexes between NAAP and the agent being tested may be measured.

Another technique for drug screening provides for high throughput screening of compounds having suitable binding affinity to the protein of interest (Geysen, et al. (1984) PCT application WO84/03564). In this method, large numbers of different small test compounds are synthesized on a solid substrate. The test compounds are reacted with NAAP, or fragments thereof, and washed. Bound NAAP is then detected by methods well known in the art. Purified NAAP can also be coated directly onto plates for use in the aforementioned drug screening techniques. Alternatively, non-neutralizing antibodies can be used to capture the peptide and immobilize it on a solid support.

In another embodiment, one may use competitive drug screening assays in which neutralizing antibodies capable of binding NAAP specifically compete with a test compound for binding NAAP. In this manner, antibodies can be used to detect the presence of any peptide which shares one or more antigenic determinants with NAAP.

In additional embodiments, the nucleotide sequences which encode NAAP may be used in any molecular biology techniques that have yet to be developed, provided the new techniques rely on properties of nucleotide sequences that are currently known, including, but not limited to, such properties as the triplet genetic code and specific base pair interactions.

Without further elaboration, it is believed that one skilled in the art can, using the preceding description, utilize the present invention to its fullest extent. The following embodiments are, therefore, to be construed as merely illustrative, and not limitative of the remainder of the disclosure in any way whatsoever.

The disclosures of all patents, applications and publications, mentioned above and below, in particular U.S. Ser. No. 60/313,111, U.S. Ser. No. 60/315,105, U.S. Ser. No. 60/314,756, U.S. Ser. No. 60/314,682, U.S. Ser. No. 60/316,856, U.S. Ser. No. 60/316,751 and U.S. Ser. No. 60/328,185 are expressly incorporated by reference herein.

### EXAMPLES

I. Construction of cDNA Libraries

Incyte cDNAs were derived from cDNA libraries described in the LIFESEQ GOLD database (Incyte Genomics, Palo Alto Calif.). Some tissues were homogenized and lysed in guanidinium isothiocyanate, while others were homogenized and lysed in phenol or in a suitable mixture of denaturants, such as TRIZOL (Invitrogen), a monophasic solution of phenol and guanidine isothiocyanate. The resulting lysates were centrifuged over CsCl cushions or extracted with chloroform RNA was precipitated from the lysates with either isopropanol or sodium acetate and ethanol, or by other routine methods.

Phenol extraction and precipitation of RNA were repeated as necessary to increase RNA purity. In some cases, RNA was treated with DNase. For most libraries, poly(A)+ RNA was isolated using oligo d(T)-coupled paramagnetic particles (Promega), OLIGOTEX latex particles (QIAGEN, Chatsworth Calif.), or an OLIGOTEX mRNA purification kit (QIAGEN). Alternatively, RNA was isolated directly from tissue lysates using other RNA isolation kits, e.g., the POLY(A)PURE mRNA purification kit (Ambion, Austin Tex.).

In some cases, Stratagene was provided with RNA and constructed the corresponding cDNA libraries. Otherwise, cDNA was synthesized and cDNA libraries were constructed with the UNIZAP vector system (Stratagene) or SUPERSCRIPT plasmid system (Invitrogen), using the recommended procedures or similar methods known in the art (Ausubel et al., supra, ch. 5). Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (Amersham Biosciences) or preparative agarose gel electrophoresis. cDNAs were ligated into compatible restriction enzyme sites of the polylinker of a suitable plasmid, e.g., PBLUESCRIPT plasmid (Stratagene), PSPORT1 plasmid (Invitrogen), PCDNA2.1 plasmid (Invitrogen, Carlsbad Calif.), PBK-CMV plasmid (Stratagene), PCR2-TOPOTA plasmid (Invitrogen), PCMV-ICIS plasmid (Stratagene), pIGEN (Incyte Genomics, Palo Alto Calif.), pRARE (Incyte Genomics), or pINCY (Incyte Genomics), or derivatives thereof. Recombinant plasmids were transformed into competent E. coli cells including XL1-Blue, XL1-BlueMRF, or SOLR from Stratagene or DH5α, DH10B, or ElectroMAX DH10B from Invitrogen.

II. Isolation of cDNA Clones

Plasmids obtained as described in Example I were recovered from host cells by in vivo excision using the UNIZAP vector system (Stratagene) or by cell lysis. Plasmids were purified using at least one of the following: a Magic or WIZARD Minipreps DNA purification system (Promega); an AGTC Miniprep purification kit (Edge Biosystems, Gaithersburg Md.); and QIAWELL 8 Plasmid, QIAWELL 8 Plus Plasmid, QIAWELL 8 Ultra Plasmid purification systems or the R.E.A.L. PREP 96 plasmid purification kit from QIAGEN. Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao, V. B. (1994) Anal. Biochem. 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes, Eugene Oreg.) and a FLUOROSKAN II fluorescence scanner (Labsystems Oy, Helsinki, Finland).

III. Sequencing and Analysis

Incyte cDNA recovered in plasmids as described in Example II were sequenced as follows. Sequencing reactions were processed using standard methods or high-throughput instrumentation such as the ABI CATALYST 800 (Applied Biosystems) thermal cycler or the PTC-200 thermal cycler (MJ Research) in conjunction with the HYDRA microdispenser (Robbins Scientific) or the MCROLAB 2200 (Hamilton) liquid transfer system. cDNA sequencing reactions were prepared using reagents provided by Amersham Biosciences or supplied in ABI sequencing kits such as the ABI PRISM BIGDYE Terminator cycle sequencing ready reaction kit (Applied Biosystems). Electrophoretic separation of cDNA sequencing reactions and detection of labeled polynucleotides were carried out using the MEGABACE 1000 DNA sequencing system (Amersham Biosciences); the ABI PRISM 373 or 377 sequencing system (Applied Biosystems) in conjunction with standard ABI protocols and base calling software; or other sequence analysis systems known in the art. Reading frames within the cDNA sequences were identified using standard methods (Ausubel et al., supra, ch. 7). Some of the cDNA sequences were selected for extension using the techniques disclosed in Example VIII.

The polynucleotide sequences derived from Incyte cDNAs were validated by removing vector, linker, and poly(A) sequences and by masking ambiguous bases, using algorithms and programs based on BLAST, dynamic programming, and dinucleotide nearest neighbor analysis. The Incyte cDNA sequences or translations thereof were then queried against a selection of public databases such as the GenBank primate, rodent, mammalian, vertebrate, and eukaryote databases, and BLOCKS, PRINTS, DOMO, PRODOM; PROTEOME databases with sequences from Homo sapiens, Rattus norvegicus, Mus musculus, Caenorhabditis elegans, Saccharomyces cerevisiae, Schizosaccharomyces pombe, and Candida albicans (Incyte Genomics, Palo Alto Calif.); hidden Markov model (HMM)-based protein family databases such as PFAM, INCY, and TIGRFAM (Haft, D. H. et al. (2001) Nucleic Acids Res. 29:4143); and HMM-based protein domain databases such as SMART (Schultz, J. et al. (1998) Proc. Natl. Acad. Sci. USA 95:5857-5864; Letunic, I. et al. (2002) Nucleic Acids Res. 30:242-244). (HMM is a probabilistic approach which analyzes consensus primary structures of gene families; see, for example, Eddy, S. R. (1996) Curr. Opin. Struct. Biol. 6:361-365.) The queries were performed using programs based on BLAST, FASTA, BLIMPS, and HMMER. The Incyte cDNA sequences were assembled to produce full length polynucleotide sequences. Alternatively, GenBank cDNAs, GenBank ESTs, stitched sequences, stretched sequences, or Genscan-predicted coding sequences (see Examples IV and V) were used to extend Incyte cDNA assemblages to full length. Assembly was performed using programs based on Phred, Phrap, and Consed, and cDNA assemblages were screened for open reading frames using programs based on GeneMark, BLAST, and FASTA. The full length polynucleotide sequences were translated to derive the corresponding full length polypeptide sequences. Alternatively, a polypeptide may begin at any of the methionine residues of the full length translated polypeptide. Full length polypeptide sequences were subsequently analyzed by querying against databases such as the GenBank protein databases (genpept), SwissProt, the PROTEOME databases, BLOCKS, PRINTS, DOMO, PRODOM, Prosite, hidden Markov model (HMM)-based protein family databases such as PFAM, INCY, and TIGRFAM; and HMM-based protein domain databases such as SMART. Full length polynucleotide sequences are also analyzed using MACDNASIS PRO software (MiraiBio Inc., Alameda Calif.) and LASERGENE software (DNASTAR). Polynucleotide and polypeptide sequence alignments are generated using default parameters specified by the CLUSTAL algorithm as incorporated into the MEGALIGN multisequence alignment program (DNASTAR), which also calculates the percent identity between aligned sequences.

Table 7 summarizes the tools, programs, and algorithms used for the analysis and assembly of Incyte cDNA and full length sequences and provides applicable descriptions, references, and threshold parameters. The first column of Table 7 shows the tools, programs, and algorithms used, the second column provides brief descriptions thereof, the third column presents appropriate references, all of which are incorporated by reference herein in their entirety, and the fourth column presents, where applicable, the scores, probability values, and other parameters used to evaluate the strength of a match between two sequences (the higher the score or the lower the probability value, the greater the identity between two sequences).

The programs described above for the assembly and analysis of full length polynucleotide and polypeptide sequences were also used to identify polynucleotide sequence fragments from SEQ ID NO:34-66. Fragments from about 20 to about 4000 nucleotides which are useful in hybridization and amplification technologies are described in Table 4, column 2.

IV. Identification and Editing of Coding Sequences from Genomic DNA

Putative nucleic acid-associated proteins were initially identified by running the Genscan gene identification program against public genomic sequence databases (e.g., gbpri and gbhtg). Genscan is a general-purpose gene identification program which analyzes genomic DNA sequences from a variety of organisms (Burge, C. and S. Karlin (1997) J. Mol. Biol. 268:78-94; Burge, C. and S. Karlin (1998) Curr. Opin. Struct. Biol. 8:346-354). The program concatenates predicted exons to form an assembled cDNA sequence extending from a methionine to a stop codon. The output of Genscan is a FASTA database of polynucleotide and polypeptide sequences. The maximum range of sequence for Genscan to analyze at once was set to 30 kb. To determine which of these Genscan predicted cDNA sequences encode nucleic acid-associated proteins, the encoded polypeptides were analyzed by querying against PFAM models for nucleic acid-associated proteins. Potential nucleic acid-associated proteins were also identified by homology to Incyte cDNA sequences that had been annotated as nucleic acid-associated proteins. These selected Genscan-predicted sequences were then compared by BLAST analysis to the genpept and gbpri public databases. Where necessary, the Genscan-predicted sequences were then edited by comparison to the top BLAST hit from genpept to correct errors in the sequence predicted by Genscan, such as extra or omitted exons. BLAST analysis was also used to find any Incyte cDNA or public cDNA coverage of the Genscan-predicted sequences, thus providing evidence for transcription. When Incyte cDNA coverage was available, this information was used to correct or confirm the Genscan predicted sequence. Full length polynucleotide sequences were obtained by assembling Genscan-predicted coding sequences with Incyte cDNA sequences and/or public cDNA sequences using the assembly process described in Example III. Alternatively, full length polynucleotide sequences were derived entirely from edited or unedited Genscan-predicted coding sequences.

V. Assembly of Genomic Sequence Data with cDNA Sequence Data

“Stitched” Sequences

Partial cDNA sequences were extended with exons predicted by the Genscan gene identification program described in Example IV. Partial cDNAs assembled as described in Example III were mapped to genomic DNA and parsed into clusters containing related cDNAs and Genscan exon predictions from one or more genomic sequences. Each cluster was analyzed using an algorithm based on graph theory and dynamic programming to integrate cDNA and genomic information, generating possible splice variants that were subsequently confirmed, edited, or extended to create a full length sequence. Sequence intervals in which the entire length of the interval was present on more than one sequence in the cluster were identified, and intervals thus identified were considered to be equivalent by transitivity. For example, if an interval was present on a cDNA and two genomic sequences, then all three intervals were considered to be equivalent. This process allows unrelated but consecutive genomic sequences to be brought together, bridged by cDNA sequence. Intervals thus identified were then “stitched” together by the stitching algorithm in the order that they appear along their parent sequences to generate the longest possible sequence, as well as sequence variants. Linkages between intervals which proceed along one type of parent sequence (cDNA to cDNA or genomic sequence to genomic sequence) were given preference over linkages which change parent type (cDNA to genomic sequence). The resultant stitched sequences were translated and compared by BLAST analysis to the genpept and gbpri public databases. Incorrect exons predicted by Genscan were corrected by comparison to the top BLAST hit from genpept. Sequences were further extended with additional cDNA sequences, or by inspection of genomic DNA, when necessary.

“Stretched” Sequences

Partial DNA sequences were extended to full length with an algorithm based on BLAST analysis. First, partial cDNAs assembled as described in Example III were queried against public databases such as the GenBank primate, rodent, mammalian, vertebrate, and eukaryote databases using the BLAST program. The nearest GenBank protein homolog was then compared by BLAST analysis to either Incyte cDNA sequences or GenScan exon predicted sequences described in Example IV. A chimeric protein was generated by using the resultant high-scoring segment pairs (HSPs) to map the translated sequences onto the GenBank protein homolog. Insertions or deletions may occur in the chimeric protein with respect to the original GenBank protein homolog. The GenBank protein homolog, the chimeric protein, or both were used as probes to search for homologous genomic sequences from the public human genome databases. Partial DNA sequences were therefore “stretched” or extended by the addition of homologous genomic sequences. The resultant stretched sequences were examined to determine whether it contained a complete gene.

VI. Chromosomal Mapping of NAAP Encoding Polynucleotides

The sequences which were used to assemble SEQ ID NO:34-66 were compared with sequences from the Incyte LIESEQ database and public domain databases using BLAST and other implementations of the Smith-Waterman algorithm. Sequences from these databases that matched SEQ ID NO:34-66 were assembled into clusters of contiguous and overlapping sequences using assembly algorithms such as Phrap (Table 7). Radiation hybrid and genetic mapping data available from public resources such as the Stanford Human Genome Center (SHGC), Whitehead Institute for Genome Research (WIGR), and Généthon were used to determine if any of the clustered sequences had been previously mapped. Inclusion of a mapped sequence in a cluster resulted in the assignment of all sequences of that cluster, including its particular SEQ ID NO:, to that map location.

Map locations are represented by ranges, or intervals, of human chromosomes. The map position of an interval, in centiMorgans, is measured relative to the terminus of the chromosome's p-arm. (The centiMorgan (cM) is a unit of measurement based on recombination frequencies between chromosomal markers. On average, 1 cM is roughly equivalent to 1 megabase (Mb) of DNA in humans, although this can vary widely due to hot and cold spots of recombination.) The cM distances are based on genetic markers mapped by Généthon which provide boundaries for radiation hybrid markers whose sequences were included in each of the clusters. Human genome maps and other resources available to the public, such as the NCBI “GeneMap'99” World Wide Web site (http:H/www.ncbi.nlm.nih.gov/genemap/), can be employed to determine if previously identified disease genes map within or in proximity to the intervals indicated above.

VII. Analysis of Polynucleotide Expression

Northern analysis is a laboratory technique used to detect the presence of a transcript of a gene and involves the hybridization of a labeled nucleotide sequence to a membrane on which RNAs from a particular cell type or tissue have been bound (Sambrook, supra, ch. 7; Ausubel et al., supra, ch. 4).

Analogous computer techniques applying BLAST were used to search for identical or related molecules in cDNA databases such as GenBank or LIFESEQ (Incyte Genomics). This analysis is much faster than multiple membrane-based hybridizations. In addition, the sensitivity of the computer search can be modified to determine whether any particular match is categorized as exact or similar. The basis of the search is the product score, which is defined as:

$$\frac{BLAST Score \times Percent Identity}{5 \times minimum \{ length ( Seq . 1 ) , length ( Seq . 2 ) \}}$$

The product score takes into account both the degree of similarity between two sequences and the length of the sequence match. The product score is a normalized value between 0 and 100, and is calculated as follows: the BLAST score is multiplied by the percent nucleotide identity and the product is divided by (5 times the length of the shorter of the two sequences). The BLAST score is calculated by assigning a score of +5 for every base that matches in a high-scoring segment pair (HSP), and −4 for every mismatch. Two sequences may share more than one HSP (separated by gaps). If there is more than one HSP, then the pair with the highest BLAST score is used to calculate the product score. The product score represents a balance between fractional overlap and quality in a BLAST alignment. For example, a product score of 100 is produced only for 100% identity over the entire length of the shorter of the two sequences being compared. A product score of 70 is produced either by 100% identity and 70% overlap at one end, or by 88% identity and 100% overlap at the other. A product score of 50 is produced either by 100% identity and 50% overlap at one end, or 79% identity and 100% overlap.

Alternatively, polynucleotides encoding NAAP are analyzed with respect to the tissue sources from which they were derived. For example, some full length sequences are assembled, at least in part, with overlapping Incyte cDNA sequences (see Example III). Each cDNA sequence is derived from a cDNA library constructed from a human tissue. Each human tissue is classified into one of the following organ/tissue categories: cardiovascular system; connective tissue; digestive system; embryonic structures; endocrine system; exocrine glands; genitalia, female; genitalia, male; germ cells; hemic and immune system; liver; musculoskeletal system; nervous system; pancreas; respiratory system; sense organs; skin; stomatognathic system; unclassified/mixed; or urinary tract. The number of libraries in each category is counted and divided by the total number of libraries across all categories. Similarly, each human tissue is classified into one of the following disease/condition categories: cancer, cell line, developmental, inflammation, neurological, trauma, cardiovascular, pooled, and other, and the number of libraries in each category is counted and divided by the total number of libraries across all categories. The resulting percentages reflect the tissue- and disease-specific expression of cDNA encoding NAAP. cDNA sequences and cDNA library/tissue information are found in the LIFESEQ GOLD database (Incyte Genomics, Palo Alto Calif.).

VIII. Extension of NAAP Encoding Polynucleotides

Full length polynucleotides are produced by extension of an appropriate fragment of the full length molecule using oligonucleotide primers designed from this fragment. One primer was synthesized to initiate 5 ′ extension of the known fragment, and the other primer was synthesized to initiate 3′ extension of the known fragment. The initial primers were designed using OLIGO 4.06 software (National Biosciences), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed.

High fidelity amplification was obtained by PCR using methods well known in the art. PCR was performed in 96-well plates using the PTC-200 thermal cycler (MJ Research, Inc.). The reaction mix contained DNA template, 200 nmol of each primer, reaction buffer containing Mg²⁺, (NH₄)₂SO₄, and 2-mercaptoethanol, Taq DNA polymerase (Amersham Biosciences), ELONGASE enzyme (Invitrogen), and Pfu DNA polymerase (Stratagene), with the following parameters for primer pair PCI A and PCI B: Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ were as follows: Step 1: 94° C., 3 min; Step 2: 94° C., 15 sec; Step 3: 57° C., 1 min; Step 4: 68° C., 2 min; Step 5: Steps 2, 3, and 4 repeated 20 times; Step 6: 68° C., 5 min; Step 7: storage at 4° C.

The concentration of DNA in each well was determined by dispensing 100 μl PICOGREEN quantitation reagent (0.25% (v/v) PICOGREEN; Molecular Probes, Eugene Oreg.) dissolved in 1×TE and 0.5 μl of undiluted PCR product into each well of an opaque fluorimeter plate (Corning Costar, Acton Mass.), allowing the DNA to bind to the reagent. The plate was scanned in a Fluoroskan II (Labsystems Oy, Helsinki, Finland) to measure the fluorescence of the sample and to quantify the concentration of DNA. A 5 μl to 10 μl aliquot of the reaction mixture was analyzed by electrophoresis on a 1% agarose gel to determine which reactions were successfiul in extending the sequence.

The extended nucleotides were desalted and concentrated, transferred to 384-well plates, digested with CviJI cholera virus endonuclease (Molecular Biology Research, Madison Wis.), and sonicated or sheared prior to religation into pUC 18 vector (Amersham Biosciences). For shotgun sequencing, the digested nucleotides were separated on low concentration (0.6 to 0.8%) agarose gels, fragments were excised, and agar digested with Agar ACE (Promega). Extended clones were religated using T4 ligase (New England Biolabs, Beverly Mass.) into pUC 18 vector (Amersham Biosciences), treated with Pfu DNA polymerase (Stratagene) to fill-in restriction site overhangs, and transfected into competent E. coli cells. Transformed cells were selected on antibiotic-containing media, and individual colonies were picked and cultured overnight at 37° C. in 384-well plates in LB/2× carb liquid media.

The cells were lysed, and DNA was amplified by PCR using Taq DNA polymerase (Amersham Biosciences) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified by PICOGREEN reagent (Molecular Probes) as described above. Samples with low DNA recoveries were reamplified using the same conditions as described above. Samples were diluted with 20% dimethysulfoxide (1:2, v/v), and sequenced using DYENAMIC energy transfer sequencing primers and the DYENAMIC DIRECT kit (Amersham Biosciences) or the ABI PRISM BIGDYE Terminator cycle sequencing ready reaction kit (Applied Biosystems).

In like manner, full length polynucleotides are verified using the above procedure or are used to obtain 5′ regulatory sequences using the above procedure along with oligonucleotides designed for such extension, and an appropriate genomic library.

IX. Identification of Single Nucleotide Polymorphisms in NAAP Encoding Polynucleotides

Common DNA sequence variants known as single nucleotide polymorphisms (SNPs) were identified in SEQ ID NO:34-66 using the LIFESEQ database (Incyte Genomics). Sequences from the same gene were clustered together and assembled as described in Example III, allowing the identification of all sequence variants in the gene. An algorithm consisting of a series of filters was used to distinguish SNPs from other sequence variants. Preliminary filters removed the majority of basecall errors by requiring a minimum Phred quality score of 15, and removed sequence alignment errors and errors resulting from improper trimming of vector sequences, chimeras, and splice variants. An automated procedure of advanced chromosome analysis analysed the original chromatogram files in the vicinity of the putative SNP. Clone error filters used statistically generated algorithms to identify errors introduced during laboratory processing, such as those caused by reverse transcriptase, polymerase, or somatic mutation. Clustering error filters used statistically generated algorithms to identify errors resulting from clustering of close homologs or pseudogenes, or due to contamination by non-human sequences. A final set of filters removed duplicates and SNPs found in immunoglobulins or T-cell receptors.

Certain SNPs were selected for further characterization by mass spectrometry using the high throughput MASSARRAY system (Sequenom, Inc.) to analyze allele frequencies at the SNP sites in four different human populations. The Caucasian population comprised 92 individuals (46 male, 46 female), including 83 from Utah, four French, three Venezualan, and two Amish individuals. The African population comprised 194 individuals (97 male, 97 female), all African Americans. The Hispanic population comprised 324 individuals (162 male, 162 female), all Mexican Hispanic. The Asian population comprised 126 individuals (64 male, 62 female) with a reported parental breakdown of 43% Chinese, 31% Japanese, 13% Korean, 5% Vietnamese, and 8% other Asian. Allele frequencies were first analyzed in the Caucasian population; in some cases those SNPs which showed no allelic variance in this population were not further tested in the other three populations.

X. Labeling and Use of Individual Hybridization Probes

Hybridization probes derived from SEQ ID NO:34-66 are employed to screen cDNAs, genomic DNAs, or mRNAs. Although the labeling of oligonucleotides, consisting of about 20 base pairs, is specifically described, essentially the same procedure is used with larger nucleotide fragments. Oligonucleotides are designed using state-of-the-art software such as OLIGO 4.06 software (National Biosciences) and labeled by combining 50 pmol of each oligomer, 250 μCi of [γ-³²P] adenosine triphosphate (Amersham Biosciences), and T4 polynucleotide kinase (DuPont NEN, Boston Mass.). The labeled oligonucleotides are substantially purified using a SEPHADEX G-25 superfine size exclusion dextran bead column (Amersham Biosciences). An aliquot containing 10⁷ counts per minute of the labeled probe is used in a typical membrane-based hybridization analysis of human genomic DNA digested with one of the following endonucleases: Ase I, Bgl II, Eco RI, Pst I, Xba I, or Pvu II (DuPont NEN).

The DNA from each digest is fractionated on a 0.7% agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under conditions of up to, for example, 0.1× saline sodium citrate and 0.5% sodium dodecyl sulfate. Hybridization patterns are visualized using autoradiography or an alternative imaging means and compared.

XI. Microarrays

The linkage or synthesis of array elements upon a microarray can be achieved utilizing photolithography, piezoelectric printing (ink-jet printing; see, e.g., Baldeschweiler et al., supra), mechanical microspotting technologies, and derivatives thereof. The substrate in each of the aforementioned technologies should be uniform and solid with a non-porous surface (Schena, M., ed. (1999) DNA Microarrays: A Practical Approach, Oxford University Press, London). Suggested substrates include silicon, silica, glass slides, glass chips, and silicon wafers. Alternatively, a procedure analogous to a dot or slot blot may also be used to arrange and link elements to the surface of a substrate using thermal, UV, chemical, or mechanical bonding procedures. A typical array may be produced using available methods and machines well known to those of ordinary skill in the art and may contain any appropriate number of elements (Schena, M. et al. (1995) Science 270:467-470; Shalon, D. et al. (1996) Genome Res. 6:639-645; Marshall, A. and J. Hodgson (1998) Nat. Biotechnol. 16:27-31).

Full length cDNAs, Expressed Sequence Tags (ESTs), or fragments or oligomers thereof may comprise the elements of the microarray. Fragments or oligomers suitable for hybridization can be selected using software well known in the art such as LASERGENE software (DNASTAR). The array elements are hybridized with polynucleotides in a biological sample. The polynucleotides in the biological sample are conjugated to a fluorescent label or other molecular tag for ease of detection. After hybridization, nonhybridized nucleotides from the biological sample are removed, and a fluorescence scanner is used to detect hybridization at each array element. Alternatively, laser desorbtion and mass spectrometry may be used for detection of hybridization. The degree of complementarity and the relative abundance of each polynucleotide which hybridizes to an element on the microarray may be assessed. In one embodiment, microarray preparation and usage is described in detail below.

Tissue or Cell Sample Preparation

Total RNA is isolated from tissue samples using the guanidinium thiocyanate method and poly(A)⁺ RNA is purified using the oligo-(dT) cellulose method. Each poly(A)⁺ RNA sample is reverse transcribed using MMLV reverse-transcriptase, 0.05 pg/μl oligo-(dT) primer (21mer), 1× first strand buffer, 0.03 units/μl RNase inhibitor, 500 μM dATP, 500 μM dGTP, 500 μM dTTP, 40 μM dCTP, 40 μM dCTP-Cy3 (BDS) or dCTP-Cy5 (Amersham Biosciences). The reverse transcription reaction is performed in a 25 ml volume containing 200 ng poly(A)⁺ RNA with GEMBRIGHT kits (Incyte). Specific control poly(A)⁺ RNAs are synthesized by in vitro transcription from non-coding yeast genomic DNA. After incubation at 37° C. for 2 hr, each reaction sample (one with Cy3 and another with Cy5 labeling) is treated with 2.5 ml of 0.5M sodium hydroxide and incubated for 20 minutes at 85° C. to the stop the reaction and degrade the RNA. Samples are purified using two successive CHROMA SPIN 30 gel filtration spin columns (Clontech, Palo Alto Calif.) and after combining, both reaction samples are ethanol precipitated using 1 ml of glycogen (1 mg/ml), 60 ml sodium acetate, and 300 ml of 100% ethanol. The sample is then dried to completion using a SpeedVAC (Savant Instruments Inc., Holbrook N.Y.) and resuspended in 14 μl 5×SSC/0.2% SDS.

Microarray Preparation

Sequences of the present invention are used to generate array elements. Each array element is amplified from bacterial cells containing vectors with cloned cDNA inserts. PCR amplification uses primers complementary to the vector sequences flanking the cDNA insert. Array elements are amplified in thirty cycles of PCR from an initial quantity of 1-2 ng to a final quantity greater than 5 μg. Amplified array elements are then purified using SEPHACRYL-400 (Amersham Biosciences).

Purified array elements are immobilized on polymer-coated glass slides. Glass microscope slides (Corning) are cleaned by ultrasound in 0.1% SDS and acetone, with extensive distilled water washes between and after treatments. Glass slides are etched in 4% hydrofluoric acid (VWR Scientific Products Corporation (VWR), West Chester Pa.), washed extensively in distilled water, and coated with 0.05% aminopropyl silane (Sigma) in 95% ethanol. Coated slides are cured in a 110° C. oven.

Array elements are applied to the coated glass substrate using a procedure described in U.S. Pat. No. 5,807,522, incorporated herein by reference. 1 μl of the array element DNA, at an average concentration of 100 ng/μl, is loaded into the open capillary printing element by a high-speed robotic apparatus. The apparatus then deposits about 5 nl of array element sample per slide.

Microarrays are UV-crosslinked using a STRATALINKER V-crosslinker (Stratagene). Microarrays are washed at room temperature once in 0.2% SDS and three times in distilled water. Non-specific binding sites are blocked by incubation of microarrays in 0.2% casein in phosphate buffered saline (PBS) (Tropix, Inc., Bedford Mass.) for 30 minutes at 60° C. followed by washes in 0.2% SDS and distilled water as before.

Hybridization

Hybridization reactions contain 9 Al of sample mixture consisting of 0.2/μg each of Cy3 and Cy5 labeled cDNA synthesis products in 5×SSC, 0.2% SDS hybridization buffer. The sample mixture is heated to 65° C. for 5 minutes and is aliquoted onto the microarray surface and covered with an 1.8 cm² coverslip. The arrays are transferred to a waterproof chamber having a cavity just slightly larger than a microscope slide. The chamber is kept at 100% humidity internally by the addition of 140 μl of 5×SSC in a corner of the chamber. The chamber containing the arrays is incubated for about 6.5 hours at 60° C. The arrays are washed for 10 min at 45° C. in a first wash buffer (1×SSC, 0.1% SDS), three times for 10 minutes each at 45° C. in a second wash buffer (0.1×SSC), and dried.

Detection

Reporter-labeled hybridization complexes are detected with a microscope equipped with an Innova 70 mixed gas 10 W laser (Coherent, Inc., Santa Clara Calif.) capable of generating spectral lines at 488 nm for excitation of Cy3 and at 632 nm for excitation of Cy5. The excitation laser light is focused on the array using a 20× microscope objective (Nikon, Inc., Melville N.Y.). The slide containing the array is placed on a computer-controlled X-Y stage on the microscope and raster-scanned past the objective. The 1.8 cm×1.8 cm array used in the present example is scanned with a resolution of 20 micrometers.

In two separate scans, a mixed gas multiline laser excites the two fluorophores sequentially. Emitted light is split, based on wavelength, into two photomultiplier tube detectors (PMT R1477, Hamamatsu Photonics Systems, Bridgewater N.J.) corresponding to the two fluorophores. Appropriate filters positioned between the array and the photomultiplier tubes are used to filter the signals. The emission maxima of the fluorophores used are 565 nm for Cy3 and 650 nm for Cy5. Each array is typically scanned twice, one scan per fluorophore using the appropriate filters at the laser source, although the apparatus is capable of recording the spectra from both fluorophores simultaneously.

The sensitivity of the scans is typically calibrated using the signal intensity generated by a cDNA control species added to the sample mixture at a known concentration. A specific location on the array contains a complementary DNA sequence, allowing the intensity of the signal at that location to be correlated with a weight ratio of hybridizing species of 1:100,000. When two samples from different sources (e.g., representing test and control cells), each labeled with a different fluorophore, are hybridized to a single array for the purpose of identifying genes that are differentially expressed, the calibration is done by labeling samples of the calibrating cDNA with the two fluorophores and adding identical amounts of each to the hybridization mixture.

The output of the photomultiplier tube is digitized using a 12-bit RTI-835H analog-to-digital (A/D) conversion board (Analog Devices, Inc., Norwood Mass.) installed in an IBM-compatible PC computer. The digitized data are displayed as an image where the signal intensity is mapped using a linear 20color transformation to a pseudocolor scale ranging from blue (low signal) to red (high signal). The data is also analyzed quantitatively. Where two different fluorophores are excited and measured simultaneously, the data are first corrected for optical crosstalk (due to overlapping emission spectra) between the fluorophores using each fluorophore's emission spectrum.

A grid is superimposed over the fluorescence signal image such that the signal from each spot is centered in each element of the grid. The fluorescence signal within each element is then integrated to obtain a numerical value corresponding to the average intensity of the signal. The software used for signal analysis is the GEMTOOLS gene expression analysis program (Incyte). Array elements that exhibited at least about a two-fold change in expression, a signal-to-background ratio of at least 2.5, and an element spot size of at least 40% were identified as differentially expressed using the GEMTOOLS program (Incyte Genomics).

Expression

For example, SEQ ID NO:43 was differentially expressed in normal brain tissue versus mild Alzheimer's diseased brain tissue based on microarray experimentation. Alzheimer's disease (AD) is a progressive dementia characterized neuropathologically by the presence of amyloid β-peptide-containing plaques and neurofibrillary tangles in specific brain regions. In addition, neurons and synapses are lost and inflammatory responses are activated in microglia and astrocytes. A cross-comparison experimental design was used to evaluate the expression of cDNAs from specific dissected regions of human brain (Dn3629 from a 68-year old female with mild AD in the posterior cingulate tissue), as compared to normal human brain tissue from equivalent regions (Dn3625 from a normal 61-year old female).

The expression of SEQ ID NO:43 was increased at least two-fold in mild AD tissue from the posterior cingulate region of the brain. These experiments indicate that SEQ ID NO:43 was significantly overexpressed in the mild AD brain tissue tested, further establishing the utility of SEQ ID NO:43 as diagnostic marker or as therapeutic target for AD.

In addition, SEQ ID NO:44 was differentially expressed in cancer cells versus normal cells based on microarray experimentation. SEQ ID NO:44 showed differential expression as determined by microarray analysis. Histological and molecular evaluation of breast tumors reveals that the development of breast cancer evolves through a multi-step process whereby pre-malignant mammary epithelial cells undergo a relatively defined sequence of events leading to tumor formation. A cross-comparison experimental design was used to evaluate the expression of cDNAs from three human breast tumor cell lines (Sk-BR-3, MDA-mb-231, and MDA-mb435S) at various stages of tumor progression, as compared to a non-malignant mammary epithelial cell line, HMEC (Clonetics, San Diego, Calif.). All cell cultures were propagated in media according to the supplier's recommendations and grown to 70-80% confluence prior to RNA isolation.

The expression of SEQ ID NO:44 was decreased at least two-fold in Sk-BR-3 cells, a breast adenocarcinoma cell line isolated from a malignant pleural effusion of a 43-year-old female. It forms poorly differentiated adenocarcinoma when injected into nude mice. The expression of SEQ ID NO:44 was also found to be decreased by at least two-fold in MDA-mb-231, a breast tumor cell line isolated from the pleural effusion of a 51-year old female. It forms poorly differentiated adenocarcinoma in nude mice and ALS treated BALB/c mice. These cells also expresses the Wnt3 oncogene, EGF, and TGF-α. The expression of SEQ ID NO:44 was also found to be decreased by at least two-fold in MDA-mb-435S cells, a spindle-shaped strain that evolved from the parent line (435) as isolated in 1976 from the pleural effusion of a 31-year old female with metastatic, ductal adenocarcinoma of the breast.

The expression of SEQ ID NO:44 was also significantly underexpressed in another experiment in which two human breast tumor cell lines, Sk-BR-3 and MDA-mb-231, were compared to a nonmalignant breast epithelial cell line (MCF-10A).

The expression of SEQ ID NO:44 was differentially expressed in a cross-comparison between a non-tumorigenic human prostate cell line, PZ-HPV-7, and three human tumorigenic cell lines: DU 145, a prostate carcinoma cell line isolated from a metastatic site in the brain of a 69-year old male with widespread metastatic prostate carcinoma; LNCaP, a prostate carcinoma cell line isolated from a lymph node biopsy of a 50-year old male with metastatic prostate carcinoma; and PC-3, a prostate adenocarcinoma cell line that was isolated from a metastatic site in the bone of a 62-year old male with grade IV prostate adenocarcinoma. The expression of SEQ ID NO:44 was significantly underexpressed by at least two-fold in these experiments.

These experiments indicate that SEQ ID NO:44 was significantly underexpressed in the breast and prostate tumor lines tested, further establishing the utility of SEQ ID NO:44 as a diagnostic marker or as a therapeutic target for cancer.

For example, SEQ ID NO:54 showed differential expression associated with inflammatory responses as determined by microarray analysis. The expression of SEQ ID NO:54 was decreased by at least two-fold in peripheral blood mononuclear cells (PBMCs; 12% B lymphocytes, 40% T lymphocytes, 20% NK cells, 25% monocytes, and 3% various cells that include dendritic and progenitor cells) treated with cyclosporin A in a mixed lymphocyte reaction as compared to untreated PBMCs. Cyclosporin A is used for immunosuppression in treating patients undergoing organ transplant operations and in treating other inflammatory conditions. Cyclosporin A interacts with cyclophilin to inhibit the phosphatase, calcineurin. Inhibition of calcineurin blocks induction of genes involved in the immune response, including interleukin-2, interleukin-3, and interferon-γ and interferes with development of the immune response. Therefore, SEQ ID NO:54 is useful in diagnostic assays for inflammatory responses.

In another example, the expression of SEQ ID NO:57 was compared in normal and cancerous tissue samples from eight patients with lung tumors. SEQ ID NO:57 showed at least a two-fold increase in expression in lung tissue from a patient with lung adenocarcinoma compared to matched microscopically normal tissue from the same donor as determined by microarray analysis. Therefore, SEQ ID NO:57 is useful in disease staging and in diagnostic assays for cell proliferative disorders, including lung cancer.

In an alternative example, the expression of SEQ ID NO:59 showed at least a two-fold decrease in MDA-Mb-231, Sk-BR-3, MDA-Mb435S, and T47D breast carcinoma cells compared to nontumorigenic MCF-10A breast mammary gland cells. MDA-Mb-231 is a breast tumor cell line isolated from the pleural effusion of a 51-year old female. Sk-BR-3 is a breast adenocarcinoma cell line isolated from a malignant pleural effusion of a 43-year old female. MDA-Mb-435S is a spindle shaped strain that evolved from the parent line (435) as isolated from the pleural effusion of a 31-year old female with metastatic, ductal adenocarcinoma of the breast. T-47D is a breast carcinoma cell line isolated from a pleural effusion obtained from a 54-year old female with an infiltrating ductal carcinoma of the breast. MCF-10A is a breast mammary gland cell line that was isolated from a 36-year old woman with fibrocystic breast disease. Therefore, SEQ ID NO:59 is useful in disease staging and in diagnostic assays for cell proliferative disorders, including breast cancer.

In an alternative example, the expression of SEQ ID NO:61 showed at least a two-fold decrease in human peripheral blood mononuclear cells (PBMCs) treated with acetaminophen compared to untreated cells. PBMCs contain the major cellular components of the immune system, including about 52% lymphocytes, 20% NK cells, 25% monocytes, and 3% various cells such as dendritic cells and progenitor cells. Acetaminophen possesses analgesic and antipyretic activity. Acetaminophen inhibits cyclooxygenase in the central nervous system, but does not show anti-inflammatory effects in peripheral tissues. Therefore, SEQ ID NO:61 is useful in disease staging and in diagnostic assays for immune disorders.

In an alternative example, SEQ ID NO:64 showed differential expression in human C3A liver cell cultures treated with steroids compared to untreated cells. Early confluent human liver C3A cells were treated with mifepristone, progesterone, beclomethasone, medroxyprogesterone, budesonide, prednisone, dexamethasone, betamethasone, or danazol at concentrations of 1 μM, 10 μM, and 100 μM for 1, 3, and 6 hours. SEQ ID NO:64 showed decreased expression in C3A cells treated with beclomethasone, medroxyprogesterone, budesonide, prednisone, or dexamethasone. Therefore, SEQ ID NO:64 is useful in disease staging and in diagnostic assays for liver disorders associated with steroid therapy.

SEQ ID NO:64 also showed differential expression associated with lung cancer. The expression of SEQ ID NO:64 was compared in normal and cancerous tissue samples from ten patients with lung tumors. SEQ ID NO:64 showed at least a two-fold decrease in expression in lung tissue from two patients with lung cancer compared to matched microscopically normal tissue from the same donors as determined by microarray analysis. Therefore, SEQ ID NO:64 is useful in disease staging and in diagnostic assays for cell proliferative disorders, including lung cancer.

XII. Complementary Polynucleotides

Sequences complementary to the NAAP-encoding sequences, or any parts thereof, are used to detect, decrease, or inhibit expression of naturally occurring NAAP. Although use of oligonucleotides comprising from about 15 to 30 base pairs is described, essentially the same procedure is used with smaller or with larger sequence fragments. Appropriate oligonucleotides are designed using OLIGO 4.06 software (National Biosciences) and the coding sequence of NAAP. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the NAAP-encoding transcript.

XIII. Expression of NAAP

Expression and purification of NAAP is achieved using bacterial or virus-based expression systems. For expression of NAAP in bacteria, cDNA is subcloned into an appropriate vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into suitable bacterial hosts, e.g., BL21(DE3). Antibiotic resistant bacteria express NAAP upon induction with isopropyl beta-D-thiogalactopyranoside (IPTG). Expression of NAAP in eukaryotic cells is achieved by infecting insect or mammalian cell lines with recombinant Autographica californica nuclear polyhedrosis virus (AcMNPV), commonly known as baculovirus. The nonessential polyhedrin gene of baculovirus is replaced with cDNA encoding NAAP by either homologous recombination or bacterial-mediated transposition involving transfer plasmid intermediates. Viral infectivity is maintained and the strong polyhedrin promoter drives high levels of cDNA transcription. Recombinant baculovirus is used to infect Spodoptera frugiperda (Sf9) insect cells in most cases, or human hepatocytes, in some cases. Infection of the latter requires additional genetic modifications to baculovirus (Engelhard, E. K. et al. (1994) Proc. Natl. Acad. Sci. USA 91:3224-3227; Sandig, V. et al. (1996) Hum. Gene Ther. 7:1937-1945).

In most expression systems, NAAP is synthesized as a fusion protein with, e.g., glutathione S-transferase (GST) or a peptide epitope tag, such as FLAG or 6-His, permitting rapid, single-step, affinity-based purification of recombinant fusion protein from crude cell lysates. GST, a 26-kilodalton enzyme from Schistosoma japonicum, enables the purification of fusion proteins on immobilized glutathione under conditions that maintain protein activity and antigenicity (Amersham Biosciences). Following purification, the GST moiety can be proteolytically cleaved from NAAP at specifically engineered sites. FLAG, an 8-amino acid peptide, enables immunoaffinity purification using commercially available monoclonal and polyclonal anti-FLAG antibodies (Eastman Kodak). 6-His, a stretch of six consecutive histidine residues, enables purification on metal-chelate resins (QIAGEN). Methods for protein expression and purification are discussed in Ausubel et al. (supra, ch. 10 and 16). Purified NAAP obtained by these methods can be used directly in the assays shown in Examples XVII, XVIII, and XIX, where applicable.

XIV. Functional Assays

NAAP function is assessed by expressing the sequences encoding NAAP at physiologically elevated levels in mammalian cell culture systems. cDNA is subcloned into a mammalian expression vector containing a strong promoter that drives high levels of cDNA expression. Vectors of choice include PCMV SPORT plasmid (Invitrogen, Carlsbad Calif.) and PCR3.1 plasmid (Invitrogen), both of which contain the cytomegalovirus promoter. 5-10 μg of recombinant vector are transiently transfected into a human cell line, for example, an endothelial or hematopoietic cell line, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins of choice include, e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP and to evaluate the apoptotic state of the cells and other cellular properties. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994; Flow Cytometry, Oxford, New York N.Y.).

The influence of NAAP on gene expression can be assessed using highly purified populations of cells transfected with sequences encoding NAAP and either CD64 or CD64-GFP. CD64 and CD64-GFP are expressed on the surface of transfected cells and bind to conserved regions of human immnunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding NAAP and other genes of interest can be analyzed by northern analysis or microarray techniques.

XV. Production of NAAP Specific Antibodies

NAAP substantially purified using polyacrylamide gel electrophoresis (PAGE; see, e.g., Harrington, M. G. (1990) Methods Enzymol. 182:488495), or other purification techniques, is used to immunize animals (e.g., rabbits, mice, etc.) and to produce antibodies using standard protocols.

Alternatively, the NAAP amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art (Ausubel et al., supra, ch. 11).

Typically, oligopeptides of about 15 residues in length are synthesized using an ABI 431A peptide synthesizer (Applied Biosystems) using FMOC chemistry and coupled to KLH (Sigma-Aldrich, St. Louis Mo.) by reaction with N-maleimidobenzoyl-N-hydroxysuccinimide ester (MBS) to increase immunogenicity (Ausubel et al., supra). Rabbits are immunized with the oligopeptide-KLH complex in complete Freund's adjuvant. Resulting antisera are tested for antipeptide and anti-NAAP activity by, for example, binding the peptide or NAAP to a substrate, blocking with 1% BSA, reacting with rabbit antisera, washing, and reacting with radio-iodinated goat anti-rabbit IgG.

XVI. Purification of Naturally Occurring NAAP Using Specific Antibodies

Naturally occurring or recombinant NAAP is substantially purified by immunoaffinity chromatography using antibodies specific for NAAP. An immunoaffinity column is constructed by covalently coupling anti-NAAP antibody to an activated chromatographic resin, such as CNBr-activated SEPHAROSE (Amersham Biosciences). After the coupling, the resin is blocked and washed according to the manufacturer's instructions.

Media containing NAAP are passed over the immunoaffinity column, and the column is washed under conditions that allow the preferential absorbance of NAAP (e.g., high ionic strength buffers in the presence of detergent). The column is eluted under conditions that disrupt antibody/NAAP binding (e.g., a buffer of pH 2 to pH 3, or a high concentration of a chaotrope, such as urea or thiocyanate ion), and NAAP is collected.

XVII. Identification of Molecules Which Interact with NAAP

NAAP, or biologically active fragments thereof, are labeled with ¹²⁵I Bolton-Hunter reagent (Bolton, A. E. and W. M. Hunter (1973) Biochem J. 133:529-539). Candidate molecules previously arrayed in the wells of a multi-well plate are incubated with the labeled NAAP, washed, and any wells with labeled NAAP complex are assayed. Data obtained using different concentrations of NAAP are used to calculate values for the number, affinity, and association of NAAP with the candidate molecules.

Alternatively, molecules interacting with NAAP are analyzed using the yeast two-hybrid system as described in Fields, S. and O. Song (1989; Nature 340:245-246), or using commercially available kits based on the two-hybrid system, such as the MATCHMAKER system (Clontech).

NAAP may also be used in the PATHCALLING process (CuraGen Corp., New Haven Conn.) which employs the yeast two-hybrid system in a high-throughput manner to determine all interactions between the proteins encoded by two large libraries of genes (Nandabalan, K. et al. (2000) U.S. Pat. No. 6,057,101).

XVIII. Demonstration of NAAP Activity

NAAP activity is measured by its ability to stimulate transcription of a reporter gene (Liu, H. Y. et al. (1997) EMBO J. 16:5289-5298). The assay entails the use of a well characterized reporter gene construct, LexAₒₚ-LacZ, that consists of LexA DNA transcriptional control elements (LexAₒₚ) fused to sequences encoding the E. coli LacZ enzyme. The methods for constructing and expressing fusion genes, introducing them into cells, and measuring LacZ enzyme activity, are well known to those skilled in the art. Sequences encoding NAAP are cloned into a plasmid that directs the synthesis of a fusion protein, LexA-NAAP, consisting of NAAP and a DNA binding domain derived from the LexA transcription factor. The resulting plasmid, encoding a LexA-NAAP fusion protein, is introduced into yeast cells along with a plasmid containing the LexAₒₚ-LacZ reporter gene. The amount of LacZ enzyme activity associated with LexA-NAAP transfected cells, relative to control cells, is proportional to the amount of transcription stimulated by the NAAP.

Alternatively, NAAP activity is measured by its ability to bind zinc. A 5-10 μM sample solution in 2.5 mM ammonium acetate solution at pH 7.4 is combined with 0.05 M zinc sulfate solution (Aldrich, Milwaukee Wis.) in the presence of 100 μM dithiothreitol with 10% methanol added. The sample and zinc sulfate solutions are allowed to incubate for 20 minutes. The reaction solution is passed through a VYDAC column (Grace Vydac, Hesperia, Calif.) with approximately 300 Angstrom bore size and 5 μM particle size to isolate zinc-sample complex from the solution, and into a mass spectrometer (PE Sciex, Ontario, Canada). Zinc bound to sample is quantified using the functional atomic mass of 63.5 Da observed by Whittal, R. M. et al. ((2000) Biochemistry 39:8406-8417).

In the alternative, a method to determine nucleic acid binding activity of NAAP involves a polyacrylamide gel mobility-shift assay. In preparation for this assay, NAAP is expressed by transforming a mammalian cell line such as COS7, HeLa or CHO with a eukaryotic expression vector containing NAAP cDNA. The cells are incubated for 48-72 hours after transformation under conditions appropriate for the cell line to allow expression and accumulation of NAAP. Extracts containing solubilized proteins can be prepared from cells expressing NAAP by methods well known in the art. Portions of the extract containing NAAP are added to [³²P]-labeled RNA or DNA. Radioactive nucleic acid can be synthesized in vitro by techniques well known in the art. The mixtures are incubated at 25° C. in the presence of RNase- and DNase-inhibitors under buffered conditions for 5-10 minutes. After incubation, the samples are analyzed by polyacrylamide gel electrophoresis followed by autoradiography. The presence of a band on the autoradiogram indicates the formation of a complex between NAAP and the radioactive transcript. A band of similar mobility will not be present in samples prepared using control extracts prepared from untransformed cells.

In the alternative, a method to determine methylase activity of NAAP measures transfer of radiolabeled methyl groups between a donor substrate and an acceptor substrate. Reaction mixtures (50 μl final volume) contain 15 mM HEPES, pH 7.9, 1.5 mM MgCl₂, 10 mM dithiothreitol, 3% polyvinylalcohol, 1.5 μCi [methyl-³H)AdoMet (0.375 μM AdoMet) (DuPont-NEN), 0.6 μg NAAP, and acceptor substrate (e.g., 0.4 μg [³⁵S]RNA, or 6-mercaptopurine (6-MP) to 1 mM final concentration). Reaction mixtures are incubated at 30° C. for 30 minutes, then 65° C. for 5 minutes.

Analysis of [methyl-³H]RNA is as follows: (1) 50 μl of 2× loading buffer (20 mM Tris-HCl, pH 7.6, 1 M LiCl, 1 mM EDTA, 1% sodium dodecyl sulphate (SDS)) and 50 μl oligo d(T)-cellulose (10 mg/ml in 1× loading buffer) are added to the reaction mixture, and incubated at ambient temperature with shaking for 30 minutes. (2) Reaction mixtures are transferred to a 96-well filtration plate attached to a vacuum apparatus. (3) Each sample is washed sequentially with three 2.4 ml aliquots of 1× oligo d(T) loading buffer containing 0.5% SDS, 0.1% SDS, or no SDS. (4) RNA is eluted with 300 μl of water into a 96-well collection plate, transferred to scintillation vials containing liquid scintillant, and radioactivity determined.

Analysis of [methyl-³H]⁶-MP is as follows: (1) 500 μl 0.5 M borate buffer, pH 10.0, and then 2.5 ml of 20% (v/v) isoamyl alcohol in toluene are added to the reaction mixtures. (2) The samples are mixed by vigorous vortexing for ten seconds. (3) After centrifugation at 700 g for 10 minutes, 1.5 ml of the organic phase is transferred to scintillation vials containing 0.5 ml absolute ethanol and liquid scintillant, and radioactivity determined. (4) Results are corrected for the extraction of 6-MP into the organic phase (approximately 41%).

In the alternative, type I topoisomerase activity of NAAP can be assayed based on the relaxation of a supercoiled DNA substrate. NAAP is incubated with its substrate in a buffer lacking Mg²⁺ and ATP, the reaction is terminated, and the products are loaded on an agarose gel. Altered topoisomers can be distinguished from supercoiled substrate electrophoretically. This assay is specific for type I topoisomerase activity because Mg²⁺ and ATP are necessary cofactors for type II topoisomerases.

Type II topoisomerase activity of NAAP can be assayed based on the decatenation of a kinetoplast DNA (KDNA) substrate. NAAP is incubated with KDNA, the reaction is terminated, and the products are loaded on an agarose gel. Monomeric circular KDNA can be distinguished from catenated KDNA electrophoretically. Kits for measuring type I and type II topoisomerase activities are available commercially from Topogen (Columbus Ohio).

ATP-dependent RNA helicase unwinding activity of NAAP can be measured by the method described by Zhang and Grosse (1994; Biochemistry 33:3906-3912). The substrate for RNA unwinding consists of ³²P-labeled RNA composed of two RNA strands of 194 and 130 nucleotides in length containing a duplex region of 17 base-pairs. The RNA substrate is incubated together with ATP, Mg²⁺, and varying amounts of NAAP in a Tris-HCl buffer, pH 7.5, at 37° C. for 30 minutes. The single-stranded RNA product is then separated from the double-stranded RNA substrate by electrophoresis through a 10% SDS-polyacrylamide gel, and quantitated by autoradiography. The amount of single-stranded RNA recovered is proportional to the amount of NAAP in the preparation.

In the alternative, NAAP function is assessed by expressing the sequences encoding NAAP at physiologically elevated levels in mammalian cell culture systems. cDNA is subcloned into a mammalian expression vector containing a strong promoter that drives high levels of cDNA expression. Vectors of choice include pCMV SPORT (Life Technologies) and pCR3.1 (Invitrogen Corporation, Carlsbad Calif.), both of which contain the cytomegalovirus promoter. 5-10 μg of recombinant vector are transiently transfected into a human cell line, preferably of endothelial or hematopoietic origin, using either liposome formulations or electroporation. 1-2 μg of an additional plasmid containing sequences encoding a marker protein are co-transfected.

Expression of a marker protein provides a means to distinguish transfected cells from nontransfected cells and is a reliable predictor of cDNA expression from the recombinant vector. Marker proteins of choice include, e.g., Green Fluorescent Protein (GFP; CLONTECH), CD64, or a CD64-GFP fusion protein. Flow cytometry (FCM), an automated laser optics-based technique, is used to identify transfected cells expressing GFP or CD64-GFP and to evaluate the apoptotic state of the cells and other cellular properties.

FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter; down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994) Flow Cytometry, Oxford, New York N.Y.

The influence of NAAP on gene expression can be assessed using highly purified populations of cells transfected with sequences encoding NAAP and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Inc., Lake Success N.Y.). mRNA can be purified from the cells using methods well known by those of skill in the art. Expression of mRNA encoding NAAP and other genes of interest can be analyzed by northern analysis or microarray techniques.

Pseudouridine synthase activity of NAAP is assayed using a tritium (³H) release assay modified from Nurse et al. ((1995) RNA 1:102-112), which measures the release of ³H from the C₅ position of the pyrimidine component of uridylate (U) when ³H-radiolabeled U in RNA is isomerized to pseudouridine (ψ). A typical 500 μl assay mixture contains 50 mM HEPES buffer (pH 7.5), 100 mM ammonium acetate, 5 mM dithiothreitol, 1 mM EDTA, 30 units RNase inhibitor, and 0.1-4.2 μM [5-³H]tRNA (approximately 1 μCi/nmol tRNA). The reaction is initiated by the addition of <5 μl of a concentrated solution of NAAP (or sample containing NAAP) and incubated for 5 min at 37° C. Portions of the reaction mixture are removed at various times (up to 30 min) following the addition of NAAP and quenched by dilution into 1 ml 0.1 M HCl containing Norit-SA3 (12% w/v). The quenched reaction mixtures are centrifuged for 5 min at maximum speed in a microcentrifuge, and the supernatants are filtered through a plug of glass wool. The pellet is washed twice by resuspension in 1 ml 0.1 M HCl, followed by centrifugation. The supernatants from the washes are separately passed through the glass wool plug and combined with the original filtrate. A portion of the combined filtrate is mixed with scintillation fluid (up to 10 ml) and counted using a scintillation counter. The amount of ³H released from the RNA and present in the soluble filtrate is proportional to the amount of peudouridine synthase activity in the sample (Ramamurthy, V. (1999) J. Biol. Chem. 274:22225-22230).

In the alternative, pseudouridine synthase activity of NAAP is assayed at 30° C. to 37° C. in a mixture containing 100 mM Tris-HCl (pH 8.0), 100 mM ammonium acetate, 5 mM MgCl₂, 2 mM dithiothreitol, 0.1 mM EDTA, and 1-2 fmol of [³²P]-radiolabeled runoff transcripts (generated in vitro by an appropriate RNA polymerase, i.e., T7 or SP6) as substrates. NAAP is added to initiate the reaction or omitted from the reaction in control samples. Following incubation, the RNA is extracted with phenol-chloroform, precipitated in ethanol, and hydrolyzed completely to 3-nucleotide monophosphates using RNase T₂. The hydrolysates are analyzed by two-dimensional thin layer chromatography, and the amount of ³²P radiolabel present in the ψMP and UMP spots are evaluated after exposing the thin layer chromatography plates to film or a PhosphorImager screen. Taking into account the relative number of uridylate residues in the substrate RNA, the relative amount ψMP and UMP are determined and used to calculate the relative amount of ψ per tRNA molecule (expressed in mol ψ/mol of tRNA or mol ψ/mol of tRNA/minute), which corresponds to the amount of pseudouridine synthase activity in the NAAP sample (Lecointe, supra).

N²,N²-dimethylguanosine transferase ((m²₂G)methyltransferase) activity of NAAP is measured in a 160 μl reaction mixture containing 100 mM Tris-HCl (pH 7.5), 0.1 mM EDTA, 10 mM MgCl₂, 20 mM NH₄Cl, 1 mM dithiothreitol, 6.2 μM S-adenosyl-L-[methyl-³H]methionine (30-70 Ci/mM), 8 μg m²₂G-deficient tRNA or wild type tRNA from yeast, and approximately 100 μg of purified NAAP or a sample comprising NAAP. The reactions are incubated at 30° C. for 90 min and chilled on ice. A portion of each reaction is diluted to 1 ml in water containing 100 μg BSA. 1 ml of 2 M HCl is added to each sample and the acid insoluble products are allowed to precipitate on ice for 20 min before being collected by filtration through glass fiber filters. The collected material is washed several times with HCl and quantitated using a liquid scintillation counter. The amount of ³H incorporated into the m²₂G-deficient, acid-insoluble tRNAs is proportional to the amount of N²,N²-dimethylguanosine transferase activity in the NAAP sample. Reactions comprising no substrate tRNAs, or wild-type tRNAs that have already been modified, serve as control reactions which should not yield acid-insoluble ³H-labeled products.

Polyadenylation activity of NAAP is measured using an in vitro polyadenylation reaction. The reaction mixture is assembled on ice and comprises 10 μl of 5 mM dithiothreitol, 0.025% (v/v) NONIDET P40, 50 mM creatine phosphate, 6.5% (w/v) polyvinyl alcohol, 0.5 unit/μl RNAGUARD (Pharmacia), 0.025 μg/μl creatine kinase, 1.25 mM cordycepin 5′-triphosphate, and 3.75 mM MgCl₂, in a total volume of 25 μl. 60 fmol of CstF, 50 fmol of CPSF, 240 fmol of PAP, 4 μl of crude or partially purified CF II and various amounts of amounts CF I are then added to the reaction mix. The volume is adjusted to 23.5 μl with a buffer containing 50 mM Tris-HCl, pH 7.9, 10% (v/v) glycerol, and 0.1 mM Na-EDTA. The final ammonium sulfate concentration should be below 20 mM. The reaction is initiated (on ice) by the addition of 15 fmol of ³P-labeled pre-mRNA template, along with 2.5 μg of unlabeled tRNA, in 1.5 μl of water. Reactions are then incubated at 30° C for 75-90 min and stopped by the addition of 75 μl (approximately two-volumes) of proteinase K mix (0.2 M Tris-HCl, pH 7.9,300 mM NaCl, 25 mM Na-EDTA, 2% (w/v) SDS), 1 μl of 10 mg/ml proteinase K, 0.25 μl of 20 mg/ml glycogen, and 23.75 μl of water). Following incubation, the RNA is precipitated with ethanol and analyzed on a 6% (w/v) polyacrylamide, 8.3 M urea sequencing gel. The dried gel is developed by autoradiography or using a phosphoimager. Cleavage activity is determined by comparing the amount of cleavage product to the amount of pre-mRNA template. The omission of any of the polypeptide components of the reaction and substitution of NAAP is useful for identifying the specific biological function of NAAP in pre-mRNA polyadenylation (Rüegsegger, supra; and references within).

tRNA synthetase activity is measured as the aminoacylation of a substrate tRNA in the presence of [¹⁴C]-labeled amino acid. NAAP is incubated with [¹⁴C]-labeled amino acid and the appropriate cognate tRNA (for example, [¹⁴C]alanine and tRNA^{ala}) in a buffered solution. ¹⁴C-labeled product is separated from free [¹⁴C]amino acid by chromatography, and the incorporated ¹⁴C is quantified by scintillation counter. The amount of ¹⁴C-labeled product detected is proportional to the activity of NAAP in this assay.

In the alternative, NAAP activity is measured by incubating a sample containing NAAP in a solution containing 1 mM ATP, 5 mM Hepes-KOH (pH 7.0), 2.5 mM KCl, 1.5 mM magnesium chloride, and 0.5 mM DTT along with misacylated [¹⁴C]-Glu-tRNAGln (e.g., 1 μM) and a similar concentration of unlabeled L-glutamine. Following the quenching of the reaction with 3 M sodium acetate (pH 5.0), the mixture is extracted with an equal volume of water-saturated phenol, and the aqueous and organic phases are separated by centrifugation at 15,000×g at room temperature for 1 min. The aqueous phase is removed and precipitated with 3 volumes of ethanol at −70° C. for 15 min. The precipitated aminoacyl-tRNAs are recovered by centrifugation at 15,000×g at 4° C. for 15 min. The pellet is resuspended in of 25 mM KOH, deacylated at 65° C. for 10 min., neutralized with 0.1 M HCl (to final pH 6-7), and dried under vacuum. The dried pellet is resuspended in water and spotted onto a cellulose TLC plate. The plate is developed in either isopropanol/formic acid/water or ammonia/water/chloroform/methanol. The image is subjected to densitometric analysis and the relative amounts of Glu and Gln are calculated based on the Rf values and relative intensities of the spots. NAAP activity is calculated based on the amount of Gln resulting from the transformation of Glu while acylated as Glu-tRNA^{Gln} (adapted from Curnow, A. W. et al. (1997) Proc. Natl. Acad. Sci. USA 94:11819-26).

Alternatively, DNA repair activity of NAAP is measured as incorporation of [³²P]dATP into a plasmid treated with a DNA damaging agent, such as cisplatin or ultraviolet irradiation, relative to a control, untreated plasmid DNA (Coudore, F. et al. (1997) FEBS Lett. 414:581-584). Cell extracts are purified from mammalian cell lines, E. coli, or S. cerevisiae having compromised endogenous repair activities due to mutations in repair enzymes. Cell extracts are prepared by hypotonic lysis of cells followed by centrifugation at 300,000×g. Extracts are treated with 63% ammonium sulfate to minimize non-specific nuclease activity. The repair synthesis assay is performed in a 50 μl reaction volume containing 200 μg protein in cell extract, 300 ng damaged plasmid, 300 ng control plasmid, 4 μM dATP, 20 μM each dCTP, dTTP, and dGTP, 0.2 μM ³²P]dATP, 20 mM HEPES-KOH (pH 7.8), 2.5 μg creatine phosphokinase, 7 MM MgCl₂, and 2 mM EGTA. Identical reactions are set up with and without purified NAAP. After a 3 h incubation at 30° C., reaction mixtures are treated with 200 μg/ml proteinase K and 0.5% SDS. Plasmid DNA is purified from reaction mixtures by phenol-chloroform extraction and ethanol precipitation. Data is quantified by gel electrophoresis of linearized plasmid followed by autoradiography, scintillation counting of excised DNA bands, and densitometry of the photographic negative of the gel to normalize for plasmid DNA recovery.

XIX. Identification of NAAP Agonists and Antagonists

Agonists or antagonists of NAAP activation or inhibition may be tested using the assays described in section XVIII. Agonists cause an increase in NAAP activity and antagonists cause a decrease in NAAP activity.

Various modifications and variations of the described compositions, methods, and systems of the invention win be apparent to those skilled in the art without departing from the scope and spirit of the invention. It will be appreciated that the invention provides novel and useful proteins, and their encoding polynucleotides, which can be used in the drug discovery process, as well as methods for using these compositions for the detection, diagnosis, and treatment of diseases and conditions. Although the invention has been described in connection with certain embodiments, it should be understood that the invention as claimed should not be unduly limited to such specific embodiments. Nor should the description of such embodiments be considered exhaustive or limit the invention to the precise forms disclosed. Furthermore, elements from one embodiment can be readily recombined with elements from one or more other embodiments. Such combinations can form a number of embodiments within the scope of the invention. It is intended that the scope of the invention be defined by the following claims and their equivalents.

TABLE 1

| | | | | Incyte | Incyte |
|---|---|---|---|---|---|
| Incyte | Polypeptide | Incyte | Polynucleotide | Polynucleotide | Full Length |
| Project ID | SEQ ID NO: | Polypeptide ID | SEQ ID NO: | ID | Clones |
| 4001873 | 1 | 4001873CD1 | 34 | 4001873CB1 | 4001873CA2, |
| | | | | | 90188609CA2, |
| | | | | | 90188617CA2, |
| | | | | | 90188625CA2, |
| | | | | | 90188633CA2, |
| | | | | | 90188641CA2, |
| | | | | | 90188650CA2, |
| | | | | | 90188690CA2, |
| | | | | | 90188701CA2, |
| | | | | | 90188709CA2, |
| | | | | | 90188717CA2, |
| | | | | | 90188725CA2, |
| | | | | | 90188741CA2, |
| | | | | | 90188841CA2, |
| | | | | | 90188945CA2, |
| | | | | | 90189241CA2, |
| | | | | | 90191237CA2 |
| 55003135 | 2 | 55003135CD1 | 35 | 55003135CB1 | 55003135CA2, |
| | | | | | 95111408CA2, |
| | | | | | 95111488CA2, |
| | | | | | 95111516CA2, |
| | | | | | 95111532CA2, |
| | | | | | 95111556CA2, |
| | | | | | 95111596CA2 |
| 5855204 | 3 | 5855204CD1 | 36 | 5855204CB1 | |
| 5778654 | 4 | 5778654CD1 | 37 | 5778654CB1 | |
| 1440126 | 5 | 1440126CD1 | 38 | 1440126CB1 | 90159891CA2 |
| 3934519 | 6 | 3934519CD1 | 39 | 3934519CB1 | |
| 2946314 | 7 | 2946314CD1 | 40 | 2946314CB1 | |
| 3617784 | 8 | 3617784CD1 | 41 | 3617784CB1 | |
| 7490869 | 9 | 7490869CD1 | 42 | 7490869CB1 | |
| 5994687 | 10 | 5994687CD1 | 43 | 5994687CB1 | 90110777CA2, |
| | | | | | 90110793CA2, |
| | | | | | 90110869CA2, |
| | | | | | 90110893CA2 |
| 2560755 | 11 | 2560755CD1 | 44 | 2560755CB1 | |
| 3217430 | 12 | 3217430CD1 | 45 | 3217430CB1 | |
| 5786832 | 13 | 5786832CD1 | 46 | 5786832CB1 | |
| 7493320 | 14 | 7493320CD1 | 47 | 7493320CB1 | |
| 2911453 | 15 | 2911453CD1 | 48 | 2911453CB1 | |
| 3029661 | 16 | 3029661CD1 | 49 | 3029661CB1 | |
| 71260474 | 17 | 71260474CD1 | 50 | 71260474CB1 | 358623CA2 |
| 7992707 | 18 | 7992707CD1 | 51 | 7992707CB1 | |
| 7974861 | 19 | 7974861CD1 | 52 | 7974861CB1 | |
| 7499710 | 20 | 7499710CD1 | 53 | 7499710CB1 | |
| 8036958 | 21 | 8036958CD1 | 54 | 8036958CB1 | |
| 3253807 | 22 | 3253807CD1 | 55 | 3253807CB1 | 6108856CA2, |
| | | | | | 90129032CA2 |
| 3626408 | 23 | 3626408CD1 | 56 | 3626408CB1 | 5913065CA2 |
| 3773014 | 24 | 3773014CD1 | 57 | 3773014CB1 | |
| 4398735 | 25 | 4398735CD1 | 58 | 4398735CB1 | |
| 7499579 | 26 | 7499579CD1 | 59 | 7499579CB1 | |
| 8178947 | 27 | 8178947CD1 | 60 | 8178947CB1 | |
| 2264652 | 28 | 2264652CD1 | 61 | 2264652CB1 | |
| 1806372 | 29 | 1806372CD1 | 62 | 1806372CB1 | |
| 2010564 | 30 | 2010564CD1 | 63 | 2010564CB1 | 90130747CA2 |
| 7364908 | 31 | 7364908CD1 | 64 | 7364908CB1 | |
| 7489960 | 32 | 7489960CD1 | 65 | 7489960CB1 | |
| 8555401 | 33 | 8555401CD1 | 66 | 8555401CB1 | |

TABLE 2

| | | GenBank ID NO: | | |
|---|---|---|---|---|
| Polypeptide SEQ | Incyte | or PROTEOME | Probability | |
| ID NO: | Polypeptide ID | ID NO: | Score | Annotation |
| 1 | 4001873CD1 | g10121865 | 1.1E−16 | [Homo sapiens] topoisomerase II alpha-4 |
| | | | | Petruti-Mot, A. S. and Earnshaw, W. C. (2000) Gene 258: 183-192 |
| 2 | 55003135CD1 | g10121865 | 2.4E−26 | [Homo sapiens] topoisomerase II alpha-4 |
| | | | | Petruti-Mot, A. S. and Earnshaw, W. C. (supra) |
| 3 | 5855204CD1 | g1894791 | 0.0 | [Mus musculus] 5′-3′ exonuclease |
| | | | | Bashkirov, V. I. (1997) J. Cell Biol. 136: 761-773 |
| 4 | 5778654CD1 | g3061308 | 5.1E−05 | [Mus musculus] topoisomerase III |
| | | | | Seki T, et al. (1998) Biochim Biophys Acta 1396: 127-31 |
| 5 | 1440126CD1 | g488555 | 2.1E−127 | [Homo sapiens] zinc finger protein ZNF135 |
| | | | | Tommerup, N. and Vissing, H. (1995) Genomics 27: 259-264 |
| 6 | 3934519CD1 | g1020145 | 4.5E−205 | [Homo sapiens] DNA binding protein |
| | | | | Bellefroid, E. J. et al. (1989) DNA: 377-387 |
| 7 | 2946314CD1 | g4557143 | 0.0 | [Rattus norvegicus] zinc finger protein RIN ZF |
| | | | | Tillotson, L. G. (1999) J. Biol. Chem. 274: 8123-8128 |
| 8 | 3617784CD1 | g6984172 | 9.1E−216 | [Homo sapiens] zinc finger protein ZNF226 |
| 9 | 7490869CD1 | g2252814 | 0.0 | [Mus musculus] FOG |
| | | | | Tsang, A. P., et al. (1997)Cell 90: 109-119 |
| 11 | 2560755CD1 | g7578595 | 0.0 | [Mus musculus] teashirt 2 |
| | | | | Caubit, X. et al. (2000) Mech. Dev. 91: 445-448 |
| 12 | 3217430CD1 | g13310782 | 3.9E−78 | [Mus musculus] myoneurin |
| | | | | Alliel, P. M. et al. (2000) Biochem. Biophys. Res. Commun. 273: 385-391 |
| 13 | 5786832CD1 | g13752754 | 1.4E−272 | [Homo sapiens] zinc finger 1111 |
| 14 | 7493320CD1 | g14150547 | 8.3E−197 | [Mus musculus] cer-d4 isoform XZ |
| | | | | Ninkina, N. N., et al. (2001) Mamm. Genome 12: 862-866 |
| 15 | 2911453CD1 | g14486069 | 4.2E−69 | [Drosophila melanogaster] (AY032609) Zn finger transcription factor lame duck |
| | | | | Duan H, et al. (2001) Development 128: 4489-4500 |
| 16 | 3029661CD1 | g190234 | 2.7E−40 | [Homo sapiens] acidic ribosomal phosphoprotein (P1) |
| | | | | Rich, B. E. and Steitz, J. A. (1987) Mol. Cell. Biol. 7: 4065-4074 |
| 17 | 71260474CD1 | g3746838 | 7.0E−11 | [Homo sapiens] 38 kDa splicing factor; SPF 38 |
| | | | | Neubauer, G. et al. (1998) Nat. Genet. 20: 46-50 |
| 18 | 7992707CD1 | g10963 | 0.0 | [Drosophila melanogaster] RNA polymerase III second-largest subunit |
| | | | | Seifarth, W. et al. (1991) Mol. Gen. Genet. 228: 424-432 |
| 19 | 7974861CD1 | g6692607 | 0.0 | [Mus musculus] MGA protein |
| | | | | Hurlin, P. J. et al. (1999) EMBO J. 18: 7019-7028 |
| 20 | 7499710CD1 | g13021892 | 0.0 | [Homo sapiens] PGC-1 related co-activator |
| | | | | Andersson, U. and Scarpulla, R. C. (2001) Mol. Cell. Biol. 21: 3738-3749 |
| 21 | 8036958CD1 | g14090215 | 2.4E−137 | [Oryza sativa] putative ATP-dependent RNA helicase A |
| 22 | 3253807CD1 | g493572 | 7.5E−191 | [Homo sapiens] zinc finger protein ZNF131 |
| | | | | Tommerup, N. and Vissing, H. (1995) Genomics 27: 259-264 |
| 23 | 3626408CD1 | g4588906 | 9.3E−90 | [Secale cereale] ribosomal protein S7 |
| | | | | Berberich, T. et al. (2000) Biochim. Biophys. Acta 1492: 276-279 |
| 24 | 3773014CD1 | g1806113 | 0.0 | [Homo sapiens] zinc finger protein Hsal2 |
| | | | | Kohlhase, J. et al. (1996) Genomics 38: 291-298 |
| 25 | 4398735CD1 | g1669689 | 0.0 | [Homo sapiens] TBP associated factor |
| | | | | Dikstein, R. et al. Cell 87: 137-146 (1996) |
| 26 | 7499579CD1 | g693937 | 1.2E−197 | [Homo sapiens] polyadenylate binding protein II |
| 27 | 8178947CD1 | g498152 | 6.3E−107 | [Homo sapiens] ha0946 protein is Kruppel-related. |
| | | | | Nomura, N. et al. DNA Res. 1: 223-229 (1994) |
| 28 | 2264652CD1 | g1894792 | 0.0 | [Mus musculus] 5′-3′ exonuclease |
| | | | | Bashkirov, V. I. et al. (1997) J. Cell Biol. 136, 761-773 |
| 29 | 1806372CD1 | g14484930 | 0.0 | [Mus musculus] DEAQ RNA-dependent ATPase DQX1 |
| | | | | Ji, W. et al. Mamm. Genome 12: 456-461 (2001) |
| 30 | 2010564CD1 | g15011452 | 1.1E−88 | [Homo sapiens] GRAIL: a novel ring finger protein upregulated in anergic T cells |
| 31 | 7364908CD1 | g15081398 | 1.7E−106 | [Homo sapiens] kruppel-like zinc finger protein |
| 32 | 7489960CD1 | g13161145 | 6.5E−34 | [Homo sapiens] zinc finger protein |
| 33 | 8555401CD1 | g13161090 | 5.5E−211 | [Homo sapiens] heat shock transcription factor 2-like protein |

TABLE 3

| | | Amino | | Potential | | |
|---|---|---|---|---|---|---|
| SEQ | | Acid | Potential | Glyco- | | |
| ID | Incyte | Resi- | Phosphorylation | sylation | | Analytical Methods |
| NO: | Polypeptide ID | dues | Sites | Sites | Signature Sequences, Domains and Motifs | and Databases |
| 1 | 4001873CD1 | 106 | | | Signal_cleavage: M1-L18 | SPSCAN |
| | | | | | Signal Peptide: M1-L18, M1-P20 | HMMER |
| | | | | | Non-cytosolic domain: M1-P106 | TMHMMER |
| 2 | 55003135CD1 | 241 | S114 S135 S217 | N101 | Signal_cleavage: M1-A18 | SPSCAN |
| | | | T29 T57 T86 T154 | | | |
| | | | T199 | | | |
| | | | | | Signal Peptide: M1-A18 | HMMER |
| | | | | | Non-cytosolic domain: M1-G241 | TMHMMER |
| | | | | | JSP NEURONAL THREAD PD003801: S131-I178 | BLAST_PRODOM |
| | | | | | PROTEIN PROTOONCOGENE NUCLEAR | BLAST_PRODOM |
| | | | | | UBIQUITOUS TPR MOTIFY ISOFORM MYB | |
| | | | | | CMYB PD015557: F179-R221 | |
| 3 | 5855204CD1 | 1023 | S11 S104 S169 | N326 N494 | Non-cytosolic domain: M1-G1023 | TMHMMER |
| | | | S187 S344 S584 | N582 N790 | | |
| | | | S682 S683 S731 | | | |
| | | | S750 S908 S941 | | | |
| | | | S981 S1005 T157 | | | |
| | | | T173 T215 T235 | | | |
| | | | T270 T400 T411 | | | |
| | | | T420 T497 T535 | | | |
| | | | T647 T661 T745 | | | |
| | | | T802 T848 T970 | | | |
| | | | Y67 Y149 Y611 | | | |
| | | | Y969 Y979 Y1000 | | | |
| | | | Y1011 | | | |
| | | | | | EXONUCLEASE PROTEIN HYDROLASE | BLAST_PRODOM |
| | | | | | NUCLEASE 5′3′ NUCLEAR EXORIBONUCLEASE | |
| | | | | | MAGNESIUM DNA RECOMBINATION | |
| | | | | | PD005946: E28-T244, M1-I39, L297-M600 | |
| | | | | | EXONUCLEASE 5′3′ HYDROLASE NUCLEASE | BLAST_PRODOM |
| | | | | | MAGNESIUM DNA RECOMBINATION | |
| | | | | | DNABINDING PROTEIN II PD014574: Y611-N1020 | |
| | | | | | MOUSE; DHM1; DM02631 | BLAST_DOMO |
| | | | | | P40383\|14-812: Y14-V354, L402-V739, Y969-Y1011 | |
| | | | | | P40848\|35-981: E28-E421, N405-M600 | |
| | | | | | I49635\|34-905: E28-C239, D414-N593, C239-E353, | |
| | | | | | N582-C601 | |
| | | | | | ATP/GTP-binding site motif A (P-loop): A707-S714 | MOTIFS |
| 4 | 5778654CD1 | 441 | S142 S152 S174 | N76 N96 | Non-cytosolic domain: M1-E441 | TMHMMER |
| | | | S180 S193 S211 | N108 N150 | | |
| | | | S219 S281 S290 | N209 | | |
| | | | S353 S376 S426 | | | |
| | | | T125 T192 T407 | | | |
| | | | | | Prokaryotic DNA Topoisomerase PR00417C: V23-D32 | BLIMPS_PRINTS |
| 5 | 1440126CD1 | 446 | S113 S238 S350 | N66 N262 | Zinc finger, C2H2 type: Y364-H386, Y392-H413, | HMMER_PFAM |
| | | | S427 T120 T219 | | Y308-H330, Y336-H358, Y196-H218, Y419-H441, | |
| | | | T275 T303 T387 | | Y224-H246, Y252-H274, Y168-H190, Y140-H162, | |
| | | | | | H280-H302 | |
| | | | | | ZINC FINGER, C2H2 type BL00028 C142-H158 | BLIMPS_BLOCKS |
| | | | | | PROTEIN ZINC FINGER META PD000066 H326-C338 | BLIMPS_{—} |
| | | | | | | PRODOM |
| | | | | | PROTEIN ZINC FINGER METAL-BINDING DNA- | BLAST_PRODOM |
| | | | | | BINDING ZINC FINGER PATERNALLY | |
| | | | | | EXPRESSED ZN FINGER PW1 PD017719: G164-H413 | |
| | | | | | ZINC FINGER DNA-BINDING PROTEIN METAL- | BLAST_PRODOM |
| | | | | | BINDING NUCLEAR ZINC FINGER | |
| | | | | | TRANSCRIPTION REGULATION REPEAT | |
| | | | | | PD000072: K306-C369 | |
| | | | | | HYPOTHETICAL ZINC FINGER PROTEIN | BLAST_PRODOM |
| | | | | | B03B8.4 IN CHROMOSOME III ZINC FINGER | |
| | | | | | DNA-BINDING METAL-BINDING NUCLEAR | |
| | | | | | PD149420: E165-H437 | |
| | | | | | ZINC FINGER PROTEIN ZINC FINGER METAL- | BLAST_PRODOM |
| | | | | | BINDING DNA-BINDING PUTATIVE REX2 | |
| | | | | | TRANSCRIPTION REGULATION PD033163: | |
| | | | | | C171-K306 | |
| | | | | | ZINC FINGER, C2H2 TYPE, DOMAIN DM00002 | BLAST_DOMO |
| | | | | | \|Q05481\|831-885: C145-E200 | |
| | | | | | \|Q05481\|789-829: Q299-K340 | |
| | | | | | \|P08042\|314-358: C313-H358 | |
| | | | | | \|P52743\|31-93: L295-H358 | |
| | | | | | Cell attachment sequence: R122-D124 | MOTIFS |
| | | | | | Zinc finger, C2H2 type, domain: C142-H162, C170-H190, | MOTIFS |
| | | | | | C198-H218, C226-H246, C254-H274, C282-H302, | |
| | | | | | C310-H330, C338-H358, C366-H386, C421-H441 | |
| 6 | 3934519CD1 | 686 | S224 S241 S280 | N176 N196 | KRAB box: V8-E70 | HMMER_PFAM |
| | | | S308 S420 S476 | | | |
| | | | S504 S588 S672 T9 | | | |
| | | | T18 T52 T106 | | | |
| | | | T657 Y662 | | | |
| | | | | | Zinc finger, C2H2 type: Y270-H292, Y354-H376, | HMMER_PFAM |
| | | | | | F634-H656, F186-H208, Y326-H348, Y662-H684, | |
| | | | | | Y522-H544, Y410-H432, Y214-H236, Y382-H404, | |
| | | | | | Y438-H460, Y466-H488, Y606-H628, Y242-H264, | |
| | | | | | Y298-H320, Y578-H600, Y494-H516, Y550-H572 | |
| | | | | | ZINC FINGER, C2H2 type BL00028 C664-H680 | BLIMPS_BLOCKS |
| | | | | | PROTEIN ZINC FINGER ZINC PD01066: F10-G48 | BLIMPS_{—} |
| | | | | | | PRODOM |
| | | | | | PROTEIN ZINC FINGER METAL-BINDING DNA- | BLAST_PRODOM |
| | | | | | BINDING ZINC FINGER PATERNALLY | |
| | | | | | EXPRESSED ZN FINGER PW1 PD017719: G350-H600 | |
| | | | | | ZINC FINGER METAL-BINDING DNA-BINDING | BLAST_PRODOM |
| | | | | | PROTEIN FINGER ZINC NUCLEAR REPEAT | |
| | | | | | TRANSCRIPTION REGULATION PD001562: V8-E70 | |
| | | | | | HYPOTHETICAL ZINC FINGER PROTEIN | BLAST_PRODOM |
| | | | | | B03B8.4 IN CHROMOSOME III ZINC FINGER | |
| | | | | | DNA-BINDING METAL BINDING NUCLEAR | |
| | | | | | PD149420: E323-F509 | |
| | | | | | ZINC FINGER DNA-BINDING PROTEIN METAL- | BLAST_PRODOM |
| | | | | | BINDING NUCLEAR ZINC FINGER | |
| | | | | | TRANSCRIPTION REGULATION REPEAT | |
| | | | | | PD000072: K296-C359 | |
| | | | | | KRAB BOX DOMAIN DM00605 | BLAST_DOMO |
| | | | | | \|I48689\|11-85: Q5-R75 | |
| | | | | | \|P52738\|3-77: Q5-R75 | |
| | | | | | \|P51523\|5-79: Q5-V73 | |
| | | | | | \|P52736\|1-72: V8-E72 | |
| | | | | | Zinc finger, C2H2 type, domain: C188-H208, C216-H236, | MOTIFS |
| | | | | | C244-H264, C272-H292, C300-H320, C328-H348, | |
| | | | | | C356-H376, C384-H404, C412-H432, C440-H460, | |
| | | | | | C468-H488, C496-H516, C524-H544, C552-H572, | |
| | | | | | C580-H600, C608-H628, C636-H656, C664-H684 | |
| 7 | 2946314CD1 | 903 | S2 S138 S179 S183 | N136 N436 | Signal Peptide: M6-T27, M6-S26 | HMMER |
| | | | S192 S197 S206 | N437 N496 | | |
| | | | S207 S210 S343 | N538 N566 | | |
| | | | S403 S428 S498 | N581 N610 | | |
| | | | S524 S660 S801 | N624 | | |
| | | | S831 S889 S900 | | | |
| | | | T184 T278 T338 | | | |
| | | | T439 T486 T603 | | | |
| | | | T606 T612 T613 | | | |
| | | | T626 T678 T687 | | | |
| | | | T711 T768 T792 | | | |
| | | | Y378 | | | |
| | | | | | BTB/POZ domain: Y380-L495 | HMMER_PFAM |
| | | | | | Zinc finger, C2H2 type: F782-H804, L754-H776 | HMMER_PFAM |
| | | | | | ZINC FINGER, C2H2 type BL00028 C784-H800 | BLIMPS_BLOCKS |
| | | | | | BTB PF00651 A409-F421 | BLIMPS_PFAM |
| | | | | | PROTEIN ZINC FINGER META PD000066 H800-C812 | BLIMPS_{—} |
| | | | | | | PRODOM |
| | | | | | PROTEIN DNA-BINDING ZINC FINGER METAL- | BLAST_PRODOM |
| | | | | | BINDING NUCLEAR ZINC FINGER | |
| | | | | | TRANSCRIPTION REGULATION | |
| | | | | | CHROMOSOME PD000632: I360-L495 | |
| | | | | | POZ DOMAIN DM00509 | BLAST_DOMO |
| | | | | | \|S59069\|1-171: Q381-K515 | |
| | | | | | \|P10074\|1-153: Q381-F490 | |
| | | | | | \|P41182\|7-213: L382-K492 | |
| | | | | | \|S44264\|27-229: S377-K492 | |
| | | | | | ATP/GTP-binding site motif A (P-loop): G599-T606 | MOTIFS |
| | | | | | Zinc finger, C2H2 type, domain: C784-H804 | MOTIFS |
| 8 | 3617784CD1 | 847 | S80 S121 S127 | | KRAB box: V30-E92 | HMMER_PFAM |
| | | | S342 S484 S607 | | | |
| | | | S751 T25 T31 T40 | | | |
| | | | T95 T394 T456 | | | |
| | | | T701 T735 Y111 | | | |
| | | | | | Zinc finger, C2H2 type: Y622-H644, Y790-H812, | HMMER_PFAM |
| | | | | | Y567-H588, Y371-H393, Y455-H477, Y734-H756, | |
| | | | | | Y483-H505, Y678-H700, Y399-H421, Y511-H533, | |
| | | | | | Y594-H616, Y539-H561, Y427-H449, Y762-H784, | |
| | | | | | Y706-H728, Y650-H672, Y818-H840 | |
| | | | | | ZINC FINGER C2H2 type BL00028 C401-H417 | BLIMPS_BLOCKS |
| | | | | | C2H2-type zinc finger signature PR00048: P398-S411, | BLIMPS_PRINTS |
| | | | | | L693-G702 | |
| | | | | | PROTEIN ZINC FINGER ZINC PD01066: F32-A70 | BLIMPS_{—} |
| | | | | | | PRODOM |
| | | | | | PROTEIN ZINC FINGER METAL-BINDING DNA- | BLAST_PRODOM |
| | | | | | BINDING ZINC FINGER PATERNALLY | |
| | | | | | EXPRESSED ZN FINGER PW1 PD017719: G395-V643 | |
| | | | | | ZINC FINGER METAL-BINDING DNA-BINDING | BLAST_PRODOM |
| | | | | | PROTEIN FINGER ZINC NUCLEAR REPEAT | |
| | | | | | TRANSCRIPTION REGULATION PD001562: V30-E92 | |
| | | | | | ZINC FINGER DNA-BINDING PROTEIN METAL- | BLAST_PRODOM |
| | | | | | BINDING NUCLEAR ZINC FINGER | |
| | | | | | TRANSCRIPTION REGULATION REPEAT | |
| | | | | | PD000072: R397-C460 | |
| | | | | | HYPOTHETICAL ZINC FINGER PROTEIN | BLAST_PRODOM |
| | | | | | B03B8.4 IN CHROMOSOME III ZINC FINGER | |
| | | | | | DNA-BINDING METAL-BINDING NUCLEAR | |
| | | | | | PD149420: S662-N844 | |
| | | | | | KRAB BOX DOMAIN DM00605\|I48689\|11-85: | BLAST_DOMO |
| | | | | | K27-C100 | |
| | | | | | ZINC FINGER, C2H2 TYPE, DOMAIN DM00002 | BLAST_DOMO |
| | | | | | \|Q05481\|789-829: V392-E431; 831-885: C376-E431 | |
| | | | | | \|P08042\|314-358: C627-H672 | |
| | | | | | Zinc finger, C2H2 type, domain: C373-H393, C401-H421, | MOTIFS |
| | | | | | C429-H449, C457-H477, C485-H505, C513-H533, | |
| | | | | | C541-H561, C596-H616, C624-H644, C652-H672, | |
| | | | | | C680-H700, C708-H728, C736-H756, C764-H784, | |
| | | | | | C792-H812, C820-H840 | |
| 9 | 7490869CD1 | 1003 | S2 S7 S15 S31 S87 | | Signal Peptide: M311-C335 | HMMER |
| | | | S128 S252 S305 | | | |
| | | | S378 S459 S598 | | | |
| | | | S668 S669 S690 | | | |
| | | | S705 S765 S795 | | | |
| | | | S836 S948 S950 | | | |
| | | | T38 T78 T157 | | | |
| | | | T330 T358 T437 | | | |
| | | | T575 T650 T680 | | | |
| | | | T737 T891 | | | |
| | | | | | Zinc finger, C2H2 type: T680-C703, H814-C837, | HMMER_PFAM |
| | | | | | R971-C994, G348-H371, A851-H874, R290-H314, | |
| | | | | | F320-H342, F241-C264, A574-C597 | |
| | | | | | ZINC FINGER, C2H2 TYPE BL00028 C682-H698 | BLIMPS_BLOCKS |
| | | | | | PROTEIN ZINC FINGER META PD000066 H310-C322 | BLIMPS_{—} |
| | | | | | | PRODOM |
| | | | | | FRIEND OF GATA1 FOG | BLAST_PRODOM |
| | | | | | PD137790: T345-D708 | |
| | | | | | PD129613: M1-C292 | |
| | | | | | PD108418: A760-P946 | |
| | | | | | HPBRII; DM05499\|S57447\|251-354: P709-A809 | BLAST_DOMO |
| | | | | | Cell attachment sequence: R863-D865 | MOTIFS |
| | | | | | Zinc finger, C2H2 type, domain: C292-H314, C322-H342 | MOTIFS |
| 10 | 5994687CD1 | 192 | S150 T26 | N21 | signal_cleavage: M1-A66 | SPSCAN |
| | | | | | Zinc finger, C3HC4 type (RING finger): C13-C59 | HMMER_PFAM |
| | | | | | ZINC FINGER C3HC4 TYPE BL00518 C32-C40 | BLIMPS_BLOCKS |
| | | | | | Zinc finger, C3HC4 type (RING finger), signature: | PROFILESCAN |
| | | | | | A11-V64 | |
| | | | | | Zinc finger, C3HC4 type (RING finger), signature: | MOTIFS |
| | | | | | C32-L41 | |
| 11 | 2560755CD1 | 1034 | S75 S104 S148 | N46 N81 | Zinc finger, C2H2 type: F926-H948, F215-H239, | HMMER_PFAM |
| | | | S150 S161 S164 | N159 N162 | L275-H299, L380-H404, F994-H1017 | |
| | | | S201 S286 S313 | N235 N570 | | |
| | | | S339 S346 S387 | | | |
| | | | S413 S431 S449 | | | |
| | | | S456 S464 S473 | | | |
| | | | S604 S618 S627 | | | |
| | | | S629 S633 S662 | | | |
| | | | S746 S753 S772 | | | |
| | | | S813 S943 S965 | | | |
| | | | T48 T50 T56 T226 | | | |
| | | | T472 T495 T572 | | | |
| | | | T639 T807 T1029 | | | |
| | | | | | ZINC FINGER C2H2 TYPE BL00028 C277-H293 | BLIMPS_BLOCKS |
| | | | | | ANTIGEN NYCO33 PD146846: S339-K1006 | BLAST_PRODOM |
| | | | | | Zinc finger, C2H2 type, domain: C217-H239, C277-H299, | MOTIFS |
| | | | | | C928-H948, C996-H1017 | |
| 12 | 3217430CD1 | 765 | S59 S85 S186 S201 | N56 N71 | BTB/POZ domain: C9-T121 | HMMER_PFAM |
| | | | S260 S317 S352 | N131 N167 | | |
| | | | S371 S391 S408 | N282 N490 | | |
| | | | S446 S606 S629 | | | |
| | | | S640 S665 S693 | | | |
| | | | T169 T245 T250 | | | |
| | | | T334 T373 T508 | | | |
| | | | T735 Y451 Y535 | | | |
| | | | | | Zinc finger, C2H2 type: F423-H445, Y395-H417, | HMMER_PFAM |
| | | | | | Y563-H585, Y535-H557, Y451-H473, F507-H529, | |
| | | | | | H479-H501 | |
| | | | | | C2H2-type zinc finger signature PR00048: P422-A435, | BLIMPS_PRINTS |
| | | | | | L550-G559 | |
| | | | | | ZINC FINGER C2H2 TYPE BL00028 C481-H497 | BLIMPS_BLOCKS |
| | | | | | BTB PF00651 A38-F50 | BLIMPS_PFAM |
| | | | | | PROTEIN ZINC FINGER METAL PD000066 H553-C565 | BLIMPS_{—} |
| | | | | | | PRODOM |
| | | | | | PROTEIN ZINC INGER METAL-BINDING DNA- | BLAST_PRODOM |
| | | | | | BINDING ZINC FINGER PATERNALLY | |
| | | | | | EXPRESSED ZN FINGER PW1 PD017719: G447-T621 | |
| | | | | | ZINC FINGER DNA-BINDING PROTEIN METAL- | BLAST_PRODOM |
| | | | | | BINDING NUCLEAR ZINC FINGER | |
| | | | | | TRANSCRIPTION REGULATION REPEAT | |
| | | | | | PD000072: K449-C512 | |
| | | | | | POZ DOMAIN DM00509\|S59069\|1-171: H7-S119 | BLAST_DOMO |
| | | | | | POZ DOMAIN DM00509 | BLAST_DOMO |
| | | | | | \|P41182\|7-213: H7-H184 | |
| | | | | | \|S44264\|27-229: V4-H184 | |
| | | | | | \|P10074\|1-153: H7-G127 | |
| | | | | | Zinc finger, C2H2 type, domain: C397-H417, C425-H445, | MOTIFS |
| | | | | | C453-H473, C481-H501, C509-H529, C537-H557, | |
| | | | | | C565-H585 | |
| 13 | 5786832CD1 | 896 | S18 S58 S93 S171 | N162 N233 | Zinc finger, C2H2 type: Y401-H423, Y653-H675, | HMMER_PFAM |
| | | | S235 S294 S414 | N292 N413 | Y597-H619, Y765-H787, Y373-H395, Y513-H535, | |
| | | | S693 S719 S873 T9 | N611 N747 | Y849-H871, F625-H647, Y457-H479, H709-H731, | |
| | | | T158 T249 T285 | N842 N884 | Y793-H815, Y737-H759, Y541-H563, L569-H591, | |
| | | | T409 T663 T732 | | Y261-H283, Y289-H311, Y345-H367, Y485-H507, | |
| | | | Y152 Y737 | | Y317-H339, Y429-H451, Y821-H843 | |
| | | | | | HNH endonuclease: Y401-E454 | HMMER_PFAM |
| | | | | | KRAB box: L8-V70 | HMMER_PFAM |
| | | | | | ZINC FINGER C2H2 TYPE BL00028 C487-H503 | BLIMPS_BLOCKS |
| | | | | | C2H2-type zinc finger signature PR00048: P400-N413, | BLIMPS_PRINTS |
| | | | | | L528-G537 | |
| | | | | | PROTEIN ZINC FINGER ZINC PD01066: F10-G48 | BLIMPS_{—} |
| | | | | | | PRODOM |
| | | | | | PROTEIN ZINC FINGER METAL-BINDING DNA- | BLAST_PRODOM |
| | | | | | BINDING ZINC FINGER PATERNALLY | |
| | | | | | EXPRESSED ZN FINGER PW1 PD017719: G649-G883 | |
| | | | | | ZINC FINGER METAL-BINDING DNA-BINDING | BLAST_PRODOM |
| | | | | | PROTEIN FINGER ZINC NUCLEAR REPEAT | |
| | | | | | TRANSCRIPTION REGULATION PD001562: L8-W67 | |
| | | | | | ZINC FINGER DNA-BINDING PROTEIN METAL- | BLAST_PRODOM |
| | | | | | BINDING NUCLEAR ZINC FINGER | |
| | | | | | TRANSCRIPTION REGULATION REPEAT | |
| | | | | | PD000072: K819-C882 | |
| | | | | | KRAB BOX DOMAIN DM00605 | BLAST_DOMO |
| | | | | | \|Q05481\|10-83: G6-V69 | |
| | | | | | \|P52738\|3-77: Q5-K74 | |
| | | | | | \|Q03923\|1-75: G6-R77 | |
| | | | | | \|I48689\|11-85: Q5-C81 | |
| | | | | | Zinc finger, C2H2 type, domain: C319-H339, C347-H367, | MOTIFS |
| | | | | | C375-H395, C403-H423, C431-H451, C459-H479, | |
| | | | | | C487-H507, C515-H535, C543-H563, C571-H591, | |
| | | | | | C599-H619, C627-H647, C655-H675, C711-H731, | |
| | | | | | C739-H759, C767-H787, C795-H815, C823-H843, | |
| | | | | | C851-H871 | |
| 14 | 7493320CD1 | 357 | S36 S138 S190 | N170 | ZINC FINGER C2H2 TYPE BL00028 C200-H216 | BLIMPS_BLOCKS |
| | | | S275 S283 S316 | | | |
| | | | S318 S338 S344 | | | |
| | | | S356 T116 T309 | | | |
| | | | Y17 | | | |
| | | | | | ZINC FINGER PROTEIN NUCLEAR NEUROD4 | BLAST_PRODOM |
| | | | | | ALTERNATIVE SPLICING UBID4 APOPTOSIS | |
| | | | | | RESPONSE ZINC PD016426: M1-E141 | |
| | | | | | PROTEIN ZINC FINGER NUCLEAR ZINC | BLAST_PRODOM |
| | | | | | FINGER NEUROD4 ALTERNATIVE SPLICING | |
| | | | | | UBID4 APOPTOSIS PD010829: R183-H292 | |
| | | | | | REQUIEM; TRANSCRIPTION; D4; NEURO; | BLAST_DOMO |
| | | | | | DM05393\|S26731\|1-177: M1-G181 | |
| | | | | | DM05393\|A55302\|1-171: I21-G178 | |
| | | | | | DM03861\|A55302\|211-313: T220-H292 | |
| | | | | | DM03861\|S26731\|217-340: T220-H292 | |
| | | | | | Zinc finger, C2H2 type, domain: C200-H221 | MOTIFS |
| 15 | 2911453CD1 | 513 | S63 S68 S73 S76 | N110 N388 | signal_cleavage: M1-M38 | SPSCAN |
| | | | S146 S217 S357 | | | |
| | | | S390 S421 S430 | | | |
| | | | S442 S465 S487 | | | |
| | | | T208 T264 T398 | | | |
| | | | T413 T417 Y405 | | | |
| | | | | | Zinc finger, C2H2 type: Y405-H429, N345-H369, | HMMER_PFAM |
| | | | | | Y375-H399, F312-H339, H278-H303 | |
| | | | | | ZINC FINGER C2H2 TYPE BL00028 F349-H365 | BLIMPS_BLOCKS |
| | | | | | Vinculin signature PR00806: L221-P231, P232-H245 | BLIMPS_PRINTS |
| | | | | | PROTEIN ZINC FINGER METAL PD00066 H365-C377 | BLIMPS_{—} |
| | | | | | | PRODOM |
| | | | | | PROTEIN ZINC FINGER METAL-BINDING DNA- | BLAST_PRODOM |
| | | | | | BINDING NUCLEAR ZINC FINGER | |
| | | | | | TRANSCRIPTION REGULATION HGLI2 | |
| | | | | | PD002819: K403-P448 | |
| | | | | | ZINC FINGER, C2H2 TYPE, DOMAIN DM00002 | BLAST_DOMO |
| | | | | | \|P46684\|274-321: C319-H365 | |
| | | | | | \|P39768\|263-310: G318-H365 | |
| | | | | | Zinc finger, C2H2 type, domain: C280-H303, C347-H369, | MOTIFS |
| | | | | | C377-H399, C407-H429 | |
| 16 | 3029661CD1 | 104 | S3 S94 T22 | | 60s Acidic ribosomal protein: M1-D104 | HMMER_PFAM |
| | | | | | RIBOSOMAL PROTEIN ACIDIC 60S | BLAST_PRODOM |
| | | | | | PHOSPHORYLATION P2 P1 L12 MULTIGENE | |
| | | | | | FAMILY PD001928: M1-D104 | |
| | | | | | RAT ACIDIC RIBOSOMAL PROTEIN P1 | BLAST_DOMO |
| | | | | | DM00632\|A53221\|1-111: A2-L102 | |
| | | | | | DM00632\|P22684\|1-112: E6-D104 | |
| | | | | | DM00632\|P49148\|1-109: S3-D104 | |
| | | | | | DM00632\|P26643\|1-108: M1-D104 | |
| 17 | 71260474CD1 | 255 | S129 S249 T203 | N191 | signal_cleavage: M1-A55 | SPSCAN |
| | | | T240 Y212 | | | |
| | | | | | WD domain, G-beta repeat: Q173-D208, P42-D78, | HMMER_PFAM |
| | | | | | A128-M164, M1-N35 | |
| | | | | | Trp-Asp (WD-40) repeats signature: T185-I230, I54-V142 | PROFILESCAN |
| | | | | | TRP-ASP (WD) REPEAT PROTEIN BL00678\|T197- | BLIMPS_BLOCKS |
| | | | | | W207 | |
| | | | | | Trp-Asp (WD) repeats signature: L195-F209 | MOTIFS |
| 18 | 7992707CD1 | 1133 | S67 S98 S123 S206 | N92 N423 | RNA polymerase beta subunit: R95-E1076 | HMMER_PFAM |
| | | | S247 S268 S425 | N577 N738 | | |
| | | | S457 S466 S628 | N784 N829 | | |
| | | | S680 S747 S816 | | | |
| | | | S864 S873 S898 | | | |
| | | | S1103 T22 T148 | | | |
| | | | T151 T229 T304 | | | |
| | | | T462 T504 T558 | | | |
| | | | T579 T607 T655 | | | |
| | | | T778 T783 T799 | | | |
| | | | T857 T888 Y49 | | | |
| | | | Y109 Y356 Y710 | | | |
| | | | | | RNA polymerases beta chain proteins BL01166: | BLIMPS_BLOCKS |
| | | | | | I1002-E1051, L131-T148, D352-R361, R469-V493, | |
| | | | | | G666-G695, G736-K759, K824-T834, G894-P935 | |
| | | | | | POLYMERASE RNA DNA DIRECTED | BLAST_PRODOM |
| | | | | | TRANSCRIPTION SUBUNIT TRANSFERASE | |
| | | | | | BETA CHAIN TRANSCRIPTASE ZINC | |
| | | | | | PD000636: N542-P997, G985-D1073 | |
| | | | | | DNA-DIRECTED RNA POLYMERASE BETA | BLAST_DOMO |
| | | | | | CHAIN DM00241 | |
| | | | | | \|P25167\|474-1132: P468-L1128 | |
| | | | | | \|Q10233\|497-1159: P468-L1128 | |
| | | | | | \|P22276\|492-1143: S474-L1128 | |
| | | | | | DNA-DIRECTED RNA POLYMERASE 132K | BLAST_DOMO |
| | | | | | POLYPEPTIDE DM01030 | |
| | | | | | \|P25167\|37-472: L29-G467 | |
| | | | | | RNA polymerases beta chain signature: G894-V906 | MOTIFS |
| 19 | 7974861CD1 | 3065 | S128 S145 S277 | N81 N183 | signal_cleavage: M1-S60 | SPSCAN |
| | | | S317 S359 S360 | N275 N283 | | |
| | | | S369 S415 S417 | N374 N516 | | |
| | | | S442 S494 S534 | N695 N764 | | |
| | | | S572 S578 S638 | N818 N941 | | |
| | | | S704 S805 S815 | N1608 | | |
| | | | S824 S876 S885 | N1644 | | |
| | | | S895 S908 S914 | N1659 | | |
| | | | S924 S969 S1030 | N2022 | | |
| | | | S1178 S1208 S1272 | N2095 | | |
| | | | S1301 S1308 S1312 | N2100 | | |
| | | | S1366 S1378 S1382 | N2126 | | |
| | | | S1392 S1401 S1412 | N2653 | | |
| | | | S1439 S1470 S1488 | N2950 | | |
| | | | S1569 S1619 S1734 | N2977 | | |
| | | | S1905 S1972 S1983 | N3029 | | |
| | | | S1996 S2024 | | Helix-loop-helix DNA-binding domain: Y2424-T2475 | HMMER_PFAM |
| | | | S2056 S2076 S2077 | | | |
| | | | S2090 S2094 S2138 | | | |
| | | | S2263 S2292 S2315 | | | |
| | | | S2376 S2386 S2395 | | | |
| | | | S2411 S2455 S2500 | | | |
| | | | S2503 S2541 S2554 | | | |
| | | | S2574 S2694 S2702 | | | |
| | | | S2748 S2763 S2786 | | | |
| | | | S2797 | | | |
| | | | S2849 S2910 S2921 | | T-box: T76-D261 | HMMER_PFAM |
| | | | S2940 S3001 S3007 | | | |
| | | | T43 | | | |
| | | | T113 T401 T407 | | Myc-type, ‘helix-loop-helix’ dimerization domain | BLIMPS_BLOCKS |
| | | | T463 T480 T493 | | proteins BL00038: E2432-K2447, S2455-T2475 | |
| | | | T510 T562 T588 | | | |
| | | | T589 T632 T649 | | | |
| | | | T682 T693 T716 | | | |
| | | | T783 T958 T1113 | | T-box domain proteins BL01283: M84-D131, W142- | BLIMPS_BLOCKS |
| | | | T1144 T1148 | | N183, L194-V207, F228-D260 | |
| | | | T1197 T1213 | | | |
| | | | T1511 T1565 | | T-Box domain signature PR00937: S92-D116, F157- | BLIMPS_PRINTS |
| | | | T1754 T1773 | | M170, V174-N183, I193-V207, T231-I244, N252-D260 | |
| | | | T1901 T1916 | | | |
| | | | T1990 T2016 | | PROTEIN DNA BINDING NUCLEAR | BLAST_PRODOM |
| | | | T2020 T2044 | | TRANSCRIPTION TBOX REGULATION | |
| | | | T2058 T2088 | | DEVELOPMENTAL BRACHYURY ACTIVATOR | |
| | | | T2133 T2183 | | T PD001585: L115-D261 | |
| | | | T2207 T2239 | | | |
| | | | T2297 T2354 | | PROTEIN PRECURSOR GLYCOPROTEIN | BLAST_PRODOM |
| | | | T2429 T2489 | | SIGNAL REPEAT ANTIGEN SURFACE | |
| | | | T2606 T2687 | | MEROZOITE CELL TRANSMEMBRANE | |
| | | | T2796 T2811 | | PD000546: T1579-N1739 | |
| | | | T2971 T3031 | | T-BOX DM01478 | BLAST_DOMO |
| | | | Y2423 Y2518 | | \|S46458\|62-380: V77-R269, P1400-Q1433, E1123-L1188 | |
| | | | | | \|A40213\|294-606: D69-P309 | |
| | | | | | \|P80492\|1-332: L50-D260 | |
| | | | | | \|P20293\|8-338: L50-D260 | |
| | | | | | Leucine zipper pattern: L2474-L2495, L2481-L2502 | MOTIFS |
| | | | | | Cell attachment sequence: R2028-D2030 | MOTIFS |
| | | | | | T-box domain signature 2: I158-F176 | MOTIFS |
| 20 | 7499710CD1 | 1400 | S47 S72 S96 S104 | N319 | RNA recognition motif. (a.k.a. RRM, RBD, or RNP | HMMER_PFAM |
| | | | S111 S118 S144 | | domain): V1281-Q1342 | |
| | | | S148 S157 S192 | | | |
| | | | S196 S201 S213 | | | |
| | | | S219 S224 S244 | | | |
| | | | S270 S304 S325 | | | |
| | | | S396 S409 S453 | | | |
| | | | S464 S482 S495 | | | |
| | | | S536 S575 S732 | | | |
| | | | S752 S781 S842 | | | |
| | | | S1097 S1144 S1174 | | | |
| | | | S1209 S1212 S1216 | | | |
| | | | S1234 S1243 S1248 | | | |
| | | | S1255 S1256 S1362 | | | |
| | | | S1364 S1368 S1380 | | | |
| | | | T173 T181 T288 | | | |
| | | | T338 T354 T617 | | | |
| | | | T679 T742 T776 | | | |
| | | | T813 T1053 T1091 | | | |
| | | | T1123 T1148 | | | |
| | | | T1291 T1323 | | | |
| | | | | | HUMAN PEROXISOME PROLIFERATOR | BLAST_PRODOM |
| | | | | | ACTIVATED RECEPTOR (PPAR) GAMMA | |
| | | | | | COACTIVATOR 1 PD056083: R1341-R1400 | |
| | | | | | H-A-P-P REPEAT DM08271\|S25299\|69-249: P913-K1067 | BLAST_DOMO |
| 21 | 8036958CD1 | 1369 | S38 S71 S116 S135 | N70 N73 | Signal Peptide: M1-A30 | HMMER |
| | | | S157 S251 S378 | N165 N695 | | |
| | | | S417 S470 S529 | N797 N1167 | | |
| | | | S580 S599 S634 | | | |
| | | | S672 S739 S755 | | | |
| | | | S830 S895 S908 | | | |
| | | | S964 S1006 S1029 | | | |
| | | | S1209 T120 T209 | | | |
| | | | T280 T306 T333 | | | |
| | | | T351 T370 T375 | | | |
| | | | T461 T484 T559 | | | |
| | | | T584 T799 T938 | | | |
| | | | T948 T1106 T1173 | | | |
| | | | T1197 T1240 Y956 | | | |
| | | | Y1154 | | | |
| | | | | | DEAD/DEAH box helicase: R641-E740, V591-S602 | HMMER_PFAM |
| | | | | | Helicase conserved C-terminal domain: D885-R986 | HMMER_PFAM |
| | | | | | DEAH-box subfamily ATP-dependent helicases | BLIMPS_BLOCKS |
| | | | | | proteins BL00690: G595-Q604, T628-E645, V699-S708 | |
| | | | | | DEAD and DEAH box families ATP-dependent | PROFILESCAN |
| | | | | | helicases signatures: L676-S726 | |
| | | | | | POLYPROTEIN PROTEIN HELICASE GENOME | BLAST_PRODOM |
| | | | | | RNA CONTAINS: NUCLEAR ENVELOPE ATP- | |
| | | | | | BINDING NONSTRUCTURAL PD000440: Y898-T995, | |
| | | | | | L566-T736, A28-R97, H835-F863 | |
| | | | | | HELICASE RNA ATP-BINDING PROTEIN ATP- | BLAST_PRODOM |
| | | | | | DEPENDENT NUCLEAR SPLICING mRNA | |
| | | | | | PROCESSING PRE-mRNA PD001259: C990-H1135 | |
| | | | | | HELICASE PD091835: R569-E762, R864-N889 | BLAST_PRODOM |
| | | | | | DEAH-BOX SUBFAMILY ATP-DEPENDENT | BLAST_DOMO |
| | | | | | HELICASES DM00649\|P24785\|374-1061: K828-R1278, | |
| | | | | | Y562-L857, A493-S558 | |
| | | | | | DM00649\|Q08211\|378-1053: K828-R1278, Q563-V776 | |
| | | | | | DM00649\|S59384\|595-1296: K844-I1231, | |
| | | | | | R569-D780 DM00649\|P34498\|432-1038: I866-V1228, | |
| | | | | | Q563-E772, A802-P824 | |
| | | | | | ATP/GTP-binding site motif A (P-loop): A371-S378, | MOTIFS |
| | | | | | G595-S602 | |
| | | | | | DEAH-box subfamily ATP-dependent helicases | MOTIFS |
| | | | | | signature: S697-E706 | |
| 22 | 3253807CD1 | 589 | S192 S196 S205 | N135 N334 | BTB/POZ domain: K18-S129 | HMMER_PFAM |
| | | | S241 S253 S284 | N410 | | |
| | | | S412 S489 S546 | | | |
| | | | S572 T91 T137 | | | |
| | | | T186 T218 T240 | | | |
| | | | T289 T317 T336 | | | |
| | | | T389 T436 Y90 | | | |
| | | | | | Zinc finger, C2H2 type: Y358-H380, F322-C344, | HMMER_PFAM |
| | | | | | H294-H316, F254-H277, N386-H409 | |
| | | | | | Zinc finger, C2H2 type BL00028 C324-H340 | BLIMPS_BLOCKS |
| | | | | | BTB/POZ domain (also known as BR-C/Ttk or ZiN) | BLIMPS_PFAM |
| | | | | | PF00651 A47-F59 | |
| | | | | | PROTEIN ZINC-FINGER METAL-BINDING DNA- | BLIMPS_{—} |
| | | | | | BINDING PD00066 H312-C324 | PRODOM |
| | | | | | POZ DOMAIN | BLAST_DOMO |
| | | | | | DM00509\|S59069\|1-171: E12-N140 | |
| | | | | | DM00509\|S41647\|11-189: E15-G183 | |
| | | | | | DM00509\|Q05516\|9-169: P14-S163 | |
| | | | | | DM00509\|P42282\|6-168: C9-P131 | |
| | | | | | Zinc finger, C2H2 type, domain: C256-H277, C296-H316, | MOTIFS |
| | | | | | C360-H380, C388-H409 | |
| 23 | 3626408CD1 | 192 | S17 S57 S89 S109 | N161 | Ribosomal protein S7e: A4-N191 | HMMER_PFAM |
| | | | T3 T98 | | | |
| | | | | | Ribosomal protein S7e proteins BL00948 | BLIMPS_BLOCKS |
| | | | | | K6-F28, R58-A110, V111-K149, I150-P187 | |
| | | | | | PROTEIN 40S RIBOSOMAL S7 S8 MULTIGENE | BLAST_PRODOM |
| | | | | | FAMILY RPS7 ZC434.2 S7A PD006276: R5-P187 | |
| | | | | | RIBOSOMAL; S7; S7E; | BLAST_DOMO |
| | | | | | DM03495\|JC4388\|1-194: M1-P187 | |
| | | | | | DM03495\|Q10101\|1-207: T3-M188 | |
| | | | | | DM03495\|P33514\|1-191: K7-P187 | |
| | | | | | DM03495\|P26786\|1-191: P16-P187 | |
| 24 | 3773014CD1 | 1007 | S2 S14 S20 S28 | N73 N74 | Zinc finger, C2H2 type: N631-H653, F659-H681, | HMMER_PFAM |
| | | | S144 S222 S234 | N352 N735 | Y401-H423, N691-H713, K911-H933, H373-H395, | |
| | | | S265 S295 S358 | | F940-H963, Q34-C56 | |
| | | | S485 S585 S669 | | | |
| | | | S757 S763 S790 | | | |
| | | | S797 S806 S895 | | | |
| | | | S908 S909 T250 | | | |
| | | | T269 T411 T589 | | | |
| | | | T701 T778 T889 | | | |
| | | | T954 | | | |
| | | | | | Zinc finger, C2H2 type BL00028: C661-H677 | BLIMPS_BLOCKS |
| | | | | | PROTEIN ZINC-FINGER METAL-BINDING DNA- | BLIMPS_{—} |
| | | | | | BINDING PD00066: H391-C403 | PRODOM |
| | | | | | PROTEIN ZINC-FINGER METAL-BINDING DNA- | BLAST_PRODOM |
| | | | | | BINDING PD017573: H681-N735 | |
| | | | | | ZINC FINGER, C2H2 TYPE, DOMAIN | BLAST_DOMO |
| | | | | | DM00002\|P39770\|466-500: L388-H423 | |
| | | | | | Zinc finger, C2H2 type, domain C375-H395, C403-H423, | MOTIFS |
| | | | | | C633-H653, C661-H681, C693-H713, C913-H933, | |
| | | | | | C942-H963 | |
| 25 | 4398735CD1 | 865 | S377 S379 S486 | N100 N146 | PROTEIN TRANSCRIPTION INITIATION | BLAST_PRODOM |
| | | | S651 S759 S807 | N167 N608 | FACTOR TFIID SUBUNIT REGULATION | |
| | | | T139 T148 T159 | N790 | NUCLEAR R119.6 P110 PD025348: L679-Y860 | |
| | | | T224 T257 T307 | | | |
| | | | T351 T436 T473 | | | |
| | | | T600 T640 T712 | | | |
| | | | | | TRANSCRIPTION INITIATION FACTOR TFIID | BLAST_PRODOM |
| | | | | | SUBUNIT REGULATION NUCLEAR PROTEIN | |
| | | | | | P110 TAFII110 PD043203: K583-E678, P61-P154, | |
| | | | | | P8-Q92, T235-K266, T548-I581 | |
| | | | | | PROTEIN TRANSCRIPTION INITIATION | BLAST_PRODOM |
| | | | | | FACTOR TFIID SUBUNIT REGULATION | |
| | | | | | NUCLEAR R119.6 TAFII135 PD025349: T235-Q345 | |
| | | | | | TRANSCRIPTION INITIATION FACTOR TFIID | BLAST_PRODOM |
| | | | | | 135 KD SUBUNIT TAFII135 TAFII135 TAFII130 | |
| | | | | | TAFII130 REGULATION NUCLEAR PROTEIN | |
| | | | | | PD143622 P2-V211 | |
| 26 | 7499579CD1 | 545 | S39 S87 S92 S175 | N135 N393 | Poly-adenylate binding protein, unique domain: E452-L523 | HMMER_PFAM |
| | | | S219 S227 S322 | | | |
| | | | S343 S511 S514 | | | |
| | | | T374 T388 | | | |
| | | | | | RNA recognition motif. (a.k.a. RRM, RBD, or RNP | HMMER_PFAM |
| | | | | | domain): V193-V263, I101-V170, L13-I84 | |
| | | | | | Eukaryotic RNA-binding region RNP-1 proteins | BLIMPS_BLOCKS |
| | | | | | BL00030: L13-F31, K231-R240 | |
| | | | | | Eukaryotic putative RNA-binding region RNP-1 | PROFILESCAN |
| | | | | | signature: S120-F169, V220-Q260 | |
| | | | | | Poly-adenylate binding protein PF00658 Y262-E308, | BLIMPS_PFAM |
| | | | | | A487-L523, R83-F122 | |
| | | | | | PROTEIN BINDING POLYA POLYADENYLATE- | BLAST_PRODOM |
| | | | | | BINDING PABP RNA-BINDING REPEAT POLY A | |
| | | | | | TESTIS ENRICHED PD012528: A362-Q451 | |
| | | | | | PROTEIN BINDING POLYA REPEAT | BLAST_PRODOM |
| | | | | | POLYADENYLATE-BINDING PABP RNA- | |
| | | | | | BINDING POLY A-BINDING NUCLEAR | |
| | | | | | POLYADENYLATE PD002964: E452-L523 | |
| | | | | | PROTEIN BINDING POLYA POLYADENYLATE- | BLAST_PRODOM |
| | | | | | BINDING PABP RNA-BINDING REPEAT | |
| | | | | | POLYADENYLATE II POLY PD150463: P316-G361 | |
| | | | | | PROTEIN RNA-BINDING NUCLEAR | BLAST_PRODOM |
| | | | | | RIBONUCLEOPROTEIN REPEAT BINDING | |
| | | | | | SPLICING FACTOR ALTERNATIVE | |
| | | | | | HETEROGENEOUS PD000013 D204-V263 | |
| | | | | | POLYADENYLATE-BINDING PROTEIN | BLAST_DOMO |
| | | | | | DM02879\|P11940\|365-620: A266-Q533 | |
| | | | | | RIBONUCLEOPROTEIN REPEAT | BLAST_DOMO |
| | | | | | DM00012\|I48718\|181-277: A181-R278, N100-L183 | |
| | | | | | DM00012\|P11940\|92-179: S92-E180, N192-Q273 | |
| | | | | | DM00012\|P11940\|181-264: A181-K268, N100-Q172 | |
| | | | | | Eukaryotic putative RNA-binding region RNP-1 | MOTIFS |
| | | | | | signature: K138-F145, K231-F238 | |
| 27 | 8178947CD1 | 429 | S18 S92 S161 S189 | N246 N333 | signal_cleavage: M1-R20 | SPSCAN |
| | | | S217 S332 S389 | | | |
| | | | T43 T52 T111 | | | |
| | | | T125 T149 T327 | | | |
| | | | T348 T398 Y407 | | | |
| | | | | | KRAB box: V42-E104 | HMMER_PFAM |
| | | | | | Zinc finger, C2H2 type: Y263-H285, Y319-H341, | HMMER_PFAM |
| | | | | | Y207-H229, Y375-H397, Y403-H425, Y347-H369, | |
| | | | | | Y235-H257, Y291-H313, Y179-H201, N151-H173 | |
| | | | | | Zinc finger, C2H2 type domain proteins BL00028 | BLIMPS_BLOCKS |
| | | | | | C209-H225 | |
| | | | | | C2H2-type zinc finger signature PR00048 P234-S247, | BLIMPS_PRINTS |
| | | | | | L334-G343 | |
| | | | | | PROTEIN ZINC FINGER ZINC PD00066 A337-C349 | BLIMPS_{—} |
| | | | | | | PRODOM |
| | | | | | PROTEIN ZINC FINGER ZINC PD01066 F44-G82 | BLIMPS_{—} |
| | | | | | | PRODOM |
| | | | | | PROTEIN ZINC FINGER METAL-BINDING DNA- | BLAST_PRODOM |
| | | | | | BINDING ZINC FINGER PATERNALLY | |
| | | | | | EXPRESSED ZN FINGER PW1 PD017719: G175-H425, | |
| | | | | | R143-F384, G287-R427 | |
| | | | | | ZINC FINGER METAL-BINDING DNA-BINDING | BLAST_PRODOM |
| | | | | | PROTEIN FINGER ZINC NUCLEAR REPEAT | |
| | | | | | TRANSCRIPTION REGULATION PD001562: V42-E103 | |
| | | | | | ZINC FINGER DNA-BINDING PROTEIN METAL- | BLAST_PRODOM |
| | | | | | BINDING NUCLEAR ZINC FINGER | |
| | | | | | TRANSCRIPTION REGULATION REPEAT | |
| | | | | | PD000072: K205-C268, P318-C380, R177-C240, | |
| | | | | | K345-C408, E152-C212, Y291-C352, K233-C296, | |
| | | | | | K373-Q428 | |
| | | | | | HYPOTHETICAL ZINC FINGER PROTEIN | BLAST_PRODOM |
| | | | | | B03B8.4 IN CHROMOSOME III ZINC FINGER | |
| | | | | | DNA BINDING METAL BINDING NUCLEAR | |
| | | | | | PD149420: E152-G315, C296-H425 | |
| | | | | | KRAB BOX DOMAIN | BLAST_DOMO |
| | | | | | DM00605\|P52736\|1-72: V42-P113 | |
| | | | | | DM00605\|I48689\|11-85: V42-P113 | |
| | | | | | DM00605\|Q05481\|10-83: G40-Q96 | |
| | | | | | DM00605\|P51786\|24-86: K39-V100 | |
| | | | | | ATP/GTP-binding site motif A (P-loop): G180-S187 | MOTIFS |
| | | | | | Zinc finger, C2H2 type, domain: C153-H173, C181-H201, | MOTIFS |
| | | | | | C209-H229, C237-H257, C265-H285, C293-H313, | |
| | | | | | C321-H341, C349-H369, C377-H397, C405-H425 | |
| 28 | 2264652CD1 | 1286 | S176 S274 S275 | N86 N174 | EXONUCLEASE 5′3′ DHM2 PROTEIN PD025656 | BLAST_PRODOM |
| | | | S323 S342 S500 | N382 N707 | Q905-S1225, G767-E924 | |
| | | | S533 S573 S597 | N763 N918 | | |
| | | | S637 S768 S835 | | | |
| | | | S871 S891 S898 | | | |
| | | | S902 S908 S981 | | | |
| | | | S1008 S1082 S1214 | | | |
| | | | S1225 S1233 S1267 | | | |
| | | | S1268 S1272 T3 | | | |
| | | | T12 T89 T127 | | | |
| | | | T239 T253 T337 | | | |
| | | | T394 T440 T562 | | | |
| | | | T671 T745 T819 | | | |
| | | | T844 T1188 Y203 | | | |
| | | | Y561 Y571 Y592 | | | |
| | | | Y603 | | | |
| | | | | | EXONUCLEASE 5′3′ HYDROLASE NUCLEASE | BLAST_PRODOM |
| | | | | | MAGNESIUM DNA RECOMBINATION DNA- | |
| | | | | | BINDING PROTEIN II PD014574: Y203-H766 | |
| | | | | | EXONUCLEASE PROTEIN HYDROLASE | BLAST_PRODOM |
| | | | | | NUCLEASE 5′3′ NUCLEAR EXORIBONUCLEASE | |
| | | | | | MAGNESIUM DNA RECOMBINATION | |
| | | | | | PD005946: D6-M192 | |
| | | | | | MOUSE; DHM1 EXONUCLEASE II; | BLAST_DOMO |
| | | | | | DM02631\|P40383\|14-812: E9-L331 | |
| | | | | | MOUSE; DHM1 STRAND EXCHANGE PROTEIN | BLAST_DOMO |
| | | | | | 1; DM02631\|P22147\|14-830: I2-K339 | |
| | | | | | MOUSE; DHM1 DHP1 PROTEIN; | BLAST_DOMO |
| | | | | | DM02631\|P40848\|35-981: D10-M192 | |
| | | | | | MOUSE; DHM1 MOUSE DHM1 PROTEIN; | BLAST_DOMO |
| | | | | | DM02631\|I49635\|34-905: D6-N185 | |
| | | | | | ATP/GTP-binding site motif A (P-loop): A299-S306 | MOTIFS |
| | | | | | ATP synthase alpha and beta subunits signature: P785- | MOTIFS |
| | | | | | S794 | |
| 29 | 1806372CD1 | 740 | S38 S55 S97 S180 | N432 N670 | DEAH-box subfamily ATP-dependent helicases | BLIMPS_BLOCKS |
| | | | S293 S365 S414 | | proteins BL00690: G93-Q102, T124-E141, L190-S199 | |
| | | | S532 S562 S655 | | | |
| | | | S729 T5 T76 T545 | | | |
| | | | T640 | | | |
| | | | | | HELICASE RNA ATP-BINDING PROTEIN ATP- | BLAST_PRODOM |
| | | | | | DEPENDENT NUCLEAR SPLICING mRNA | |
| | | | | | PROCESSING PREmRNA PD001259: C407-S550 | |
| | | | | | DEAH-BOX SUBFAMILY ATP-DEPENDENT | BLAST_DOMO |
| | | | | | HELICASES | |
| | | | | | DM00649\|P53131\|84-705: E60-N677 | |
| | | | | | DM00649\|P24384\|473-1078: Q64-T630 | |
| | | | | | DM00649\|A56236\|555-1160: L61-5674 | |
| | | | | | DM00649\|P34498\|432-1038: K63-L637 | |
| | | | | | ATP/GTP-binding site motif A (P-loop): G93-S100 | MOTIFS |
| 30 | 2010564CD1 | 376 | S330 S332 S346 | N11 N43 | Signal Peptide: M1-A34 | HMMER |
| | | | S367 T8 T67 T113 | N312 | | |
| | | | T120 T273 T336 | | | |
| | | | | | Signal Peptide: M19-A34 | HMMER |
| | | | | | PA domain: S65-I167 | HMMER_PFAM |
| | | | | | Zinc finger, C3HC4 type (RING finger): C256-C296 | HMMER_PFAM |
| | | | | | Cytosolic domain: M1-N188 | TMHMMER |
| | | | | | Transmembrane domain: H189-H211 | |
| | | | | | Non-cytosolic domain: R212-P376 | |
| | | | | | Zinc finger, C3HC4 type (RING finger), signature: | PROFILESCAN |
| | | | | | N252-V303 | |
| | | | | | ZINC FINGER, C3HC4 TYPE, | BLAST_DOMO |
| | | | | | DM00063\|Q06003\|119-171: N252-K302 | |
| 31 | 7364908CD1 | 400 | S9 S52 S58 S83 | | KRAB box: V8-K70 | HMMER_PFAM |
| | | | S136 S208 S230 | | | |
| | | | S286 S314 S335 | | | |
| | | | S342 T18 T154 | | | |
| | | | T158 T172 | | | |
| | | | | | Zinc finger, C2H2 type: Y304-H326, Y332-H354, | HMMER_PFAM |
| | | | | | Y276-H298, Y248-H270, Y360-H381 | |
| | | | | | Zinc Finger, C2H2-type BL00028: C306-H322 | BLIMPS_BLOCKS |
| | | | | | PROTEIN ZINC FINGER ZINC PD01066: F10-G48 | BLIMPS_{—} |
| | | | | | | PRODOM |
| | | | | | PROTEIN ZINC-FINGER METAL-BINDING DNA- | BLIMPS_{—} |
| | | | | | BINDING PD00066: H294-C306 | PRODOM |
| | | | | | ZINC FINGER METAL-BINDING DNA-BINDING | BLAST_PRODOM |
| | | | | | PROTEIN FINGER ZINC NUCLEAR REPEAT | |
| | | | | | TRANSCRIPTION REGULATION PD001562: V8-K70 | |
| | | | | | PROTEIN ZINC FINGER METAL-BINDING DNA- | BLAST_PRODOM |
| | | | | | BINDING ZINC FINGER PATERNALLY | |
| | | | | | EXPRESSED ZN FINGER PW1 PD017719: G244-R379, | |
| | | | | | C225-F396, C197-E384 | |
| | | | | | ZINC FINGER DNA-BINDING PROTEIN METAL- | BLAST_PRODOM |
| | | | | | BINDING NUCLEAR ZINC FINGER | |
| | | | | | TRANSCRIPTION REGULATION REPEAT | |
| | | | | | PD000072: K274-C337, K246-C309, K302-E361 | |
| | | | | | KRAB BOX DOMAIN | BLAST_DOMO |
| | | | | | DM00605\|I48689\|11-85: Q5-I73 | |
| | | | | | DM00605\|P52738\|3-77: Q5-I73 | |
| | | | | | DM00605\|P51523\|5-79: Q5-N75 | |
| | | | | | DM00605\|P51786\|24-86: Q5-W67 | |
| | | | | | Zinc finger, C2H2 type, domain: C250-H270, C278-H298, | MOTIFS |
| | | | | | C306-H326, C334-H354 | |
| 32 | 7489960CD1 | 472 | S103 S116 S310 | N139 | Zinc finger C-x8-C-x5-C-x3-H type (and similar): | HMMER_PFAM |
| | | | T133 | | K119-I144, K148-D173, P175-P197 | |
| | | | | | Zinc finger C-x8-C-x5-C-x3-H type (and similar) | BLIMPS_PFAM |
| | | | | | PF00642: C132-H142 | |
| | | | | | PROTEIN SUPPRESSOR OF SABLE EG: 115C2.3 | BLAST_PRODOM |
| | | | | | RNA BINDING NUCLEAR HOMOLOG | |
| | | | | | PD032978: P147-K208 | |
| 33 | 8555401CD1 | 401 | S12 S23 S41 S55 | N101 N261 | HSF-type DNA-binding domain: S133-Q147, N177-I195, | HMMER_PFAM |
| | | | S67 S97 S254 S353 | | F80-L117 | |
| | | | S391 T18 T374 | | | |
| | | | | | HSF-type DNA-binding domain proteins BL00434 | BLIMPS_BLOCKS |
| | | | | | G102-I146, F174-R194 | |
| | | | | | HSF-TYPE DNA-BINDING DOMAIN | BLAST_DOMO |
| | | | | | DM00610\|P38533\|1-208: F80-I259 | |

TABLE 4

| Polynucleotide | |
|---|---|
| SEQ ID NO:/ | |
| Incyte ID/ Sequence | |
| Length | Sequence Fragments |
| 34/4001873CB1/ | 1-620, 1-971, 98-597, 98-711, 98-768, 98-778, 98-867, 98-987, 266-727, 287-961, 332-965, 414-986, 419-1357, 420-986, |
| 1357 | 425-987, 458-799, 458-946, 490-986, 540-663, 554-653 |
| 35/55003135CB1/ | 1-665, 34-913, 365-942, 616-673, 751-808 |
| 942 | |
| 36/5855204CB1/ | 1-622, 1-641, 1-644, 1-652, 1-663, 1-674, 1-692, 1-717, 1-730, 1-735, 1-777, 10-661, 30-754, 44-750, 67-897, 88-610, |
| 3288 | 161-743, 174-703, 195-702, 207-672, 256-1065, 290-1050, 324-1082, 370-949, 377-609, 389-1139, 402-852, |
| | 422-1052, 471-1061, 552-1145, 658-1166, 691-1229, 709-1215, 716-1229, 749-1229, 804-1229, 811-1220, 890-1229, |
| | 918-1217, 976-1229, 1099-1237, 1190-1288, 1190-1778, 1190-1875, 1190-2089, 1194-1625, 1224-1647, 1224-1744, |
| | 1224-1761, 1224-1789, 1224-1803, 1224-1809, 1224-1860, 1226-1478, 1289-1833, 1311-1971, 1445-1920, |
| | 1510-2375, 1708-2372, 1775-2778, 1775-3088, 2058-2841, 2066-2393, 2186-2393, 2242-2785, 2252-2743, 2326-2825, |
| | 2326-2827, 2359-2623, 2483-2682, 2779-3288, 2786-3077, 2897-3084, 3040-3082 |
| 37/5778654CB1/ | 1-736, 1-737, 1-1422, 5-737, 18-685, 77-737, 95-1422, 100-737, 609-873, 706-967, 710-1247, 885-1183, 900-1137, |
| 1422 | 1291-1402 |
| 38/1440126CB1/ | 1-1341, 1-2129, 125-653, 129-1033, 412-458, 412-542, 412-543, 412-609, 412-626, 412-678, 412-710, 412-1118, |
| 2129 | 412-1176, 413-626, 430-458, 430-543, 430-672, 431-458, 431-617, 431-679, 433-535, 433-548, 433-634, 433-678, |
| | 456-729, 456-730, 496-710, 496-794, 496-1257, 520-626, 520-718, 540-710, 540-794, 546-694, 565-710, 565-1092, |
| | 580-800, 580-846, 580-1283, 597-678, 601-1008, 602-678, 635-990, 635-1249, 770-1018, 770-1071, 853-1035, 853-1054, |
| | 853-1098, 853-1257, 856-962, 856-1008, 856-1054, 880-1095, 880-1098, 887-1098, 900-1184, 903-979, 934-1130, |
| | 934-1176, 966-1008, 987-1063, 987-1088, 987-1149, 987-1176, 987-1257, 1019-1219, 1019-1264, 1021-1130, |
| | 1021-1227, 1021-1257, 1021-1283, 1022-1253, 1022-1257, 1049-1118, 1069-1098, 1081-1118, 1195-1712, |
| | 1205-1711, 1212-1708, 1212-1711, 1215-1711, 1219-1566, 1320-1709, 1364-1791, 1364-2026, 1500-2129, 1747-2045 |
| 39/3934519CB1/ | 1-449, 3-594, 6-541, 7-588, 12-281, 12-293, 12-351, 12-431, 12-527, 12-666, 12-2165, 61-398, 357-625, 386-900, |
| 3103 | 407-1176, 407-1476, 417-632, 451-627, 463-912, 532-813, 532-819, 537-1276, 537-1376, 601-886, 601-897, 601-910, |
| | 633-2465, 644-929, 745-1176, 807-1116, 995-1101, 995-1137, 995-1208, 995-1220, 995-1994, 995-2246, 996-1315, |
| | 996-1488, 996-1777, 1001-1220, 1001-1403, 1001-1709, 1003-1063, 1003-1517, 1003-1575, 1003-1604, 1003-1651, |
| | 1004-1063, 1004-1072, 1004-1630, 1004-1651, 1012-1043, 1012-1111, 1012-1147, 1012-1567, 1012-1731, |
| | 1012-2155, 1014-1063, 1016-1063, 1073-1321, 1116-1651, 1116-2044, 1157-1220, 1157-1289, 1157-1315, 1157-2076, |
| | 1157-2323, 1163-1269, 1163-1316, 1163-1388, 1163-1483, 1163-1630, 1163-2330, 1163-2414, 1178-1231, |
| | 1178-1456, 1241-1359, 1241-1389, 1241-1399, 1241-1557, 1241-2247, 1241-2407, 1264-1363, 1264-1399, 1264-1625, |
| | 1264-1651, 1264-1731, 1264-2407, 1267-1315, 1267-1818, 1268-1324, 1268-1456, 1339-1399, 1351-1403, |
| | 1351-1992, 1352-1456, 1352-1628, 1375-1399, 1376-1456, 1391-1710, 1395-2133, 1424-1792, 1424-1818, 1424-2071, |
| | 1427-1710, 1436-1628, 1452-1556, 1452-1632, 1452-1709, 1452-1731, 1452-2380, 1465-1628, 1498-1710, |
| | 1507-1567, 1507-1779, 1507-1877, 1507-1903, 1507-2155, 1514-2160, 1516-1557, 1520-1710, 1520-1777, |
| | 1525-1710, 1542-1725, 1559-1651, 1564-1651, 1581-1651, 1583-1651, 1591-1699, 1591-1761, 1591-1864, 1591-1961, |
| | 1591-2064, 1591-2239, 1660-1893, 1668-1809, 1668-1987, 1668-2145, 1668-2449, 1684-2241, 1687-2328, |
| | 1697-1731, 1697-1777, 1757-1818, 1757-1932, 1757-1976, 1757-2229, 1757-2466, 1765-1818, 1765-2380, 1772-1818, |
| | 1795-1977, 1852-1987, 1852-2292, 1852-2468, 1855-2071, 1865-1903, 1865-1912, 1865-1988, 1868-2044, |
| | 1878-1974, 1938-1987, 1939-1980, 1939-2155, 1940-2044, 1940-2155, 1949-2044, 1979-2246, 1983-2239, 2012-2071, |
| | 2016-2246, 2023-2071, 2024-2155, 2053-2155, 2086-2246, 2087-2246, 2095-2155, 2096-2449, 2099-2323, |
| | 2102-2142, 2102-2155, 2102-2239, 2102-2380, 2107-2155, 2113-2246, 2117-2151, 2144-2551, 2144-2991, 2145-2832, |
| | 2183-2466, 2187-2323, 2192-2238, 2192-2239, 2197-2323, 2231-2407, 2236-2407, 2250-2468, 2255-2519, |
| | 2255-2847, 2281-2527, 2281-3103, 2315-2414, 2319-2380, 2348-2416, 2360-2416 |
| 40/2946314CB1/ | 1-2658, 423-1024, 560-1024, 561-1024, 2026-2319, 2027-2314, 2170-2446, 2196-2455, 2471-3204, 2487-3092, |
| 8810 | 2517-3092, 2591-3163, 2594-3371, 2609-2876, 2609-3152, 2656-2763, 2663-2727, 2691-2799, 2697-2799, 2735-2799, |
| | 2771-2796, 2915-3397, 2947-3465, 2996-3212, 2996-3220, 3111-3822, 3233-3444, 3281-3561, 3281-3754, |
| | 3440-4260, 3459-4047, 3476-4036, 3525-4047, 3821-4066, 3823-4128, 3890-4588, 3993-4353, 4018-4559, 4075-4691, |
| | 4097-4311, 4097-4591, 4119-4368, 4127-4312, 4127-4830, 4153-4935, 4324-4788, 4333-4873, 4348-4789, |
| | 4376-4644, 4379-4657, 4420-4658, 4431-4735, 4482-5117, 4507-4859, 4507-4919, 4620-4808, 4620-4921, 4641-4907, |
| | 4693-5373, 4853-5465, 4963-5541, 5003-5776, 5049-5655, 5101-5341, 5142-5748, 5164-5561, 5183-5563, |
| | 5185-5817, 5214-5514, 5231-5483, 5231-5567, 5261-5490, 5261-5543, 5261-5548, 5261-5563, 5262-5550, 5326-5907, |
| | 5379-6034, 5394-5682, 5446-5914, 5447-5726, 5532-6124, 5709-6147, 5758-6406, 5812-6266, 5874-6162, |
| | 6005-6697, 6012-6217, 6012-6627, 6021-6262, 6021-6308, 6044-6683, 6090-6629, 6179-6435, 6225-6537, 6225-6704, |
| | 6244-6505, 6244-6749, 6252-6514, 6329-6579, 6339-6909, 6339-6958, 6487-7045, 6577-6853, 6604-6936, |
| | 6604-7111, 6604-7222, 6604-7244, 6629-6885, 6650-6955, 6650-7193, 6650-7194, 6668-6956, 6680-7117, 6697-7225, |
| | 6706-6996, 6716-6946, 6728-7222, 6747-6988, 6747-7262, 6748-6998, 6765-7025, 6765-7037, 6775-7035, |
| | 6813-7036, 6820-7401, 6823-7093, 6833-7297, 6849-7607, 6890-7374, 6916-7410, 6922-7387, 6953-7541, 6963-7840, |
| | 7003-7247, 7003-7551, 7015-7327, 7034-7509, 7063-7511, 7109-7397, 7134-7599, 7145-7365, 7164-7392, |
| | 7169-7816, 7192-7403, 7204-7790, 7207-7452, 7218-7843, 7221-7831, 7227-7492, 7227-7620, 7234-7524, 7244-7500, |
| | 7327-7792, 7328-7562, 7342-7843, 7383-7843, 7395-7637, 7399-7843, 7410-7843, 7413-7807, 7419-7842, |
| | 7420-7842, 7421-7816, 7428-7838, 7434-7837, 7435-7779, 7441-7838, 7443-7691, 7443-7843, 7454-7843, 7456-7716, |
| | 7480-7716, 7488-7754, 7489-7837, 7501-7839, 7503-7840, 7530-8153, 7531-7710, 7568-7787, 7580-7841, |
| | 7586-7799, 7586-7818, 7586-7840, 7586-7843, 7589-7806, 7589-7843, 7591-7762, 7750-8124, 7800-8502, 7855-8306, |
| | 8160-8785, 8320-8575, 8320-8810, 8376-8640 |
| 41/3617784CB1/ | 1-360, 240-2606, 678-835, 678-907, 678-1039, 678-1083, 678-1136, 678-1154, 678-1159, 680-1312, 715-1276, 862-1506, |
| 4070 | 965-1566, 1012-1626, 1062-1623, 1108-1582, 1119-1754, 1194-1820, 1228-1869, 1250-1366, 1250-1459, |
| | 1265-1873, 1267-1940, 1268-1460, 1268-1517, 1268-2102, 1296-1793, 1308-1517, 1311-1366, 1311-1459, 1311-1870, |
| | 1316-1369, 1316-1378, 1316-1433, 1316-1434, 1316-1473, 1317-1366, 1317-1460, 1317-1470, 1317-1544, |
| | 1317-2017, 1324-1508, 1324-1518, 1324-1543, 1324-1630, 1324-1934, 1333-1544, 1343-1378, 1343-1473, 1343-1954, |
| | 1359-1460, 1390-1504, 1390-1518, 1395-1518, 1397-1473, 1398-1460, 1399-1517, 1399-1518, 1400-1960, |
| | 1401-1424, 1401-1438, 1401-1453, 1401-1458, 1401-1459, 1401-1508, 1401-1515, 1401-1516, 1401-1518, 1401-1544, |
| | 1401-1715, 1401-1723, 1401-1752, 1401-2179, 1405-1544, 1408-1544, 1409-1544, 1410-1473, 1411-1457, |
| | 1419-1517, 1420-1517, 1431-2060, 1434-1513, 1434-1518, 1434-1840, 1434-1854, 1434-1859, 1434-1877, 1434-2270, |
| | 1443-1961, 1445-1479, 1483-1544, 1501-1719, 1506-2125, 1563-1630, 1581-1639, 1587-1752, 1587-2128, |
| | 1622-2331, 1648-2201, 1665-1713, 1665-1752, 1665-2095, 1665-2182, 1665-2213, 1674-2174, 1696-1752, |
| | 1741-2332, 1741-2371, 1770-1840, 1770-1854, 1770-2152, 1770-2183, 1770-2192, 1770-2639, 1771-1854, 1772-1961, |
| | 1772-2018, 1772-2689, 1773-1858, 1773-1960, 1773-2005, 1773-2215, 1773-2293, 1781-1854, 1781-2038, |
| | 1781-2041, 1781-2088, 1781-2178, 1781-2185, 1781-2303, 1782-1837, 1782-1859, 1782-1925, 1782-1960, 1782-2034, |
| | 1782-2046, 1794-1851, 1812-2018, 1817-1933, 1817-2086, 1817-2098, 1817-2170, 1817-2522, 1821-1862, |
| | 1821-1889, 1821-1961, 1821-2140, 1821-2169, 1821-2170, 1821-2599, 1824-1854, 1825-2303, 1831-1854, 1831-1961, |
| | 1831-2018, 1833-1933, 1833-2213, 1837-2098, 1840-2170, 1855-2050, 1884-1961, 1884-2002, 1884-2010, |
| | 1884-2187, 1890-2497, 1898-1940, 1898-2182, 1898-2254, 1898-2398, 1898-2632, 1900-2187, 1901-1961, 1901-2109, |
| | 1901-2128, 1901-2170, 1901-2179, 1902-2009, 1902-2010, 1902-2041, 1902-2046, 1902-2078, 1902-2118, |
| | 1902-2129, 1902-2143, 1902-2185, 1902-2634, 1906-2101, 1906-2293, 1909-2129, 1910-2046, 1912-2568, 1914-2179, |
| | 1914-2293, 1918-2473, 1921-2550, 1944-2017, 1972-2581, 1976-2293, 1976-2303, 1977-2177, 1977-2182, |
| | 1977-2303, 1985-2098, 1985-2170, 1989-2185, 1993-2148, 1993-2187, 1993-2299, 1993-2633, 1994-2144, |
| | 1998-2046, 1998-2518, 1998-2623, 2002-2215, 2003-2212, 2007-2506, 2022-2107, 2022-2212, 2022-2426, 2022-2446, |
| | 2022-2463, 2022-2467, 2022-2650, 2030-2548, 2031-2277, 2031-2303, 2039-2169, 2052-2129, 2052-2341, |
| | 2052-2355, 2064-2119, 2064-2212, 2065-2212, 2067-2129, 2068-2424, 2069-2215, 2069-2254, 2069-2398, 2069-2629, |
| | 2069-2632, 2070-2093, 2070-2179, 2073-2139, 2074-2632, 2077-2212, 2078-2179, 2079-2185, 2082-2169, |
| | 2082-2398, 2082-2622, 2089-2355, 2098-2185, 2106-2765, 2113-2169, 2114-2639, 2122-2177, 2127-2192, 2133-2613, |
| | 2150-2212, 2153-2303, 2153-2335, 2158-2298, 2158-2379, 2158-2582, 2158-2673, 2163-2303, 2170-2650, |
| | 2175-2650, 2228-2650, 2247-2303, 2247-2650, 2250-2308, 2250-2458, 2250-2463, 2250-2497, 2250-2542, 2250-2548, |
| | 2254-2513, 2256-2868, 2271-2896, 2282-2347, 2282-2355, 2282-2647, 2282-2689, 2283-2523, 2283-2548, |
| | 2286-2529, 2288-2791, 2302-2831, 2313-2639, 2321-2381, 2321-2548, 2321-2590, 2321-2599, 2321-2650, 2325-2355, |
| | 2325-2434, 2326-2381, 2326-2639, 2329-2792, 2334-2547, 2334-2632, 2341-2398, 2352-2821, 2367-2434, |
| | 2367-2444, 2367-2597, 2367-2632, 2367-2634, 2376-3028, 2402-3028, 2404-2548, 2406-2498, 2406-2521, |
| | 2406-2634, 2406-2636, 2406-2639, 2409-2545, 2418-2548, 2450-2689, 2476-3121, 2488-2604, 2488-2607, 2488-2632, |
| | 2489-2639, 2489-2646, 2490-2548, 2490-2599, 2490-2633, 2493-2631, 2493-2689, 2494-2634, 2494-2639, |
| | 2494-2689, 2497-2646, 2498-2671, 2511-2634, 2574-2639, 2574-2689, 2583-3155, 2598-2639, 2598-2662, 2602-3263, |
| | 2610-2689, 2647-3206, 2653-3180, 2656-3404, 2700-3086, 2700-3245, 2706-3196, 2706-3249, 2730-3026, |
| | 2815-3245, 2901-3449, 2909-3141, 2909-3403, 2909-3411, 2934-3411, 2949-3409, 2952-3409, 2982-3411, 3083-3501, |
| | 3158-3839, 3182-3661, 3187-3709, 3213-3516, 3260-3881, 3271-3833, 3290-3562, 3447-4020, 3448-3969, |
| | 3467-4067, 3468-4026, 3469-3988, 3488-4070 |
| 42/7490869CB1/ | 1-432, 2-388, 2-432, 2-493, 21-432, 23-436, 41-432, 302-986, 515-1112, 851-918, 863-1439, 865-1439, 980-1351, |
| 3250 | 990-1256, 1321-1867, 1508-3042, 1867-1965, 1925-2189, 2408-2557, 2777-3227, 2849-2961, 2849-3061, 2861-3234, |
| | 2863-3226, 2880-2989, 2883-3080, 2883-3114, 2883-3129, 2883-3222, 2883-3250, 2905-3229, 2917-3060, |
| | 2933-3192, 2934-3231, 2957-3229, 2983-3232, 3023-3231, 3101-3225, 3151-3245 |
| 43/5994687CB1/ | 1-227, 12-1345, 86-231, 86-551, 86-574, 86-892, 86-897, 205-492, 215-686, 215-908, 231-967, 327-869, 328-811, |
| 1345 | 331-606, 340-559, 462-949, 469-685, 525-984, 535-985, 536-975, 538-984, 614-1142, 618-985, 636-982, 681-984, |
| | 689-1280, 767-985, 797-1344, 825-1239, 919-1308, 1009-1343, 1009-1345, 1044-1345 |
| 44/2560755CB1/ | 1-99, 1-260, 1-388, 1-549, 1-591, 12-537, 22-456, 105-300, 105-669, 109-3173, 235-851, 240-608, 518-1081, 553-1223, |
| 3313 | 687-1183, 719-1333, 753-1318, 791-1318, 810-1363, 829-1455, 829-1461, 839-1429, 936-1453, 961-1593, |
| | 969-1560, 971-1543, 973-1638, 975-1640, 1023-1650, 1065-1737, 1134-1644, 1184-1782, 1198-1333, 1297-1786, |
| | 1375-1902, 1433-1974, 1461-1734, 1461-1891, 1461-1984, 1461-2003, 1461-2014, 1461-2107, 1485-1902, 1531-2157, |
| | 1548-2170, 1602-1902, 1615-2200, 1627-2214, 1689-2048, 1689-2317, 1720-2155, 1811-2380, 1892-2297, |
| | 1972-2562, 2026-2540, 2157-2846, 2311-2592, 2311-2701, 2311-2724, 2311-2769, 2311-2812, 2311-2997, 2329-2786, |
| | 2358-2696, 2391-2648, 2391-2979, 2413-2703, 2462-3055, 2482-2952, 2496-2769, 2520-3157, 2529-2959, |
| | 2529-2967, 2529-2997, 2529-3052, 2531-2997, 2556-2997, 2559-3052, 2598-3052, 2605-3052, 2611-2898, 2621-3052, |
| | 2657-3052, 2664-2942, 2664-3047, 2664-3309, 2664-3313, 2675-3052, 2688-3308, 2695-3301, 2702-2952, |
| | 2702-3052, 2706-3300, 2709-3292, 2726-3052, 2736-3052, 2744-3178, 2753-2983, 2755-3052, 2773-3303, 2776-3313, |
| | 2780-3313, 2787-3052, 2788-3052, 2796-3003, 2802-3299, 2828-3071, 2840-3310, 2856-3313, 2880-3313, |
| | 2887-3311, 2902-3313, 2933-3052, 2986-3244 |
| 45/3217430CB1/ | 1-635, 22-810, 26-355, 84-879, 91-608, 267-792, 361-793, 395-579, 395-780, 421-979, 421-982, 421-2894, 428-888, |
| 3451 | 428-1283, 469-1060, 495-1134, 633-1213, 786-1280, 789-1345, 816-1331, 922-1148, 1040-1292, 1040-1302, |
| | 1040-1387, 1067-1612, 1110-1823, 1128-1387, 1131-1699, 1133-1689, 1141-1387, 1228-1462, 1228-1747, 1280-1782, |
| | 1316-1824, 1431-1700, 1431-1724, 1431-1806, 1431-1807, 1431-1812, 1431-1820, 1431-1833, 1431-1916, |
| | 1431-1925, 1450-1603, 1463-2103, 1481-2042, 1490-2023, 1490-2026, 1501-1793, 1506-1857, 1511-2068, 1517-1778, |
| | 1528-1781, 1538-1983, 1574-2084, 1577-1818, 1577-2068, 1577-2098, 1580-1991, 1618-1853, 1620-2052, |
| | 1626-2253, 1638-2301, 1661-2256, 1677-2183, 1677-2276, 1699-2347, 1711-2269, 1713-2157, 1730-2040, 1730-2309, |
| | 1756-2332, 1760-2173, 1760-2425, 1773-2155, 1791-2063, 1802-2353, 1806-2422, 1826-2358, 1839-2424, |
| | 1840-2137, 1843-2501, 1845-2477, 1864-2391, 1887-2477, 1889-2167, 1907-2363, 1931-2332, 1938-2195, 1938-2393, |
| | 1938-2435, 1940-2648, 1946-2543, 1962-2446, 1965-2186, 1978-2242, 1983-2397, 1987-2212, 2000-2520, |
| | 2000-2541, 2010-2525, 2026-2551, 2028-2640, 2037-2393, 2045-2627, 2070-2549, 2088-2755, 2103-2560, |
| | 2108-2351, 2110-2344, 2117-2441, 2129-2403, 2130-2440, 2157-2682, 2171-2669, 2179-2786, 2199-2438, 2221-2655, |
| | 2233-2873, 2237-2894, 2259-2829, 2268-2760, 2269-2529, 2270-2771, 2281-2881, 2284-2882, 2311-2875, |
| | 2312-2650, 2320-2641, 2323-2541, 2324-2541, 2324-2716, 2327-2881, 2332-2593, 2332-2682, 2342-2894, 2346-2894, |
| | 2362-2896, 2391-2854, 2394-2866, 2400-2894, 2401-2890, 2402-2749, 2402-2856, 2403-2607, 2411-2894, |
| | 2416-2894, 2423-2894, 2427-2570, 2429-2871, 2431-2894, 2434-2899, 2437-2894, 2440-2894, 2447-2894, 2464-2891, |
| | 2468-2898, 2472-2894, 2480-2896, 2498-2895, 2499-2893, 2507-2898, 2513-2899, 2545-2894, 2549-2727, |
| | 2549-2729, 2549-2894, 2551-2895, 2551-2896, 2560-2801, 2565-2860, 2573-2894, 2587-2894, 2608-2894, 2618-2894, |
| | 2627-2896, 2628-3451, 2706-2897 |
| 46/5786832CB1/ | 1-778, 352-632, 352-636, 413-730, 413-897, 413-2874, 458-1097, 587-842, 643-857, 645-1091, 792-1519, 985-1302, |
| 3220 | 985-1589, 985-1653, 988-1533, 989-1652, 990-1653, 1207-1254, 1213-1248, 1213-1307, 1216-1312, 1220-1250, |
| | 1220-1251, 1220-1254, 1220-1255, 1220-1256, 1220-1260, 1220-1312, 1220-1339, 1220-1422, 1279-1321, |
| | 1279-1501, 1279-1759, 1279-2499, 1280-1339, 1283-1592, 1283-2340, 1291-1338, 1292-1339, 1292-1413, 1292-1415, |
| | 1292-1422, 1292-1759, 1292-2094, 1300-1570, 1310-1759, 1344-2013, 1359-1485, 1359-1668, 1359-1926, |
| | 1359-2256, 1364-1415, 1367-1665, 1367-1668, 1367-2429, 1376-1571, 1376-1573, 1376-1581, 1376-1665, 1376-1668, |
| | 1376-1986, 1376-2012, 1379-1868, 1383-1907, 1388-1569, 1388-1581, 1388-2014, 1388-2760, 1401-1653, |
| | 1439-1635, 1447-1665, 1447-1676, 1447-1895, 1447-1920, 1451-1555, 1451-1566, 1451-1573, 1451-1676, 1453-1499, |
| | 1453-1571, 1453-1581, 1453-1585, 1453-1668, 1453-1676, 1453-1838, 1453-2013, 1453-2094, 1453-2256, |
| | 1455-1581, 1459-1568, 1459-1677, 1459-1758, 1459-1836, 1459-2001, 1459-2088, 1463-1571, 1466-1655, 1466-1833, |
| | 1466-1891, 1466-1926, 1466-1991, 1466-2003, 1466-2096, 1467-2170, 1472-1585, 1472-1664, 1472-1665, |
| | 1472-1668, 1472-1669, 1472-1842, 1472-1928, 1472-1949, 1472-2101, 1472-2936, 1477-2003, 1483-1842, 1488-1665, |
| | 1521-1668, 1527-1667, 1527-1669, 1527-1676, 1527-2925, 1531-1573, 1531-1665, 1531-2013, 1532-1584, |
| | 1532-1667, 1532-1668, 1532-1833, 1532-1922, 1532-1997, 1537-1665, 1540-2075, 1540-2090, 1542-1581, 1543-1668, |
| | 1543-1833, 1546-2075, 1550-1665, 1551-2149, 1555-2004, 1615-1668, 1615-1835, 1615-1837, 1615-1921, |
| | 1615-1933, 1615-1988, 1615-1989, 1615-3188, 1616-1666, 1616-1667, 1616-1668, 1620-2169, 1626-1665, 1626-1668, |
| | 1626-1727, 1627-1667, 1627-1676, 1627-1821, 1628-1668, 1628-2088, 1630-1668, 1634-2004, 1635-2275, |
| | 1640-1674, 1645-2223, 1690-1759, 1700-1758, 1700-2001, 1706-1758, 1706-2015, 1706-2101, 1718-2243, 1723-1759, |
| | 1724-1832, 1724-1837, 1724-1887, 1724-1905, 1724-2094, 1724-2256, 1724-3096, 1735-2095, 1737-1989, |
| | 1740-1836, 1756-1833, 1775-1971, 1780-2090, 1784-1971, 1787-1917, 1787-1919, 1787-2000, 1787-2001, 1787-2003, |
| | 1787-2004, 1787-2006, 1787-2077, 1787-2096, 1787-2931, 1787-3009, 1790-2264, 1791-1917, 1791-2094, |
| | 1791-2253, 1791-2481, 1792-1836, 1792-2001, 1792-2076, 1792-2078, 1792-2097, 1792-2238, 1794-2328, |
| | 1795-1843, 1795-2001, 1795-2088, 1796-1842, 1796-1927, 1796-2085, 1798-2318, 1802-2099, 1802-2227, 1803-2424, |
| | 1810-2101, 1810-2263, 1814-1836, 1824-2001, 1827-2088, 1868-2001, 1871-1927, 1873-2096, 1873-2256, |
| | 1875-1920, 1876-2256, 1876-2391, 1878-2256, 1879-2078, 1879-2101, 1880-2003, 1882-2411, 1886-2001, 1887-2495, |
| | 1891-2256, 1891-2347, 1892-1929, 1897-1920, 1908-2096, 1911-2101, 1952-2264, 1952-2492, 1956-2421, |
| | 1962-2001, 1962-2004, 1962-2063, 1963-2003, 1963-2088, 1963-2096, 1963-2256, 1964-2086, 1964-2094, 1964-2245, |
| | 1964-2256, 1964-3162, 1966-2424, 1970-2395, 1971-2505, 1975-2340, 1976-2010, 1982-2424, 2008-2767, |
| | 2036-2073, 2036-2095, 2036-2256, 2039-2353, 2041-2169, 2041-2171, 2041-2241, 2041-2256, 2041-2844, 2042-2168, |
| | 2043-2094, 2043-2101, 2044-2078, 2046-2088, 2047-2101, 2047-2340, 2050-2424, 2052-2088, 2055-2256, |
| | 2059-2838, 2070-2725, 2085-2725, 2125-2245, 2125-2505, 2130-2256, 2131-2169, 2131-2171, 2131-2179, 2132-2340, |
| | 2134-2353, 2162-2724, 2194-2263, 2194-2427, 2203-2766, 2204-2243, 2204-2258, 2204-2262, 2204-2399, |
| | 2204-2424, 2204-2505, 2204-2589, 2209-2318, 2210-2263, 2210-2427, 2212-2246, 2212-2498, 2216-2256, |
| | 2216-2340, 2216-2413, 2216-2420, 2216-2424, 2216-2766, 2216-2843, 2216-2925, 2218-2424, 2220-2256, 2222-2262, |
| | 2222-2328, 2222-2409, 2222-2589, 2222-2852, 2223-2852, 2227-2424, 2227-2505, 2228-2256, 2228-2264, |
| | 2238-2424, 2295-2346, 2295-2353, 2295-2413, 2295-2424, 2295-2785, 2295-2854, 2298-2424, 2298-2611, 2299-2333, |
| | 2299-2505, 2300-2424, 2300-2766, 2302-2831, 2331-2424, 2371-2497, 2371-2505, 2371-2577, 2371-2767, |
| | 2371-3014, 2372-2423, 2372-2424, 2372-2673, 2380-2415, 2380-2424, 2380-2685, 2380-2757, 2380-2794, 2382-2841, |
| | 2383-2402, 2383-2426, 2384-2499, 2384-2505, 2384-2757, 2384-2844, 2384-2845, 2386-2505, 2388-2815, |
| | 2395-2424, 2396-2488, 2405-2852, 2460-3049, 2460-3214, 2463-2766, 2464-2497, 2464-2498, 2464-2501, 2464-3014, |
| | 2466-2505, 2467-2501, 2467-2505, 2467-2844, 2467-3096, 2468-2589, 2470-2589, 2481-3220, 2541-2589, |
| | 2541-2731, 2541-2935, 2546-2589, 2554-2589, 2554-2600, 2619-2685, 2624-2673, 2624-2676, 2624-2684, 2624-2745, |
| | 2624-2760, 2624-2844, 2624-2925, 2624-3009, 2624-3014, 2625-2673, 2625-2766, 2625-2844, 2625-3019, |
| | 2626-2685, 2629-2761, 2629-2829, 2629-3014, 2630-2756, 2631-3009, 2636-2682, 2636-2684, 2636-2753, |
| | 2636-2757, 2636-2915, 2636-2925, 2636-2930, 2636-3011, 2636-3093, 2636-3096, 2638-2852, 2642-3014, 2654-2766, |
| | 2698-2747, 2698-2844, 2698-2931, 2698-2936, 2698-3019, 2699-2757, 2704-2999, 2704-3014, 2708-2757, |
| | 2708-2760, 2708-2766, 2708-2925, 2708-3014, 2708-3019, 2711-2759, 2711-2833, 2711-2846, 2711-2913, 2711-2936, |
| | 2714-2757, 2714-2840, 2716-3096, 2718-2760, 2719-2844, 2719-3009, 2720-2924, 2720-2925, 2720-2930, |
| | 2720-3012, 2722-2925, 2726-3009, 2727-2936, 2731-2925, 2731-3012, 2732-2767, 2738-2760, 2751-2844, 2796-2925, |
| | 2797-2844, 2801-3009, 2801-3096, 2801-3188, 2802-3014, 2803-2843, 2804-2841, 2804-2851, 2804-2852, |
| | 2804-2925, 2806-2841, 2806-3014, 2810-3009, 2811-2853, 2815-2852, 2816-2893, 2816-2935, 2822-3014, 2875-2915, |
| | 2875-2925, 2875-2931, 2875-3012, 2876-2925, 2879-2925, 2879-3014, 2888-2925, 2888-2936, 2888-3009, |
| | 2888-3096, 2890-3096, 2890-3188, 2891-2930, 2891-2999, 2894-2931, 2900-2925, 2900-2933, 2960-2999, 2960-3012, |
| | 2960-3014, 2965-3013, 2972-3019, 2972-3087, 2972-3093, 2974-3014, 2978-3087, 3051-3096, 3051-3120, |
| | 3051-3188, 3052-3096, 3052-3162, 3054-3096, 3061-3096, 3068-3093, 3068-3096, 3068-3162 |
| 47/7493320CB1/ | 1-593, 174-515, 174-995, 196-422, 196-437, 196-458, 196-465, 196-547, 196-592, 196-737, 196-880, 196-910, 197-371, |
| 3268 | 286-456, 422-710, 422-711, 572-1080, 583-1081, 584-1018, 586-1081, 593-1081, 596-1081, 604-1063, 613-1064, |
| | 613-1081, 615-1063, 660-1081, 668-1081, 695-1035, 860-1207, 890-1402, 901-1051, 907-1063, 909-1035, |
| | 921-1485, 1009-1142, 1082-1660, 1107-1572, 1117-1655, 1119-1524, 1133-1479, 1205-1813, 1211-1524, 1346-1603, |
| | 1468-1712, 1471-1945, 1512-2129, 1663-1840, 1684-2242, 1706-2191, 1718-2094, 1724-2309, 1783-2381, |
| | 1825-2353, 1829-2426, 1945-2212, 1957-2335, 1960-2332, 1961-2445, 2078-2157, 2333-2939, 2344-2936, 2560-2935, |
| | 2560-3067, 2560-3070, 2560-3114, 2586-3173, 2653-3214, 2683-2951, 2705-3192, 2739-2818, 2767-3235, |
| | 2802-3179, 2951-3235, 3056-3268, 3084-3235, 3187-3234 |
| 48/2911453CB1/ | 1-305, 1-587, 1-588, 1-647, 1-2430, 3-311, 104-258, 305-590, 310-585, 530-1089, 614-1039, 614-1044, 901-1090, |
| 2434 | 932-1647, 934-1096, 934-1131, 934-1237, 934-1647, 947-1096, 949-1044, 1281-2063, 1696-1806, 1696-1868, 1703-2219, |
| | 1953-2218, 1986-2405, 1992-2434, 2068-2267 |
| 49/3029661CB1/ | 1-528, 245-528, 370-684 |
| 684 | |
| 50/71260474CB1/ | 1-267, 1-419, 1-832, 318-914 |
| 914 | |
| 51/7992707CB1/ | 1-684, 4-729, 7-736, 12-736, 36-727, 39-733, 40-736, 50-730, 52-736, 57-710, 61-729, 68-730, 69-729, 78-724, 81-729, |
| 4713 | 82-729, 84-731, 96-729, 97-729, 101-736, 108-694, 112-729, 115-729, 124-729, 128-729, 133-729, 134-729, |
| | 142-729, 148-729, 157-736, 168-729, 169-729, 172-729, 173-727, 177-729, 180-729, 181-729, 193-729, 201-729, |
| | 204-729, 212-729, 213-729, 214-729, 216-736, 220-729, 231-729, 237-729, 246-729, 247-729, 293-730, 297-729, |
| | 316-736, 318-729, 340-719, 583-1206, 587-892, 620-1402, 621-970, 632-1005, 672-1458, 752-997, 752-1017, 752-1024, |
| | 756-1003, 777-1422, 858-1340, 1263-1772, 1266-1513, 1266-1724, 1286-1366, 1293-1572, 1323-1595, 1323-1817, |
| | 1463-1966, 1463-2103, 1600-2201, 1731-2202, 1740-2345, 1771-3565, 1804-2380, 1919-2380, 1926-2176, |
| | 1942-2560, 1995-2254, 1995-2528, 2171-2445, 2229-2732, 2359-3104, 2527-2761, 2549-2806, 2633-3075, 2676-2908, |
| | 2740-2959, 2740-3173, 2764-2974, 2834-3048, 2834-3420, 2963-3146, 3001-3501, 3008-3199, 3008-3445, |
| | 3069-3589, 3072-3598, 3126-3377, 3133-3605, 3138-3393, 3144-3396, 3153-3398, 3169-3806, 3206-3812, 3242-3619, |
| | 3263-3823, 3275-3793, 3303-3536, 3303-3794, 3303-3845, 3323-3967, 3328-3526, 3328-3806, 3328-3863, |
| | 3343-3731, 3377-3966, 3379-4043, 3385-3690, 3387-3646, 3400-4059, 3407-3966, 3498-3803, 3499-3728, 3508-3972, |
| | 3514-3783, 3521-3765, 3533-3681, 3538-3604, 3538-4079, 3553-4199, 3557-4073, 3572-4199, 3590-4216, |
| | 3597-4116, 3610-4199, 3611-3962, 3614-3787, 3619-4184, 3649-4216, 3670-4223, 3671-3967, 3683-4520, 3687-3884, |
| | 3691-4047, 3730-4024, 3732-4023, 3734-3888, 3739-4170, 3739-4389, 3747-4371, 3780-4198, 3814-4259, |
| | 3817-4361, 3873-4207, 3883-4198, 3952-4558, 3960-4326, 3976-4310, 3976-4521, 4024-4231, 4072-4564, 4144-4628, |
| | 4163-4713, 4213-4314, 4241-4573, 4243-4560, 4394-4682, 4490-4628 |
| 52/7974861CB1/ | 1-533, 1-537, 50-338, 50-624, 113-260, 118-5329, 2437-2544, 2703-3388, 2794-3388, 2893-3444, 2914-3786, 2948-3486, |
| 9885 | 2971-3398, 3007-3524, 3007-3762, 3009-3291, 3009-3346, 3009-3479, 3010-3527, 3014-3452, 3044-3519, |
| | 3048-3524, 3053-3519, 3057-3517, 3070-3894, 3085-3800, 3159-3989, 3168-3519, 3198-3887, 3205-3396, 3215-3461, |
| | 3215-3463, 3215-3666, 3215-3859, 3226-3978, 3257-3750, 3258-3958, 3299-3556, 3364-4108, 3376-3918, |
| | 3391-3513, 3401-4155, 3551-3929, 3609-4331, 3652-3920, 4376-4900, 4469-4740, 4741-5190, 4741-5375, 4929-5495, |
| | 5129-5645, 5129-5688, 5246-5522, 5289-5748, 5448-5670, 5594-5997, 5617-9397, 5809-6193, 5827-6308, |
| | 5895-6525, 6020-6279, 6020-6548, 6063-6592, 6120-6733, 6248-6866, 6273-6725, 6352-6613, 6352-6968, 6424-6864, |
| | 6429-6711, 6450-6973, 6469-7197, 6574-6821, 6574-6943, 6574-6968, 6588-6848, 6708-6923, 6744-6993, |
| | 6754-7221, 6759-7354, 6834-7370, 6919-7170, 6937-7196, 6987-7293, 6992-7311, 7065-7295, 7065-7335, 7065-7513, |
| | 7065-7688, 7123-7687, 7159-7709, 7165-7258, 7217-7751, 7222-7709, 7223-7690, 7243-7552, 7265-7549, |
| | 7279-7635, 7282-7636, 7303-7636, 7368-7636, 7416-7518, 7429-7636, 7568-7636, 7724-7994, 7724-8141, |
| | 7936-8189, 8046-8291, 8046-8363, 8046-8544, 8046-8702, 8181-8793, 8294-9011, 8414-8970, 8453-8815, 8539-9103, |
| | 8599-9205, 8630-9234, 8682-9352, 8682-9384, 8799-9587, 8843-9446, 8850-9540, 8901-9159, 8951-9575, |
| | 8959-9514, 8962-9232, 8999-9575, 9035-9316, 9053-9579, 9074-9271, 9086-9698, 9101-9368, 9150-9463, 9152-9505, |
| | 9160-9770, 9235-9435, 9313-9885 |
| 53/7499710CB1/ | 1-4543, 87-658, 191-387, 191-893, 193-4539, 198-1074, 300-1081, 328-1135, 414-528, 420-974, 421-1279, 519-1217, |
| 4607 | 560-1228, 580-1228, 631-1168, 631-1214, 670-1278, 688-1123, 798-1431, 808-1275, 898-1754, 921-1382, |
| | 929-1503, 1015-1661, 1020-1595, 1097-1766, 1103-1393, 1126-1447, 1147-1835, 1495-2148, 1602-2406, 1682-2319, |
| | 1699-2233, 1736-2263, 1744-2405, 1844-2212, 2007-2630, 2161-2469, 2194-2735, 2202-2585, 2202-2591, |
| | 2202-2619, 2207-2706, 2275-2683, 2277-2554, 2282-2797, 2341-3170, 2344-2974, 2360-3152, 2667-3188, 2734-3198, |
| | 2829-3423, 2872-3513, 2933-3602, 2948-3589, 2949-3609, 2997-3697, 2997-3738, 2998-3250, 2998-3678, |
| | 2998-3707, 2998-3790, 2998-3806, 2998-3880, 3002-3692, 3002-3715, 3004-3506, 3017-3679, 3026-3589, 3042-3568, |
| | 3076-3705, 3117-3713, 3145-3399, 3169-3847, 3174-3818, 3187-3818, 3202-3455, 3206-4075, 3220-3804, |
| | 3226-4073, 3236-3905, 3276-3713, 3282-3697, 3282-3725, 3286-3926, 3286-4025, 3289-4038, 3315-3703, 3422-3524, |
| | 3422-3525, 3445-3528, 3468-3943, 3480-4234, 3493-3932, 3495-3787, 3561-4058, 3645-4390, 3647-3893, |
| | 3674-3931, 3674-3991, 3674-4327, 3705-3871, 3731-3983, 3742-3905, 3747-4393, 3756-4393, 3761-4375, 3791-4266, |
| | 3794-4538, 3798-3989, 3821-4278, 3824-4393, 3838-4393, 3854-4514, 3860-4400, 3870-4355, 3874-4219, |
| | 3888-4369, 3890-4297, 3897-4439, 3906-4387, 3912-4534, 3913-4552, 3915-4536, 3918-4403, 3918-4423, 3920-4313, |
| | 3927-4390, 3928-4391, 3930-4453, 3947-4392, 3952-4411, 3954-4526, 3962-4564, 3970-4393, 3978-4467, |
| | 3986-4152, 3986-4392, 3990-4453, 3997-4249, 4026-4309, 4026-4313, 4026-4527, 4026-4539, 4037-4607, 4039-4564, |
| | 4041-4222, 4048-4507, 4055-4357, 4068-4208, 4068-4294, 4070-4132, 4088-4554, 4104-4537, 4108-4539, |
| | 4118-4518, 4119-4310, 4128-4414, 4138-4543, 4146-4539, 4149-4401, 4149-4402, 4155-4360, 4155-4410, 4291-4551, |
| | 4367-4539 |
| 54/8036958CB1/ | 1-578, 7-137, 8-639, 9-488, 24-563, 26-693, 27-636, 50-679, 137-395, 137-568, 137-608, 137-616, 137-620, 137-622 |
| 4486 | 137-629, 137-650, 139-490, 141-594, 143-720, 145-620, 145-672, 146-668, 146-753, 146-761, 146-772, 155-500, |
| | 159-614, 161-624, 166-613, 187-645, 263-405, 277-750, 294-744, 343-491, 343-558, 343-561, 343-624, 359-762, |
| | 377-558, 439-774, 451-1139, 513-1106, 525-1002, 538-951, 578-818, 732-1604, 885-1133, 885-1492, 885-1511, |
| | 885-1536, 885-1620, 885-1675, 885-1679, 891-1650, 911-1490, 965-1574, 995-1529, 1045-1514, 1084-1299, |
| | 1133-1608, 1136-1416, 1136-1427, 1189-1619, 1230-1466, 1230-1691, 1254-1837, 1272-2124, 1295-1868, 1324-1999, |
| | 1331-1459, 1337-1785, 1337-1935, 1337-1977, 1337-2012, 1339-2051, 1372-2069, 1537-2155, 1652-1837, |
| | 1788-1979, 1788-2183, 1790-2138, 1790-2367, 1937-2344, 1991-2585, 2019-2801, 2022-2587, 2136-2369, 2169-2396, |
| | 2276-2919, 2285-2512, 2313-2577, 2313-2964, 2352-2890, 2483-2747, 2483-3107, 2655-3123, 2694-2855, |
| | 2694-2967, 2719-3024, 2726-3033, 2794-2979, 2812-3084, 2819-3132, 2819-3369, 2905-3151, 2938-3558, 2954-3230, |
| | 2954-3543, 2982-3229, 2982-3530, 2984-3252, 3006-3249, 3020-3622, 3050-3661, 3065-3329, 3175-3442, |
| | 3181-3429, 3241-3516, 3252-3522, 3266-3551, 3269-3527, 3279-3583, 3279-3875, 3288-3503, 3292-3587, 3320-3590, |
| | 3395-3667, 3427-3678, 3463-3668, 3466-3707, 3473-3725, 3511-3815, 3536-3750, 3538-3796, 3587-3877, |
| | 3597-3834, 3597-4228, 3647-3919, 3679-3860, 3749-4440, 3770-3989, 3808-4044, 3808-4052, 3823-4076, 3847-4417, |
| | 3891-4031, 3932-4127, 3942-4443, 4010-4280, 4047-4296, 4055-4454, 4066-4447, 4093-4358, 4166-4473, |
| | 4193-4227, 4210-4400, 4291-4486 |
| 55/3253807CB1/ | 1-646, 182-634, 185-833, 200-1969, 325-808, 329-956, 408-630, 421-805, 424-897, 433-942, 453-1150, 504-1209, |
| 2559 | 523-962, 531-1219, 618-1086, 629-887, 630-1253, 636-1241, 644-1235, 653-1200, 719-1488, 728-1022, 730-1373, |
| | 754-1513, 784-995, 841-906, 1280-2010, 1383-2167, 1459-2129, 1462-2203, 1486-1980, 1500-2248, 1510-2105, |
| | 1565-2363, 1615-2312, 1621-2434, 1634-2055, 1648-1936, 1680-2109, 1681-2553, 1685-2081, 1688-2053, 1691-1968, |
| | 1692-1946, 1717-1926, 1721-2224, 1732-2381, 1739-2400, 1748-2115, 1754-2559, 1774-2076, 1778-2066, |
| | 1827-2116, 1859-2122, 1863-2502, 1904-2559 |
| 56/3626408CB1/ | 1-369, 1-474, 1-511, 1-567, 1-570, 1-590, 1-591, 1-608, 1-610, 1-630, 1-663, 1-674, 1-686, 1-831, 8-304, 38-664, 84-751, |
| 839 | 207-839, 276-839, 277-834, 325-823, 347-839, 382-836, 429-836, 432-839 |
| 57/3773014CB1/ | 1-283, 1-4602, 30-271, 55-180, 99-504, 159-3315, 259-517, 369-744, 534-659, 628-685, 694-984, 705-984, 895-1525, |
| 4898 | 942-1527, 943-1457, 979-1552, 979-1814, 980-1886, 981-1626, 981-1697, 981-1731, 1110-1708, 1127-1715, |
| | 1148-1414, 1288-1843, 1439-1532, 1553-1717, 1581-2268, 1583-2296, 1646-2062, 1739-2519, 1792-2519, 1802-2519, |
| | 1896-2291, 1899-2462, 1901-2519, 1960-2492, 2000-2627, 2010-2728, 2011-2448, 2015-2705, 2088-2835, |
| | 2287-2373, 2295-2818, 2434-2701, 2459-2781, 2501-3009, 2550-2947, 2562-3170, 2647-2855, 2726-3108, 2728-2998, |
| | 2732-3412, 2773-3053, 2775-3016, 2775-3227, 2775-3308, 2775-3341, 2775-3441, 2775-3513, 2834-3433, |
| | 2901-3693, 2962-3245, 2962-3365, 2962-3372, 2962-3760, 2962-3785, 2962-3869, 2963-3784, 3086-3460, 3092-3911, |
| | 3094-3321, 3171-3989, 3176-3995, 3196-3247, 3213-3500, 3241-3528, 3258-3549, 3307-4044, 3318-3633, |
| | 3365-4068, 3377-4026, 3423-3693, 3457-3725, 3468-3892, 3473-3891, 3495-3771, 3529-4256, 3536-3864, 3561-4345, |
| | 3566-4071, 3607-3857, 3639-3883, 3678-4155, 3693-4233, 3704-4364, 3744-4459, 3773-4231, 3827-4284, |
| | 3837-4426, 3860-4441, 3861-3946, 3861-4039, 3918-4233, 3954-4784, 4007-4135, 4007-4204, 4007-4395, 4022-4807, |
| | 4028-4856, 4123-4757, 4166-4423, 4166-4458, 4181-4487, 4187-4873, 4191-4861, 4202-4453, 4211-4395, |
| | 4213-4849, 4249-4872, 4272-4489, 4282-4881, 4286-4530, 4286-4821, 4334-4585, 4356-4890, 4361-4881, 4369-4898, |
| | 4372-4846, 4392-4747, 4393-4856, 4396-4856, 4398-4856, 4405-4877, 4412-4704, 4415-4898, 4422-4857, |
| | 4424-4852, 4427-4679, 4432-4856, 4440-4857, 4449-4784, 4462-4679, 4466-4877, 4467-4810, 4474-4852, 4477-4739, |
| | 4484-4852, 4491-4852, 4514-4706, 4525-4856, 4526-4856, 4527-4786, 4559-4856, 4582-4770 |
| 58/4398735CB1/ | 1-626, 1-977, 510-976, 678-3229, 1274-1662, 1871-2282, 1871-2547, 1914-2038, 2067-2194, 2226-2806, 2228-2469, |
| 3537 | 2299-2547, 2336-2556, 2397-2905, 2871-2966, 2882-2977, 2962-3233, 2962-3250, 3041-3281, 3041-3537, |
| | 3140-3418, 3149-3387, 3158-3368 |
| 59/7499579CB1/ | 1-755, 28-736, 40-553, 577-1037, 600-1312, 1045-1285, 1045-1297, 1045-1327, 1045-1641, 1045-1670, 1045-1786, |
| 1822 | 1045-1822, 1046-1778 |
| 60/8178947CB1/ | 1-320, 1-395, 1-1352, 2-232, 88-338, 223-653, 667-826, 667-951, 678-965, 718-980, 718-1317, 718-1353, 718-1528, |
| 2497 | 721-791, 731-1034, 731-1353, 734-791, 745-791, 745-848, 745-849, 745-876, 752-881, 760-847, 760-881, |
| | 775-881, 823-919, 835-932, 835-933, 835-1026, 837-965, 844-965, 920-1033, 948-1352, 1172-1285, 1184-1285, |
| | 1237-1285, 1249-1375, 1249-1380, 1250-1375, 1255-1375, 1255-1446, 1280-1591, 1309-1379, 1322-1379, 1334-1379, |
| | 1359-1605, 1366-1605, 1368-1743, 1411-1507, 1424-1507, 1430-1507, 1431-1507, 1435-2028, 1526-2262, |
| | 1719-1922, 1983-2497 |
| 61/2264652CB1/ | 1-459, 412-510, 412-1000, 412-1097, 412-1311, 416-847, 446-869, 446-966, 446-983, 446-1011, 446-1025, 446-1031, |
| 4943 | 446-1082, 446-1138, 448-700, 511-1055, 533-1193, 667-1142, 721-994, 732-1597, 930-1594, 1280-2063, |
| | 1288-1615, 1290-1388, 1377-1906, 1389-1494, 1391-1921, 1408-1615, 1464-2007, 1474-1965, 1548-2047, 1548-2049, |
| | 1581-1845, 1626-2684, 1707-1904, 1941-2116, 2008-2299, 2119-2306, 2262-2304, 2369-2600, 2386-2888, |
| | 2391-2996, 2391-3154, 2419-3127, 2537-3002, 2586-3228, 2616-3173, 2678-2749, 2743-3155, 2780-3280, 3184-3455, |
| | 3184-3769, 3279-4036, 3346-3620, 3355-4098, 3483-3539, 3590-3894, 3676-3910, 3744-3804, 3750-4222, |
| | 3784-4043, 3803-3901, 3857-4302, 3906-4080, 3933-4279, 3965-4261, 4156-4932, 4168-4442, 4193-4490, 4198-4661, |
| | 4233-4943, 4238-4474 |
| 62/1806372CB1/ | 1-2223, 552-1041, 963-1127, 963-1159, 963-1214, 963-1235, 963-1277, 963-1281, 963-1294, 966-1147, 973-1391, |
| 2585 | 998-1246, 998-1282, 998-1341, 998-1364, 998-1368, 998-1379, 1083-1441, 1116-1203, 1116-1220, 1116-1280, |
| | 1116-1381, 1116-1454, 1116-1507, 1116-1514, 1116-1521, 1144-1466, 1156-1488, 1156-1498, 1445-2047, 1449-1599, |
| | 1481-1971, 1490-1632, 1496-1996, 1499-2155, 1501-2067, 1509-2033, 1540-2224, 1563-2060, 1565-1933, |
| | 1601-2264, 1607-1933, 1607-2174, 1608-2080, 1634-1907, 1640-2266, 1660-2061, 1673-2080, 1676-2293, 1688-2245, |
| | 1693-2245, 1700-2080, 1700-2233, 1701-2006, 1713-2080, 1713-2188, 1723-2379, 1730-2302, 1745-2188, |
| | 1745-2217, 1784-2415, 1795-2360, 1811-2245, 1837-2492, 1851-2522, 1854-2428, 1868-2428, 1871-2428, 1877-2428, |
| | 1878-2450, 1880-2342, 1880-2428, 1880-2453, 1893-2428, 1894-2474, 1900-2427, 1934-2245, 1939-2428, |
| | 1947-2313, 1953-2428, 1958-2517, 1966-2548, 1972-2428, 1973-2313, 1973-2428, 1978-2521, 1991-2550, 2003-2523, |
| | 2012-2546, 2014-2282, 2050-2462, 2070-2332, 2076-2311, 2081-2427, 2081-2428, 2081-2551, 2098-2254, |
| | 2107-2552, 2162-2534, 2191-2531, 2209-2538, 2310-2539, 2346-2585, 2369-2528 |
| 63/2010564CB1/ | 1-652, 220-1347, 953-1400, 959-1410, 963-1429, 965-1429, 974-1412, 1001-1262, 1001-1416, 1014-1410, 1039-1410, |
| 1888 | 1051-1429, 1123-1426, 1238-1426, 1261-1346, 1348-1888 |
| 64/7364908CB1/ | 1-259, 1-572, 1-1787, 93-633, 93-635, 502-701, 595-1208, 841-1074, 841-1388, 891-1432, 943-1228, 943-1267, 943-1573, |
| 2991 | 1256-1341, 1256-1350, 1256-1354, 1256-1357, 1256-1384, 1256-1398, 1256-1412, 1256-1457, 1256-1458, |
| | 1256-1462, 1256-1489, 1256-1496, 1256-1514, 1256-1593, 1256-1630, 1256-1674, 1256-1717, 1275-1425, 1283-1425, |
| | 1283-1674, 1284-1593, 1285-1674, 1305-1630, 1306-1438, 1307-1476, 1307-1674, 1309-1674, 1317-1603, |
| | 1317-1630, 1319-1437, 1320-1501, 1320-1531, 1320-1532, 1320-1553, 1320-1580, 1320-1641, 1322-1674, 1322-1717, |
| | 1324-1384, 1324-1593, 1324-1630, 1331-1533, 1331-1598, 1332-1356, 1332-1506, 1334-1593, 1336-1593, |
| | 1340-1515, 1340-1641, 1340-1713, 1349-1570, 1369-1596, 1369-1674, 1371-1778, 1391-1717, 1398-1684, 1398-1967, |
| | 1398-1991, 1403-1674, 1408-1641, 1408-1717, 1413-1642, 1415-1677, 1430-1593, 1433-1641, 1434-1476, |
| | 1434-1674, 1451-1593, 1452-1717, 1455-1713, 1473-1713, 1484-1586, 1484-1615, 1484-1616, 1484-1641, 1484-1647, |
| | 1484-1661, 1484-1718, 1485-1717, 1488-1682, 1488-1683, 1488-1706, 1488-1720, 1490-1904, 1492-1683, |
| | 1492-1713, 1503-1717, 1504-1717, 1509-1686, 1517-1718, 1523-1718, 1525-1718, 1528-1593, 1534-1674, |
| | 1536-1641, 1568-1718, 1569-1641, 1592-1717, 1620-1717, 1620-1718, 1641-1720, 1657-1712, 1657-1923, 1657-1933, |
| | 1707-2278, 1709-1982, 1722-1832, 1722-1834, 1722-1850, 1722-1859, 1722-1888, 1722-1920, 1722-1955, |
| | 1722-1957, 1722-2045, 1733-1989, 1737-1775, 1737-1904, 1742-1918, 1742-2046, 1742-2206, 1748-1904, 1779-2045, |
| | 1792-1904, 1794-1904, 1802-1872, 1802-1914, 1802-1934, 1802-2001, 1802-2039, 1802-2240, 1806-2049, |
| | 1806-2075, 1808-2367, 1810-2045, 1815-2017, 1819-1992, 1819-2056, 1826-1992, 1826-2046, 1826-2247, 1832-1992, |
| | 1835-2112, 1835-2287, 1835-2379, 1838-2550, 1843-2383, 1850-2383, 1853-1992, 1858-2383, 1866-2413, |
| | 1877-2206, 1893-1992, 1894-2172, 1894-2206, 1899-2056, 1910-2055, 1910-2166, 1910-2214, 1910-2247, 1932-2383, |
| | 1939-1992, 1941-2046, 1941-2247, 1971-2563, 1973-2247, 1978-2247, 1983-2166, 1985-2247, 1988-2279, |
| | 1990-2387, 2025-2247, 2030-2247, 2070-2111, 2070-2170, 2070-2244, 2070-2247, 2071-2175, 2071-2176, 2071-2234, |
| | 2071-2244, 2073-2111, 2073-2238, 2073-2240, 2073-2563, 2084-2284, 2097-2247, 2098-2244, 2105-2244, |
| | 2105-2569, 2145-2240, 2145-2244, 2155-2247, 2175-2588, 2191-2244, 2236-2475, 2236-2570, 2416-2991, |
| | 2547-2877 |
| 65/7489960CB1/ | 1-281, 1-289, 1-711, 1-1315, 216-810, 313-592, 321-647, 368-586, 368-768, 391-565, 391-666, 711-1313, 848-1336, |
| 3874 | 849-1336, 857-1426, 859-1336, 862-1462, 1085-1674, 1221-1512, 1342-1965, 1555-2153, 1584-2178, 1613-2166, |
| | 1621-2167, 1681-1714, 1703-2547, 1956-2547, 2006-2177, 2007-2177, 2012-2177, 2131-2386, 2131-2387, |
| | 2159-2723, 2231-2403, 2425-2891, 2430-2545, 2433-2679, 2547-2897, 2663-2942, 2663-2990, 2664-2926, 2670-3018, |
| | 2719-2895, 2719-3212, 2915-3874, 2984-3209, 2989-3234 |
| 66/8555401CB1/ | 1-643, 22-1447, 51-681, 51-794, 51-795, 776-1378, 816-1388, 818-1342, 1053-1447, 1086-1438, 1086-1670, 1119-1447 |
| 1670 | |

TABLE 5

| Polynucleotide SEQ | | |
|---|---|---|
| ID NO: | Incyte Project ID: | Representative Library |
| 34 | 4001873CB1 | TLYMNOT06 |
| 36 | 5855204CB1 | SINTFEE01 |
| 37 | 5778654CB1 | ESOGTUC02 |
| 38 | 1440126CB1 | BRAWTDR02 |
| 39 | 3934519CB1 | KIDNNOC01 |
| 40 | 2946314CB1 | FTUBTUE01 |
| 41 | 3617784CB1 | BRATNOR01 |
| 42 | 7490869CB1 | BRAITUT12 |
| 43 | 5994687CB1 | BLADNOT08 |
| 44 | 2560755CB1 | BLADNOR01 |
| 45 | 3217430CB1 | PANCNOT08 |
| 46 | 5786832CB1 | LIVRTMR01 |
| 47 | 7493320CB1 | BRAUTDR02 |
| 48 | 2911453CB1 | LUNGTUT08 |
| 49 | 3029661CB1 | BLADTUT04 |
| 50 | 71260474CB1 | SYNORAB01 |
| 51 | 7992707CB1 | BRSTNOT14 |
| 52 | 7974861CB1 | TESTNOT03 |
| 53 | 7499710CB1 | TESTTUT02 |
| 54 | 8036958CB1 | BRAUNOR01 |
| 55 | 3253807CB1 | COLDNOT01 |
| 56 | 3626408CB1 | BRAIFEN03 |
| 57 | 3773014CB1 | BMARTXE01 |
| 58 | 4398735CB1 | COLNNOT27 |
| 59 | 7499579CB1 | BRAMDIT02 |
| 60 | 8178947CB1 | PLACNOT02 |
| 61 | 2264652CB1 | SINTFEE01 |
| 62 | 1806372CB1 | SININOT04 |
| 63 | 2010564CB1 | TESTNOT03 |
| 64 | 7364908CB1 | PROSTUT13 |
| 65 | 7489960CB1 | SKIRNOR01 |
| 66 | 8555401CB1 | LUNGNOT30 |

TABLE 6

| Library | Vector | Library Description |
|---|---|---|
| BLADNOR01 | PCDNA2.1 | This random primed library was constructed using RNA isolated from |
| | | the bladder tissue of an 11-year-old Black male who |
| | | died from a gunshot wound. Serology was positive for CMV. |
| BLADNOT08 | pINCY | Library was constructed using RNA isolated from the bladder tissue of an |
| | | 11-year-old black male, who died from a gunshot |
| | | wound. |
| BLADTUT04 | pINCY | Library was constructed using RNA isolated from bladder tumor tissue |
| | | removed from a 60-year-old Caucasian male during |
| | | a radical cystectomy, prostatectomy, and vasectomy. Pathology indicated grade 3 |
| | | transitional cell carcinoma in the left |
| | | bladder wall. Carcinoma in-situ was identified in the dome and trigone. |
| | | Patient history included tobacco use. Family history |
| | | included type I diabetes, malignant neoplasm of the stomach, atherosclerotic |
| | | coronary artery disease, and acute myocardial |
| | | infarction. |
| BMARTXE01 | pINCY | This 5′ biased random primed library was constructed using RNA |
| | | isolated from treated SH-SY5Y cells derived from a |
| | | metastatic bone marrow neuroblastoma, removed from a 4-year-old |
| | | Caucasian female (Schering AG). The medium was |
| | | MEM/HAM'S F12 with 10% fetal calf serum. After reaching |
| | | about 80% confluency cells were treated with 6- |
| | | Hydroxydopamine (6-OHDA) at 100 microM for 8 hours. |
| BRAIFEN03 | pINCY | This normalized fetal brain tissue library was constructed |
| | | from 3.26 million independent clones from a fetal brain library. |
| | | Starting RNA was made from brain tissue removed from a Caucasian male |
| | | fetus, who was stillborn with a hypoplastic left |
| | | heart at 23 weeks' gestation. The library was normalized in 2 |
| | | rounds using conditions adapted from Soares et al., PNAS |
| | | (1994) 91: 9228 and Bonaldo et al., Genome Research (1996) |
| | | 6: 791, except that a significantly longer (48 hours/round) |
| | | reannealing hybridization was used. |
| BRAITUT12 | pINCY | Library was constructed using RNA isolated from brain tumor |
| | | tissue removed from the left frontal lobe of a 40-year-old |
| | | Caucasian female during excision of a cerebral meningeal lesion. Pathology |
| | | indicated grade 4 gemistocytic astrocytoma. |
| BRAMDIT02 | pINCY | Library was constructed using RNA isolated from diseased medulla |
| | | tissue removed from the brain of a 74-year-old |
| | | Caucasian female who died from respiratory arrest due to amyotrophic |
| | | lateral sclerosis (ALS). Serologies were negative. |
| | | Patient history included amyotrophic lateral sclerosis, hypertension, |
| | | arthritis, and alcohol use. Previous surgeries included |
| | | insertion of gastrointestinal tubes and cataract extraction. |
| | | Patient medications included lorazepam and amitriptyline. |
| BRATNOR01 | PCDNA2.1 | This random primed library was constructed using |
| | | RNA isolated from temporal cortex tissue removed from a 45-year-old |
| | | Caucasian female who died from a dissecting aortic aneurysm and |
| | | ischemic bowel disease. Pathology indicated mild |
| | | arteriosclerosis involving the cerebral cortical white matter |
| | | and basal ganglia. Grossly, there was mild meningeal fibrosis |
| | | and mild focal atherosclerotic plaque in the middle cerebral |
| | | artery, as well as vertebral arteries bilaterally. Microscopically, |
| | | the cerebral hemispheres, brain stem and cerebellum reveal |
| | | focal areas in the white matter that contain blood vessels that |
| | | were barrel-shaped, hyalinized, with hemosiderin-laden macrophages |
| | | in the Virchow-Robin space. In addition, there were |
| | | scattered neurofibrillary tangles within the basolateral nuclei |
| | | of the amygdala. Patient history included mild atheromatosis |
| | | of aorta and coronary arteries, bowel and liver infarct due to |
| | | aneurysm, physiologic fatty liver associated with obesity, mild |
| | | diffuse emphysema, thrombosis of mesenteric and portal veins, |
| | | cardiomegaly due to hypertrophy of left ventricle, arterial |
| | | hypertension, acute pulmonary edema, splenomegaly, obesity |
| | | (300 lb.), leiomyoma of uterus, sleep apnea, and iron |
| | | deficiency anemia. |
| BRAUNOR01 | pINCY | This random primed library was constructed using RNA |
| | | isolated from striatum, globus pallidus and posterior putamen |
| | | tissue removed from an 81-year-old Caucasian female who died |
| | | from a hemorrhage and ruptured thoracic aorta due to |
| | | atherosclerosis. Pathology indicated moderate atherosclerosis |
| | | involving the internal carotids, bilaterally; microscopic |
| | | infarcts of the frontal cortex and hippocampus; and scattered |
| | | diffuse amyloid plaques and neurofibrillary tangles, consistent |
| | | with age. Grossly, the leptomeninges showed only mild thickening |
| | | and hyalinization along the superior sagittal sinus. The |
| | | remainder of the leptomeninges was thin and contained some |
| | | congested blood vessels. Mild atrophy was found mostly in |
| | | the frontal poles and lobes, and temporal lobes, bilaterally. |
| | | Microscopically, there were pairs of Alzheimer type II |
| | | astrocytes within the deep layers of the neocortex. |
| | | There was increased satellitosis around neurons in the deep gray matter |
| | | in the middle frontal cortex. The amygdala contained rare |
| | | diffuse plaques and neurofibrillary tangles. The posterior |
| | | hippocampus contained a microscopic area of cystic cavitation |
| | | with hemosiderin-laden macrophages surrounded by |
| | | reactive gliosis. Patient history included sepsis, cholangitis, |
| | | post-operative atelectasis, pneumonia CAD, cardiomegaly due |
| | | to left ventricular hypertrophy, splenomegaly, |
| | | arteriolonephrosclerosis, nodular colloidal goiter, emphysema, CHF, |
| | | hypothyroidism, and peripheral vascular disease. |
| BRAUTDR02 | PCDNA2.1 | This random primed library was constructed using RNA |
| | | isolated from pooled amygdala and entorhinal cortex tissue |
| | | removed from a 55-year-old Caucasian female who died from |
| | | cholangiocarcinoma. Pathology indicated mild meningeal |
| | | fibrosis predominately over the convexities, scattered axonal |
| | | spheroids in the white matter of the cingulate cortex and the |
| | | thalamus, and a few scattered neurofibrillary tangles in the |
| | | entorhinal cortex and the periaqueductal gray region. Pathology |
| | | for the associated tumor tissue indicated well-differentiated |
| | | cholangiocarcinoma of the liver with residual or relapsed |
| | | tumor. Patient history included cholangiocarcinoma, |
| | | post-operative Budd-Chiari syndrome, biliary ascites, hydrothorax, |
| | | dehydration, malnutrition, oliguria and acute renal failure. |
| | | Previous surgeries included cholecystectomy and resection of |
| | | 85% of the liver. |
| BRAWTDR02 | PCDNA2.1 | This random primed library was constructed using RNA |
| | | isolated from dentate nucleus tissue removed from a 55-year-old |
| | | Caucasian female who died from cholangiocarcinoma. |
| | | Pathology indicated mild meningeal fibrosis predominately over the |
| | | convexities, scattered axonal spheroids in the white matter of the |
| | | cingulate cortex and the thalamus, and a few scattered |
| | | neurofibrillary tangles in the entorhinal cortex and the |
| | | periaqueductal gray region. Pathology for the associated tumor tissue |
| | | indicated well-differentiated cholangiocarcinoma of the liver |
| | | with residual or relapsed tumor. Patient history included |
| | | cholangiocarcinoma, post-operative Budd-Chiari syndrome, biliary |
| | | ascites, hydrothorax, dehydration, malnutrition, oliguria |
| | | and acute renal failure. Previous surgeries included |
| | | cholecystectomy and resection of 85% of the liver. |
| BRSTNOT14 | pINCY | Library was constructed using RNA isolated |
| | | from breast tissue removed from a 62-year-old Caucasian female during a |
| | | unilateral extended simple mastectomy. Pathology for the |
| | | associated tumor tissue indicated an invasive grade 3 (of 4), |
| | | nuclear grade 3 (of 3) adenocarcinoma, ductal type. Ductal |
| | | carcinoma in situ, comedo type, comprised 60% of the tumor |
| | | mass. Metastatic adenocarcinoma was identified in one (of 14) |
| | | axillary lymph nodes with no perinodal extension. The |
| | | tumor cells were strongly positive for estrogen receptors and |
| | | weakly positive for progesterone receptors. Patient history |
| | | included a benign colon neoplasm, hyperlipidemia, cardiac |
| | | dysrhythmia, and obesity. Family history included |
| | | atherosclerotic coronary artery disease, myocardial infarction, |
| | | colon cancer, ovarian cancer, lung cancer, and |
| | | cerebrovascular disease. |
| COLDNOT01 | pINCY | Library was constructed using RNA isolated from diseased |
| | | descending colon tissue removed from a 16-year-old Caucasian |
| | | male during partial colectomy, temporary ileostomy, and colonoscopy. |
| | | Pathology indicated innumerable (greater than 100) |
| | | adenomatous polyps with low grade dysplasia involving the |
| | | entire colonic mucosa in the setting of familial polyposis coli. |
| | | The patient presented with abdominal pain and flatulence. |
| | | The patient was not taking any medications. Family history |
| | | included benign colon neoplasm in the father; benign colon |
| | | neoplasm in the sibling(s); and benign hypertension, |
| | | cerebrovascular disease, breast cancer, uterine cancer, |
| | | and type II diabetes in the grandparent(s). |
| COLNNOT27 | pINCY | Library was constructed using RNA isolated from diseased |
| | | cecal tissue removed from 31-year-old Caucasian male during a |
| | | total intra-abdominal colectomy, appendectomy, and permanent |
| | | ileostomy. Pathology indicated severe active Crohn's |
| | | disease involving the colon from the cecum to the rectum. |
| | | There were deep rake-like ulcerations which spared the |
| | | intervening mucosa. The ulcers extended into the muscularis, |
| | | and there was transmural inflammation. Patient history |
| | | included an irritable colon. Previous surgeries included a colonoscopy. |
| ESOGTUC02 | PSPORT1 | This large size fractionated library was constructed using |
| | | pooled cDNA from two different donors. cDNA was generated |
| | | using mRNA isolated from esophageal tissue removed from a |
| | | 53-year-old Caucasian male (donor A) during a partial |
| | | esophagectomy, proximal gastrectomy, and regional lymph |
| | | node biopsy and from esophagus tumor tissue removed from a |
| | | 61-year-old Caucasian male (donor B) during proximal |
| | | gastrectomy and partial esophagectomy. Pathology indicated no |
| | | significant abnormality in the non-neoplastic esophagus |
| | | for donor A. For donor B, pathology indicated invasive grade 3 |
| | | adenocarcinoma forming an ulcerated, plaque-like mass |
| | | situated at the lower esophagus just proximal to the |
| | | gastroesophageal junction, with partial involvement of |
| | | cardiac mucosa. The tumor invaded through muscularis propria and |
| | | focally into adventitial soft tissue. Donor A presented with |
| | | dysphagia. Donor B presented with heartburn, abnormal weight |
| | | loss, and anxiety. Patient history for donor A included |
| | | membranous nephritis, hyperlipidemia, benign hypertension, and |
| | | anxiety state. Patient history for donor B included a benign |
| | | colon neoplasm and hyperlipidemia. Previous surgeries |
| | | included an adenotonsillectomy, appendectomy, and inguinal |
| | | hernia repair for donor A and polypectomy for donor B. |
| | | Donor A was not taking any medications and donor B's medications |
| | | included Prilosec, ferrous sulfate, and vitamins. Family |
| | | history (A) included atherosclerotic coronary artery disease, |
| | | alcoholic cirrhosis, alcohol abuse, and an abdominal aortic |
| | | aneurysm rupture in the father; breast cancer in the mother; |
| | | a myocardial infarction and atherosclerotic coronary artery |
| | | disease in the sibling(s); and myocardial infarction and |
| | | atherosclerotic coronary artery disease in the grandparent(s). Family |
| | | history (B) included type II diabetes in the mother; |
| | | accessory sinus cancer, atherosclerotic coronary artery disease, and |
| | | acute myocardial infarction in the father. |
| FTUBTUE01 | pINCY | This 5′ biased random primed library was constructed |
| | | using RNA isolated from right fallopian tube tumor tissue removed |
| | | from an 85-year-old Caucasian female during bilateral |
| | | salpingo-oophorectomy and hysterectomy. Pathology indicated |
| | | poorly differentiated mixed endometrioid (80%) and serous |
| | | (20%) adenocarcinoma of the right fallopian tube, which was |
| | | confined to the mucosa without mural involvement. |
| | | Endometrioid carcinoma in situ was also present. Pathology for the |
| | | associated uterus tumor indicated focal endometrioid |
| | | adenocarcinoma in situ and moderately differentiated invasive |
| | | adenocarcinoma arising in an endometrial polyp. A metastatic |
| | | endometrioid and serous adenocarcinoma was present in the |
| | | cul-de-sac tumor. The patient presented with a pelvic mass |
| | | and ascites. Patient history included medullary carcinoma of the |
| | | thyroid and myocardial infarction. Patient medications |
| | | included Nitro-Dur, Lescol, Lasix and Cardizem. |
| KIDNNOC01 | pINCY | This large size-fractionated library was constructed using |
| | | RNA isolated from pooled left and right kidney tissue removed |
| | | from a Caucasian male fetus, who died from Patau's syndrome |
| | | (trisomy 13) at 20-weeks' gestation. |
| LIVRTMR01 | PCDNA2.1 | This random primed library was constructed |
| | | using RNA isolated from liver tissue removed from a 62-year-old Caucasian |
| | | female during partial hepatectomy and exploratory laparotomy. |
| | | Pathology for the matched tumor tissue indicated metastatic |
| | | intermediate grade neuroendocrine carcinoma, consistent with |
| | | islet cell tumor, forming nodules ranging in size, in the |
| | | lateral and medial left liver lobe. The pancreas showed fibrosis, |
| | | chronic inflammation and fat necrosis consistent with |
| | | pseudocyst. The gallbladder showed mild chronic cholecystitis. |
| | | Patient history included malignant neoplasm of the |
| | | pancreas tail, pulmonary embolism, hyperlipidemia, thrombophlebitis, |
| | | joint pain in multiple joints, type II diabetes, benign |
| | | hypertension, cerebrovascular disease, and normal delivery. |
| | | Previous surgeries included distal pancreatectomy, total |
| | | splenectomy, and partial hepatectomy. Family history included |
| | | pancreas cancer with secondary liver cancer, benign |
| | | hypertension, and hyperlipidemia. |
| LUNGNOT30 | pINCY | Library was constructed using RNA isolated from lung |
| | | tissue removed from a Caucasian male fetus, who died from Patau's |
| | | syndrome (trisomy 13) at 20-weeks' gestation. |
| LUNGTUT08 | pINCY | Library was constructed using RNA isolated from lung |
| | | tumor tissue removed from a 63-year-old Caucasian male during a |
| | | right upper lobectomy with fiberoptic bronchoscopy. Pathology |
| | | indicated a grade 3 adenocarcinoma. Patient history |
| | | included atherosclerotic coronary artery disease, an acute |
| | | myocardial infarction, rectal cancer, an asymtomatic abdominal |
| | | aortic aneurysm, tobacco abuse, and cardiac dysrhythmia. |
| | | Family history included congestive heart failure, stomach cancer, |
| | | and lung cancer, type II diabetes, atherosclerotic coronary |
| | | artery disease, and an acute myocardial infarction. |
| PANCNOT08 | pINCY | Library was constructed using RNA isolated from |
| | | pancreatic tissue removed from a 65-year-old Caucasian female during |
| | | radical subtotal pancreatectomy. Pathology for the associated |
| | | tumor tissue indicated an invasive grade 2 adenocarcinoma. |
| | | Patient history included type II diabetes, osteoarthritis, |
| | | cardiovascular disease, benign neoplasm in the large bowel, and a |
| | | cataract. Previous surgeries included a total splenectomy, |
| | | cholecystectomy, and abdominal hysterectomy. Family history |
| | | included cardiovascular disease, type II diabetes, and stomach cancer. |
| PLACNOT02 | pINCY | Library was constructed using RNA isolated from |
| | | the placental tissue of a Hispanic female fetus, who was prematurely |
| | | delivered at 21 weeks' gestation. Serologies of |
| | | the mother's blood were positive for CMV (cytomegalovirus). |
| PROSTUT13 | pINCY | Library was constructed using RNA isolated from prostate |
| | | tumor tissue removed from a 59-year-old Caucasian male during |
| | | a radical prostatectomy with regional lymph node excision. |
| | | Pathology indicated adenocarcinoma (Gleason grade 3 + 3). |
| | | Adenofibromatous hyperplasia was present. The patient |
| | | presented with elevated prostate-specific antigen (PSA). Patient |
| | | history included colon diverticuli, asbestosis, and |
| | | thrombophlebitis. Family history included multiple myeloma, |
| | | hyperlipidemia, and rheumatoid arthritis. |
| SININOT04 | pINCY | Library was constructed using RNA isolated from diseased |
| | | ileum tissue obtained from a 26-year-old Caucasian male during |
| | | a partial colectomy, permanent colostomy, and an incidental |
| | | appendectomy. Pathology indicated moderately to severely |
| | | active Crohn's disease. Family history included enteritis |
| | | of the small intestine. |
| SINTFEE01 | pINCY | This 5′ biased random primed library was constructed |
| | | using RNA isolated from small intestine tissue removed from a |
| | | Caucasian male fetus who died from fetal demise. |
| SKIRNOR01 | PCDNA2.1 | This random primed library was constructed using RNA |
| | | isolated from skin tissue removed from the breast of a 17-year-old |
| | | Caucasian female during bilateral reduction mammoplasty. Patient |
| | | history included breast hypertrophy. Family history |
| | | included benign hypertension. |
| SYNORAB01 | PBLUESCRIPT | Library was constructed using RNA isolated from the |
| | | synovial membrane tissue of a 68-year-old Caucasian female with |
| | | rheumatoid arthritis. |
| TESTNOT03 | PBLUESCRIPT | Library was constructed using RNA isolated from |
| | | testicular tissue removed from a 37-year-old Caucasian male, who died |
| | | from liver disease. Patient history included cirrhosis, jaundice, |
| | | and liver failure. |
| TESTTUT02 | pINCY | Library was constructed using RNA isolated from |
| | | testicular tumor removed from a 31-year-old Caucasian male during |
| | | unilateral orchiectomy. Pathology indicated embryonal carcinoma. |
| TLYMNOT06 | pINCY | Library was constructed using RNA isolated from activated |
| | | Th2 cells. These cells were differentiated from umbilical cord |
| | | CD4 T cells with IL-4 in the presence of anti-IL-12 antibodies |
| | | and B7-transfected COS cells, and then activated for six |
| | | hours with anti-CD3 and anti-CD28 antibodies. |

TABLE 7

| Program | Description | Reference | Parameter Threshold |
|---|---|---|---|
| ABI FACTURA | A program that removes vector sequences and | Applied Biosystems, Foster City, CA. | |
| | masks ambiguous bases in nucleic acid sequences. | | |
| ABI/PARACEL FDF | A Fast Data Finder useful in comparing and | Applied Biosystems, Foster City, CA; | Mismatch < 50% |
| | annotating amino acid or nucleic acid sequences. | Paracel Inc., Pasadena, CA. | |
| ABI AutoAssembler | A program that assembles nucleic acid sequences. | Applied Biosystems, Foster City, CA. | |
| BLAST | A Basic Local Alignment Search Tool useful in | Altschul, S. F. et al. (1990) J. Mol. Biol. | ESTs: Probability value = 1.0E−8 |
| | sequence similarity search for amino acid and | 215: 403-410; Altschul, S. F. et al. (1997) | or less |
| | nucleic acid sequences. BLAST includes five | Nucleic Acids Res. 25: 3389-3402. | Full Length sequences: Probability |
| | functions: blastp, blastn, blastx, tblastn, and tblastx. | | value = 1.0E−10 or less |
| FASTA | A Pearson and Lipman algorithm that searches for | Pearson, W. R. and D. J. Lipman (1988) Proc. | ESTs: fasta E value = 1.06E−6 |
| | similarity between a query sequence and a group of | Natl. Acad Sci. USA 85: 2444-2448; Pearson, | Assembled ESTs: fasta Identity = |
| | sequences of the same type. FASTA comprises as | W. R. (1990) Methods Enzymol. 183: 63-98; | 95% or greater and |
| | least five functions: fasta, tfasta, fastx, tfastx, and | and Smith, T. F. and M. S. Waterman (1981) | Match length = 200 bases or greater; |
| | ssearch. | Adv. Appl. Math. 2: 482-489. | fastx E value = 1.0E−8 or less |
| | | Full Length sequences: | |
| | | fastx score = 100 or greater | |
| BLIMPS | A BLocks IMProved Searcher that matches a | Henikoff, S. and J. G. Henikoff (1991) Nucleic | Probability value = 1.0E−3 or less |
| | sequence against those in BLOCKS, PRINTS, | Acids Res. 19: 6565-6572; Henikoff, J. G. and | |
| | DOMO, PRODOM, and PFAM databases to search | S. Henikoff (1996) Methods Enzymol. | |
| | for gene families, sequence homology, and structural | 266: 88-105; and Attwood, T. K. et al. (1997) J. | |
| | fingerprint regions. | Chem. Inf. Comput. Sci. 37: 417-424. | |
| HMMER | An algorithm for searching a query sequence against | Krogh, A. et al. (1994) J. Mol. Biol. | PFAM, INCY, SMART, or TIGRFAM |
| | hidden Markov model (HMM)-based databases of | 235: 1501-1531; Sonnhammer, E. L. L. et al. | hits: Probability value = 1.0E−3 or less |
| | protein family consensus sequences, such as PFAM, | (1988) Nucleic Acids Res. 26: 320-322; | Signal peptide hits: Score = 0 or |
| | INCY, SMART, and TIGRFAM. | Durbin, R. et al. (1998) Our World View, in a | greater |
| | | Nutshell, Cambridge Univ. Press, pp. 1-350. | |
| ProfileScan | An algorithm that searches for structural and sequence | Gribskov, M. et al. (1988) CABIOS 4: 61-66; | Normalized quality score ≧ GCG- |
| | motifs in protein sequences that match sequence patterns | Gribskov, M. et al. (1989) Methods Enzymol. | specified “HIGH” value for that |
| | defined in Prosite. | 183: 146-159; Bairoch, A. et al. (1997) | particular Prosite motif. |
| | | Nucleic Acids Res. 25: 217-221. | Generally, score = 1.4-2.1. |
| Phred | A base-calling algorithm that examines automated | Ewing, B. et al. (1998) Genome Res. | |
| | sequencer traces with high sensitivity and probability. | 8: 175-185; Ewing, B. and P. Green | |
| | | (1998) Genome Res. 8: 186-194. | |
| Phrap | A Phils Revised Assembly Program including SWAT and | Smith, T. F. and M. S. Waterman (1981) Adv. | Score = 120 or greater; |
| | CrossMatch, programs based on efficient implementation | Appl. Math. 2:482-489; Smith, T. F. and M. S. | Match length = 56 or greater |
| | of the Smith-Waterman algorithm, useful in searching | Waterman (1981) J. Mol. Biol. 147: 195-197; | |
| | sequence homology and assembling DNA sequences. | and Green, P., University of Washington, | |
| | | Seattle, WA. | |
| Consed | A graphical tool for viewing and editing Phrap assemblies. | Gordon, D. et al. (1998) Genome Res. 8: 195-202. | |
| SPScan | A weight matrix analysis program that scans protein | Nielson, H. et al. (1997) Protein Engineering | Score = 3.5 or greater |
| | sequences for the presence of secretory signal peptides. | 10: 1-6; Claverie, J. M. and S. Audic (1997) | |
| | | CABIOS 12: 431-439. | |
| TMAP | A program that uses weight matrices to delineate | Persson, B. and P. Argos (1994) J. Mol. Biol. | |
| | transmembrane segments on protein sequences and | 237:182-192; Persson, B. and P. Argos (1996) | |
| | determine orientation. | Protein Sci. 5: 363-371. | |
| TMHMMER | A program that uses a hidden Markov model (HMM) to | Sonnhammer, E. L. et al. (1998) Proc. Sixth Intl. | |
| | delineate transmembrane segments on protein sequences | Conf. on Intelligent Systems for Mol. Biol., | |
| | and determine orientation. | Glasgow et al., eds., The Am. Assoc. for Artificial | |
| | | Intelligence Press, Menlo Park, CA, pp. 175-182. | |
| Motifs | A program that searches amino acid sequences for patterns | Bairoch, A. et al. (1997) Nucleic Acids Res. 25: 217-221; | |
| | that matched those defined in Prosite. | Wisconsin Package Program Manual, version 9, page | |
| | | M51-59, Genetics Computer Group, Madison, WI. | |

1. An isolated polypeptide selected from the group consisting of:
a) a polypeptide comprising an amino acid sequence selected from the group consisting of SEQ ID NO: 1-33,
b) a polypeptide comprising a naturally occurring amino acid sequence at least 90% identical to an amino acid sequence selected from the group consisting of SEQ ID NO:1-2, SEQ ID NO:4-13, SEQ ID NO:15-19, SEQ ID NO:21, SEQ ID NO:26, SEQ ID NO:28-29, and SEQ ID NO:31,
c) a polypeptide comprising a naturally occurring amino acid sequence at least 93% identical to an amino acid sequence selected from the group consisting of SEQ ID NO:23 and SEQ ID NO:25,
d) a polypeptide comprising a naturally occurring amino acid sequence at least 95% identical to an amino acid sequence selected from the group consisting of SEQ ID NO:3, SEQ ID NO:22, and SEQ ID NO:27,
e) a polypeptide comprising a naturally occurring amino acid sequence at least 97% identical to the amino acid sequence of SEQ ID NO:30,
f) a polypeptide comprising a naturally occurring amino acid sequence at least 99% identical to the amino acid sequence of SEQ ID NO:33,
g) a biologically active fragment of a polypeptide having an amino acid sequence selected from the group consisting of SEQ ID NO:1-33, and
h) an immunogenic fragment of a polypeptide having an amino acid sequence selected from the group consisting of SEQ ID NO:1-33.

2. An isolated polypeptide of claim 1 comprising an amino acid sequence selected from the group consisting of SEQ ID NO:1-33.

3. An isolated polynucleotide encoding a polypeptide of claim 1.

4. An isolated polynucleotide encoding a polypeptide of claim 2.

5. An isolated polynucleotide of claim 4 comprising a polynucleotide sequence selected from the group consisting of SEQ ID NO:34-66.

6. A recombinant polynucleotide comprising a promoter sequence operably linked to a polynucleotide of claim 3.

7. A cell transformed with a recombinant polynucleotide of claim 6.

8. (canceled)

9. A method of producing a polypeptide of claim 1, the method comprising:
a) culturing a cell under conditions suitable for expression of the polypeptide, wherein said cell is transformed with a recombinant polynucleotide, and said recombinant polynucleotide comprises a promoter sequence operably linked to a polynucleotide encoding the polypeptide of claim 1, and
b) recovering the polypeptide so expressed.

10. A method of claim 9, wherein the polypeptide comprises an amino acid sequence selected from the group consisting of SEQ ID NO:1-33.

11. An isolated antibody which specifically binds to a polypeptide of claim 1.

12. An isolated polynucleotide selected from the group consisting of:
a) a polynucleotide comprising a polynucleotide sequence selected from the group consisting of SEQ ID NO:34-66,
b) a polynucleotide comprising a naturally occurring polynucleotide sequence at least 90% identical to a polynucleotide sequence selected from the group consisting of SEQ ID NO:34-56 and SEQ ID NO:58-66,
c) a polynucleotide complementary to a polynucleotide of a),
d) a polynucleotide complementary to a polynucleotide of b), and
e) an RNA equivalent of a)-d).

13. (canceled)

14. A method of detecting a target polynucleotide in a sample, said target polynucleotide having a sequence of a polynucleotide of claim 12, the method comprising:
a) hybridizing the sample with a probe comprising at least 20 contiguous nucleotides comprising a sequence complementary to said target polynucleotide in the sample, and which probe specifically hybridizes to said target polynucleotide, under conditions whereby a hybridization complex is formed between said probe and said target polynucleotide or fragments thereof, and
b) detecting the presence or absence of said hybridization complex, and, optionally, if present, the amount thereof.

15. (canceled)

16. A method of detecting a target polynucleotide in a sample, said target polynucleotide having a sequence of a polynucleotide of claim 12, the method comprising:
a) amplifying said target polynucleotide or fragment thereof using polymerase chain reaction amplification, and
b) detecting the presence or absence of said amplified target polynucleotide or fragment thereof, and, optionally, if present, the amount thereof.

17. A composition comprising a polypeptide of claim 1 and a pharmaceutically acceptable excipient.

18. A composition of claim 17, wherein the polypeptide comprises an amino acid sequence selected from the group consisting of SEQ ID NO:1-33.

19. (canceled)

20. A method of screening a compound for effectiveness as an agonist of a polypeptide of claim 1, the method comprising:
a) exposing a sample comprising a polypeptide of claim 1 to a compound, and
b) detecting agonist activity in the sample.

21.-22. (canceled)

23. A method of screening a compound for effectiveness as an antagonist of a polypeptide of claim 1, the method comprising:
a) exposing a sample comprising a polypeptide of claim 1 to a compound, and
b) detecting antagonist activity in the sample.

24.-25. (canceled)

26. A method of screening for a compound that specifically binds to the polypeptide of claim 1, the method comprising:
a) combining the polypeptide of claim 1 with at least one test compound under suitable conditions, and
b) detecting binding of the polypeptide of claim 1 to the test compound, thereby identifying a compound that specifically binds to the polypeptide of claim 1.

27. (canceled)

28. A method of screening a compound for effectiveness in altering expression of a target polynucleotide, wherein said target polynucleotide comprises a sequence of claim 5, the method comprising:
a) exposing a sample comprising the target polynucleotide to a compound, under conditions suitable for the expression of the target polynucleotide,
b) detecting altered expression of the target polynucleotide, and
c) comparing the expression of the target polynucleotide in the presence of varying amounts of the compound and in the absence of the compound.

29. A method of assessing toxicity of a test compound, the method comprising:
a) treating a biological sample containing nucleic acids with the test compound,
b) hybridizing the nucleic acids of the treated biological sample with a probe comprising at least 20 contiguous nucleotides of a polynucleotide of claim 12 under conditions whereby a specific hybridization complex is formed between said probe and a target polynucleotide in the biological sample, said target polynucleotide comprising a polynucleotide sequence of a polynucleotide of claim 12 or fragment thereof,
c) quantifying the amount of hybridization complex, and
d) comparing the amount of hybridization complex in the treated biological sample with the amount of hybridization complex in an untreated biological sample, wherein a difference in the amount of hybridization complex in the treated biological sample is indicative of toxicity of the test compound.

30.-121. (canceled)


SEQUENCE LISTING (66 sequences)

SEQ ID NO: 1 - protein, 106 aa, Homo sapiens [sequence omitted]

SEQ ID NO: 2 - protein, 241 aa, Homo sapiens [sequence omitted]

SEQ ID NO: 3 - protein, 1023 aa, Homo sapiens
  misc_feature: Incyte ID No 5855204CD1
MGVPKFYRWISERYPCLSEVVKEHQIPEFDNLYLDMNGIIHQCSHPNDDDVHFRISDDKIFTDIFHYLEVLFRIIKPRKVFFMAVDGVAPRAKMNQQRGRRFRSAKEAEDKIKKAIEKGETLPTEARFDSNCITPGTEFMARLHEHLKYFVNMKISTDKSWQGVTIYFSGHETPGEGEHKIMEFIRSEKAKPDHDPNTRHCLYGLDADLIMLGLTSHEAHFSLLREEVRFGGKKTQRVCAPEETTFHLLHLSLMREYIDYEFSVLKEKITFKYDIERIIDDWILMGFLVGNDFIPHLPHLHINHDALPLLYGTYVTILPELGGYINESGHLNLPRFEKYLVKLSDFDREHFSEVFVDLKWFESKVGNKYLNEAAGVAAEEARNYKEKKKLKGQENSLCWTALDKNEGEMITSKDNLEDETEDDDLFETEFRQYKRTYYMTKMGVDVVSDDFLADQAACYVQAIQWILHYYYHGVQSWSWYYPYHYAPFLSDIHNISTLKIHFELGKPFKPFEQLLAVLPAASKNLLPACYQHLMTNEDSPIIEYYPPDFKTDLNGKQQEWEAVVLIPFIDEKRLLEAMETCNHSLKKEERKRNQHSECLMCWYDRDTEFIYPSPWPEKFPAIERCCTRYKIISLDAWRVDINKNKITRIDQKALYFCGFPTLKHIRHKFFLKKSGVQVFQQSSRGENMMLEILVDAESDELTVENVASSVLGKSVFVNWPHLEEARVVAVSDGETKFYLEEPPGTQKLYSGRTAPPSKVVHLGDKEQSNWAKEVQGISEHYLRRKGIIINETSAVVYAQLLTGRKYQINQNGEVRLEKQWSKQVVPFVYQTIVKDIRAFDSRFSNIKTLDDLFPLRSMVFMLGTPYYGCTGEVQDSGDVITEGRIRVIFSIPCEPNLDALIQNQHKYSIKYNPGYVLASRLGVSGYLVSRFTGSIFIGRGSRRNPHGDHKANVGLNLKFNKKNEEVPGYTKKVGSEWMYSSAAEQLLAEYLERAPELFSYIAKNSQEDVFYEDDIWPGENENG

SEQ ID NO: 4 - protein, 441 aa, Homo sapiens
  misc_feature: Incyte ID No 5778654CD1
MKKSRSLENENLQRLSLLSRTQVPLITLPRTDGPPDLDSHSYMINSNTYESSGSPMLNLCEKSAVLSFSIEPEDQNETFFSEESREVNPGDVSLNNISTQSKWLKYQNTSQCNVATPNRVDKRITDGFFAEAVSGMHFRDTSERQSDAVNESSLDSVHLQMIKGMLYQQRQDFSSQDSVSRKKVLSLNLKQTSKTEEIKNVLGGSTCYNYSVKDLQEISGSELCFPSGQKIKSAYLPQRQIHIPAVFQSPAHYKQTFTSCLIEHLNILLFGLAQNLQKALSKVDISFYTSLKGEKLKNAENNVPSCHHSQPAKLVMVKKEGPNKGRLFYTCDGPKADRCKFFKWLEDVTPGYSTQEGARPGMVLSDIKSIGLYLRSQKIPLYEECQLLVRKGFDFQRKQYGKLKKFTTVNPEFYNEPKTKLYLKLSRKERSSAYSKIPTYE

SEQ ID NO: 5 - protein, 446 aa, Homo sapiens
  misc_feature: Incyte ID No 1440126CD1
MEVPPATKFGETFAFENRLESQQGLFPGEDLGDPFLQERGLEQMAVIYKEIPLGEQDEENDDYEGNFSLCSSPVQHQSIPPGTRPQDDELFGQTFLQKSDLSMCQIIHSEEPSPCDCAETDRGDSGPNAPHRTPQPAKPYACRECGKAFSQSSHLLRHLVIHTGEKPYECCECGKAFSQSSHLLRHQIIHTGEKPYECRECGKAFRQSSALTQHQKIHTGKRPYECRECGKDFSRSSSLRKHERIHTGERPYQCKECGKSFNQSSGLSQHRKIHTLKKPHECDLCGKAFCHRSHLIRHQRIHTGKKPYKCDECGKAFSQSSNLIEHRKTHTGEKPYKCQKCGKAFSQSSSLIEHQRIHTGEKPYECCQCGKAFCHSSALIQHQRIHTGKKPYTCECGKAFRHRSALIEHYKTHTREKPYVCNLCGKSFRGSSHLIRHQKIHSGEKL

SEQ ID NO: 6 - protein, 686 aa, Homo sapiens
  misc_feature: Incyte ID No 3934519CD1
MTESQGTVTFKDVAIDFTQEEWKRLDPAQRKLYRNVMLENYNNLITVGYPFTKPDVIFKLEQEEEPWVMEEEVLRRHWQGEIWGVDEHQKNQDRLLRQVEVKFQKTLTEEKGNECQKKFANVFPLNSDFFPSRHNLYEYDLFGKCLEHNFDCHNNVKCLMRKEHCEYNEPVKSYGNSSSHFVITPFKCNHCGKGFNQTLDLIRHLRIHTGEKPYECSNCRKAFSHKEKLIKHYKIHSREQSYKCNECGKAFIKMSNLIRHQRIHTGEKPYACKECEKSFSQKSNLIDHEKIHTGEKPYECNECGKAFSQKQSLIAHQKVHTGEKPYACNECGKAFPRIASLALHMRSHTGEKPYKCDKCGKAFSQFSMLIIHVRIHTGEKPYECNECGKAFSQSSALTVHMRSHTGEKPYECKECRKAFSHKKNFITHQKIHTREKPYECNECGKAFIQMSNLVRHQRIHTGEKPYICKECGKAFSQKSNLIAHEKIHSGEKPYECNECGKAFSQKQNFITHQKVHTGEKPYDCNECGKAFSQIASLTLHLRSHTGEKPYECDKCGKAFSQCSLLNLHMRSHTGEKPYVCNECGKAFSQRTSLIVHMRGHTGEKPYECNKCGKAFSQSSSLTIHIRGHTGEKPFDCSKCGKAFSQISSLTLHMRKHTGEKPYHCIECGKAFSQKSHLVRHQRIHTH

SEQ ID NO: 7 - protein, 903 aa, Homo sapiens
  misc_feature: Incyte ID No 2946314CD1
MSFSEMNRRTLAFRGGGLVTASGGGSTNNNAGGEASAWPPQPQPRQPPPPAPPALQPPNGRGADEEVELEGLEPQDLEASAGPAAGAAEEAKELLLPQDAGGPTSLGGGAGGPLLAERNRRTLAFRGGGGGGLGNNGSSRGRPETSVWPLRHFNGRGPATVDLELDALEGKELIQDGASLSDSTEDEEEGASLGDGSGAEGGSCSSSRRSGGDGGDEVEGSGVGAGEGETVQHFPLARPKSLMQKLQCSFQTSWLKDFPWLRYSKDTGLMSCGWCQKTPADGGSVDLPPVGHDELSRGTRNYKKTLLLRHHVSTEHKLHEANAQFPKNKNKGITHEWTCFDSSIRDLTVFAELDLLESEIPSEEGYCDFNSRPNENSYCYQLLRQLNEQRKKGILCDVSIVVSGKIFKAHKNILVAGSRFFKTLYCFSNKESPNQNNTTHLDIAAVQGFSVILDFLYSGNLVLTSQNAIEVMTVASYLQMSEVVQTCRNFIKDALNISIKSEAPESVVVDYNNRKPVNRDGLSSSRDQKIASFWATRNLTNLASNVKIENDGCNVDEGQIENYQMNDSSWVQDGSPEMAENESEGQTKVFIWNNMGSQGIQETGKTRRKNQTTKRFIYNIPPNNETNLEDCSVMQPPVAYPEENTLLIKEEPDLDGALLSGPDGDRNVNANLLAEAGTSQDGGDAGTSHDFKYGLMPGPSNDFKYGLIPGTSNDFKYGLIPGASNDFKYGLLPESWPKQETWENGESSLIMNKLKCPHCSYVAKYRRTLKRHLLIHTGVRSFSCDICGKLFTRREHVKRHSLVHKKDKKYKCMVCKKIFMLAASVGIRHGSRRYGVCVDCADKSQPGGQEGVDQGQDTEFPRDEEYEENEVGEADEELVDDGEDQNDPSRWDESGEVCMSLDD

SEQ ID NO: 8 - protein, 847 aa, Homo sapiens
  misc_feature: Incyte ID No 3617784CD1
MISPSLELLHSGLCKFPEVEGKMTTFKEAVTFKDVAVVFTEEELGLLDPAQRKLYRDVMLENFRNLLSVAHQPFKPDLISQLEREEKLLMVETETPRDGCSGDKNGKDTEYIQDEELRFFSHKELSSCKIWEEVAGELPGSQDCRVNLQGKDFQFSEDAAPHQGWEGASTPCFPIENFLDSLQGDGLIGLENQQFPAWRAIRPIPIQGSWAKAFVNQLGDVQERCKNLDTEDTVYKCNWDDDSFCWISCHVDHRFPEIDKPCGCNKCRKDCIKNSVLHRINPGENGLKSNEYRNGFRDDADLPPHPRVPLKEKLCQYDEFSEGLRHSAHLNRHQRVPTGEKSVKSLERGRGVRQNTHICNHPRAPVGDMPYRCDVCGKGFRYKSVLLIHQGVHTGRRPYKCEECGKAFGRSSNLLVHQRVHTGEKPYKCSECGKGFSYSSVLQVHQRLHTGEKPYTCSECGKGFCAKSALHKHQHIHPGEKPYSCGECGKGFSCSSHLSSHQKTHTGERPYQCDKCGKGFSHNSYLQAHQRVHMGQHLYKCNVCGKSFSYSSGLLMHQRLHTGEKPYKCECGKSFGRSSDLHIHQRVHTGEKPYKCSECGKGFRRNSDLHSHQRVHTGERPYVCDVCGKGFIYSSDLLIHQRVHTGEKPYKCAECGKGFSYSSGLLIHQRVHTGEKPYRCQECRKGFRCTSSLHKHQRVHTGKKPYTCDQCGKGFSYGSNLRTHQRLHTGEKPYTCCECGKGFRYGSGLLSHKRVHTGEKPYRCHVCGKGYSQSSHLQGHQRVHTGEKPYKCEECGKGFGRNSCLHVHQRVHTGEKPYTCGVCGKGFSYTSGLRNHQRVHLGENPYK

SEQ ID NO: 9 - protein, 1003 aa, Homo sapiens
  misc_feature: Incyte ID No 7490869CD1
MSRRKQSNPRQIKRSLGDMEAREEVQLVGASHMEQKATAPEAPSPPSADVNSPPPLPPPTSPGGPKELEGQEPEPRPTEEEPGSPWSGPDELEPVVQDGQRRIRARLSLATGLSWGPFHGSVQTRASSPRQAEPSPALTLLLVDEACWLRTLPQALTEAEANTEIHRKDDALWCRVTKPVPAGGLLSVLLTAEPHSTPGHPVKKEPAEPTCPAPAHDLQLLPQQAGMASILATAVINKDVFPCKDCGIWYRSERNLQAHLLYYCASRQGTGSPAAAATDEKPKETYPNERVCPFPQCRKSCPSASSLEIHMRSHSGERPFVCLICLSAFTTKANCERHLKVHTDTLSGVCHSCGFISTTRDILYSHLVTNHMVCQPGSKGEIYSPGAGHPATKLPPDSLGSFQQQHTALQGPLASADLGLAPTPSPGLDRKALAEATNGEARAAPQNGGSSEPPAAPRSIKVEAVEEPEAAPSWARRAWAPGPVADAVAAQPRPGQVKAELSSPTPGSSPVPGELGLAGALFLPQYVFGPDAAPPASEILAKMSELVHSRLQQGAGAGAGGAQTGLFPGAPKGATCFECEITFSNVNNYYVHKRLYCSGRRAPEDAPAARRPKAPPGPARAPPGQPAEPDAPRSSPGPGAREEGAGGAATPEDGAGGRGSEGSQSPGSSVDDAEDDPSRTLCEACNIRFSRHETYTVHKRYYCASRHDPPPRRPAAPPGPPGPAAPPAPSPAAPVRTRRRRKLYELHAAGAPPPPPPGHAPAPESPRPGSGSGSGPGLAPARSPGPAADGPIDLSKKPRRPLPGAPAPALADYHECTACRVSFHSLEAYLAHKKYSCPAAPPPGALGLPAAACPYCPPNGPVRGDLLEHFRLAHGLLLGAPLAGPGVEARTPADRGPSPAPAPAASPQPGSRGPRDGLGPEPQEPPPGPPPSPAAAPEAVPPPPAPPSYSDKGVQTPSKGTPAPLPNGNHRYCRLCNIKFSSLSTFIAHKKYYCSSHAAEHVK

SEQ ID NO: 10 - protein, 192 aa, Homo sapiens
  misc_feature: Incyte ID No 5994687CD1
MAEQQGRELEAECPVCWNPFNNTFHTPKMLDCCHSFCVECLAHLSLVTPARRRLLCPLCRQPTVLASGQPVTDLPTDTAMLALLRLEPHHVILEGHQLCLKDQPKSRYFLRQPQVYTLDLGPQPGGQTGPPPDTASATVSTPILIPSHHSLRECFRNPQFRIFAYLMAVILSVTLLLIFSIFWTKQFLWGVG

SEQ ID NO: 11 - protein, 1034 aa, Homo sapiens [sequence omitted]

SEQ ID NO: 12 - protein, 765 aa, Homo sapiens
  misc_feature: Incyte ID No 3217430CD1
MDPVATHSCHLLQQLHEQRIQGLLCDCMLVVKGVCFKAHKNVLAAFSQYFRSLFQNSSSQKNDVFHLDVKNVSGIGQILDFMYTSHLDLNQDNIQVMLDTAQCLQVQNVLSLCHTFLKSATVVQPPGMPCNSTLSLQSTLTPDATCVISENYPPHLLQECSADAQQNKTLDESHPHASPSVNRHHSAGEISKQAPDTSDGSCTELPFKQPNYYYKLRNFYSKQYHKHAAGPSQERVVEQPFAFSTSTDLTTVESQPCAVSHSECILESPEHLPSNFLAQPVNDSAPHPESDATCQQPVKQMRLKKAIHLKKLNFLKSQKYAEQVSEPKSDDGLTKRLESASKNTLEKASSQSAEEKESEEVVSCENFNCISETERPEDPAALEDQSQTLQSQRQYACELCGKPFKHPSNLELHKRSHTGEKPFECNICGKHFSQAGNLQTHLRRHSGEKPYICEICGKRFAASGDVQRHIIIHSGEKPHLCDICGRGFSNFSNLKEHKKTHTADKVFTCDECGKSFNMQRKLVKHRIRHTGERPYSCSACGKCFGGSGDLRRHVRTHTGEKPYTCEICNKCFTRSAVLRRHKKMHCKAGDESPDVLEELSQAIETSDLEKSQSSDSFSQDTSVTLMPVSVKLPVHPVENSVAEFDSHSGGSYCKLRSMIQPHGVSDQEKLSLDPGKLAKPQMQQTQPQAYAYSDVDTPAGGEPLQADGMAMIRSSLAALDNHGGDPLGSRASSTTYRNSEGQFFSSMTLWGLAMKTLQNENELDQ

SEQ ID NO: 13 - protein, 896 aa, Homo sapiens
  misc_feature: Incyte ID No 5786832CD1
MALPQGSLTFRDVAVEFSQEEWKCLDPVQKALYRDVMLENYRNLGFLGLCLPDLNIISMLEQGKEPWTVVSQVKIARNPNCGECMKGVITGISPKCVIKELPPIQNSNTGEKFQAVMLEGHESYDTENFYFREIRKNLQEVDFQWKDGEINYKEGPMTHKNNLTGQRVRHSQGDVENKHMENQLILRFQSGLGELQKFQTAEKIYGCNQIERTVNNCFLASPLQRIFPGVQTNISRKYGNDFLQLSLPTQDEKTHIREKPYIGNECGKAFRVSSSLINHQMIHTTEKPYRCNESGKAFHRGSLLTVHQIVHTRGKPYQCDVCGRIFRQNSDLVNHRRSHTGDKPYICNECGKSFSKSSHLAVHQRIHTGEKPYKCNRCGKCFSQSSSLATHQTVHTGDKPYKCNECGKTFKRNSSLTAHHIIHAGKKPYTCDVCGKVFYQNSQLVRHQIIHTGETPYKCNECGKVFFQRSRLAGHRRIHTGEKPYKCNECGKVFSQHSHLAVHQRVHTGEKPYKCNECGKAFNWGSLLTVHQRIHTGEKPYKCNVCGKVFNYGGYLSVHMRCHTGEKPLHCNKCGMVFTYYSCLARHQRMHTGEKPYKCNVCGKVFIDSGNLSIHRRSHTGEKPFQCNECGKVFSYYSCLARHRKIHTGEKPYKCNDCGKAYTQRSSLTKHLVIHTGENPYHCNEFGEAFIQSSKLARYHRNPTGEKPHKCSECGRTFSHKTSLVYHQRRHTGEMPYKCIECGKVFNSTTTLARHRRIHTGEKPYKCNECGKVFRYRSGLARHWSIHTGEKPYKCNECGKAFRVRSILLNHQMMHTGEKPYKCNECGKAFIERSNLVYHQRNHTGEKPYKCMECGKAFGRRSCLTKHQRIHSSEKPYKCNECGNLTLVAQASLNIR

SEQ ID NO: 14 - protein, 357 aa, Homo sapiens
  misc_feature: Incyte ID No 7493320CD1
MATVIHNPLKALGDQFYKEAIEHCRSYNSRLCAERSVRLPFLDSQTGVAQNNCYIWMEKRHRGPGLAPGQLYTYPARCWRKKRRLHPPEDPKLRLLEIKPEVELPLKKDGFTSESTTLEALLRGEGVEKKVDAREEESIQEIQRVLENDENVEEGNEEEDLEEDIPKRKNRTRGRARGSAGGRRRHDAASQEDHDKPYVCDICGKRYKNRPGLSYHYAHTHLASEEGDEAQDQETRSPPNHRNENHRPQKGPDGTVIPNNYCDFCLGGSNMNKKSGRPEELVSCADCGRSAHLGGEGRKEKEAAAAARTTEDLFGSTSESDTSTFHGFDEDDLEEPRSCRGRRSGRGSPTADKKGSC

SEQ ID NO: 15 - protein, 513 aa, Homo sapiens
  misc_feature: Incyte ID No 2911453CD1
MKQEWSQGYRALPSLSNHGSQNGLDLGDLLSLPPGTSMSSNSVSNSLPSYLFGTESSHSPYPSPRHSSTRSHSARSKKRALSLSPLSDGIGIDFNTIIRTSPTSLVAYINGSRASPANLSPQPEVYGHFLGVRGSCIPQPRPVPGSQKGVLVAPGGLALPAYGEDGALEHERMQQLEHGGLQPGLVNHMVVQHGLPGPDSQPAGLFKTERLEEFPGSTVDLPPAPPLPPLPPPPGPPPPYHAHAHLHHPELGPHAQQLALPQATLDDDGEMDGIGGKHCCRWIDCSALYDQQEELVRHIEKVHIDQRKGEDFTCFWAGCPRRYKPFNARYKLLIHMRVHSGEKPNKCTFEGCEKAFSRLENLKIHLRSHTGEKPYLCQHPGCQKAFSNSSDRAKHQRTHLDTKPYACQIPGCTKRYTDPSSLRKHVKAHSSKEQQARKKLRSSTELHPDLLTDCLTVQSLQPATSPRDAAAEGTVGRSPGPGPDLYSGKDSKGRANHNYPFLGLLLWLKGMLT

SEQ ID NO: 16 - protein, 104 aa, Homo sapiens
  misc_feature: Incyte ID No 3029661CD1
MASISELACIYSALILHDNEVTVTEYKIKALIKAAGVNVEPFRPGLFAKAPANVNIRSLICNVGAGGPAPAAGAAPAGAEEKKMEAKKEEFEDSDDDMGFGLSD

SEQ ID NO: 17 - protein, 255 aa, Homo sapiens
  misc_feature: Incyte ID No 71260474CD1
MTSFNIAQGIHAFDYHSRLNLIATAGINNKVCLWNPYVVSKPVGVLWGHSASVIAVQFFVERKQLFSFSKDKVLRLWDIQHQLSIQRIACSFPKSQDFRCLFHFDEAHGRLFISFNNQLALLAMKSEASKRVKSHEKAVTCVLYNSILKQVISSDTGSTVSFWMIDTGQKIKQFTGCHGNAEISTMALDANETRLLTGSTDGTVKIWDFNGYCHHTLNVGQDGAVDISQILILKKKILVTGWERYDYASWKTIGR

SEQ ID NO: 18 - protein, 1133 aa, Homo sapiens
  misc_feature: Incyte ID No 7992707CD1
MDVLAEEFGNLTPEQLAAPIPTVEEKWRLLPAFLKVKGLVKQHIDSFNYFINVEIKKIMKANEKVTSDADPMWYLKYLNIYVGLPDVEESFNVTRPVSPHECRLRDMTYSAPITVDIEYTRGSQRIIRNALPIGRMPIMLRSSNCVLTGKTPAEFAKLNECPLDPGGYFIVKGVEKVILIQEQLSKNRIIVEADRKGAVGASVTSSTHEKKSRTNMAVKQGRFYLRHNTLSEDIPIVIIFKAMGVESDQEIVQMIGTEEHVMAAFGPSLEECQKAQIFTQMQALKYIGNKVRRQRMWGGGPKKTKIEEARELLASTILTHVPVKEFNFRAKCIYTAVMVRRVILAQGDNKVDDRDYYGNKRLELAGQLLSLLFEDLFKKFNSEMKKIADQVIPKQRAAQFDVVKHMRQDQITNGMVNAISTGNWSLKRFKMDRQGVTQVLSRLSYISALGMMTRISSQFEKTRKVSGPRSLQPSQWGMLCPSDTPEGEACGLVKNLALMTHITTDMEDGPIVKLASNLGVEDVNLLCGEELSYPNVFLVFLNGNILGVIRDHKKLVNTFRLMRRAGYINEFVSISTNLTDRCVYISSDGGRLCRPYIIVKKQKPAVTNKHMEELAQGYRNFEDFLHESLVEYLDVNEENDCNIALYEHTINKDTTHLEIEPFTLLGVCAGLIPYPHHNQSPRNTYQCAMGKQAMGTIGYNQRNRIDTLMYLLAYPQKPMVKTKTIELIEFEKLPAGQNATVAVMSYSGYDIEDALVLNKASLDRGFGRCLVYKNAKCTLKRYTNQTFDKVMGPMLDAATRKPIWRHEILDADGICSPGEKVENKQVLVNKSMPTVTQIPLEGSNVPQQPQYKDVPITYKGATDSYIEKVMISSNAEDAFLIKMLLRQTRRPEIGDKFSSRHGQKGVCGLIVPQEDMPFCDSGICPDIIMNPHGFPSRMTVGKLIELLAGKAGVLDGRFHYGTAFGGSKVKDVCEDLVRHGYNYLGKDYVTSGITGEPLEAYIYFGPVYYQKLKHMVLDKMHARARGPRAVLTRQPTEGRSRDGGLRLGEMERDCLIGYGASMLLLERLMISSDAFEVDVCGQCGLLGYSGWCHYCKSSCHVSSLRIPYACKLLFQELQSMNIIPRLKLSKYNE

SEQ ID NO: 19 - protein, 3065 aa, Homo sapiens
  misc_feature: Incyte ID No 7974861CD1
MEEKQQIILANQDGGTVAGAAPTFFVILKQPGNGKTDQGILVTNQDACALASSVSSPVKSKGKICLPADCTVGGITVTLDNNSMWNEFYHRSTEMILTKQGRRMFPYCRYWITGLDSNLKYILVMDISPVDNHRYKWNGRWWEPSGKAEPHVLGRVFIHPESPSTGHYWMHQPVSFYKLKLTNNTLDQEGHIILHSMHRYLPRLHLVPAEKAVEVIQLNGPGVHTFTFPQTEFFAVTAYQNIQITQLKIDYNPFAKGFRDDGLNNKPQRDGKQKNSSDQEGNNISSSSGHRVRLTEGQGSEIQPGDLDPLSRGHETSGKGLEKTSLNIKRDFLGFMDTDSALSEVPQLKQEISESLIASSFEDDSRVASPLDQNGSFNVVIKEEPLDDYDYELGECPEGVTVKQEETDEETDVYSNSDDDPILEKQLKRHNKVDNPEADHLSSKWLPSSPSGVAKAKMFKLDTGKMPVVYLEPCAVTRSTVKISELPDNMLSTSRKDKSSMLAELEYLPTYIENSNETAFCLGKESENGLRKHSPDLRVVQKYPLLKEPQWKYPDISDSISTERILDDSKDSVGDSLSGKEDLGRKRTTMLKIATAAKVVNANQNASPNVPGKRGRPRKLKLCKAGRPPKNTGKSLISTKNTPVSPGSTFPDVKPDLEDVDGVLFVSFESKEALDIHAVDGTTEESSSLQASTTNDSGYRARISQLEKELIEDLKTLRHKQVIHPGLQEVGLKLNSVDPTMSIDLKYLGVQLPLAPATSFPFWNLTGTNPASPDAGFPFVSRTGKTNDFTKIKGWRGKFHSASASRNEGGNSESSLKNRSAFCSDKLDEYLENEGKLMETSMGFSSNAPTSPVVYQLPTKSTSYVRTLDSVLKKQSTISPSTSYSLKPHSVPPVSRKAKSQNRQATFSGRTKSSYKSILPYPVSPKQKYSHVILGDKVTKNSSGIISENQANNFVVPTLDENIFPKQISLRQAQQQQQQQQGSRPPGLSKSQVKLMDLEDCALWEGKPRTYITEERADVSLTTLLTAQASLKTKPIHTIIRKRAPPCNNDFCRLGCVCSSLALEKRQPAHCRRPDCMFGCTCLKRKVVLVKGGSKTKHFQRKAAHRDPVFYDTLGEEAREEEEGIREEEEQLKEKKKRKKLEYTICETEPEQPVRHYPLWVKVEGEVDPEPVYIPTPSVIEPMKPLLLPQPEVLSPTVKGKLLTGIKSPRSYTPKPNPVIREEDKDPVYLYFESMMTCARVRVYERKKEDQRQPSSSSSPSPSFQQQTSCHSSPENHNNAKEPDSEQQPLKQLTCDLEDDSDKLQEKSWKSSCNEGESSSTSYMHQRSPGGPTKLIEIISDCNWEEDRNKILSILSQHTNSNMPQSLKVGSFIIELASQRKSRGEKNPPVYSSRVKISMPSCQDQDDMAEKSGSETPDGPLSPGKMEDISPVQTDALDSVRERLHGGKGLPFYAGLSPAGKLVAYKRKPSSSTSGLIQVASNAKVAASRKPRTLLPSTSNSKMASSSGTATNRPGKNLKAFVAAKRPIAARPSPGGVFTQFVMSKVGALQQKIPGVSTPQTLAGTQKFSIRPSPVMVVTPVVSSEPVQVCSPVTAAVTTTTPQVFLENTTAVTPMTAISDVETKETTYSSGATTTGVVEVSETNTSTSVTSTQSTATVNLTKTTGITTPVASVAFPKSLVASPSTITLPVASTASTSLVVVTAAASSSMVTTPTSSLGSVPIILSGINGSPPVSQRPENAAQIPVATPQVSPNTVKRAGPRLLLIPVQQGSPTLRPVSNTQLQGHRMVLQPVRSPSGMNLFRHPNGQIVQLLPLHQLRGSNTQPNLQPVMFRNPGSVMGIRLPAPSKPSETPPSSTSSSAFSVMNPVIQAVGSSSAVNVITQAPSLLSSGASFVSQAGTLTLRISPPEPQSFASKTGSETKITYSSGGQPVGTASLIPLQSGSFALLQLPGQKPVPSSILQHVASLQMKRESQNPDQKDETNSIKREQETKKVLQSEGEAVDPEANVIKQNSGAATSEETLNDSLEDRGDHLDEECLPEEGCATVKPSEHSCITGSHTDQDYKDVNEEYGARNRKSSKEKVAVLEVRTISEKASNKTVQNLSKVQHQKLGDVKVEQQKGFDNPEENSSEFPVTFKEESKFELSGSKVMEQQSNLQPEAKEKECGDSLEKDRERWRKHLKGPLTRKCVGASQECKKEADEQLIKETKTCQENSDVFQQEQGISDLLGKSGITEDARVLKTECDSWSRISNPSAFSIVPRRAAKSSRGNGHFQGHLLLPGEQIQPKQEKKGGRSSADFTVLDLEEDDEDDNEKTDDSIDEIVDVVSDYQSEEVDDVEKNNCVEYIEDDEEHVDIETVEELSEEINVAHLKTTAAHTQSFKQPSCTHISADEKAAERSRKAPPIPLKLKPDYWSDKLQKEAEAFAYYRRTHTANERRRRGEMRDLFEKLKITLGLLHSSKVSKSLILTRAFSEIQGLTDQADKLIGQKNLLTRKRNILIRKVSSLSGKTEEVVLKKLEYIYAKQQALEAQKRKKKMGSDEFDISPRISKQQEGSSASSVDLGQMFINNRRGKPLILSRKKDQATENTSPLNTPHTSANLVMTPQGQLLTLKGPLFSGPVVAVSPDLLESDLKPQVAGSAVALPENDDLFMMPRIVNVTSLATEGGLVDMGGSKYPHEVPDSKPSDHLKDTVRNEDNSLEDKGRISSRGNRDGRVTLGPTQVFLANKDSGYPQIVDVSNMQKAQEFLPKKISGDMRGIQYKWKESESRGERVKSKDSSFHKLKMKDLKDSSIEMELRKVTSAIEEAALDSSELLTNMEDEDDTDETLTSLLNEIAFLNQQLNDDSVGLAELPSSMDTEFPGDARRAFISKVPPGSRATFQVEHLGTGLKELPDVQGESDSISPLLLHLEDDDFSENEKQLAEPASEPDVLKIVIDSEIKDSLLSNKKAIDGGKNTSGLPAEPESVSSPPTLHMKTGLENSNSTDTLWRPMPKLAPLGLKVANPSSDADGQSLKVMPCLAPIAAKVGSVGHKMNLTGNDQEGRESKVMPTLAPVVAKLGNSGASPSSAGK

SEQ ID NO: 20 - protein, 1400 aa, Homo sapiens
  misc_feature: Incyte ID No 7499710CD1
MAARRGRRDGVAPPPSGGPGPDPVGGARGSGWGSRSQAPYGTLGAVSGGEQVLLHEEAGDSGFVSLSRLGPSLRDKDLEMEELMLQDETLLGTMQSYMDASLISLIEDFGSLGESRLSLEDQNEVSLLTALTEILDNADSENLSPFDSIPDSELLVSPREGSSLHKLLTLSRTPPERDLITPVDPLGPSTGSSRGSGVEMSLPDPSWDFSPPSFLETSSPKLPSWRPPRSRPRWGQSPPPQQRSDGEEEEEVASFSGQILAGELDNCVSSIPDFPMHLACPEEEDKATAAEMAVPAAGDESISSLSELVRAMHPYCLPNLTHLASLEDELQEQPDDLTLPEGCVVLEIVGQAATAGDDLEIPVVVRQVSPGPRPVLLDDSLETSSALQLLMPTLESETEAAVPKVTLCSEKEGLSLNSEEKLDSACLLKPREVVEPVVPKEPQNPPANAAPGSQRARKGRKKKSKEQPAACVEGYARRLRSSSRGQSTVGTEVTSQVDNLQKQPQEELQKESGPLQGKGKPRAWARAWAAALENSSPKNLERSAGQSSPAKEGPLDLYPKLADTIQTNPIPTHLSLVDSAQASPMPVDSVEADPTAVGPVLAGPVPVDPGLVDLASTSSELVEPLPAEPVLINPVLADSAAVDPAVVPISDNLPPVDAVPSGPAPVDLALVDPVPNDLTPVDPVLVKSRPTDPRRGAVSSALGGSAPQLLVESESLDPPKTIIPEVKEVVDSLKIESGTSATTHEARPRPLSLSEYRRRRQQRQAETEERSPQPPTGKWPSLPETPTGLADIPCLVIPPAPAKKTALQRSPETPLEICLVPVGPSPASPSPEPPVSKPVASSPTEQVPSQEMPLLARPSPPVQSVSPAVPTPPSMSAALPFPAGGLGMPPSLPPPPLQPPSLPLSMGPVLPDPFTHYAPLPSWPCYPHVSPSGYPCLPPPPTVPLVSGTPGAYAVPPTCSVPWAPPPAPVSPYSSTCTYGPLGWGPGPQHAPFWSTVPPPPLPPASIGRAVPQPKMESRGTPAGPPENVLPLSMAPPLSLGLPGHGAPQTEPTKVEVKPVPASPHPKHKVSALVQSPQMKALACVSAEGVTVEEPASERLKPETQETRPREKPPLPATKAVPTPRQSTVPKLPAVHPARLRKLSFLPTPRTQGSEDVVQAFISEIGIEASDLSSLLEQFEKSEAKKECPPPAPADSLAVGNSGSSCSSSGRSRRCSSSSSSSSSSSSSSSSSSSSRSRSRSPSPRRRSDRRRRYSSYRSHDHYQRQRVLQKERAIEERRVVFIGKIPGRMTRSELKQRFSVFGEIEECTIHFRVQGDNYGFVTYRYAEEAFAAIESGHKLRQADEQPFDLCFGGRRQFCKRSYSDLDSNREDFDPAPVKSKFDSLDFDTLLKQAQKNLRR

SEQ ID NO: 21 - protein, 1369 aa, Homo sapiens
  misc_feature: Incyte ID No 8036958CD1
MGGKNKKHKAPAAAVVRAAVSASRAKSAEAGIAGEAQSKKPVSRPATAAAAAAGSREPRVKQGPKIYSFNSTNDSSGPANLDKSILKVVINNKLEQRIIGVINEHKKQNNDKGMISGRLTAKKLQDLYMALQAFSFKTKDIEDAMTNTLLYGGDLHSALDWLCLNLSDDALPEGFSQEFEEQQPKSRPKFQSPQIQATISPPLQPKTKTYEEDPKSKPKKEEKNMEVNMKEWILRYAEQQNEEEKNENSKSLEEEEKFDPNERYLHLAAKLLDAKEQAATFKLEKNKQGQKEAQEKIRKFQREMETLEDHPVFNPAMKISHQQNERKKPPVATEGESALNFNLFEKSAAATEEEKDKKKEPHDVRNFDYTARSWTGKSPKQFLIDWVRKNLPKSPNPSFEKVPVGRYWKCRVRVIKSEDDVLVVCPTILTEDGMQAQHLGATLALYRLVKGQSVHQLLPPTYRDVWLEWSDAEKKREELNKMETNKPRDLFIAKLLNKLKQQQQQQQQHSENKRENSEDPEESWENLVSDEDFSALSLESANVEDLEPVRNLFRKLQSTPKYQKLLKERQQLPVFKHRDSIVETLKRHRVVVVAGETGSGKSTQVPHFLLEDLLLNEWEASKCNIVCTQPRRISAVSLANRVCDELGCENGPGGRNSLCGYQIRMESRACESTRLLYCTTGVLLRKLQEDGLLSNVSHVIVDEVHERSVQSDFLLIILKEILQKRSDLHLILMSATVDSEKFSTYFTHCPILRISGRSYPVEVFHLEDIIEETGFVLEKDSEYCQKFLEEEEEVTINVTSKAGGIKKYQEYIPVQTGAHADLNPFYQKYSSRTQHAILYMNPHKINLDLILELLAYLDKSPQFRNIEGAVLIFLPGLAHIQQLYDLLSNDRRFYSERYKVIALHSILSTQDQAAAFTLPPPGVRKIVLATNIAETGITIPDVVFVIDTGRTKENKYHESSQMSSLVETFVSKASALQRQGRAGRVRDGFCFRMYTRERFEGFMDYSVPEILRVPLEELCLHIMKCNLGSPEDFLSKALDPPQLQVISNAMNLLRKIGACELNEPKLTPLGQHLAALPVNVKIGKMLIFGAIFGCLDPVATLAAVMTEKSPFTTPIGRKDEADLAKSALAMADSDHLTIYNAYLGWKKARQEGGYRSEITYCRRNFLNRTSLLTLEDVKQELIKLVKAAGFSSSTTSTSWEGNRASQTLSFQEIALLKAVLVAGLYDNVGKIIYTKSVDVTEKLACIVETAQGKAQVHPSSVNRDLQTHGWLLYQEKIRYARVYLRETTLITPFPVLLFGGDIEVQHRERLLSIDGWIYFQAPVKIAVIFKQLRVLIDSVLRKKLENPKMSLENDKILQIITELIKTENN

SEQ ID NO: 22 - protein, 589 aa, Homo sapiens
  misc_feature: Incyte ID No 3253807CD1
MEAEETMECLQEFPEHHKMILDRLNEQREQDRFTDITLIVDGHHFKAHKAVLAACSKFFYKFFQEFTQEPLVEIEGVSKMAFRHLIEFTYTAKLMIQGEEEANDVWKAAEFLQMLEAIKALEVRNKENSAPLEENTTGKNEAKKRKIAETSNVITESLPSAESEPVEIEVEIAEGTIEVEDEGIETLEEVASAKQSVKYIQSTGSSDDSALALLADITSKYRQGDRKGQIKEDGCPSDPTSKQEHMKSHSTESFKCEICNKRYLRESAWKQHLNCYHLEEGGVSKKQRTGKKIHVCQYCEKQFDHFGHFKEHLRKHTGEKPFECPNCHERFARNSTLKCHLTACQTGVGAKKGRKKLYECQVCNSVFNSWDQFKDHLVIHTGDKPNHCTLCDLWFMQGNELRRHLSDAHNISERLVTEEVLSVETRVQTEPVTSMTIIEQVGKVHVLPLLQVQVDSAQVTVEQVHPDLLQDSQVHDSHMSELPEQVQVSYLEVGRIQTEEGTEVHVEELHVERVNQMPVEVQTELLEADLDHVTPEIMNQEERESSQADAAEAAREDHEDAEDLETKPTVDSEAEKAENEDRTALPVLE

SEQ ID NO: 23 - protein, 192 aa, Homo sapiens
  misc_feature: Incyte ID No 3626408CD1
MYTARKKIQKEKGLEPSEFEDSVAQAFFDLENGNQELKSDLKDLYINNAIQMDVTGSRKAVVIHVPYRLRKAFRKIHVRLVRELEKKFSGKDVVIVATRRIVRPPKKGSAVLRPRTRTLTAVHDGILEDVVYPAEIVGKRVRYRLDGSKIIKIFLDPKERNNTEYKLETCTAVYRRLCGKDVVFEYPMTENA

SEQ ID NO: 24 - protein, 1007 aa, Homo sapiens [sequence omitted]

SEQ ID NO: 25 - protein, 865 aa, Homo sapiens
  misc_feature: Incyte ID No 4398735CD1
MPAGLTEPAGAAPPAAVSASGTVTMAPAGALPVRVESTPVALGAVTKAPVSVCVEPTASQPLRSPVGTLVTKVAPVSAPPKVSSGPRLPAPQIVAVKAPNTTTIQFPANLQLPPGMLGTVLIKSNSGPLMLVSPQQTVTRAETTSNITSRPAVPANPQTVKICTVPNSSSQLIKKVAVTPVKKLAQIGTTVVTTVPKPSSVQSVAVPTSVVTVTPGKPLNTVTTLKPSSLGASSTPSNEPNLKAENSAAVQINLSPTMLENVKKCKNFLAMLIKLACSGSQSPEMGQNVKKLVEQLLDAKIEAEEFTRKLYVELKSSPQPHLVPFLKKSVVALRQLLPNSQSFIQQCVQQTSSDMVIATCTTTVTTSPVVTTTVSSSQSEKSIIVSGATAPRTVSVQTLNPLAGPVGAKAGVVTLHSVGPTAATGGTTAGTGLLQTSKPLVTSVANTVTTVSLQPEKPVVSGTAVTLSLPAVTFGETSGAAICLPSVKPVVSFCWDHICKPVIGTPVQIKLAQPGPVLSQPAGIPQAVQVKQLVVQQPSGGNEKQVTTISHSSTLTIQKCGQKTMPVNTIIPTSQFPPASILKQITLPGNKILSLQASPTQKNRIKENVTSCFRDEDDINDVTSMAGVNLNEENACILATNSELVGTLIQSCKDEPFLFIGALQKRILDIGKKHDITELNSDAVNLISQATQERLRGLLEKLTAIAQHRMTTYKASENYILCSDTRSQLKFLEKLDQLEKQRKDLEEREMLLKAAKSRSNKEDPEQLRLKQKAKELQQLELAQIQHRDANLTALAAIGPRKKRPLESGIEGLKDNLLASGTSSLTATKQLHRPRITRICLRDLIFCMEQEREMKYSRALYLALLK

SEQ ID NO: 26 - protein, 545 aa, Homo sapiens
  misc_feature: Incyte ID No 7499579CD1
MNPSTPSYPTASLYVGDLHPDVTEAMLYEKFSPAGPILSIRICRDLITSGSSNYAYVNFQHTKDAEHALDTMNFDVIKGKPVRIMWSQRDPSLRKSGVGNIFVKNLDKSINNKALYDTVSAFGNILSCNVVCDENGSKGYGFVHFETHEAAERAIKKMNGMLLNGRKVFVGQFKSRKEREAELGARAKEFPNVYIKNFGEDMDDERLKDLFGKFWPALSVKVMTDESGKSKGFGFVSFERHEDAQKAVDEMNGKELNGKQIYVGRAQKKVERQTELKRKFEQMKQDRITRYQGVNLYVKNLDDGIDDERLRKGFSPFGTITSAKTQNRAAYYPPSQIAQLRPSPRWTAQGARPHPFQNMPGAIRPAAPRPPFSTMRPASSQVPRVMSTQRVANTSTQTMGPRPAAAAAAATPAVRTVPQYKYAAGVRNPQQHLNAQPQVTMQQPAVHVQGQEPLTASMLASAPPQEQKQMLGERLFPLIQAMHPTLAGKITGMLLEIDNSELLHMLESPESLRSKVDEAVAVLQAHQAKEAAQKAVNSATGVPTV

SEQ ID NO: 27 - protein, 429 aa, Homo sapiens
  misc_feature: Incyte ID No 8178947CD1
MAVVLPPTAALSSLFPASQREGHTEGGELVNELLKSWLKGLVTFEDVAVEFTQEEWALLDPAQRTLYRDVMLENCRNLASLGNQVDKPRLISQLEQEDKVMTEERGILSGTCPDVENPFKAKGLTPKLHVFRKEQSRNMKMERNHLGATLNECNQCFKVFSTKSSLTRHRKIHTGERPYGCSECGKSYSSRSYLAVHKRIHNGEKPYECNDCGKTFSSRSYLTVHKRIHNGEKPYECSDCGKTFSNSSYLRPHLRIHTGEKPYKCNQCFREFRTQSIFTRHKRVHTGEGHYVCNQCGKAFGTRSSLSSHYSIHTGEYPYECHDCGRTFRRRSNLTQHIRTHTGEKPYTCNECGKSFTNSFSLTIHRRIHNGEKSYECSDCGKSFNVLSSVKKHMRTHTGKKPYECNYCGKSFTSNSYLSVHTRMHNRQM

SEQ ID NO: 28 - protein, 1286 aa, Homo sapiens
  misc_feature: Incyte ID No 2264652CD1
MITSKDNLEDETEDDDLFETEFRQYKRTYYMTKMGVDVVSDDFLADQAACYVQAIQWILHYYYHGVQSWSWYYPYHYAPFLSDIHNISTLKIHFELGKPFKPFEQLLAVLPAASKNLLPACYQHLMTNEDSPIIEYYPPDFKTDLNGKQQEWEAVVLIPFIDEKRLLEAMETCNHSLKKEERKRNQHSECLMCWYDRDTEFIYPSPWPEKFPAIERCCTRYKIISLDAWRVDINKNKITRIDQKALYFCGFPTLKHIRHKFFLKKSGVQVFQQSSRGENMMLEILVDAESDELTVENVASSVLGKSVFVNWPHLEEARVVAVSDGETKFYLEEPPGTQKLYSGRTAPPSKVVHLGDKEQSNWAKEVQGISEHYLRRKGIIINETSAVVYAQLLTGRKYQINQNGEVRLEKQWSKQVVPFVYQTIVKDIRAFDSRFSNIKTLDDLFPLRSMVFMLGTPYYGCTGEVQDSGDVITEGRIRVIFSIPCEPNLDALIQNQHKYSIKYNPGYVLASRLGVSGYLVSRFTGSIFIGRGSRRNPHGDHKANVGLNLKFNKKNEEVPGYTKKVGSEWMYSSAAEQLLAEYLERAPELFSYIAKNSQEDVFYEDDIWPGENENGAEKVQEIITWLKGHPVSTLSRSSCDLQILDAAIVEKIEEEVEKCKQRKNNKKVRVTVKPHLLYRPLEQQHGVIPDRDAEFCLFDRVVNVRENFSVPVGLRGTIIGIKGANREANVLFEVLFDEEFPGGLTIRCSPGRGYRLPTSALVNLSHGSRSETGNQKLTAIVKPQPAVHQHSSSSSVSSGHLGALNHSPQSLFVPTQVPTKDDDEFCNIWQSLQGSGKMQYFQPTIQEKGAVLPQEISQVNQHHKSGFNDNSVKYQQRKHDPHRKFKEECKSPKAECWSQKMSNKQPNSGIENFLASLNISKENEVQSSHHGEPPSEEHLSPQSFAMKGTRMLKEILKIDGSNTVDHKNEIKQIANEIPVSSNRRDEYGLPSQPKQNKKLASYMNKPHSANEYHNVQSMDNMCWPAPSQIPPVSTPVTELSRICSLVGMPQPDFSFLRMPQTMTVCQVKLSNGLLVHGPQCHSENEAKEKAALFALQQLGSLGMNFPLPSQVFANYPSAVPPGTIPPAFPPPTANIMPSSSHLFGSMPWGPSVPVPGKPFHHTLYSGTMPMAGGIPGGVHNQFIPLQVTKKRVANKKNFENKEAQSSQATPVQTSQPDSSNIVKVSPRESSSASLKSSPIAQPASSFQVETASQGHSISHHKSTPISSSRRKSRKLAVNFGVSKPSE

SEQ ID NO: 29 - protein, 740 aa, Homo sapiens
  misc_feature: Incyte ID No 1806372CD1
MVSVTKYDLTGCSAFCRSCQRATMTSQPLRLAEEYGPSPGESELAVNPFDGLPFSSRYYELLKQRQALPIWAARFTFLEQLESNPTGVVLVSGEPGSGKSTQIPQWCAEFALARGFQKGQVTVTQPYPLAARSLALRVADEMDLTLGHEVGYSIPQEDCTGPNTLLRFCWDRLLLQEVASTRGTGAWGVLVLDEAQERSVASDSLQGLLQDARLEKLPGDLRVVVVTDPALEPKLRAFWGNPPIVHIPREPGERPSPIYWDTIPPDRVEAACQAVLELCRKELPGDVLVFLPSEEEISLCCESLSREVESLLLQGLPPRVLPLHPDCGRAVQAVYEDMDARKVVVTHWLADFSFSLPSIQHVIDSGLELRSVYNPRIRAEFQVLRPISKCQAEARRLRARGFPPGSCLCLYPKSFLELEAPPLPQPRVCEENLSSLVLLLKRRQIAEPGECHFLDQPAPEALMQALEDLDYLAALDDDGDLSDLGVILSEFPLAPELAKALLASCEFDCVDEMLTLAAMLTAAPGFTRPPLSAEEAALRRALEHTDGDHSSLIQVYEAFIQSGADEAWCQARGLNWAALCQAHKLRGELLELMQRIELPLSLPAFGSEQNRRDLQKALVSGYFLKVARDTDGTGNYLLLTHKHVAQLSSYCCYRSRRAPARPPPWVLYHNFTISKDNCLSIVSEIQPQMLVELAPPYFLSNLPPSESRDLLNQLREGMADSTAGSKSSSAQEFRDPCVLQ

SEQ ID NO: 30 - protein, 376 aa, Homo sapiens
  misc_feature: Incyte ID No 2010564CD1
MHLLKVGTWRNNTASSWLMKFSVLWLVSQNCCRASVVWMAYMNISFHVGNHVLSELGETGVFGRSSTLKRVAGVIVPPEGKIQNACNPNTIFSRSKYSETWLALIERGGCTFTQKIKVATEKGASGVIIYNVPGTGNQVFPMFHQAFEDVVVVMIGNLKGTEIFHLIKKGVLITAVVEVGRKHIIWMNHYLVSFVIVTTATLAYFIFYHIHRLCLARIQNRRWQRLTTDLQNTFGQLQLRVVKEGDEEINPNGDSCVICFERYKPNDIVRILTCKHFFHKNCIDPWILPHGTCPICKCDILKVLGIQVVVENGTEPLQVLMSNELPETLSPSEEETNNEVSPAGTSDKVIHVEENPTSQNNDIQPHSVVEDVHPSP

SEQ ID NO: 31 - protein, 400 aa, Homo sapiens
  misc_feature: Incyte ID No 7364908CD1
MIRSQGPVSFEDVAVDFTQEEWQQLDYAQRTLYRDVMLEIYSHLVSMGYPVSKPDVISKLEQGEEPWIIKRHIPNWIYPDRESRLDTPQLDIFRDVFFHKETLESITGGHSLYSILKVWQDKFVRQVVVINNKRISEESGHPYNIFGKIFHDCTDLDTSKQRLCKCDSFEKTLKPNINLVSYNRNFARKNIDENFRCGKTPSYSSCYSKHEKIHSGMIHCEATHCGKILSHKQSLIHYVNVETGEKTYVCVECGKSFLKKSQIIIHQRIHTGEKPYDCGACGKAFSEKSHLIAHQRTHTGEKPYDCSECGKGFSQKSSLIIHQRVHSGEKPYECSECEKAFSQKSPLIIHQRIHTGEKPYECRVWESLFPESQLIIHHRAHTGEKPCKCTECGKAFCFIH

SEQ ID NO: 32 - protein, 472 aa, Homo sapiens
  misc_feature: Incyte ID No 7489960CD1
MGGDHPEDEEDFYEEEMDYGESEEPMGDDDYDEYSKELNQYRRSKDSRGRGLSRGRGRGSRGRGKGMGRGRGRGGSRGGMNKGGMNDDEDFYDEDMGDGGGGSYRSRDHDKPHQQSDKKGKVICKYFVEGRCTWGDHCNFSHDIELPKKRELCKFYITGFCARAENCPYMHGDFPCKLYHTTGNCINGDDCMFSHDPLTEETRELLDKMLADDAEAGAEDEKEVEELKKQGINPLPKPPPGVGLLPTPPRPPGPQAPTSPNGRPMQGGPPPPPPPPPPPPGPPQMPMPVHEPLSPQQLQQQDMYNKKIPSLFEIVVRPTGQLAEKLGVRFPGPGGPPGPMGPGPNMGPPGPMGGPMHPDMHPDMHPDMHPDMHADMHADMPMGPGMNPGPPMGPGGPPMMPYGPGDSPHSGMMPPIPPAQNFYENFYQQQEGMEMEPGLLGDAEDYGHYEELPGEPGEHLFPEHPLEPRQLL

SEQ ID NO: 33 - protein, 401 aa, Homo sapiens
  misc_feature: Incyte ID No 8555401CD1
MAHVSSETQDVSPKDELTASEASTRSPLCEHTFPGDSDLRSMIEEHAFQVLSQGSLLESPSYTVCVSEPDKDDDFLSLNFPRKLWKIVESDQFKSISWDENGTCIVINEELFKKEILETKAPYRIFQTDAIKSFVRQLNLYGFSKIQQNFQRSAFLATFLSEEKESSVLSKLKFYYNPNFKRGYPQLLVRVKRRIGVKNASPISTLFNEDFNKKHFRAGANMENHNSALAAEASEESLFSASKNLNMPLTRESSVRQIIANSSVPIRSGFPPPSPSTSVGPSEQIATDQHAILNQLTTIHMHSHSTYMQARGHIVNFITTTTSQYHIISPLQNGYFGLTVEPSAVPTRYPLVSVNEAPYRNMLPAGNPWLQMPTIADRSAAPHSRLALQPSPLDKYHPNYN

SEQ ID NO: 34 - DNA, 1357 bp, Homo sapiens
  misc_feature: Incyte ID No 4001873CB1
ggtgtgcgagtgtctaagtctgtacttttacttcttttgggtatacacctagaagtagaattgctggatcatacggtagttctgtgtttaacttttcgaggaactgtattccacaatggccgcgccattttacgttcccatcagcagtgcccattggtcccagtttctccatatcctcaccagtgcttgttatttttcattttagaaaatgtttttgatttaaaataattattggtttcttctccacttttctgtgtgtgggtgtgtgtatttttttctttttaaactggtcatttagactttgaacctcttgtactactattacagttttctgattacttccctcctttttataaatgtctttaaacaatgcacctttaaaaaagagtgttaaatggtgccatcattgagttagtagttttgttttattttaactcctcctcttccctccaaatctatagtttttgtgtgtgtagaattttccaggtaacgcagaacacttgctgactattattactcaggctatggagattcctaccagttagtggacacatattttcctccaatttcttagtcttcaagaatcctagctccatttgataactatgagtagtgctttttgaataacttcttttggagggatgagactgcagcaagctatagttttgttttgttttttagagactgttcctcttttgcccaggctgcagtgcagtggcacagtcatagctcactgcagccttgaacttctgggttcaagcaatcctcctacctcagcctcccaggtagctaggattataggtacatgccaccatgcctggataatttttaaattttttgtagagatgggatctcgctatgttgcccaggctggtctcaaactcctgggctcaagcagtcttcctgcctcagccttccaaagtgttggggttacaggcattggctactctgcctggccctagttttgttcttaaaaaaaaaaaaaaaggggggccgtctaaaggatccaagtatannnnnnnnnnnnnnnnnnnnnnnntcctttccagcccaagggggcgaagattaccaagaatactggccgcgggactcccctataagggaagtccggaataaaattccgaaaaagcgccggggttaacccctggctttatggcatcgccgtgacaacaagtcagaaagagccagagaccatatcccaccaaccacagcacctagagtcataaaaaggcaaaaacacgcagtcggacaaccatcaacaagacagccaacgcaaatacaccctcacaacaccaacgatccaaataagcacgcaaccaccaacaaaaaaaacgaatacaacacaacatgccacaga

SEQ ID NO: 35 - DNA, 942 bp, Homo sapiens
  misc_feature: Incyte ID No 55003135CB1
gccttccaccattccctcaactcctcaccaaagactcagccatagaactcactgcagaggaattgactgggctgtcctggggaggggctggatgagtaccatcatctgtcatgcaacaggaggccccgcccaggacagcgtgatcatttgcagggagacacaatgctctgtgctccctgtctccattgggccaccctcccagcccctgcaaatgcatacactctttccaccttccttacttcccacacaaaaaggaatgtccttctgcaacaagctccacatggaaagtcagcattcaatatcagaagagaggctttgttttggggagggacgaggagagaattgaagagtcatgtgaccctaatatatctgtggttctggcagggactttgtatatatgcatcatctcatcctcacacgtctgacgaaataggtggcattatccccattttacagaggggaaatctaagttctcacagctggggagtccggaggccaggatcctcaagggtctatccagtcccaaagttactcactttctttttgagatggagtcttgctctgtcacccaggctggaatgcaatggtgagatctcggctcactgcaacctctgcctcccgacttcaagcgattctcctgcctcagcctcccaagtagctgggattacaggtgcctgccactatgcccagctaatttttgtatttttagtagagatagggtttcaccatgttggtcaggctggtctcaaactcctgacctcaggtgatccacccgccttggcctcccaaagtgctgggattacaggtgagagccaccgcgcccggccaagtcattcactttccaccacaccacactttcagaataactggaagctccccccaggctaagcggggcctcccacaaagtgcagaagatgagtggaggaaggaatggtcgaaagc

SEQ ID NO: 36 - DNA, 3288 bp, Homo sapiens
  misc_feature: Incyte ID No 5855204CB1
ggagccccggcggggcgcttggtttcggtttggccctgactgggattagtgttgacgatcgaaatgggagtccccaagttttacagatggatctcagagcggtatccctgtctcagcgaagtggtgaaagagcatcagattcctgaatttgacaacttgtacctggatatgaatggaattatacatcagtgctcccatcctaatgatgatgatgttcactttagaatttcagatgataaaatctttactgatatttttcactacctggaggtgttgtttcgcattattaaacccaggaaagtgttctttatggctgtagatggtgtggctcctcgagcaaaaatgaaccagcagcgtgggaggcgttttaggtcagcaaaggaggcagaagacaaaattaaaaaggcaatagagaagggagaaactcttcctacagaggccagatttgattccaactgtatcacaccaggaactgaatttatggccaggttacatgaacatctgaagtattttgtaaatatgaaaatttccacagacaagtcatggcaaggagttaccatctacttctcaggccatgagactcctggagaaggagagcataaaatcatggaatttatcagatccgagaaagcaaagccagatcatgatccaaacaccagacactgtctttatggtttagatgctgacttgattatgcttggattaacaagtcatgaggcacatttttctctcttaagagaagaagttcgatttggtggcaaaaaaacacaacgggtatgtgctccagaagaaactacatttcaccttctacacttgtctttaatgagagagtatattgactatgagttttcagtattaaaagaaaagatcacatttaaatatgatattgaaaggataatagatgattggattttgatggggtttcttgttggtaatgattttatccctcatctacctcatttacatattaatcatgatgcactgcctcttctttatggaacatatgttaccatcctgccagaacttgggggttatattaatgaaagtgggcacctcaacttacctcgatttgagaaataccttgtgaaactatcagattttgatcgggagcacttcagtgaagtttttgtggacctaaaatggtttgaaagcaaagttggtaacaagtacctcaatgaagcagcaggtgtcgcagcagaagaagccaggaactacaaggaaaagaaaaagttaaagggccaggaaaattctctgtgttggactgctttagacaaaaatgaaggcgaaatgataacttctaaggataatttagaagatgagactgaagatgatgacctatttgaaactgagtttagacaatataaaagaacatattacatgacgaagatgggggttgacgtagtatctgatgactttctggctgatcaagctgcatgttatgttcaggcaatacagtggattttgcactattactatcatggagttcagtcctggagctggtattatccttatcattatgcacctttcctgtctgatatacacaacatcagtacactcaaaatccattttgaactaggaaaaccttttaagccatttgaacagcttcttgctgtacttccagcagccagcaaaaatttacttcctgcatgctaccagcatttgatgaccaatgaagactcaccaattatagaatattacccacctgattttaaaactgacctaaatgggaaacaacaggaatgggaagctgtggtgttaatcccttttattgatgagaagcgattattggaagccatggagacatgtaaccactccctcaaaaaggaagagaggaaaagaaaccaacatagtgagtgcctaatgtgctggtatgatagagacacagagtttatctatccttctccatggccagaaaagttccctgccatagaacgatgttgtacaaggtataaaataatatccttagatgcttggcgtgtagacataaacaaaaacaaaataaccagaattgaccagaaagcattatatttctgtggatttcctactctgaaacacatcagacacaaattttttttgaagaaaagtggtgttcaagtattccagcaaagcagtcgtggagaaaacatgatgttggaaatcttagtggatgcagaatcagatgaacttaccgtagaaaatgtagcttcatcagtgcttggaaaatctgtctttgttaattggcctcaccttgaggaagctagagtcgtggctgtatcagatggagaaactaagttttacttggaagaacctccaggaacacagaagctttattcaggaagaactgccccaccatctaaagtggttcatcttggagataaagaacaatctaactgggcaaaagaagtacaaggaatttcagaacactacctgagaagaaaaggaataataataaatgaaacatctgcagttgtgtatgctcagttactcacaggtcgtaaatatcaaataaatcaaaatggtgaagttcgtctagagaaacagtggtcaaaacaagttgttccttttgtttatcaaactattgtcaaggacatccgagctttcgactcccgtttctccaatatcaaaacattggatgatttgtttcctctgagaagtatggtctttatgctgggaactccctattatggctgcactggagaagttcaggattcaggtgatgtgattacagaaggtaggattcgtgtgattttcagcattccatgtgaacccaatcttgatgctttaatacagaaccagcataaatattctataaagtacaacccaggatatgtgttggccagtcgccttggagtgagtggataccttgtttcaaggtttacaggaagtatttttattggaagaggatctaggagaaaccctcatggagaccataaagcaaatgtgggtttaaatctcaaattcaacaagaaaaatgaggaggtacctggatatactaagaaagttggaagtgaatggatgtattcatctgcagcagaacaacttctggcagagtacttagagagagctccagaactatttagttatatagccaaaaatagccaagaggatgtgttctatgaagatgacatttggcctggagaaaatgagaatgggtaagcaagatttatgatatttattacctcatagttcaattcagtgttttcaatgacaactgcagctggtttctcaagaatttcatattacttttttttttttttttttgagacaggttcttactctcttgcccgaatgcagtggctcaaatgtggc

SEQ ID NO: 37 - DNA, 1422 bp, Homo sapiens
  misc_feature: Incyte ID No 5778654CB1
gaactcaagagatgaagacttcatggtagaattctctgagacgtccctgaaagcaagaactttacctgatgatcttcattttctcaacttggagggatgaaaaaatcccgttctctggagaatgagaaccttcaaaggctttcattattaagtagaacccaggttccacttattactttgccacgtactgatgggccacctgacttagactctcattcgtatatgatcaactctaacacatacgagtcttctggctcccccatgctcaatttgtgtgaaaagtcagcagttctttcgtttagcattgagcctgaggaccaaaatgaaacctttttctctgaagaatctagggaagtgaatccaggggatgtttcacttaataatatatctactcagagcaagtggctgaaatatcaaaacacatcccaatgcaacgtggctactccaaacagagttgataagagaataactgatggcttctttgctgaggctgtttctgggatgcattttagagacacaagtgaaagacagagtgatgctgtcaatgaaagctctttagactctgtgcatttgcaaatgataaaaggcatgctctatcaacagcggcaggattttagcagtcaagattcggtttccagaaagaaagtactttctctgaatttaaagcagacttctaagacagaggaaattaaaaatgtattaggagggtctacctgctacaactacagtgtaaaggatttacaggagataagtggctctgagctgtgctttccaagtgggcagaaaataaaatctgcttatcttccccaaaggcaaattcacataccagctgtttttcagtctcctgctcattataagcagactttcacatcttgcctcatagaacatctaaatatattgctgtttgggttagcacaaaacctgcagaaagctctttcaaaagttgacatatcattttatacatcattgaagggagagaaactgaaaaacgcagaaaataatgtaccatcctgccatcatagtcaacctgcaaaacttgtcatggttaaaaaggaaggtccaaataagggtcgtctcttttatacatgtgatggacccaaagctgatcgatgtaaattctttaaatggcttgaggacgtgactccaggatattcaacacaggaaggagctcgacctggcatggttttaagtgatattaagagtattggcttatatttaagaagtcaaaagataccactttatgaggaatgccagcttttggtgagaaaaggatttgattttcagagaaaacagtatggcaaactaaagaagtttactactgtaaatcctgagttttataatgaaccaaaaaccaaactttatcttaagctaagtcggaaggaaagatcttcagcttatagcaaaattcccacctatgagtga

SEQ ID NO: 38 - DNA, 2129 bp, Homo sapiens
  misc_feature: Incyte ID No 1440126CB1
atggaggttcccccagcaacaaagtttggtgagacctttgcatttgagaacaggttagagtcacaacaagggcttttcccaggggaggacctgggggacccttttcttcaggaaagaggtttggagcaaatggctgtgatctacaaggagatccctcttggtgagcaggacgaagaaaatgatgattacgaggggaatttcagtttgtgctcaagccctgttcagcatcaaagtatccccccaggaaccagaccccaggatgatgagctcttcggacaaaccttcctccagaaatccgacctcagcatgtgtcagataatccacagtgaagagcccagtccatgcgattgtgcagaaacagacagaggggactcaggacctaacgcacctcacagaaccccacaaccagccaagccctatgcgtgtcgagagtgtgggaaggccttcagccagagctcgcacctgctccgacacctggtgatccacactggggagaagccctatgagtgctgtgagtgcgggaaggccttcagccagagctcccacctgctcaggcaccagatcatccacaccggggagaagccctacgagtgccgggagtgtgggaaggccttccgccagagctcagccctcacgcaacaccaaaagatccacaccggaaagaggccctacgagtgcagggaatgcgggaaagatttcagccggagctccagcctcagaaaacacgagagaattcatacaggagagagaccttatcagtgtaaggaatgtgggaaatccttcaaccagagctcaggcctgagccagcatcggaagatccacaccctaaagaaacctcacgagtgcgatctctgtgggaaagccttttgtcacaggtcacacctcatccgacaccagcggatccacactgggaagaaaccatacaaatgcgatgagtgcgggaaggccttcagccagagctccaacctcattgagcaccgcaagacccacactggcgagaagccctacaaatgccagaagtgtgggaaagccttcagccagagctcctccctcattgagcaccagcgcatccacaccggtgagaagccctacgagtgctgtcagtgtggcaaggccttttgccacagctctgcgctgatccagcaccagagaatccacaccggcaagaagccctacacctgcgagtgtggcaaagccttccggcaccggtcagccctcattgagcactataaaacccacaccagagagaagccctacgtgtgcaatctgtgcggcaagtccttccgggggagctcgcacctgattcgccatcagaagattcattctggggagaagctatagaaagaggagcccacacaaagcttgaaagcctgtgccagatggagcctttattccacgtcgcgtggtctccaagaccccacctacctccctgatgctgaatggaaaccttcccacctaagcgctcttgaacatcccactagcaggaaggccctgtgtggccctgggccaagcacgccagctctcagcaggttttctgcatggaagggaaggtggggtgatagggagggcagccagaaaagacagctggccttcagtttctccttcttgcatttgactcccaagcctacaggatttcattttgccctgtcttcacatttcccagaatctagaagaataaatgccaagagggagaaagttgaattaaacccaggaaaatatcagaagaactctttaaaacaggctgcagtctggcacttttagaagacagaaaccacactcacagtccaaagcagtagaagaaaaatgaattcaaaagtagatgtgtcagcagcaaagtagaattttctcactgcgttgggcattggtggggacaaccaaaacaggtctgcagagcaaaggcaggtccggagggtgggtcttgagattggggatgagggtggtgagcccctgggcgtggcctcttaaccctctccttagccctagggcagtttccctcagacagctctgttacaggaggatagagatttgatcctgttggccagaggatgtttgacacccagccgtgactcagcctggacaccggacatgactgccctccaggaataacccctccctgagacatagtgtcc

SEQ ID NO: 39 - DNA, 3103 bp, Homo sapiens
  misc_feature: Incyte ID No 3934519CB1
ttcggctcgaggcagcggaacgattcgattcttctcagcaccaagttgcgctcccaatctctcagagctgggctcgcgggaggccgctcgtgcaaaacctaggctgagctcccctgcgcggagctgtgagccctggaacaccgtggtctgcttctcaggacgcgcaaacagtgaagccagtcccgcccggagttcttcatatattaaggattcattcattcatagactcatttattgaaggctgtctgtgtaacaggcacaatcctaggtgcttgggatatagcagtgaacaagagacaaaccccctactatcatggtacttacatttttgtgggctggataataaacaagctctacttctgcaggccccatcccttcccagaaagaagaggaaatgactgagtcccagggaacagtaacattcaaagatgtggctatcgacttcactcaggaggagtggaagagattggatcctgctcagagaaaactgtaccggaatgtgatgctagaaaactataacaacttaatcacagtaggctatccgttcaccaaacctgatgtgattttcaaattggagcaagaagaagaaccatgggtgatggaggaagaagtattaaggagacactggcaaggagaaatatggggagttgatgagcatcagaaaaaccaggacagacttttgagacaagttgaagttaaattccagaaaacactgactgaagaaaaaggcaatgaatgtcaaaagaaatttgcaaatgtatttcctctgaactctgattttttcccttccagacacaatctctatgagtatgacttatttggaaagtgtttagaacataattttgactgtcataataatgtgaaatgccttatgagaaaggagcattgtgaatataatgaacctgtgaaatcatatggtaatagctcatcccattttgtcattaccccctttaagtgtaatcattgtggaaaaggcttcaatcagactttggacctcatcagacatctgagaattcatactggagagaagccctatgaatgtagtaactgtagaaaagccttcagtcacaaggaaaaacttattaaacattataaaattcacagtagggagcagtcttacaaatgtaatgaatgtggtaaagctttcattaaaatgtcaaatctcattagacatcaaagaattcatactggagagaagccctatgcatgtaaggaatgtgagaagtccttcagccagaaatcaaatcttattgatcatgaaaaaattcatactggagagaaaccttatgaatgtaatgagtgtggaaaagcattcagccagaagcaaagcctcattgcacatcagaaagttcatactggggagaaaccttatgcatgtaatgaatgtggtaaagccttccctcgaattgcatcccttgctcttcatatgagaagtcatacaggagaaaaaccttataaatgtgataaatgtggtaaagccttctctcagttttccatgcttattatacatgttagaattcatacaggtgaaaaaccctatgaatgtaatgagtgtggaaaagccttctctcaaagctcagcccttactgtacatatgagaagtcacactggtgagaaaccctatgaatgtaaggaatgcagaaaagccttcagccacaagaaaaacttcattacacaccagaaaattcatactagagagaaaccttatgagtgtaatgaatgtgggaaagcttttatacagatgtcaaatcttgttagacaccagagaattcatactggggaaaaaccctatatatgtaaggaatgtgggaaagcctttagccagaaatcaaatctcattgctcatgaaaaaattcattctggagagaaaccctatgaatgcaatgaatgtggtaaagccttcagccaaaagcaaaacttcattacacatcaaaaagttcatactggagagaaaccttatgattgtaatgaatgtggtaaagccttctctcaaattgcatcccttacccttcatttgagaagtcatacaggggaaaagccttatgaatgtgataaatgtggtaaagccttctctcagtgctcactgcttaatttacatatgagaagtcacacaggtgagaagccctatgtatgtaatgaatgtgggaaagccttctctcaaagaacttcccttattgtgcacatgagaggccatacaggtgaaaaaccctatgaatgtaataaatgtggaaaagccttctcccaaagctcatcccttactatacatatacgaggacatacaggtgagaaacccttcgactgtagtaaatgtggaaaagccttctctcaaatctcatctcttacccttcatatgagaaaacatacaggtgagaagccctatcactgtattgagtgtggcaaggctttcagccaaaagtcgcaccttgttagacaccagagaattcatactcattagaaaccctatgaatattgtgaatatggcaaggccatctgaaggaattaacacctcattgcacattacatgatcacttccagagtagaaaactatgaatgtgggatagccttctgaaaaagccacaaatttatgaaacattagagaattcttccaaggtgacaaattatataatgaaaaagctgttaccagaaactttcagcagacatcttattgataatatttaatcagcattctcattgaaatctgaaacaaggtatcttccatcagcataacaacacaacacttgagctcctagccaatgtattaagtaaggaaaacaagtatcaacgaagaactataaaaataacttttatacaagaaataattagaaaatgtgttgatagaaaacctaagaataaacacaacaatgagttaggtctttatcaagttataacaatatgttgacaaggcacaaaagaacacctgaaaaatgatatttgtctgaaaaagacttgacattgttaaaatataatttctctccccaatcactaagtttaatatacttctgaagttacatcagtttatcttaaggaactcacaaattatccaaatttctgtggcagttagaaccaatagtgacaactggaagcagtgagaaaacccttttta

SEQ ID NO: 40 - DNA, 8810 bp, Homo sapiens
  misc_feature: Incyte ID No 2946314CB1
cggttactgctaccccggtgcattgtgggcgcacggtccgttgttcatttgccattcgacctccgccagggcctggtcggacggaaacgctccgccggctttattgtcgcttcgttatgtggcggagccgagcagtttagcgtgcctctcaccctcagcgcctgcgaagccggcggcggcgtcgggactcctcggcgcgcggaggaaggatatctgtgtggaggatcgagggggcagcggagggtctccccgcactccgctgctcaacttcgaaggcctcgctcgctgcaggctcgctcctcacctctccgccgcccgcccccttctccgcgcgggacgctgcccggagcgcggcggggcgggggtggaggacgagagagcggtcggaggcgtcggcccggcagcggcagcggcagcggacgcgtgcagcagacccgggagcgagcgcgagccgggctgccgggcgagagggcgaggccgagccccgcgagaccggaacgccgggggcgggggcgagacagagggggagccgcggggagcgcgcgggacgcggcccgaggccgtgcgcgagccggggcaccgggcggcggcggcggcggcgcgcgccatgtcgttcagtgaaatgaaccgcaggacgctggcgttccgaggaggcgggttggtcaccgctagcggcggcggctccacgaacaataacgctggcggggaggcctcagcttggcctccgcagccccagccgagacagcccccgccgccagcgccgcccgcgcttcagccgcctaatgggcggggggccgacgaggaagtggaattggagggcctggagccccaagacctggaggcctccgccgggccggccgccggcgccgccgaggaagccaaggagttgctgctcccccaagacgcgggcggccccacctcgcttggcggtggcgcggggggccccctgctagcggaaaggaaccgtcggactctggccttccgaggcggcggcggcgggggtctcggcaacaatggcagtagccgcggccgccccgagacctcggtgtggcccttgaggcatttcaatgggcgagggccggcgactgtggatctggagctggacgcgctggaggggaaggagttgatacaggacggcgcgtccctgagcgacagcaccgaggacgaggaggagggggcgagcctgggcgacggcagcggggcggaaggcggcagctgcagcagcagcaggcggtcgggcggcgatggcggggacgaagtggagggcagcggtgtgggagctggcgaaggagagactgtccagcacttcccgctcgcgcggcccaagtctctaatgcagaagctccaatgctccttccagacctcctggctcaaggactttccctggctgcgctattccaaggatactggtcttatgtcttgcggctggtgccaaaagacccctgcagatgggggaagcgtggaccttcccccagtggggcatgatgagctttcgcgagggacccgcaactacaagaaaaccctcctcctgaggcaccacgtctctaccgagcacaaactccacgaagccaacgcccagttccccaaaaacaaaaataagggtattactcatgaatggacttgttttgattcctctatccgtgacttaacagtttttgcagaattggatcttttggagtcagaaataccatcagaggaggggtactgtgactttaatagtaggccaaatgagaactcttattgctatcaacttctgcgacaactaaatgaacagagaaagaaaggtattctttgtgatgtcagcattgtggtaagcggaaaaatcttcaaagctcataagaacatcctggttgcaggcagccgtttctttaagactttatattgcttttcaaacaaagaaagccctaaccaaaacaatactacccacttagatattgctgcagttcaaggtttttcagtcatcttggacttcttgtattctggtaacctggtgctcacaagccaaaatgccattgaagttatgaccgtggccagctatcttcaaatgagtgaagttgttcaaacttgccgaaatttcattaaagatgccttaaatataagcattaaatcagaagctccagagtctgtagttgtggactataataatagaaaaccagttaatagagatggtctgtcttcatcacgggatcaaaaaattgccagtttttgggcaacacggaatcttaccaatttggcaagtaatgtaaagattgaaaatgatggttgtaatgtcgacgagggccaaatagaaaactaccaaatgaatgacagtagttgggtccaggatggatctcctgaaatggctgaaaatgaatctgaaggtcaaacaaaagtgtttatttggaataatatgggctcccagggaattcaagagactggcaaaacaaggaggaaaaaccaaactacaaaaagatttatttataatattccacctaataatgaaacgaatttagaagattgctcagtaatgcagccacctgttgcctatccagaagaaaatacactactcatcaaggaagaaccagatttagatggtgctctactctcggggccagatggtgataggaatgtgaatgcaaatttattggctgaagctggcactagtcaagatggaggtgatgctggtacttcacatgatttcaagtatggtttgatgcctggtccttcaaatgatttcaagtatggattgataccaggtacttcaaatgatttcaagtatggattgataccaggtgcttcaaatgatttcaagtatggattattgccagaatcttggccaaaacaagaaacctgggaaaatggtgaatcatctctaatcatgaacaagttaaaatgccctcattgtagctatgtagccaaatacagacgaacactaaaaaggcacttgctcattcacacaggagtgagatcatttagctgtgatatttgtggaaaactgtttactcgaagagaacatgtaaaaagacattccctggtgcataaaaaggataaaaaatacaaatgtatggtgtgtaagaagatcttcatgttagcagccagtgttggaataagacatggatctcgacgttatggtgtttgtgtagactgtgcagataaatcacagccaggagggcaagaaggtgtagatcagggacaggatacagaattccctcgggatgaagaatacgaggagaatgaagtaggagaagctgatgaagagctagttgatgatggagaagatcagaatgatccctctcgatgggatgaatcaggagaagtttgtatgtctctagatgattaactgacctactatactcctcaaggatgctgcatttggacctaatatgaatcgacaatttggattgttgaacttgaaggcttgcaaaatatggtacatgctggatagtagttatgttgctgtgaaaactgtagggtcaaagccttatagcaaaaaaaatttttttttatatttgcacaggactatacagcaaacaaccatgtggttggattacatggagtccccacatactcagtcagttatcaaagtaaaatattttttatttataggatatacagtaactatttgggtcctatgaaaatagtccttaaagagcttacattcatgtgctactttaacatgaatggagaaaatccgtttatggaagtacagtgacaattgacccaatcactctgtccatcaaaccactcaggctagtttgtactagtagagttttgtttctatttttatttttattaattttattttttttaatacagattttcagtgaggggctttttcaaccccattggttctattttcttgtatttttccatttaatttgcttcataacttaaaccaagtctcttctagtcttaggtattatttctcgattttgtgctgatgggcatgtttataagaactggagaggtaatttattggaatgaactaactgacttcctccattcccctcttcctttttgacatgaattttactacttcacaaatgaagaatgatgttatgaagttaccgtggcgaagttgacaaataccactaaaatgattatgatttagaagtaactttcttctgctgctctaatctgataaatggttactatatgatattttctgacatggtatttggttttgggtatctgttactttggcactattcttgtttgcctctttatttttgccagtgtactatacctcagtcctatttgaaagacctaattgaaagaaaaatcatagaaataagtaaaatgatttgttttataatttatccaccatgtccagtttggttagcttgttatgcaatataagtgaaatatcaggtttttacccgtgtcccttttatcatgaaaattaacacaaaatgtgcattctcttttgtttcatacttagcgggatattgattgttttgaaattatgatcaatacttaatttatttttttctgtccttgaagtcactacaccttgatacagtctttctagtagtcaccatgattcaacagtctcaaaaatcttacaaataaaattctgagaagctttattattctataaattcttcagggtacaaagggtgacatattgaggtgaaattgtcagatttacttagcctggtgacatgaattagctgattgtggaactctcagcagcatttcacgtattgttaacatgtatggcatttattagcatattgtatattatataagattgagggatgtcatattttcagctttcttttaaataaaaattgagtatattttcctattttatatcaggtaactaaattatgctagttatgtggaaaaaattgcaaagatgctttgacagatataatccctttggtgctccatctgatcaaagttgaaaagttgatgttttttaagagacaagctttatcattgtctctgatttcagtagaataatggtttattgttagacaatgatgtttctgcttggataaatcaaagataaattacagttacatggttagatgcatctttgtcatctatatgtagtttctacataactgtaaacctgacagataagaatagtttcggtttgatttttgtttaaaacagatgagtacttcttaatgttctagacaaatgagtaaaatgtatttctggtagaaattagttgggcaaagatactatgaatgaaaaagtattttaaacgtaaaatgttctttgtgaatatgtaagtatagtataaagatttctttcatcccatttatccccttcctttaaataaactagtttacaattggtagatctgtctatatataaatatatattaaagaacaaagatatatatctaaaaatcacctaaatctgctttattagactacagttcacttattgcatagaaactggacttggctttacattcttaatgtacttttacttttcctcaagatatgaacttactctcttgaagctgaattttcttttactacttaaatcatttatgtatatctggtaaattatgaccaaatttttgttagattgcatacagtaaattgaaatacacacttggtacactacgggattgttgtgcttttgtttggttttagttggaaataaggtttgatgtagatggcttgttactgttaatttaaaatattctataattgtccattactttatgttgtgttgtgacagatttggctatatttttagtcaattgaaatatgatactttaaaagtttgtttttaggtaatccaaaggaaaagtgtttatactcttgaatatattagcctcagcctatataaaaatttctttgaagtaaacattttaccggaaacagaattgacagatttttctacttgctgactgcctaacttattttgtttcatgatctcgagggactgccttttcccatctagatcttttaaaaaaatagttttagtactaaggtatactactggacatttctatttttttaagtgtattctttttctgacttacagtaaaatttcaggaagttgatagatactaagaacttatgattggtctcagaggtaacaatgaaatactcataattttgtctgtggaactctggcagaattatgttacacatttggcgaagttgtctcttgtaatttatattttaaatgaaaaagtattttagagcttttaatgaaggggaagagtataaactttctttcatattcactctgtagttacagaccgtctcataaatcaagattgtgcattattagagttctcagagacagatacttccatatggacccctgttcatttttcttatttactagatattttgctaaatttagcacactagcaattaagagtttaaaaaagaagaaacgttataggaaagggaaactgaacagtaatgagtagctttgaaattggggaaaacacactagctgggttaggtacagctatcctaatggagaagagtgagctaaatgcgttaattcatgtaaagtgcttagaacactggtacataataggtgcataacacatcttaaagttgtttaataatagataagaaccaagaggaaaaagaaataagaaaagatgggggaaaggaaggagaaaataggaaatgaaaggattaggacttgggcatttatattaatacaaaatggtgtaaacggcctgaatatcactgtagtagtatcatacgggggaaagtgtgatggaaaataggtttaaaaatcttaatttttaaaactttagtaagctttatttatattttttaggattttctgaacatagcatgaagggtttagattcatttttctgaaaaacgattaaaaaaactaccaattttttttttgagtgcccacacttgccgttgtgctgtatacatgattttaccttaatcctctataaggtaggtgctaattgtctccattttatagatgaggaaaggctcagagaaattcagaaacttggctaatttaacacagctgtgacagagctcgaatttgaccacgtctgattctgaagcccatactctttcagggaggctctattgctgcctcattaaacaactcttgtagaatttcagctcttaatggagtctatggattatatttttcctataagacagctgtgtttagaattttgagtgtctttcctccccccaataaatttccttactactctttcttcataaagcagttgttttgtcactttgactatattaagacacatcagtgtagcacatgctcttaactggctgtggcattttaattttataaaagaatggtttttatctgaagaactagatagaaaccccagtttttgtgccgttcagtcacccagatccttaaacttaactatggtacataatatccacacttagaactaacaggtgtttagtcactttaggacttaaaatggaaagtttttttttttttcctgctgtatctctgctttcaactgaagttaaagaattctagatagtttaaaatgtgttagggataagcacacttcaaaagatgttttctagccctaaattttatgacattgggtacttaaatttggagtacactagctattgagaacaatattttgggttgaggagagatggttgaggccattatttagtgggagcattggcattttgaacactgtaaacaaaagaacacttccgcctgctgtttgtaaaagctctttgggaagatcttttacgaagaccaactttttaaaatgtaaattacctacctaatataagtgtcctaagacatgtatgtaaacttccggaactgttttgtctgtgtatttagaaaatacacaagaatctttattcaaacctaaaaatataaacttgtgagcactaaactgcttctggcttgtgaattttattgactgacatttaattcagatttctttgcaagtgtactaatttagactaattggggtaaggggaacaccttaatgaactttgacttatgtataattcagatatctttgtacttaaggcttacaagtttgaattatggaatactataagggttttttgtttatttgtatgtttttatatgaaagtacataaatgacagactacctccaagtaatcctgctttaattaatagcagtgatttgtaatcagtgtatcttttaagattctctacaggttttagaaaatattaaaaattccctgtaatttacatttgtgcataatcttggaaatgggttgaaaagcaaaggtaaactgcttcatcccatgttgtatatttgtggactgattgactacaagtgatgtgatgttataaatttgaagtctttgaaactttattaatgtagagaaaacataactagctttttaggttatttcaaagttagtaatagagcaaaggaaatcagaactggccagatattcagacttgacttagaaaaacaggagtttgttgattttgtttggagaagggcacaataaaggggagcccacatagcattataatgcctagtatctttaaacatgtaaagagctatcatgaggcagaagaattaagaaactttatatgtttctaaggggtcatgattgggaccattggctcaagattgaaaatcggtttttaattatccaaagatgacatggggctgcttcagaaaaaggtctaccactggagataacgctatgaaaagtgaaagagattacttaatcattttgacaaaggcagtattgaccagacatttctatttcagagttggatttgtgcttagtttttaataggtgttggcgaccttggagctgcttattattttgcttagtcaacaaacactactatgatacactttggtatgg

SEQ ID NO: 41 - DNA, 4070 bp, Homo sapiens
  misc_feature: Incyte ID No 3617784CB1
caagtaattcggcacgagaccctttattctgtgatattttgagctgttctgcgtctcgcaggcccttctgaatttcctggacctactctcggaaccctcaacgagctagcgatagatttagaggaaaggaagcaccgtcggaaagcaaaggcatgatttcaccttcacttgaactgcttcattcaggactctgcaaattccctgaagtagaaggaaaaatgaccacattcaaagaggcagtgacattcaaggatgtggctgtggtcttcactgaggaggagctggggctgctggaccctgcccagaggaagctgtaccgagatgtgatgctagagaacttcaggaacctgctctcagtagcacatcagcccttcaagccagacctaatatcccagctggagagagaagaaaagcttttgatggtggagacagaaaccccaagggatggatgttcaggcgacaagaatggaaaggatacggagtatattcaagatgaagaattaaggttcttttcacacaaagagctctcctcatgcaaaatctgggaagaggtggcaggtgaattacctgggagccaagactgtagagtaaatctgcaaggaaaagacttccagttctcagaagatgctgctccccatcaagggtgggaaggagcatctacgccgtgttttccaattgagaatttcctggacagtctacaaggggatgggcttatcggtctagaaaatcaacagtttccagcctggagagctataagaccaatccccattcaaggatcttgggcaaaagcgtttgtgaaccagttaggggatgttcaagaaagatgtaaaaatctcgacacagaagacacagtatataaatgtaactgggatgatgacagcttttgctggatatcttgtcatgttgatcacagattccctgaaatagacaagccgtgtggttgcaataaatgcagaaaagactgcattaaaaactctgtacttcatcgcattaaccctggagagaatggcttgaaaagtaacgaatacagaaatggcttcagggacgatgcagaccttcccccgcatccaagagtacctttgaaagagaaactctgtcaatatgatgagtttagtgagggcttgaggcacagtgcccatcttaacagacatcaaagagttcccacaggagagaaatctgttaagagtcttgagcgtggtcggggcgtcagacagaacacgcacatatgtaaccaccccagagcccctgtgggagacatgccctatagatgtgatgtctgtggaaaggggttcaggtataaatcggttcttcttattcatcaaggggtgcacacaggaaggagaccctataaatgtgaggagtgtgggaaggcatttggtcgaagttcaaacctgcttgtccatcagagggtccacactggagagaaaccatataaatgcagcgagtgtgggaagggcttcagttacagctcagtgcttcaagtccatcagaggctgcacacaggggagaagccctacacctgcagcgagtgtggcaaaggcttctgtgccaagtctgcactgcacaaacaccagcacattcaccctggagaaaagccctacagctgtggcgagtgtggaaagggattcagctgcagctcccacctcagcagtcatcagaagacacacaccggcgagaggccctaccagtgtgacaagtgtggcaaaggtttcagtcacaactcgtaccttcaagctcaccagagagttcacatggggcagcatctgtacaaatgtaacgtgtgtggtaagagtttcagttacagctcagggcttctcatgcatcagagactgcacacaggagagaaaccctacaaatgcgagtgcgggaagagctttggccggagctccgacctccacatccatcagagggtccacacaggagagaaaccctataaatgcagtgagtgtgggaagggcttccggcggaattcagaccttcacagccaccagagggtccacacgggagagaggccctacgtgtgtgacgtgtgtgggaagggtttcatctacagctccgacctccttatccatcagagggtccacactggagagaaaccctataaatgtgctgagtgtggcaaaggcttcagttacagctcagggcttctcattcaccagagagtccacacaggcgagaaaccttacagatgccaagagtgcagaaagggctttaggtgcacatcaagccttcacaaacatcagcgagtccacacgggaaaaaagccctatacgtgtgatcagtgtggcaagggattcagttatggctctaatcttcgcacccaccagaggttgcacacaggagagaaaccctacacttgttgtgaatgtgggaagggtttcagatatggctcaggtctccttagtcataagagagtgcacactggcgagaagccatacagatgccacgtgtgtgggaagggctatagtcagagctcacatcttcaaggtcatcagagggtccacactggtgagaaaccctataaatgtgaggagtgtgggaagggctttggccgcaactcctgtcttcatgttcatcagagagtccacactggagagaagccctatacgtgtggtgtgtgtgggaaaggcttcagttatacctcaggtctgcggaaccaccaaagagtgcatttaggcgagaacccttataagtagatgtacatagaggattccatctgggactcagagctttctatccatctgagagccaacacaggagaaaaaccataggaaagtgacatgtaggagggctataggagaaactgatgattttacattttctagtctgcataggaagggaacacctggacgtgtgataaataggttagcacttcagttacaaacttcagtctttaagagtctgttgtgcagcggagtaggctttaaagaaacagtgcagcagtggtttcagtaataatcctcacttccatcagaatctgcctgggagggagcttttacaatgtacaagtggtgagaatgtttccaaggtgctaattatggacatagattctttgtgtgggttgatagctaccaatgggacagtgtagaaacaagtttccacctttctaaaaccagtttggtacatatctttttaaagatcgctttataaaaacaaaaagataacattaacatttattgaaacaagtcactctgagaagaccaggaacaaagttctcttggcagatctgggtaatgtgtgtgtctcacatttttatcatgtttttatgactgttttatcttgcaggtaagctttacagctttcagaggagttgtagtacactacagaaatatgtcttctttccaggttcaaaaagcaaaatctgattggcattgcattgaattattaattaaataaatttattaattggcattgcattgaatttataggttaattttgaaagaatgtggcgtctttgaaatgctgattttttttcactttaaaaaaatgacttttttttttaccactgatagtatttcatatggtccctcagtaatatttcttagagatggttaaaaataggttctgagtcatcttaagcatatttctaggaatttgttttattgctgtcatgaatataatggttttgctccagttcatttcatagtttaacatatcaaatattaggtatcttaaaaatcacaaatgctatttcaccctgcaggttagcagtattaaaatagaatcatcaaatcccccatcagtgggggcggggggggggttgtagaaacagtctgatatggtttggctcttgtccccacccaaatctcatctcaattttaatccccacgtgtcagaggagggccttggtgggagatgattggatcacgagggcagatttctcccttgctgttctcatgatagtgagttctcatgagagctgatggttttaaggtgtagcccttcctcgctgtctctctcctgtccccttgtgaagaaggtccttatttctcttttgcgttctgccatgactaagttttcggaggcctccccagccatgtggacctgtgtgtgagttaacctctttcctg

SEQ ID NO: 42 - DNA, 3250 bp, Homo sapiens
  misc_feature: Incyte ID No 7490869CB1
tgcggccgccgggagggcgcgcggcgccggagacatgtccaggcggaaacagagcaacccccggcagatcaagcgttccctcggagacatggaggccagagaggaggtgcagttggtgggtgccagccacatggagcaaaaggccacggcacctgaagccccgagccctcccagcgcagatgttaactcacccccaccgctgccgccccccacatccccaggaggccccaaggagctggaaggacaggaaccagaacccaggcccacggaggaagagccgggcagtccctggagcgggccagacgagctggagccggtggtgcaggatgggcagaggcgcatacgggcccgactcagcctcgccacgggcctgtcctggggcccgttccatgggagtgtccagaccagagcctcatcccccaggcaggcggagccgagcccagccctgaccctgctgctggtggacgaggcctgctggctgaggacgctgccccaggccctgactgaggccgaggccaacacagagatccacaggaaggatgacgcactctggtgcagggtcaccaagccggtgcctgcggggggactcctgagcgtgctcctcacggccgagccccacagcacccccggccaccctgtgaagaaggagccagcagagcccacgtgcccggcccctgcacacgacctccagctcctgccccagcaggccgggatggcctccatccttgccaccgcagtgatcaacaaagacgtcttcccctgcaaggactgtggcatctggtaccgcagcgagcgcaacctgcaggcgcacctgctctactactgcgccagccgccagggcaccggctccccggccgcagccgccacagacgagaagcccaaagagacctaccccaacgagcgcgtctgccccttcccccagtgccgcaagagctgccccagcgccagctccctggagatccacatgcgcagccacagcggagagcggcccttcgtgtgcctgatctgcctgtcggccttcaccaccaaggccaactgcgagcggcacctcaaggtgcacacggacacgctgagcggtgtctgccacagctgtggcttcatctccaccacaagggacatcctctacagccacttggtcaccaaccacatggtctgccagcctggctccaagggtgagatctactcgccaggggccggacacccagcaaccaagctgcccccagacagtctgggcagcttccagcagcagcacacggccctgcaaggccccctggcctccgcggacctgggcctggcgcccaccccatcgccaggactggacagaaaggccctggccgaggccaccaacggagaggccagagcggccccccagaatggaggcagcagcgagcccccggcggcccccaggagcatcaaggtggaggcggtggaggagccggaggcggccccatcctgggcccggagagcctgggccccaggccccgtcgcggacgccgtcgccgcgcagccccgccccggccaggtcaaggccgagctgtccagccccacgccgggctccagcccggtgcccggcgagctgggcctggccggggccctgttccttccgcagtacgtgttcgggcccgacgcggcgccccccgcctcggagatcctggccaagatgtccgagctggtgcacagccggctgcagcagggcgcgggcgcgggcgccggcggcgcgcagaccgggctcttccccggggcccccaagggcgctacgtgcttcgagtgcgagatcaccttcagcaacgtcaacaactactacgtgcacaagcgcctctactgttcaggccgccgtgcgcccgaggacgcgcctgccgcgcgcaggcccaaggcgccccccggcccggcccgcgcgccccccggccagcccgccgaacccgacgcgccgcgctcgtccccgggccccggagcgcgcgaggagggggctgggggcgcggccacgcccgaggacggcgcgggcggccggggcagcgagggcagccagagcccgggtagctccgtggacgacgcggaggacgaccccagccgcacgctgtgcgaggcctgcaacatccgcttcagccgccacgagacctacaccgtgcacaagcggtactactgcgcctcgcgccacgacccgccgccgcgccgaccggccgcgcccccgggaccccctgggccggccgcgcccccggccccctctcccgccgcgcctgtgcgcacgcgcagacgccgcaagctctacgagctgcacgcggccggcgccccgccccccccgccgcccggccacgcccccgcgcccgagtcgccgcggcccggaagcggaagcggaagcggccccggcctcgcccctgcgcgctcgcccggccccgcggccgacggccccatcgacctgagcaagaagccgcggcgcccgctccccggagccccggcaccggcgctggccgactaccacgagtgcacggcctgccgcgtgagcttccacagcctcgaggcctacctggcgcacaagaagtactcgtgccccgctgcgccaccgcccggcgcgctcggcctgcccgccgccgcctgcccctactgccccccgaacggcccggtgcgcggggacctgctggagcatttccgcctggcgcacggcctgctgctcggcgcgcccctggccggcccgggggtcgaggcccggacgccggccgaccgcggcccctcgcccgctcccgcccccgccgcctccccgcagcccgggtcccgtggcccccgggacggcctcggcccggagccccaggagccgccgcccggcccgcccccgtccccggccgccgcgcccgaggccgtgccgcccccgccggcgcccccctcctactcggacaagggcgtccagactcccagcaagggcacgccggcgccgctgcccaacggcaaccaccggtactgccgtctttgcaacatcaagttcagcagcctgtccaccttcatcgcccacaagaagtattactgctcctcgcacgccgccgagcacgtgaagtgagcgcccacactacagccgcagacgctttgcacgccccgctgcgatgcggggagggggccgcccccaggccgcacggactgccgctcctgggaatcccgccacgcacaggcctcggcggagggggccgcagggggcagcgcccgcctggacccttggcacttaataaagaagttcagtttgatgagaaaaaaaaaaaaaaaaaaa

SEQ ID NO: 43 - DNA, 1345 bp, Homo sapiens
  misc_feature: Incyte ID No 5994687CB1
ctcgagccgctgcagatcaactcttcaaattattcgaatgacccatatgagaaacagagaaaagccaagtatttctggttgaaacggagaagctgggctggagtgcgcctccctccaactccccgctttaactccctaaggttggtgggatggccagctctaagatctcagaaggctgaccagctgtgtgctaagaacaaagcagaaggcagcgaggacgtctcccgcgaagcctccccgtgtgtggctgaggatggctgagcagcagggccgggagcttgaggctgagtgccccgtctgctggaaccccttcaacaacacgttccatacccccaaaatgctggattgctgccactccttctgcgtggaatgtctggcccacctcagccttgtgactccagcccggcgccgcctgctgtgcccactctgtcgccagcccacagtgctggcctcagggcagcctgtcactgacttgcccacggacactgccatgctcgccctgctccgcctggagccccaccatgtcatcctggaaggccatcagctgtgcctcaaggaccagcccaagagccgctacttcctgcgccagcctcaagtctacacgctggaccttggcccccagcctgggggccagactgggccgcccccagacacggcctctgccaccgtgtctacgcccatcctcatccccagccaccactctttgagggagtgtttccgcaaccctcagttccgcatctttgcctacctgatggccgtcatcctcagtgtcactctgttgctcatattctccatcttttggaccaagcagttcctttggggtgtggggtgagtgctgttcccagacaagaaaccaaacctttttcggttgctgctgggtatggtgactacggagcctcatttggtattgtcttcctttgtagtgttgtttattttacaatccagggattgttcaggccatgtgtttgcttctgggaacaattttaaaaaaaaacaaaaaaacgaaaagcttgaaggactgggagatgtggagcgacctccgggtgtgagtgtggcgtcatggaagggcagagaagcggttctgaccacagagctccacagcaagttgtgccaaagggctgcacagtggtatccaggaacctgactagcccaaatagcaagttgcatttctcactggagctgcttcaaaatcagtgcatatttttttgagttgctcttttactatgggttgctaaaaaaaaaaaaaaaattgggaagtgagcttcaattctgtgggtaaatgtgtgtttgtttctctttgaatgtcttgccactggttgcagtaaaagtgttctgtattcattaa

SEQ ID NO: 44 - DNA, 3313 bp, Homo sapiens
  misc_feature: Incyte ID No 2560755CB1
gagacagcgggccccagcgcgcggctcggggctggggcgccagaagtgggactggagcgaagtagaggatgccgaggagaaaacagcaggcacccaagcgggcggcaggctacgcccaggaggaacagctgaaagaagaggaggaaataaaagaagaggaggaggaggaggacagcggttcagtagctcaactgcagggtggcaatgacacagggacggacgaggagctagaaacgggcccagagcaaaaaggctgcttcagctaccagaactctccaggaagtcatttgtccaatcaggatgccgagaacgagtctctgctgagtgacgccagtgatcaggtgtcggacatcaagagtgtctgcggcagagatgcctcagacaagaaagcacacactcacgtcagccttccaaacgaagcacacaattgcatggataaaatgaccgctgtctacgccaacatcctgtcggattcctactggtcaggcctgggccttggcttcaagctgtccaatagtgagaggaggaactgtgacacccgaaacggcagcaacaagagtgattttgattggcaccaagacgctctgtccaaaagcctgcagcagaacttgccttctcggtccgtctcgaaacccagcctgttcagctcggtgcagttgtaccgacagagcagcaagatgtgcgggactgtgttcacaggggccagcagattccgatgccgacagtgcagcgcggcctatgacaccctagtcgagctgactgtgcacatgaatgaaacgggccactatcaagatgacaaccgcaaaaaggacaagctcagacccacgagctattcaaagcccaggaaaagggctttccaggatatggacaaagaggatgctcaaaaggttctgaaatgtatgttttgtggcgactccttcgattccctccaagatttgagcgtccacatgattaaaacaaaacattaccaaaaagtgcctttgaaggagccagtcccaaccatttcctcgaaaatggtcaccccggctaagaaacgcgtttttgatgtcaatcggccgtgttcccccgattcaaccacaggatcttttgcagattctttttcttctcagaagaacgccaacttgcagttgtcctccaacaaccgctatggctaccaaaatggagccagctacacctggcagtttgaggcctgcaagtcccagatcttaaagtgcatggagtgtgggagctcccatgacaccttgcagcagctcaccacccacatgatggtcacaggtcactttctcaaggtcaccagctctgcctccaagaaagggaagcagctggtattagacccgttagcagtggagaaaatgcagtcgttgtctgaggccccaaacagtgattctctggctcccaagccatccagtaactcagcatcagattgtacagcctctacaactgagttaaagaaagagagtaaaaaagaaaggccagaggaaaccagcaaggatgagaaagtcgtgaaaagcgaggactatgaagatcctctacaaaaacctttagaccctacaatcaaatatcaatacctaagggaggaagacttggaagatggctcaaagggtggaggggacattttgaaatctttggaaaatactgtcaccacagccatcaacaaagcccaaaacggggcccccagctggagtgcctaccccagcatccacgcagcctaccagctgtctgagggcaccaagccgcctttgcctatgggatcccaggtactgcagatccggcctaatctcaccaacaagctgaggcccattgcaccaaagtggaaagtgatgccactggtttctatgcccacacacctggccccttacactcaagtcaagaaagagtcagaagacaaagatgaagcggtgaaggagtgtgggaaagaaagtccccacgaagaggcctcatctttcagccacagtgagggcgattctttccgcaaaagtgaaacacctccagaagccaaaaagaccgagctgggtcccctgaaggaggaggagaagctgatgaaagagggcagcgagaaggagaaaccccagcccctggagcccacatctgctctgagcaatgggtgcgccctcgccaaccacgccccggccctgccatgcatcaacccactcagcgccctgcagtccgtcctgaacaatcacttgggcaaagccacggagcccttgcgctcaccttcctgctccagcccaagttcaagcacaatttccatgttccacaagtcgaatctcaatgtcatggacaagccggtcttgagtcctgcctccacaaggtcagccagcgtgtccaggcgctacctgtttgagaacagcgatcagcccattgacctgaccaagtccaaaagcaagaaagccgagtcctcgcaagcacaatcttgtatgtccccacctcagaagcacgctctgtctgacatcgccgacatggtcaaagtcctccccaaagccaccaccccaaagccagcctcctcctccagggtcccccccatgaagctggaaatggatgtcaggcgctttgaggatgtctccagtgaagtctcaactttgcataaaagaaaaggccggcagtccaactggaatcctcagcatcttctgattctacaagcccagtttgcctcgagcctcttccagacatcagagggcaaatacctgctgtctgatctgggcccacaagagcgtatgcaaatctctaagtttacgggactctcaatgaccactatcagtcactggctggccaacgtcaagtaccagcttaggaaaacgggcgggacaaaatttctgaaaaacatggacaaaggccaccccatcttttattgcagtgactgtgcctcccagttcagaaccccttctacctacatcagtcacttagaatctcacctgggtttccaaatgaaggacatgacccgcttgtcagtggaccagcaaagcaaggtggagcaagagatctcccgggtatcgtcggctcagaggtctccagaaacaatagctgccgaagaggacacagactctaaattcaagtgtaagttgtgctgtcggacatttgtgagcaaacatgcggtaaaactccacctaagcaaaacgcacagcaagtcacccgaacaccattcacagtttgtaacagacgtggatgaagaatagctctgcaggacgaatgccttagtttccactttccagcctggatcccctcacactgaacccttcttcgttgcaccatcctgcttctgacattgaactcattgaactcctcctgacaccctggctctgagaagactgccaaa

SEQ ID NO: 45 - DNA, 3451 bp, Homo sapiens
  misc_feature: Incyte ID No 3217430CB1
gcaagggggcgttgtcgtgatgattccgcggccagcggatcgctgcgagtggccttgaaggcagctgctgcaggtgaagagtaggcggcggggcagagagcggcctccgagggtcacctgaatggttgagcatggaccctgttgctacccacagctgccatctgctccagcaactgcatgagcagcgaatccaaggcctgctttgtgactgtatgttggtggtaaaaggagtctgctttaaagcgcataagaatgtcctggcagcattcagccagtattttaggagcctctttcagaattcttcaagccagaagaatgatgtttttcacttggatgttaaaaatgtcagtggcatagggcagatcctggacttcatgtacacttctcatctagatcttaaccaggacaatatacaagtaatgctggacacagcacagtgtttgcaagttcaaaatgttctgagtctgtgtcacacatttttaaaatcagccactgtagtacagccacctggcatgccttgtaatagtacattgtctctacaaagcaccctgaccccagatgccacttgtgttatcagtgaaaactacccccctcatttactgcaggaatgttcagcagatgcacagcagaacaaaacgttggatgaatcgcatccgcatgcttcaccatcagttaatcgtcatcactccgcaggtgaaatctcaaaacaagctcctgatacttcagatggcagctgcacagaactgcctttcaaacagccaaattactattacaaactcagaaacttttacagtaagcagtaccataaacacgcagctggtcccagtcaggagagagttgttgagcagccttttgctttcagcacctctacagaccttaccacggtagagagccagccttgtgctgtcagtcattctgaatgcatcctggagtctcccgagcacttaccttccaacttcctggcccagcctgtgaatgactctgccccacaccctgagtcagacgccacatgccaacaacctgtcaagcagatgaggctcaaaaaggccattcatctgaagaagctcaatttcctgaagtcacagaaatacgcagagcaagtatctgaacccaagtcagatgatggtttgacaaagaggttggaatctgctagtaaaaataccctagagaaagctagcagccaaagtgctgaagaaaaagaaagtgaagaagtcgtcagttgtgagaattttaattgcattagtgagacggagaggcctgaagacccggctgccctggaagaccagtcccagacacttcagtcccagagacaatacgcgtgtgaattatgcgggaaaccttttaaacacccaagcaacttggagcttcacaaacggtctcatacaggtgagaaaccttttgaatgtaacatttgtgggaaacatttctctcaggcaggtaacttgcagactcacttacgacggcattctggtgaaaaaccatacatctgcgagatctgtggaaagaggtttgcagcctctggcgacgtccagcgtcacattattattcactcaggagaaaaaccacacttgtgtgacatctgtggtcgagggtttagtaacttcagtaatttgaaggagcacaaaaagacacacacggctgataaagtcttcacctgtgatgagtgtggaaagtcttttaatatgcaaaggaagttagtaaagcacagaattcggcacacgggggagcggccttacagctgctctgcctgcgggaaatgttttgggggatcaggtgacctccgcaggcatgtccgcactcacactggggagaagccgtacacatgtgagatctgtaacaagtgctttacccgctctgcggtgctccggcggcacaagaagatgcactgcaaagctggtgacgagagcccagatgtgctggaggagctcagccaagccatcgagacctccgacctcgagaaatctcagagctcagactctttctcccaagacacgtctgtgacgctgatgccagtgtcggttaaactccctgtccacccagtggaaaattccgtggcagaatttgatagccactctggcggctcctattgtaagttacggtccatgatccaacctcatggagttagtgaccaggagaagctgagtttggatcctggcaaacttgccaagccccagatgcagcagacacagcctcaggcctatgcttactcggatgtggacaccccagccggtggcgaaccactgcaggccgatggcatggccatgatccgttcctctctggctgctttggacaaccacggcggtgaccccctgggcagtcgagcatcttccaccacttataggaactcagagggtcagtttttctccagcatgactctctgggggctagcgatgaagacgctgcagaatgaaaacgagttagaccagtgatgtaccgcgcttctccacggtagaggcgtgttctcagtttagcaggctggtgttaaggctgtaggaggacccagtttccccatgacagtgccttctaactagccagagaataggtagcttccctcctgatgatggctcataatctgaagcatcttgagctgggggtgtgagggggagggcctgctggctcaccgtgaggcagccgcgggagggagcgctgacgtcacagaagcgaaggcttgatgctgtctcagcagcctcagctgtgggggggaagcgcgtgtgcatcgtgtcaactactgtacatgttggtcatgtgaaaggaattatatatgtatagtattacaagtatttttgcatttttacaagattgaaatttgtagcattttgtattatttacacagaatttatttgtatatgaaactcataccataatttaattcgaataaatgaaacttttctatatattatatgtttcctctagcatttttattaatctaaagactataggggtataaaaataaatagcagcgaggactcactctgcaaggataagaacctcattggtcagtctccctcctggtacagcgggtttctcagtggtcagaactgcctgactcctggctgccacttactggcaaggtcatgggtgagctgttcaccctctcaggatcttctctggagtttcttttttgcagtatgagaggaacatcttccagtattttcacaaggcttgtctgatctgagcactcacttgaaaatacctgcccggcgcctggcccttgacagggcgttctaaatatttacttccccttcccagcgggcttgacagcctctgcaggaaggagatgtgcccgtggctctgtcgctggaatatcaaggtgagactggggatgtggcacgtcacaggtgatctgcttatagcactgcctgcacggaagactggagagcaccctacacggaggactgtgtggagagcacctcgaatactcgtgagcagctctaactcaaacgtacaagat

SEQ ID NO: 46 - DNA, 3220 bp, Homo sapiens
  misc_feature: Incyte ID No 5786832CB1
ctcgagccggggaaaggccacgtcgctatgagtgtgtttcagtctacctggattagacgttgcttctcttcgtctaccttgattaaacgtgcacttcgcagtcctcggttctccatacccgtgacctggggatcgctacggaccttaaaatacccgcaacagccccttcgtcccaagctggagagcagtggcatgatctcggctcactgcagcttctacttcctggcctcaagcagtctttccacctcagcctctcaacgcactggaattacagatgtgagccaccacactaggcctacaagtggtccttacaccagattaatttatcttgaaatggcaggcaactgaatcgcacacctcaatctatgatttgacttttaaagaattaattatattgactgagagagaggcccaggagagaaagaagaaagaaaaggagccagggatggctcttcctcagggatctttgacattcagggatgtggctgtagaattctctcaggaggagtggaaatgcctggaccctgttcagaaagctttgtacagggacgtgatgttggagaactacaggaacctcggcttcctgggactctgtcttcctgacctgaatattatctccatgttggagcaagggaaagagccctggactgtggtgagccaagtgaaaatagcgaggaacccaaactgtggggaatgcatgaaaggcgtgatcaccggtatctctcctaaatgtgtgatcaaggaattaccaccaatacagaacagtaacacaggagaaaaattccaagcagtgatgttggaaggacatgaaagctatgacactgaaaatttttacttcagggaaatccggaaaaatctacaggaagttgactttcaatggaaagatggtgaaataaattataaagaagggccgatgacccataaaaacaatcttactggtcaaagagttcgacatagtcaaggggacgtagaaaacaagcatatggaaaatcagcttatattaaggtttcagtccggtctgggtgaattgcagaaatttcaaactgcagagaaaatttatggatgtaatcaaattgagaggacagttaataattgttttttagcttcaccacttcaaagaatttttcctggtgtccaaaccaacatttctaggaaatatgggaatgattttttgcaactttcgttacctacacaagacgagaaaacacatattagggaaaaaccttacataggtaatgagtgtggcaaagccttcagagtgtcttcaagtcttattaatcatcagatgatacatactacagagaaaccttacagatgcaatgagtctggtaaagcctttcatcggggctcactactaacagtacatcagatagtccatacaagagggaaaccataccaatgtgatgtatgtggcaggatcttcagacaaaattcagatcttgtaaatcaccggagaagtcacactggagacaaaccctacatatgtaatgaatgtggcaagtcctttagtaaaagttcccaccttgcagttcatcagagaattcatactggagagaaaccttacaaatgtaatcgatgtgggaagtgctttagtcaaagttcctctcttgcaactcatcagacagttcatactggagacaaaccctacaaatgtaatgaatgtggcaaaacctttaaacggaactcaagcctcactgcacatcatataatccatgcaggaaagaaaccatatacatgtgatgtatgtggcaaggtcttttatcagaattcacaacttgtaaggcaccagataattcatactggagagacaccttacaaatgtaatgaatgtggcaaggtcttctttcaacgttcacgtcttgcagggcaccggagaattcatactggagagaaaccctacaaatgtaatgaatgtggcaaggtcttcagtcaacattcacatcttgcagtgcatcagagagttcatactggagagaaaccttacaaatgtaatgaatgtggcaaagcctttaattggggctcattactaactgtacatcagagaattcataccggagagaaaccttacaaatgtaatgtgtgtggcaaggtctttaattacggtggatacctttcggttcatatgagatgtcatactggagagaaacctctccattgtaataaatgtggcatggtcttcacttactattcatgcctagcacgtcatcaaagaatgcataccggagagaaaccttacaaatgtaatgtgtgtggcaaggtcttcattgacagtggaaacctttcaattcataggcgaagtcataccggagagaaacctttccagtgtaacgaatgcggcaaggtcttcagttactactcatgcctagcacgtcatcggaaaattcataccggagagaaaccttataaatgtaatgattgtggcaaagcctatactcagcgttcaagcctcactaaacatctggtaattcatactggagagaacccttaccactgtaatgaatttggtgaggcttttatccaaagttcaaaacttgcaagatatcacagaaatcctactggggagaaaccacacaaatgtagtgaatgtggtagaacttttagtcataaaacaagtctggtgtaccatcagagaagacatactggagagatgccatacaaatgtattgaatgtgggaaagtctttaactccactacaaccctggcaaggcatcggagaattcatactggagagaaaccttacaaatgtaatgaatgtggcaaggtcttccgttatcgctcaggcctcgcacgtcattggagtattcatactggagagaaaccttacaaatgtaatgagtgtggcaaagcctttagagtacgttcaattctgcttaatcatcagatgatgcatactggagagaaaccttataaatgtaatgaatgtggtaaagcttttatcgaaaggtcaaacttggtttaccatcagagaaaccatactggagagaagccatacaaatgtatggaatgtggcaaggcgtttgggcggcggtcttgcctcactaaacaccaacgaattcattctagtgaaaaaccttataaatgtaatgagtgtggcaatcttacattagtcgctcaggcctcactaaacatcagataaaacatgctggagagaaccttacaactaaactcaatgtggaaaggccgttagatgttgtcctaacctctgggatccccaaatagc

SEQ ID NO: 47 - DNA, 3268 bp, Homo sapiens
  misc_feature: Incyte ID No 7493320CB1
tttgacagctctcctaataactaccacggcgcaggccccgcccacctagctaccagcagcgccgattggccggcgggccggtatcccgcgctgtgattggcccgtcgcttcccctgagcgaacctttagaactctgagacaatattctgttacattgtagcaaaatggcgactgtcattcacaaccccctgaaagcgctcggggaccagttctacaaggaagccattgagcactgccggagttacaactcacggctgtgtgcagagcgcagcgtgcgtcttcccttcctggactcacagactggggtggcccagaacaactgctacatctggatggagaagaggcaccgaggcccaggccttgccccgggccagctgtatacataccctgcccgctgctggcgcaagaagagacgattgcacccacctgaagatccaaaactgcggctgctggagataaaacctgaagtggagcttcccctgaagaaggatgggttcacctcagagagcaccacgctggaagccttgctccgtggcgagggggttgagaagaaggtggatgccagggaggaggaaagcatccaggaaatacagagggttttggaaaatgatgaaaatgtagaagaagggaatgaagaagaggatttggaagaggatattcccaagcgaaagaacaggactagaggacgggctcgcggctctgcagggggcaggaggaggcacgacgccgcctctcaggaagaccacgacaaaccttacgtctgtgacatctgtggcaagcgctacaagaaccgaccggggctcagctaccactatgctcacactcacctggccagcgaggagggggatgaagctcaagaccaggagactcggtccccacccaaccacagaaatgagaaccacaggccccagaaaggaccggatggaacagtcattcccaataactactgtgacttctgcttggggggctccaacatgaacaagaagagtgggcggcctgaagagctggtgtcctgcgcagactgtggacgctctgctcatttgggaggagaaggcaggaaggagaaggaggcagcggccgcagcacgtaccacggaggacttattcggttccacgtcagaaagtgacacgtcaactttccacggctttgatgaggacgatttggaagagcctcgctcctgtcgaggacgccgcagtggccggggttcgcccacagcagataaaaagggcagttgctaaacccacggaacagactctctgggcaattagccatccccctctgactttggtcattgtgctggttctgatatatattttttttaatgaaaggcaactttagattttccctctatccttgctttttttcccttcacctcccacgtgtccctccatccctccccccacccctctgttttgggtatgtacaacagaagcacaaactactgaaacaaaacaaaacagcagaatgagcgttcttccgagagatggcatcgtgatgcgctatttattttccatagaaataggaagttagacggattgtctcttttctgaggggagggggtctttttgacaggagcagagttgatgtcctcaattttcatatttattggcaaaaggaagagaagaggaactttgggttggaaacaaagaaccaataacattaaaacattattatttatatattctagctgttattagaatcagactttttttgcgagagagagagagagagagagagaagggaaatcaaagaaatcgaagcaatatcctgtttagaggcaagccgcccggtggggagaatttcctcaatgggagacggttgcactttctgtgccccacggagtttgtggctccccgcggcagacccctccctcattctcctccctgacctttccatcttcctctctgcttgcgagaaaatgtcagtagttccagagaagtcggggtgcctatgcctggcctccctccacacctgggccctgaccagccgcctcctgggctcctcctcctccgtcagtagagctgctgttttgttattgctggtttttcctcactttcctcctggcaaagaacgacttccaaatgcagggatggaatataagcagaacgtcatgggctcagcagtgactccaccacccgaggccgaggccgtgcttctggaagatagaaggagacatcatcgtgtgtttcccctccccttgcccctgttaagaaacgtatcaatacccattggatgatcaaggctaccgtatttcttctatttttttttatagtgcctgccaggcactttgttttatgtttccaatagcacttcctgaaataaaccaaagcaacactgctcaaggcccctggggcgatggagaaggccacccacctcactgacagtcccaagaatgaccggctgcgaggtcctagtcaaaagtcaacattatgacctggggactccagcatccttcaagcaagccatttccgaagaaggtgaaaagaagccaggatgattggcacctcctcctcctcctcctcttcttcctcttcccttgcccagccccctcctgtgcgtgtgttgctttggtttatttcaggaagtgctattggaaacataaaacaaagtgcctggcaggcactataaaaaaaaatagaagaaatacggtagccttgatcatccaatgggtattgatacgtttcttaacagggggcaaggggaggggaaacacacgatgatgtctccttctatcttccagaagcacggcctcggcctcgggtggtggagtcactgctgagcccatgacgttctgcttatattccatccctgcatttggaagtcgttctttgccaggaggaaagtgaggaaaaaccagcaataacaaaacagcagctctactgacggaggaggaggagcccaggaggcggctggtcagggcccaggtgtggagggaggccaggcataggcaccccgacttctctggaactactgacattttctcgcaagcagagaggaagatggaaaggtcagggaggagaatgagggaggggtctgccgcggggagccgcaaactccgtggggcacagaatagtgcaaccgtctcccattgaggaaattctccccaccgggcggcttgcctctaaacaggatattgcttcgatttctttgatttcccttctctctctctctctctctctctctcgcaaaaaagtctgattctaataacagctagaatatataaataataatgtttaaggggatccactagttctaacgcgcaccgtgg

SEQ ID NO: 48 - DNA, 2434 bp, Homo sapiens
  misc_feature: Incyte ID No 2911453CB1
gtgtgtagcaggggagaatgagctgatgccgagggtccagccaccccgcctctgcctcctcctccccctgccgccgctgccctcgcagacgcgcgcgcacacacggcacttgggccgggtttccgcgctccgtccccccgtttggatgggggttttcatttccgaaggaggcacagcccgcggagcgctctgaagggctggagccccaagttactcctcgccagcgccggccgcccgctgtcactcgcgctggccggccgggggaagggacccgcacaccgggctttgttgtggaaatcccggttacctggtcccttatttcgcgtgagtctttggcgtccacgaccttgagtctgacggaaagtcagtcggcctcaagcatgaagcaggagtggtcccagggctacagggccctcccttcgctctccaaccacggctctcagaatggccttgatctaggggatctccttagccttcctcccgggacatccatgtccagcaatagtgtctctaactcattaccatcctacctttttggcacggaaagtagccactctccttaccctagtcctcggcactcatccaccaggtcccactcggcccgctccaagaagagagcgctgtccttgtccccgctgtccgatggcatcgggatagatttcaataccatcatccgcacgtcgcccacgtccttggtggcctacatcaacgggtcgagggcttcgccggccaacctgtccccgcagccggaggtctacgggcatttcctgggcgtgcgcggcagctgcattccccagccgcgcccggtgcccggcagccagaagggcgtgctggtggcccctggaggcctggcgctgccggcctacggcgaggacggggccctggagcacgagcgcatgcaacagctggagcacggcggcctgcagccaggcctggtcaaccacatggtggtgcagcatggcctgccgggccccgacagccagccggccggcctgttcaagaccgaacgcctggaggagttcccgggcagcaccgtagacctaccccccgcgcctccgctccctcctctgccgccgcccccaggccccccacccccttaccatgcccatgcgcaccttcaccacccggagctcgggccccacgcccagcagctggccttgccccaggccaccctggacgacgacggggagatggacggcatcgggggcaagcattgctgccgctggatcgactgcagcgccctgtacgaccagcaggaggagctcgtgcggcacatcgagaaggtccacatcgaccagcgcaaaggggaggacttcacttgcttctgggccggttgccctcgaagatacaagcccttcaacgcccgctataaactgctgatccacatgagagtccactctggggagaagcccaacaagtgtacgtttgaaggttgcgagaaggccttttcaaggcttgaaaatctcaagatccacttgcggagccacacaggcgagaagccgtatttgtgccagcatccgggttgtcagaaggccttcagtaactccagtgaccgcgccaaacaccagcggacgcatctggacaccaaaccttatgcttgtcaaattccaggatgtaccaaacgctacacagacccaagttccctaagaaagcatgtgaaggcacattcttccaaagagcaacaagcaaggaaaaagttgcggtccagcacagagctccatccagacctgctcacagattgcctcaccgtgcagtccctgcagccggccacttcccctagagatgctgctgctgaagggaccgtgggacgctcccctggacccgggcctgacctctattcaggtaaagacagcaaaggcagagccaaccacaactacccttttttaggccttttactctggttaaaaggaatgctcacgtgagaaaaattttcctaagatcgtctgtgttgttctttatcaatgggctgcaaattgctctccaccccttctgattcctgagctgggtgtggcagcagattgttatttctcataaagaaataaaataaaataatgcattacctcttttagttatggacttctgccagaaattcagtgcacccaaccctcgcagttatgttacatttactcacttgatttagactcgaaggaaatgtttcctccttaagcaggtagcaggctgcgtgtggatttcttaatgaaacagttgttgtcatctttatgttttaaaaggaagccccaggtctctaaggaaattttcacatcagcaaaccaacttataagaatgttcattcatgtttgccaaccattgtgggaaaggagatgggatgacactcccagaccaccttccatctgcctaggtttgaataataggtgactatatttggaaaattacatgtatgtagtaaaaaacaaaaaaaaaaaaaaagatcttt

SEQ ID NO: 49 - DNA, 684 bp, Homo sapiens
  misc_feature: Incyte ID No 3029661CB1
cttgagcccgggagtttgaggttgcagcgagctgtgatagcatcgctgcactccagcctgggtgacagagtgagaccctgactcaaaaacaaaagtaactctgacttaggaacaggcacagaggagatcatggtacactgttgataaaacaatttgcagaataatttgtagaatacgacccaagttttttaaaacaataaaaacaaaacacacaacataagcatatagaaaattgcctgagctttcctcagctgctgccaaggtgttcggtccttccgaggaagctagggacacattgaggtgaggccctcacttcatccagtgactggcactgcgtccggcagcgccagtcccacactcgcccgcgccatggcctccatctccgagctcgcctgcatctactcagccctcattctgcacgacaatgaggtgactgtcacagagtataagatcaaggccctcattaaagcagctggtgtaaatgttgaaccttttcggcctggcttgtttgcaaaggccccggccaatgtcaacattaggagcctcatctgcaatgtaggggctggaggacctgctccagcagctggtgctgcaccagcaggagctgaggagaagaaaatggaagcaaagaaagaagaatttgaggactctgatgatgacatgggctttggtctttctgactaa

SEQ ID NO: 50 - DNA, 914 bp, Homo sapiens
  misc_feature: Incyte ID No 71260474CB1
agaaatcaaaaaagcgtcttaatatgacatccttcaacattgcccagggcattcatgcttttgattatcactctcggctcaatttaattgcaactgctggcattaacaataaagtttgcctttggaatccctatgttgtctctaaaccagtgggtgtcctttggggccactcagccagtgtaatagccgtccaattctttgtggaaagaaaacaacttttcagcttctccaaggataaagttttgagactctgggatattcaacaccagctgtccatccagaggatagcttgttctttccccaaaagtcaggacttcagatgtctcttccactttgatgaagcccatggacgacttttcatctcgtttaataaccagctagcattgttggcaatgaaaagtgaagccagcaagagggtgaaaagccatgagaaagcagtcacttgtgttctttacaattctatcttgaagcaggtaatcagctctgatacagggtctactgtttccttctggatgatagacactgggcagaaaatcaaacagtttactggttgccacggcaacgcagaaatcagcactatggcccttgatgcaaatgagactcggcttttgactggcagcacagatgggactgtaaagatatgggacttcaatggatattgtcaccatacactaaatgttgggcaagatggagctgtggatatttcacaaatcctcattcttaagaagaaaatactggttacaggctgggagaggtatgattatgcctcatggaaaactataggaaggtaaaaaggagaatgttttattttaatcaaaatctaaaagaaaaaagaacataaaaatcttggttgactctgtgtcattaaaactttgctggtgacctaggtgagtgatacctagttggaattgacg

SEQ ID NO: 51 - DNA, 4713 bp, Homo sapiens
  misc_feature: Incyte ID No 7992707CB1
tgtattttttccccaaatgggtaatcagctcttcaaacattatttactggacaggccatccttcccctactaacgtaaatgtcatcttttcatattaaatttctccatgcgctgggtatttactcctacatcttccatctagtgcttttgagaattttcaacactcttccattacacgcatgtaccatcatttatttaatcaatcttctattggtaggcatttgggacgtttcctgctcttccatactaaacaatgcatagaattttggactacattaaataattctctgcctcagtggttcaagttgcgtaaattatgcgtttacacttttcttcggattacttttaaaatgacaaccactatgttttgctggtctcaattaccagagaattggcaagaaacctgacagctggggcttaggattccctgcgccagacctccgagctacaccagagggcgtactttccttactcggcctcggccacatccgggttccaccgcagattcgggcagggagcgggcggaacctttctaccgcgtctctagctaacacgcacggcggggacagtttaggcctccgcgcaccgttcgccgggagtcttgcagtttgcttggtgcagggaaggcgggcgcggaggttctatctgtttcttcctccttcgtgagcagcatggacgtgctagcggaggagtttgggaacctgactccggagcagctggcggcgccgatcccgactgtagaggaaaaatggaggctgcttccagcatttttaaaggtgaaaggccttgtgaaacagcatatagattcatttaactatttcattaatgtagagataaagaagataatgaaagccaatgaaaaggttacaagtgacgctgaccctatgtggtacttaaaatatcttaatatctatgttgggcttcctgatgttgaagaaagcttcaatgtaactagaccagtgtcccctcatgagtgccgtttgagagacatgacatactctgcccctattacagtggatattgaatatacccgaggcagccagaggatcatccgcaatgccttacctatcggcagaatgcccataatgctacgtagttcaaactgtgttcttacaggaaaaacgccagcagaatttgccaaactgaacgaatgtcccttagatccaggtggctacttcattgttaaaggagtagaaaaagttattcttatccaagagcagctgtctaagaacaggatcatcgtggaggctgatagaaaaggggctgttggagcttcagttaccagctctacccatgagaaaaaaagcagaaccaatatggctgtgaaacaaggacgattttatttgaggcataatactttgtcagaagatatacccattgtcatcatatttaaggccatgggtgttgagagtgaccaggaaattgtgcagatgattggaacagaggagcacgtgatggctgcatttgggcccagtctggaagagtgccagaaagctcagattttcacacagatgcaggcattaaaatatatagggaacaaagtaagaaggcaaaggatgtggggaggtggaccaaagaaaaccaaaatagaagaagcaagagagctcctggcttccaccattctgacccatgtcccagttaaggaattcaatttccgagccaaatgtatctatactgcagtgatggtgcgaagagttattctggcccaaggagataataaagttgacgacagagattattatggtaacaagcgactggaattggcaggacagcttttatctcttctttttgaagacttgttcaaaaaatttaattctgaaatgaaaaagattgccgaccaggtgattcctaagcaaagagcagcccagtttgatgttgtcaaacacatgcgccaagaccagatcaccaatggcatggtgaatgctatttctaccggaaattggtctttaaagagatttaaaatggaccgccagggtgtaacccaagtgctgtctcgcttgtcatatatatccgcactgggcatgatgacaagaatctcttcccagtttgaaaaaacgagaaaagtgagtggtcctcgctccctccagccatctcagtggggaatgctgtgtccttcggacactcctgaaggagaggcatgtggtttggttaaaaacttggcccttatgacacacatcacaactgatatggaagatggacccattgttaaattagccagtaacttgggagtagaagatgtgaatttattatgtggggaagagctctcttacccaaatgtgtttcttgtctttcttaatggtaacatcttaggtgtcattcgagaccacaaaaagctagtgaatacatttcgactcatgagaagagcaggatatatcaatgaatttgtttccatctcaacaaatcttacagatcgatgtgtctatatttcttctgatgggggaaggctatgcagaccctacataattgtcaagaaacagaagccagcagtcacaaataaacatatggaagagctggcccaagggtacaggaattttgaagatttcttacatgagagtctggttgaatatttagatgtgaatgaagaaaatgattgtaacattgcactgtacgaacacacaattaataaagacaccacccacttggagattgaacccttcactcttctcggcgtgtgtgctggacttatcccataccctcaccataaccagtcaccgagaaacacttaccagtgtgccatggggaaacaagccatgggtactataggatacaaccagcgaaacagaattgatactctcatgtatctactagcatatccacaaaaacccatggttaagacaaaaaccattgaattgatagaatttgagaaactgccagctggacagaatgcaacagttgctgtgatgagctatagtggctatgatattgaagatgctcttgttttaaacaaggcctctttagacagaggctttgggcgttgccttgtatataaaaatgctaaatgtacgttgaaacgatacaccaatcagacttttgataaagtgatggggcccatgttggatgctgctacaaggaaacctatctggcgacatgaaatcttagatgcagatggtatttgttctccaggtgagaaagtagaaaacaaacaagtgcttgtaaataagtccatgcccacagtgactcagattcctttggaaggaagtaatgtaccacagcaaccacagtacaaagatgtacccataacctacaaaggagcaacagactcatatattgaaaaagtgatgatatcttcaaatgctgaagatgcttttctgatcaaaatgctgctgagacagacaaggcgtccagaaattggagacaaattcagcagtcgtcatgggcaaaaaggtgtttgtggcttgatcgtcccccaggaagacatgccattttgtgattctggcatctgtccggacatcatcatgaacccacacggcttcccatcacgaatgacggtggggaagctcattgagctgctggctggcaaggccggtgtgctggacggcagattccactacggcactgcgtttggaggcagtaaagtgaaggatgtgtgtgaggacctcgttcgccatggttataactacttggggaaagactatgttacatccggcatcacaggtgagcccttagaagcatacatctattttggccccgtgtactatcagaagctgaaacacatggtgctagataaaatgcatgcccgggcccggggcccacgagccgtccttaccaggcaacccactgaaggacggtctcgtgatggtggcttgcgtctcggggaaatggaacgtgactgtttaatcggttatggagccagtatgcttttgctagagagactaatgatttcaagtgatgcctttgaggttgatgtctgtgggcagtgtggacttctggggtattctggctggtgccattactgcaagtcatcctgccacgtgtcttccctccgtattccgtatgcctgcaagctgctcttccaggaactacagtctatgaacatcatccccaggttaaaactgtccaagtacaacgaatgaggatggaaaaaatgattattaaagagaacaagtgatacatccaatgcaacggaaagcagaagggatttaggactacgtctcctcctgtgaagaattcccttgcgtattctctctctaaaacaaccaaaaaaaaatggagaggctttttatatactctaagactggctaaacaaccttgatcattgagcctcgagccatgggagagatgctgaccatgtggactgcaaggctgcttgattcacagatggatgtgacctaaaggataaataagctattacttatgtgctgatctcttgacattcactcattagaagaccttactccttcaagcaaatgtttggggtcagatttaccatatcttctggctaaccatattcaagattcttctgaaacttggaggatgtaaagaatccatttgatttggtcagcctggcttttgtcgtggtggctggctcggataaattttcccaacaattaaatcttgcctttacacacccaaactttgtaattttagtcttggtgaaatataatgaatttgttcctaccttgtcaagcaagaatgtcgtcttctcctatggactcaattgctattattttaaacctgcatgattgtaccatgaaatactattcgttaaatatt

SEQ ID NO: 52 - DNA, 9885 bp, Homo sapiens
  misc_feature: Incyte ID No 7974861CB1
ggacccgcgagcggagcggcgcgtgggtcggttgcggtcggccccggcaggatgggaaggccattgtgactatgtggtgattacagttgtcttactactgagtttcctactgaaatcatggaggagaaacagcagattatattggctaatcaagatggtggaacagtggcaggagcagcacctaccttctttgtcatcttaaagcagccaggaaatggcaaaactgatcaaggaattttggttactaatcaggatgcctgtgctttggctagtagtgtgtcatcaccagtaaaatctaaagggaagatttgccttccagctgattgtactgtgggtggaatcactgttaccctcgataacaatagtatgtggaatgagttctatcatcgaagcacagagatgattctgaccaagcaaggaagacgcatgtttccttactgtcgttattggataacaggtttagattcaaatttgaagtatattcttgtcatggatatatctcctgtggataaccatcgttataagtggaatggtcgttggtgggaacctagtgggaaggctgaacctcatgttttggggagggttttcattcatccagaatctccttccacaggtcattattggatgcatcaaccagtatctttctataaactcaaacttaccaacaatacactggaccaagaagggcatatcatcttgcactctatgcatcgttacctgccgaggcttcatttggtgcctgcagaaaaggctgtggaggtgatacaattaaatggccctggtgtccacacttttaccttcccacagactgaattctttgcagtaacagcttatcagaacattcagattactcagctgaaaatagattacaatccatttgccaaaggctttcgggatgatgggctgaataataagccccagagagatggaaaacaaaagaacagctctgaccaagaagggaataatatttccagttcttctggtcatcgggtccgtcttacagaaggtcaggggtcagagatacaaccaggtgatttggatcctttgtcaaggggtcatgaaacatcaggcaagggtttggagaagacttcccttaatataaaacgagactttcttggtttcatggatactgattcagcacttagtgaagttcctcaattgaagcaagagatttctgaaagtcttattgccagcagttttgaagatgactcccgtgtagcctcaccgttagaccagaacggaagcttcaatgttgttattaaagaggaacctctagatgattatgactacgaacttggtgagtgcccagaaggggtcactgtgaaacaggaagagacagatgaagagacggatgtatactcaaacagtgatgatgatcctatactagagaaacagctaaagaggcacaataaagttgacaacccagaagctgaccatctatcttctaaatggcttccaagcagcccatcaggtgttgctaaagctaaaatgttcaaattagacactggaaagatgccagtagtctatctggagccctgtgctgtcaccagaagcacagttaagatttctgaactccccgataacatgctttccacatctcgaaaggataaatcttctatgttggcagaattggaatatttgcctacatacattgaaaattccaatgagactgccttctgcttaggcaaggaatcagaaaatggtcttagaaaacattcaccagatctcagagtggtacaaaaatatcccttactgaaagagcctcagtggaaatatcctgatatatctgacagcattagcacagaaagaatactcgacgattcaaaggattcagttggagactcactttcaggaaaagaggacttgggcagaaagagaacaactatgcttaagattgcaacagccgcaaaggtagtgaatgctaatcagaatgcctctccaaatgtccctggaaaaagaggaaggccacgaaaattgaaactctgtaaggcaggacgaccacctaagaacacaggaaagtctttaatttctacaaagaatacacctgtaagccctgggagtacctttccagatgtgaagcctgatctggaagatgtggatggtgttctctttgtttcctttgaatcaaaggaagctctagacattcatgcagttgatgggacaacagaagaatcttctagtctccaggcatcaaccacaaatgactcaggttacagagcaagaatttcccagttggaaaaggaattgatagaagatttgaagactttgcggcacaagcaggtgatacatcctggtcttcaagaagtgggcttaaaattgaattcagtggatccaacaatgagcattgatcttaaatacttgggagtacagttacctttggctccagctactagctttcctttttggaaccttacaggaaccaaccctgcctctcctgatgcgggatttccctttgtttctaggacagggaaaaccaatgatttcactaagatcaagggatggaggggaaaatttcatagtgcttctgcatctaggaatgaaggtggaaattcagaaagttcactgaaaaatcgttctgctttctgtagtgataagctagatgaatacttggaaaatgaaggcaagctgatggaaacaagcatgggtttttcttctaatgctcccacatctcctgtggtgtaccagcttcccactaagagtaccagttatgtacgaacacttgatagtgtactaaagaagcaatctactatttccccttctacctcttattctttgaaacctcattctgtaccccctgtctctcgaaaggcaaagtctcaaaacagacaggcaactttcagtggccgaactaaatcatcttataaatccattttaccataccctgtttcaccaaagcagaaatactctcatgtgattctaggagataaggttaccaagaattcttcgggcatcatctcagaaaatcaggcgaataactttgttgtgccaactttggatgaaaatatatttccaaagcagattagtttgcggcaggcacagcagcagcagcaacagcaacagggaagtcgccctccaggcttgtctaaatctcaggtgaagctaatggacctggaagactgtgcactttgggaaggaaaaccaaggacatacatcacagaagagcgagcagatgtatccttaacaactctacttacagctcaagcatccctcaaaactaaacctatccacacaatcataaggaaacgagcccctccctgcaacaatgacttctgtcgactgggttgtgtatgttccagtctagctttggagaagcgccaacctgctcactgccgccgaccagactgcatgtttggttgtacttgtttgaaaagaaaagttgtacttgttaaaggaggatccaaaactaagcattttcagaggaaggctgctcatcgagatccagtattttatgatactctgggagaggaggcaagggaggaggaagaaggaatcagggaggaggaggaacaattgaaagagaaaaagaagagaaagaagctagaatacactatatgtgagacagagcctgaacagcctgttcgacattacccattatgggtaaaagtagaaggtgaagtagatccagaaccagtttatatccccacgccttctgtcattgagcctatgaaaccattgttattgcctcagccagaagttttatctcctactgtgaagggcaaactgctcactggaattaaatctccacggtcatatactcccaaacccaatcctgtgattcgggaagaggacaaagatccagtctacttgtactttgaaagtatgatgacttgtgctcgagttcgagtatatgagcgaaaaaaagaggaccagagacaaccatcttcctcctcctccccatctccatcatttcagcagcaaacttcatgtcattctagccctgagaaccataataatgcaaaggaacctgattctgaacagcagcccttaaaacaactcacctgtgatttggaggatgattctgataaattacaagaaaagagctggaagtcttcctgcaatgaaggagaatcctcttctacttcttatatgcatcagaggtcacctggtggtcccaccaaactgattgagatcatctcagactgcaactgggaggaagatcggaacaagattttgagcatcttatcccagcacaccaatagcaacatgccacaatcacttaaggtgggcagcttcatcattgagttggcttctcagcgaaagagccggggtgagaagaaccctcctgtttattcttctcgtgtgaaaatctctatgccatcatgtcaagaccaagatgatatggctgagaaatctggatcagagactcctgatggtccattgtcccctgggaaaatggaggatatctctcctgtgcagacagatgccctggattcagtgagggagagattacatggaggcaaaggtctgcctttttatgcagggctttctcctgcagggaagcttgtggcctataaacgtaaacccagttcaagtacatctgggcttatccaggtagcatccaatgccaaggtggctgcatccaggaaaccacgtaccctgttgccttcaacatccaattccaaaatggcatcctcctctggcactgcaacaaatcgccctgggaagaatctgaaggcgtttgtcgcagcaaaacggccaattgcggctcgaccctctcctggtggtgtgttcacacagtttgtgatgagtaaagttggagccttgcagcagaagatacctggagttagcacaccccaaaccctggcagggacacagaagttcagtatcagaccttctccagtaatggtcgtcacacctgtggtttcttctgagccagttcaggtgtgcagccctgtgactgctgctgtcactactaccacccctcaagtgtttttagaaaatactactgctgtgacacctatgactgctatttctgacgtggaaactaaagaaactacttattcttctggtgccaccactacaggggttgttgaggtctctgaaactaataccagcacctctgtaacatctacccagtctacagccactgtgaaccttaccaaaaccactgggataactacccctgtggcttcagttgcttttcctaagtctttggtagcatctccttcaaccataactcttcctgttgcttccactgcttccacctccttagtcgtggtgactgcagctgcatcttcctccatggtgaccacaccaacttcatctctgggctctgttcctattatactctcaggaattaatgggagtccaccagtgagccagagaccagaaaatgctgctcaaattccagtggctactccacaggtctctcctaacacagtgaaacgtgctggacctcgattgttgttgattccagtgcagcagggttctcctactcttagacctgtctcaaacacacaacttcagggacatcggatggtcttgcagcctgttaggagtccaagtggaatgaacttattcaggcaccctaatgggcagattgtccagcttctacctttgcatcagcttcgaggctctaatacccagcccaacttacagcctgtcatgtttcggaacccagggtctgtgatgggaatccggttacctgctccttccaaaccctctgagactccgccatcttccacttcgtcctctgctttctctgtcatgaatcctgtaattcaagctgttgggtcttcttcagcagtgaatgttatcactcaggcaccatcattgctttcctctggagctagttttgtgtctcaggctggtacattgaccctgaggatttctcctcctgaaccacaaagctttgcaagtaaaacaggctctgaaaccaaaataacttatagctcaggaggacagcctgttggtacagccagtcttattcctctccagtctggtagttttgccttgttacagctcccaggacaaaagcctgttcctagctccattcttcagcatgttgcttcccttcagatgaaaagagaatctcagaatccagaccagaaagatgaaacaaactcaataaaaagagagcaagaaacgaagaaggttctacagtcagaaggagaggctgtagaccctgaggctaatgtaataaaacaaaactcaggagctgctacctcagaagaaactctgaatgattccttggaagataggggtgatcatttggatgaagaatgccttccagaagaaggttgtgcaactgtcaaaccatctgagcattcctgtatcactgggtcacatacagatcaagattataaagatgttaatgaagaatatggggctaggaatcgtaagagttccaaagaaaaagtggctgttctggaagttaggaccatttctgaaaaagccagtaataagacagtccaaaatttaagtaaagtacagcatcaaaaacttggtgatgtgaaggtggaacagcagaaaggatttgacaatccagaagaaaactcaagtgaatttccagtcacctttaaggaagaaagtaaatttgaattgtcaggaagcaaagttatggagcagcaatctaatctacagccagaggccaaagagaaggaatgtggagactctctggagaaagacagggaaagatggagaaaacatctgaagggccccttaaccaggaaatgtgttggagcttcacaggaatgtaagaaagaggcagacgagcagttaattaaagaaacaaagacatgtcaggaaaattcagatgtgtttcagcaagaacaaggcatctctgacttacttggaaaaagtggaattactgaagatgccagagttttgaaaactgaatgtgattcttggagtaggatttctaatccttcagccttctccattgttcctaggagagctgcaaaaagcagcagagggaatggacattttcagggtcacttactgctacctggagaacagatacaaccaaagcaagagaagaagggtgggagaagcagtgctgacttcactgttttggatttggaagaagatgatgaagatgataatgagaaaactgatgattctattgatgaaattgtggatgttgtttctgactaccagagtgaggaggttgatgatgtagaaaagaataactgtgtagaatacattgaggatgatgaggagcacgtggacattgagactgtagaagagctctcagaggaaattaatgttgctcacctgaagaccacagcggcccacacacagtcattcaaacagccgtcctgtactcacatctctgcagatgaaaaagcagctgaaaggagtcgaaaggctccaccaattcctctaaaactgaagcctgattactggagtgacaaactacagaaagaagcagaagcgtttgcttattatcgccggacacacactgccaatgagcggcggcggcgtggtgaaatgagggatctctttgagaaattaaagatcacattgggattacttcattcttccaaggtttccaaaagtctcattcttactcgagccttcagtgaaattcagggactaacagatcaggcagacaaattgataggacagaaaaatctcctgactcgaaaacggaatattctgatacggaaagtatcgtctctttcaggtaagacagaagaagtggtcctgaagaagctagagtatatttatgcaaaacagcaagcactagaggcacaaaaaagaaaaaagaagatgggatcagatgagtttgacatatctcccagaattagcaaacagcaggaaggatcttctgcatcatctgtagatcttggacagatgtttataaataacaggagggggaaacctttgattctttccagaaaaaaagaccaggccacagaaaatacctcacccttgaacactccacacacctctgccaaccttgtgatgactccgcaagggcaattgctcaccctaaaaggtcccctattctcaggaccagtggtagctgtttctcctgatctcttagaatctgatcttaagcctcaagttgccggtagtgctgtggctctaccagaaaatgacgacttatttatgatgccacgaattgttaatgtgacatcattggccacagagggaggtttggtagatatgggtggcagcaaatatcctcatgaagttcctgatagcaagccatctgaccatctgaaagacaccgtcaggaatgaagataattccttagaggataagggtagaatctcttccagaggaaacagagatggcagagtgacgttgggtccaacgcaggtttttctggcaaacaaagattctggttatccacaaatagttgacgtttccaatatgcagaaagcacaagagttcttacctaaaaagatttctggtgatatgagagggattcagtataaatggaaagagagtgaatcaagaggggagagagtgaagtcaaaggattcttcatttcataaattaaagatgaaagatctcaaggactcaagcatagagatggaactgaggaaagtaacatcagctatagaggaagcagctcttgattccagtgaactgctgactaacatggaagatgaggatgatactgatgagacactgacttcactgctcaatgaaattgcctttcttaatcaacaactaaatgatgactctgttggcctggctgaactacccagctctatggatacagagttcccaggggatgctcggcgggcttttattagtaaggttcctcctggaagcagagcaactttccaggttgagcacttgggaactggtttgaaagagttgcctgatgttcaaggggagagtgactctatcagtcccctcctcttgcacttggaagacgatgacttttctgagaatgaaaaacaacttgcagaaccagcctctgagccagatgtccttaagattgttattgactctgaaataaaggattccctcctttccaacaagaaagctattgatggagggaagaatacttctggcctccctgcagagcccgaaagtgtgtcctcaccccccaccctacacatgaagactggcttggagaacagcaacagcacagacactttgtggaggcctatgccaaagttggcccctctaggtttaaaagtagctaatccttccagtgatgcagatggtcagagtctcaaggtgatgccttgtttggcacctatagctgccaaagttgggtcagttggacacaaaatgaacttaacagggaatgaccaggaaggccgggaaagcaaggtgatgcctacattggcacctgttgtggctaaattgggcaactcgggggcctcaccaagttctgcagggaaatgaacttacttgtccttaagcagaagccaggctgtgaggggaaattgatctcacctcctttctctgcaggcatctgtttgtttgtgtcttagaacttggatccttgacttcaatgatgcagtggataatgatgggagaaagggggtagggtgctgaccctggtataagaagtactctgaaattctgatcatgttaaaatgtgttacctcacttgtggtgctgggtcccctcatcctctctaagaagatgtgatccattactgaacatgaggtgcccctcttacccaaggaatttataacatgactctggctccacagtggtttctagttcttcccccacaagcgaaagagctgtttgcaactttggagttgctgtagactgaactgtagcttgtagctgttgaattaagtccaaaatctaaggaatgcgatggacttgtgcaaagggatccagaagagacactttttgacgttcgatgttctcaatgaataagggagagagaggatgggtcccatgggaataaccaaagtgaaggactttgtgtccttcagtaaaattttcttttttcatatc

SEQ ID NO: 53 - DNA, 4607 bp, Homo sapiens
  misc_feature: Incyte ID No 7499710CB1
gccagcgatcagagcagcgctgggtgttcaggggccaagatggcggcgcgccggggacggagagacggagtcgcgccgcccccgagtgggggccccggtccggaccctgtcgggggagcccgcggcagtggttggggaagtcgaagccaagcgccgtatgggactttgggcgctgtgagcggcggcgagcaggtgctgctgcatgaggaggcgggtgattctggctttgtcagtctctctcggctgggcccatctctgagggacaaggacctggaaatggaggagctaatgctgcaggatgagacactgctggggaccatgcagagctacatggatgcctcccttatctccctcattgaggattttgggagccttggagagagcaggttatctctggaggaccagaatgaagtgtcgctgctcacggctctgacggagatcttggacaatgcagattctgagaacctttctccatttgacagcattcctgattcggagctgcttgtgtcaccccgggagggctcctctctgcacaagctgcttactctctctcggacacccccagaacgtgacctcatcaccccagttgacccactggggcccagtacaggcagcagtagagggagtggggttgaaatgtctcttccagatccctcttgggacttctccccaccctctttcttagagacctcttcccccaagcttcctagctggagacccccaagatcaagaccacgctggggccaatccccacctccccagcagcgcagtgatggagaagaagaggaggaggtggccagcttcagtggccagattcttgccggggagcttgacaactgtgtgagcagtatcccggacttccccatgcatttggcctgccctgaggaggaagataaagcaacagcagcagagatggcagtgccagcagctggtgatgagagcatctcctccctgagtgagctggtgcgggccatgcacccatactgcctgcccaacctcacccacctggcatcacttgaggatgagcttcaggagcagccagatgatttgacactgcctgagggctgcgtagtgctggagattgtggggcaggcagccacagctggcgatgacctggagatcccagttgtggtgcgacaggtctctcctggaccccggcctgtgctcctggatgactcgctagagactagttctgccttgcagctgcttatgcctacactggagtcagagacagaggctgctgtgcccaaggtaaccctctgctctgagaaagaggggttgtcattgaactcagaggagaagctggactcagcctgcttattgaagcccagggaggtcgtggagccggtggtgcccaaggagcctcagaacccacctgccaatgcagcaccaggttcccagagagctcgaaagggcaggaagaagaagagcaaggagcagccagcagcctgtgtggaaggctatgccaggaggctgaggtcatcttctcgcgggcagtctactgtaggtacagaagtgacctctcaggtagacaacttgcagaaacagcctcaggaagaacttcaaaaagagtctgggcctctccagggtaaggggaagccccgggcttgggctcgggcctgggcagctgccttggagaattctagccctaagaacttggagagaagtgctggacaaagtagtcctgctaaagaaggccctctagacctctacccaaagctggctgacactatccaaaccaatcctataccaacccatctctcattggtcgactctgcccaagccagccccatgccagttgactctgttgaagctgatcccactgcagttggccctgttctagctggccctgtacctgttgaccctgggttggttgaccttgcttcaaccagctcagaactggttgagcctctcccggctgagccagtgctgatcaacccagtcctggctgactcagcagcagttgaccctgcagtggttcccatctcagataacttgccaccagttgatgctgtcccgtctggcccagcaccagttgatctagcactggttgaccctgttcctaatgacctgactccagttgacccagtgctagttaagtccagaccaactgatcccagacgtggtgcagtgtcatcagccctggggggttcagcaccccagctcctcgtggagtcagagtccttggacccaccaaagaccatcatccctgaagtcaaagaggttgtggattctctgaaaattgaaagtggtaccagtgctacaacccatgaagccagacctcggcctctcagcttatctgagtaccggcgacgaaggcagcaacgccaagcagaaacagaagagagaagtccacagcccccaactgggaagtggcctagccttccagagactcccacagggctggcagacatcccttgtcttgtcatcccaccagccccagccaagaagacagctctgcagagaagccctgaaacaccccttgagatttgccttgtgcctgtaggtcccagccctgcttctcctagtcctgagccacctgtaagcaaacctgtggcctcatctcccactgagcaggtgccatcccaggagatgccactgttggcgagaccttcccctcctgtgcagtctgtgtcccctgctgtgcccacacctccctcgatgtctgctgccctgcctttccctgcaggtgggcttggcatgccccccagtctgcccccacctcccttgcagcctcctagtcttccattgtctatggggccagtactacctgatccgtttactcactatgcccccttgccatcctggccttgttatcctcatgtgtccccttctggctatccttgcctgccccccccaccaacggtgcccctagtgtctggtactcctggtgcctatgccgtgcctcccacttgcagtgtgccttgggcaccccctcctgccccagtctcaccttacagttccacatgtacctatgggcccttgggatggggcccagggcctcaacatgctccattctggtctactgttcccccacctcctttgcctccagcctccattgggagagctgttccccaacctaaaatggagtctaggggcactccagctggccctcctgaaaatgtacttcccttgtcgatggctcctcccctcagtcttgggctacctggccatggagctcctcagacagagcctaccaaggtggaggtcaagccagtgcctgcatctccccatccgaaacacaaggtgtctgccctggtgcaaagtccccagatgaaggctctagcatgtgtgtctgctgaaggtgtgactgttgaggagcctgcatcagagaggctaaagcctgagacccaagagaccaggcccagggagaagccccccttgcctgctaccaaggctgttcccacaccaaggcagagcactgtccccaagctgcctgctgtccacccagcccgtctaaggaagctgtccttcctgcctaccccacgtactcagggttctgaagatgtggtacaggctttcatcagtgagattggaattgaggcatcggacctgtccagtctgctggagcagtttgagaaatcagaagccaaaaaggagtgtcctcctccggctcctgctgacagcttggctgtaggaaactcagggtccagctgtagttcctctggacgttctcgaagatgctcttcctcttcttcgtcatcatcttcctcttcgtcttcctcatcctcatcatccagttctcgaagccgctcacgatccccatccccccgccggagaagtgacaggaggcggcggtacagctcttatcgttcacatgaccattaccaaaggcaaagagtgctacaaaaggagcgtgcaatagaagaaagaagggtggtcttcattggaaagatacctggccgcatgactcgatcagagctgaaacagaggttctccgtttttggagagattgaggagtgcaccatccacttccgtgtccaaggggacaactacggcttcgtcacttatcgctatgctgaggaggcatttgcagccattgagagtggccacaagctgcggcaggcagatgagcagccctttgatctctgctttgggggccgaaggcagttctgcaagaggagctattctgatcttgactccaaccgggaagactttgacccagcacctgtaaagagcaaatttgattctcttgactttgacacattgttgaaacaggcccagaagaacctcaggaggtaaccttgggcccttccctgctatcctttttctcctttggaggtgcccaacctcctccacccccttcccctactctaggggagagagctgctagtgagatgactgttttataaagaaatggaaaaaagtgaaataaaaaatatgttgaatcagattttttaaaaggggtatttgtttttttataacaggtattgaaacaagttaacttgcattcctatgtaagataggaggggctgaggggatccccagtgtttggaacataagtcactatgcagactaataaacatcaactagagagaactccaaaaaaaaaaaaaaacaaaaacccaccaaacaaaacaaacacacaccccaaaccaaaaaaaaga

SEQ ID NO: 54 - DNA, 4486 bp, Homo sapiens
  misc_feature: Incyte ID No 8036958CB1
gcgcacgtggcagccccggagccggggaatgtgaagagctctcggctgtgcagtggtaccgtcggggcctgggccgcgaaggatcttctgccacagctgcaacatgggcggcaagaacaagaaacacaaggctccagcggccgcggtggtccgggccgccgtgtctgcttccagagccaaatctgccgaggctggaattgccggggaggcccaaagcaagaagccagtgtccaggccggccaccgctgccgctgccgctgccggctccagggagccccgtgtcaagcaaggtccaaaaatttatagttttaattctacaaatgattctagtggtcctgcaaatctggataaatctattttgaaagtggtaattaataacaaactagagcaaagaattattggagtgatcaatgagcataaaaagcaaaataatgacaaaggaatgatttctggaagacttactgccaaaaaattgcaggatttatacatggctttacaagcattttcatttaagacaaaggacattgaagatgccatgaccaatacactcttatatggaggtgaccttcattctgccttggattggctctgtttaaacctttcagatgatgcacttcctgaaggattcagtcaggaatttgaagagcagcaacctaaaagtaggcctaaatttcagtctcctcaaatacaagccactatttcacctccattgcaacctaaaacaaaaacatatgaagaggaccctaagagtaagccaaaaaaggaagaaaaaaatatggaagtaaatatgaaagagtggattttacgatatgctgaacaacaaaatgaagaagaaaagaatgagaattctaaaagtttagaagaggaggaaaaatttgaccctaatgaaaggtacttacatcttgcagcaaaactgctggatgcaaaagaacaagcagctacctttaaactagaaaaaaacaagcaaggccaaaaagaggctcaggaaaaaataaggaaatttcaaagagaaatggaaactttagaagaccatccagtatttaacccagccatgaagatttcacatcaacaaaatgaaaggaaaaagcctcctgtagccacagaaggagaaagtgcattgaattttaatttatttgaaaaatctgcagctgctactgaagaagagaaagataaaaagaaagaacctcatgatgtaagaaattttgactatactgctcgaagttggactggaaaatctcccaaacaatttctgattgattgggtcaggaagaatcttcccaagagtccaaatccttcctttgaaaaagttccagtaggtagatactggaaatgtagggttagggtaatcaagtctgaagatgatgtcctggtagtatgccctacaatcttaacagaagatggcatgcaagctcagcacctgggagctactttagccctttaccgtttagttaaagggcagtcagttcatcagttacttcctcccacttaccgagatgtttggctggagtggagtgatgcagaaaagaaaagggaagaattaaataaaatggaaaccaataaaccacgtgatctttttattgccaaacttctgaataaactgaaacagcagcaacagcagcaacaacagcattctgaaaataagagagaaaactctgaagatcccgaggaatcttgggaaaatttagtttcggatgaggatttttctgcactgtccttggaatcagcaaatgtggaagatttggaacctgttagaaacctctttagaaagttgcaaagcacacctaagtatcagaaacttctaaaggaaagacaacagctacctgtatttaaacatcgggactcaattgttgaaactcttaaaaggcatcgggtagtggttgtggcaggtgaaacagggagtggtaaaagtactcaggtaccacattttctattggaagatttgcttctaaatgagtgggaagcaagtaaatgtaacattgtctgtacccaaccccgaagaatctcagcagttagtttagccaacagagtatgtgatgaattgggctgtgaaaatggacctggaggaaggaattccttgtgtggatatcagatccggatggaatctcgagcttgtgaatctaccaggttactctattgtacaacaggggttttgctaaggaaacttcaagaagatggtcttctaagtaatgtgtctcatgttattgtagatgaggttcatgaaagaagtgtccagtcagacttcctactaattatcttgaaggaaattttacagaaacgttctgatctacacttgattctaatgagtgccactgtggacagcgaaaaattttctacatatttcacacactgccccattctcagaatttcaggaagaagttatcctgttgaggtttttcatcttgaagatataatagaagaaacaggctttgtactggaaaaagactcagaatattgtcagaaatttctggaagaggaagaagaagtaaccattaatgttacaagcaaagcagggggaataaaaaaatatcaggaatacatcccagttcagactggagcacatgctgatttaaatccattttaccaaaagtacagcagccgcactcagcatgctattctatacatgaatcctcataaaatcaacctggatctcattttggaacttcttgcatacttagataaaagtccccaattcagaaatattgaaggagcagtattgatctttttaccaggacttgctcatattcagcagttgtatgatcttctatcaaatgatagaagattttattctgaacgatataaagtgatagctctgcattctattctttcaacccaagatcaagctgcagcattcacacttccccctccaggagtcaggaagattgttttagcaaccaatattgcagagacgggtatcactattcctgatgttgtatttgtaattgatactggaagaacaaaagaaaataagtaccatgaaagcagtcagatgagttctttggtggagacgtttgtcagtaaagccagtgctttgcagcgccagggaagagctgggcgggtcagagatggcttctgtttccgaatgtacacaagagaaagatttgaaggctttatggattattctgttcctgaaatcttacgtgtacctttggaggaattatgccttcatattatgaaatgtaatcttggttctcctgaagatttcctctccaaagccttagatcctcctcagctccaagtgatcagcaatgcaatgaatttgctccgaaaaattggagcttgtgaattaaatgagcctaaactgactccgttgggccaacaccttgcagctttacctgtgaatgtcaagattggcaagatgcttatttttggtgccatatttggctgccttgacccagtggcaacactagctgcagttatgacagagaagtctccttttaccacaccaattggtcgaaaagatgaagcagatcttgcaaaatcagctttggccatggcggattcagaccacctgacgatctacaatgcatatctaggatggaagaaagcacgacaagaaggaggttatcgttctgaaatcacatactgccggaggaactttcttaatagaacatcactgttaaccctagaggatgtaaagcaggagttaataaagttggttaaggcagcaggattttcatcttccacaacttctaccagctgggaaggaaacagagcctcacagaccctctcattccaagaaattgcccttcttaaagctgtactggtggctggactgtatgacaatgtggggaagataatctatacaaagtcagtggatgttacagaaaaattggcttgcattgtggagacggcccaaggcaaagcacaagtacacccatcctcagtaaatcgagatttgcaaactcatggatggctcttataccaggagaagataaggtatgccagagtgtatttgagagaaactaccctaataaccccttttccagttttactttttggtggtgatatagaagttcagcaccgagaacgtcttctttctattgatggctggatctattttcaggcccctgtaaagatagctgtcattttcaagcagctgagagttctcattgattcagttttaagaaaaaagcttgaaaatccaaagatgtcccttgaaaatgacaagattctgcagatcattacggaattgataaaaacagagaataactgaaactgaaattcatggtcaactgctttaaaaattaagatgaagatacagtcatgaaattatctgaaaatgggtcatcacattaagtatttcattacttaaaatgttggtactagccattaacttaaaggtggtgggaaaaaagcacatactttaaacatgtataattttctagttcctttttaatgatgattattctgaatgtatttgccactacatttacaataaattctttggtattatgaaaaaaaaaaaaaaaaaaaagggcggccgctc

SEQ ID NO: 55 - DNA, 2559 bp, Homo sapiens
  misc_feature: Incyte ID No 3253807CB1
gtcgcgggccggaccgggtcccggggcggtgggagccccggccgggcagaagggcttggcgggccgttagaggaccgccacggctgtcgagtcccctcccttgttggacttgcgcgccctggcgctcggaacctccggcgctgtgcccaccccgctctagctcgcgtctcccgactccaattagagcagcccgacggccatggaggctgaagagacgatggaatgccttcaggagttccctgaacatcataaaatgatcctcgaccgattgaatgaacagcgagagcaggaccggtttactgacatcaccctaattgtcgacggacaccattttaaggctcacaaggctgttttggctgcttgtagtaagttcttctacaaattctttcaggagtttacccaagaaccattggtggagatagaaggtgttagtaaaatggcctttcgccatttaattgagttcacatatacagcaaaattaatgatacaaggagaagaagaagccaatgatgtatggaaagcagcagagtttctacaaatgctagaagctatcaaagcccttgaagtcaggaacaaagaaaactcagctcccttagaggaaaataccacaggaaaaaatgaggccaaaaaaaggaagattgcagaaacttcaaatgttatcactgagtcattgccatctgcagaatcagaacctgttgaaattgaggtagagattgccgaaggcaccattgaagtggaagatgaaggcatcgaaacattagaggaagtggcttctgccaagcagtccgtaaagtacatacagagcacaggttcctctgatgattctgctctagcactgttggcagatattaccagcaagtaccgtcaaggtgacagaaaagggcagattaaagaagatggctgtccatctgaccccacgagcaaacaggagcacatgaaatcacactccactgagagtttcaagtgtgaaatatgcaataaacgatatcttcgagagagcgcatggaaacagcacctaaattgttaccaccttgaagaaggtggagtcagtaagaagcaaagaactgggaaaaaaattcatgtatgtcagtactgtgagaaacagtttgaccattttggacattttaaagaacatcttcgaaaacatacaggtgaaaaaccttttgaatgtccaaattgtcatgaacgatttgctagaaatagcactctgaaatgtcacctcactgcatgccaaactggagtaggggcaaaaaaaggaaggaagaagctctacgaatgccaggtctgcaacagtgtgtttaacagctgggaccagttcaaagatcacttggtaatacacactggagataaacccaaccattgtactttatgtgatttgtggtttatgcaaggaaatgaattaaggaggcatctcagtgatgctcacaatatttcagagcgtctagtaacggaagaagttctttcagtagaaacacgtgtgcaaactgaacctgtaacatcaatgactattatagaacaagttgggaaggtgcatgtgctaccattgcttcaggttcaggtggattcagcacaagtgactgtggaacaagtccatccagatctgctccaggacagccaggtgcacgattcacacatgagtgagcttccagagcaggtccaagtgagttatctagaagtgggccgaattcagactgaagaaggtactgaagtacatgtagaggagctgcatgttgaacgggtcaatcaaatgccagtggaagtacaaactgaacttctagaagcagatttggaccacgtgaccccagaaatcatgaaccaagaggagagagagtctagccaagcagatgctgctgaggctgccagggaagatcacgaagatgctgaggatttagagaccaagccaacagtggattctgaagcagaaaaggcagagaatgaggacagaacagctctgccagttttagaatgaaattacacatgaatatatttttaaatttacttgttgggtttttgaactgattatgggcagtttgactgtccttaattaagcctaacagacaagtggaccaaagttaagctgtttcctgttgtgctgaactgttgtccgttgaaacacattgattcccctccccctacttattgccacagaggagggatcttttccataactgaaggggagttttgagaagtatatttctggaaacttaaatggattatattcttattatatagttgggtacgaatgtatctattttcattgtggtaaaagttcttccttttctctttcccaggtcatgttcttcctcaaattttttccatattgtaaaatcaaacttaaatcattagaatacaagtttatgtattctaatgcatgttagaaaattgaataatataggaaacacaaggctgcatgatgaaaagtgcattgttactgtgcagttaaattttggcttctggctttctttagtttgaacaaacgttcttgtctaccccagtagtcacagatgccatctttgcaacagaaagagtggtggtggcaaaatttctagaatgtaaaaa

SEQ ID NO: 56 - DNA, 839 bp, Homo sapiens
  misc_feature: Incyte ID No 3626408CB1
ctagcgccggcgcctccacttcgttttcctcactctctcctccagctcagggtccggcggcgaagggaaggcaagatgtacaccgcgaggaagaagatccagaaggagaagggtcttgagccctccgagttcgaggactccgttgcccaggctttctttgatctggagaacgggaaccaggagctcaagagcgacctcaaggacctgtacatcaacaatgctatccagatggatgttaccgggagtaggaaggctgttgtcattcacgtcccataccgcctgcgcaaggccttcaggaagatccatgtcagactcgtcagggagctggagaagaaattcagcggcaaggatgtggtaattgttgctacacggaggattgtgaggccacccaagaagggttcagctgttctgcgccctcgcaccaggactctgactgctgttcacgatggcatcttggaggatgttgtctacccagctgagattgtggggaagcgtgtcagataccgtctggatggttccaagattatcaagattttcttggacccaaaggagaggaacaacactgaatacaagctggagacctgcactgcggtctaccgcaggctgtgtgggaaagatgtggtctttgagtaccctatgaccgaaaatgcataaatatgatgccctctggatatctccactctatttcgttgattctgaatgttatgttgggtctccctatgtacttcaaaaaacatagttttgatgctaaagttgcctaggaatttggtacggtgaagtggcagtggacgtgaacaatgttaagttttgagcatttaaattaaaaaaaaaaaaaaagg

SEQ ID NO: 57 - DNA, 4898 bp, Homo sapiens
  misc_feature: Incyte ID No 3773014CB1
attgagagagaaagagagagagtcaagagcagagaatcagagagagagagagagtctgtgtctctgggaaagaagaacatctctgcttcacagtgatttgcgctgggggagaggcatcaattggcttcggacccaagggggagacgagaccaggtcaccccggttaagaccaagtgagcgttgcccctccctctcccaactctctacccgggaatgtctcggcgaaagcagcggaaaccccaacagttaatctcggactgcgaaggtcccagcgcgtctgagaacggtgatgctagcgaggaggatcacccccaagtctgtgccaagtgctgcgcacaattcactgacccaactgaattcctcgcccaccagaacgcatgttctactgaccctcctgtaatggtgataattgggggccaggagaaccccaacaactcttcggcctcctctgaaccccggcctgagggtcacaataatcctcaggtcatggacacagagcatagcaaccccccagattctgggtcctccgtgcccacggatcccacctggggcccagagaggagaggagaggagtcttcagggcatttcctggtcgctgccacaggtacagcggctgggggaggcgggggcctgatcttggccagtcccaagctgggagcaaccccattacctccagaatcgacccctgcaccccctcctcctccaccaccccctccgcccccaggggtaggcagtggccacttgaatatccccctgatcttggaagagctacgggtgctgcagcagcggcagatccatcagatgcagatgactgagcaaatctgcaggcaggtgctgttgcttggctccttaggccagacggtgggtgcccctgccagtccctcagagctacctgggacagggactgcctcttccaccaagcccctactacccctcttcagccccatcaagcctgtccaaaccagcaagacactggcatcttcctcctcctcctcctcttcctcttcaggggcagaaacgcccaagcaggccttcttccacctttaccacccactggggtcacagcatcctttctctgctggaggggttgggcgaagccacaaacccacccctgccccttccccagccttgccaggcagcacagatcagctgattgcctcgcctcatctggcattcccaagcaccacgggactactggcagcacagtgtcttggggcagcccgaggccttgaggccactgcctccccagggctcctgaagccaaagaatggaagtggtgagctgagctacggagaagtgatgggtcccttggagaagcctggtggaaggcacaaatgccgcttctgtgccaaagtatttggcagtgacagtgccctgcagatccaccttcgttcccacacgggtgagaggccctataagtgcaatgtctgtggaaaccgttttaccacccgtggcaacctcaaagtgcatttccaccggcatcgtgagaagtacccacatgtgcagatgaacccacacccagtaccagagcacctagactatgtcattaccagcagtggcttgccttatggtatgtccgtgccaccagagaaggccgaggaggaggcagccactccaggtggaggggttgagcgcaagcctctggtggcctccacaacagcactcagtgccacagagagcctgactctgctctccaccagtgcaggcacagccacggctccaggactccctgctttcaataagtttgtgctcatgaaagcagtggaacccaagaataaagctgatgaaaacacccccccagggagtgagggctcagccatcagtggagtggcagaaagtagcacggcaactcgcatgcaactaagtaagttggtgacttcactaccaagctgggcactgcttaccaaccacttcaagtccactggcagcttccccttcccctatgtgctagagcccttgggggcctcaccctctgagacatcaaagctgcagcaactggtagaaaagattgaccggcaaggagctgtggcggtgacctcagctgcctcaggagcccccaccacctctgcccctgcaccttcatcctcagcctcttctggacctaaccagtgtgtcatctgtctccgagtgcttagctgtcctcgggccctacgccttcattatggccaacatggaggtgagaggcccttcaaatgcaaagtgtgtggcagagccttctccaccaggggtaatctgcgtgcacatttcgtgggccacaaggccagtccagctgcccgggcacagaattcctgccccatctgccagaagaagttcaccaatgctgtcactctgcagcagcatgtccggatgcacctggggggccagatccccaacggtggtactgcactccctgaaggtggaggagctgctcaggagaatggctccgagcaatctacagtctccggggcagggagtttcccccagcagcagtcccagcagccatcaccggaagaggagttgtctgaggaggaggaagaggaggatgaggaagaagaggaagatgtgactgatgaagattccctggcagggagaggctcagagagtggaggtgagaaggcaatatcagtgagaggtgattcagaagaggcatctggggcagaggaggaggtggggacagtggcggcagcagccacagctgggaaggagatggacagtaatgagaaaactactcaacagtcttctttgccaccaccaccaccacctgacagcctggatcagcctcagccaatggagcagggaagcagtggtgttttaggaggcaaggaagaggggggcaaaccggagagaagctcaagtccggcatcagcactcaccccagaaggggaagccaccagcgtgaccttggtagaggagctgagcctgcaggaggcaatgagaaaggagccaggagagagcagcagcagaaaggcctgcgaagtgtgtggccaggcctttccctcccaggcagctctggaggagcatcagaagacccaccccaaggaggggccgctcttcacttgtgttttctgcaggcagggctttcttgagcgggctaccctcaagaagcatatgctcctggcacaccaccaggtacagccctttgccccccatggccctcagaatattgctgctctttctctagtccctggctgttcgccttccatcacctccacagggctctccccctttccccgaaaagatgaccccacgatcccatgagcctgtttttctgtacctgctgctctttgtcccacagagcagaaacagcttcacaaaaggacctcccagagttatgagccctgattttgtctttttctctaagttcttaacatgttatgtccctagtggcttttctgtagtccctgagcttggaaattactgtgcttacaaggggatggccccctaaggaatttttcttccctcctcattctttgtacctgaggaacatagattctctgcagctttctcaaggggaaccctctccagcttccctggtgtgacccttcttccccctcctctctcctctccctttccctttggtaggtgcacctgagcacctacatttggcattgcagcctagccaaaaagggctggcagctgtctctggagggcccagtgccactcctctggggtgacctttctgctcagctggtgggtatgggtcccctatctttctagaaccagtatgtggcattcctgtcaaatggcctgcccatgaagccctggaattccagctccacctccactaccactccaagcctggccccaccagtgctgtttggcctaggaactgtggctgggaaggtgcctccaacaatgggatccagggaagccaaggagaagacagcccccctcctatttcagcctcctgcacccaaggcagtgcctgagaagcccatcatagacaagaagtagcaaactgtacattccttcttcctccccctgctccagaaggtgccggtactgaagatgctccagtaattggtgacccaaccctaggaagtagggagaaatgaaggaagggcataggaaaattttcccagtaaatcccctgatggtcacattaaggtaaaggttttggctggtcagtgtgccaagacctctccagcttctcattcatgatgacctctcaaagttgggaaacaagctgatttcttgccaagaggtctcccaggagatatttgggaaatgtgaagttcgtatctttaaggagcatttttggtcagcatggttgatgaactaatgatgagagagttaaggaatgttgctagaacatagggcttgctggtacctatgtgactaagaaagggacatgatgtaagggaaaaggcctcaaattcttgtgaatgtggacattctcgttaatattcttttgggctaatagtgacatagtgtgcagaggtgtaccagggatcatgggggatttcctagcactagtatgcttctagttttagataactccctcctttattccctggccccttgtattttccttatcttcctctttcaagacccctacccattttgcctatccgtaggctggggcttgtgtctttgtcattgtctggttcttaagagtcccagactttgggagaccagctccaggtggcgtcctccctgcctctccgtcttgtaatgagttgtagtatttactcttaacataggatcatttggaacaggagttctgaggaggagagagtgagggttttgctattgactgacttgaacgatggcttctcctcaagctgtaggctccagagcttcctaacctagtaaaatgtcaagaacagacgggagatattagtgtctttccctctatcattaaaggtgttttaaccaaaaaaaaaaaaaaaaaaaaaaaaaaaaaaaaaattgcggtc

SEQ ID NO: 58 - DNA, 3537 bp, Homo sapiens
  misc_feature: Incyte ID No 4398735CB1
catggctggcgtggcccgcgcggcggggcccgtgccaatcgcgcgtagggggctgtgggcactcggggttcgtagttttgaaatttctggcgggggagcagctgcgcagttaggctcgaggtgtgagcgacgaccttccgggagccgcaagtccaggctcccccgcagcgggacccgagcctgaggcgcagggctgaggcagcgcacgtgtgagcgccgctgaggaagctgcgagaggtcgggcgggtgtctgctccgggggcagcccaggctcgcgcggacgagaggaaggtccgggacgcgcgtgtcctgccgtgcagcgggcgcccgtcactgacttcgctgctgcggcccccgcgcctctccccagcgatgctgtggaacccgaaccgcaccggagtcggctgccgcgcgccaagcctcccctcacctctgctcccggaaccgcagcgccaaagctgccgctgagcccctgggggatgcccgccggcctcaccgaacccgccggcgccgctcccccggctgctgtgagcgcctcggggaccgtgaccatggccccggccggggcgctgccggtgcgggtggagagcactccggtggccctgggcgccgtgactaaggctcctgtcagcgtctgcgtggagcccacggcgtcccagcccctgcggtcccccgtggggaccctggtgaccaaagtggctccggtcagcgcccctcctaaagtcagcagcggccctaggctgcctgctcctcagatagtcgccgtgaaagcccccaacaccacgacaatccagtttcctgctaatttgcagcttcctccaggtatgttaggaaccgttttgattaaaagtaacagtggtccgttgatgttggtatctcctcagcaaactgtaacaagagccgagaccacaagtaacataacctcaaggccagcagtaccagcgaatcctcaaacagtcaaaatctgtacagtgccgaactctagctcacaattaatcaagaaagtggcagtgacacctgttaaaaaattggcacaaataggaactactgtggtaaccactgttccgaagccttcctcagtacaatctgtggctgtgccaaccagtgtcgtcacagttactcctggaaagccattgaatactgtaactaccctgaagccttcaagtttgggagcatcatccactccttcaaatgagcccaatcttaaagcagagaactcagcagctgttcagattaatctttctccgacaatgctagaaaatgtgaagaaatgcaagaacttccttgcaatgttaataaaactagcatgtagtggatcacagtcccctgaaatggggcaaaatgtgaagaagctggtggaacaacttttggatgcaaaaatcgaagcagaagaatttactaggaaactgtatgttgaactcaagtcttcacctcagcctcacctggttccttttcttaagaaaagcgtggttgccttacgacaacttctgcctaactcccagagcttcatccagcaatgtgttcagcagacttctagtgacatggtcattgctacctgtactacaacagtaacaacttctcctgtggtgacaactacagtgtcctcaagccagtctgaaaagtcaattattgtttctggagcaacagcacccagaactgtgtcagtgcaaactttgaacccacttgctggtccagtgggagcaaaagctggagttgtgacacttcattctgtgggcccaactgctgcaacaggaggaacaacagctggaactggtttgcttcagacttcaaaaccacttgtgacatctgtggcaaacacagtgaccacggtctcactgcaacctgaaaagccagttgtctctggaacagcagtaacactgtcccttccagcagtaacttttggagaaacttcaggtgcagctatttgtcttccatctgtgaaacctgttgtttccttctgctgggaccacatctgcaagcctgttattgggactccagttcaaatcaaacttgcccagccgggccctgtcctttcacaaccagctgggattccacaggcagttcaagtcaagcaactagtagttcagcagccttcaggaggcaatgaaaaacaagtgaccacaatttcacattcctcaacattgaccattcagaaatgtggacagaagacgatgccagtgaacaccataatacctactagtcagtttcctccagcttccattctaaagcaaattactctgcctggaaataaaattctgtcacttcaagcatctcctactcagaaaaatagaataaaagagaatgtaacatcatgcttccgagatgaggatgacatcaatgatgtgacttctatggcaggggtcaaccttaatgaagaaaatgcctgcatcttagcaacaaactctgaattggttggcacactcattcagtcatgtaaagatgaaccatttctttttattggagctctacaaaagagaattttagacattggtaaaaagcatgacattacagaacttaactctgatgctgtgaacttgatctcccaagcaacacaggaacgactacgaggccttctagaaaaactgactgcaattgctcagcatcgaatgactacttacaaggcaagtgaaaattacatcctgtgtagtgataccaggtcacagctcaaatttcttgaaaagctggatcaattggagaagcagagaaaggatttggaagaaagagaaatgttacttaaggcagccaagagtcgttctaataaagaagatccagaacagctgagattaaagcagaaagccaaagagttacagcaattggaacttgcacagatacagcatagagacgctaatctcacagctcttgcagctattggaccaaggaagaagagaccactagaatctggaattgagggcttaaaagacaaccttcttgcttctgggacatccagcctgacagccaccaaacagttgcatcgtccaagaatcacgagaatctgcctcagggacttgatattttgtatggaacaggaacgggagatgaagtattctcgagctctatacctggcccttctgaagtgaccactccactcttccatccagatccttgctatttactgccaaagaagacacaaagcattgttgcactgtcctgaaatttcaatttctggaaaataatcaccaacatgaaagagcattgtttacagttagaaactttattaactcttacctatccatctcatgggactcttacagactcagattcatctttgtcttctgaaaatcagttatgaaatacactttgcacagaattaggcatctgcctatctgtgcattaaattaaagcaagttaaggccctatttgttactacctgcctcatttgccaaattgtagcaggtgaggtgtccttccctaatacagcatgcattgagatgcaggagaaaggaagaggcataaggaaatactagaatgactatttcaatctatattgaacctgtcccaaagtggttaggtttctgggctggaaacagaagttgctta

SEQ ID NO: 59 - DNA, 1822 bp, Homo sapiens
  misc_feature: Incyte ID No 7499579CB1
agcgggcgctctactcctgtaacggaaaggtcgcggcttgtgtgcctgcgggcagccgtgccgagaatgaaccccagcacccccagctacccaacggcctcgctctacgtgggggacctccaccccgacgtgactgaggcgatgctctacgagaagttcagcccggcagggcccatcctctccatccggatctgcagggacttgatcaccagcggctcctccaactacgcgtatgtgaacttccagcatacgaaggacgcggagcatgctctggacaccatgaattttgatgttataaagggcaagccagtacgcatcatgtggtctcagcgtgatccatcacttcgaaaaagtggagtgggcaacatattcgttaaaaatctggataagtccattaataataaagcactgtatgatacagtttctgcttttggtaacatcctttcgtgtaacgtggtttgtgatgaaaatggttccaagggttatggatttgtacactttgagacacacgaagcagctgaaagagctattaaaaaaatgaacggaatgctcctaaatggtcgcaaagtatttgttggacaatttaagtctcgtaaagaacgagaagctgaacttggagctagggcaaaagagttccccaatgtttacatcaagaattttggagaagacatggatgatgagcgccttaaggatctctttggcaagttctggcctgccttaagtgtgaaagtaatgactgatgaaagtggaaaatccaaaggatttggatttgtaagctttgaaaggcatgaagatgcacagaaagctgtggatgagatgaacggaaaggagctcaatggaaaacaaatttatgttggtcgagctcagaaaaaggtggaacggcagacggaacttaagcgcaaatttgaacagatgaaacaagataggatcaccagataccagggtgttaatctttatgtgaaaaatcttgatgatggtattgatgatgaacgtctccggaaagggttttctccatttggtacaatcactagtgcaaagactcagaaccgtgctgcatactatcctcctagccaaattgctcaactaagaccaagtcctcgctggactgctcagggtgccagacctcatccattccaaaatatgcccggtgctatccgcccagctgctcctagaccaccatttagtactatgagaccagcttcttcacaggttccacgagtcatgtcaacacagcgtgttgctaacacatcaacacagacaatgggtccacgtcctgcagctgcagccgctgcagctactcctgctgtccgcaccgttccacagtataaatatgctgcaggagttcgcaatcctcagcaacatcttaatgcacagccacaagttacaatgcaacagcctgctgttcatgtacaaggtcaggaacctttgactgcttccatgttggcatctgcccctcctcaagagcaaaagcaaatgttgggtgaacggctgtttcctcttattcaagccatgcaccctactcttgctggtaaaatcactggcatgttgttggagattgataattcagaacttcttcatatgctcgagtctccagagtcactccgttctaaggttgatgaagctgtagctgtactacaagcccaccaagctaaagaggctgcccagaaagcagttaacagtgccaccggtgttccaactgtttaaacatcgatcagggggccatgaaaagaaacttgtgcttcaccgaaagaaaaatatctaaacatcgaaaaacttaatattctggcagaacaaacacttcgcaatcttcaaacaaacagag

SEQ ID NO: 60 - DNA, 2497 bp, Homo sapiens
  misc_feature: Incyte ID No 8178947CB1
cgcgcttgcagcgtctgggagaatctttcggtctccgcgagaggtgcttcattccacgaaaaaagtataattcaagctcagatttgtgttgaaaccagcctcaagtttcacctatcctcactgatccgtggacttctgtatgatcagggtgctgtcctgagagcgctgcgggataaaggaggagcgtcctgcttcccggctgccctgttgctgtcggagtcacaggatggcggctgtcgtcctgcccccaactgccgctctgtcttccctgttcccagcctctcagcgagaaggacacacagagggcggagagctggttaatgagctcctgaaaagctggctaaagggcttggtgacctttgaggatgtggccgtggagttcacccaggaggagtgggcattgctggaccctgcccaaaggacactgtacagggacgtgatgctggagaactgcaggaacctggcctcactggggaaccaagttgataaacctaggctgatctcccagctggagcaagaagataaagtgatgacagaagagagaggaattctctcaggtacctgtccagatgtggagaatccatttaaagccaaagggttaactcctaagctgcatgtttttcgaaaagaacaatctagaaatatgaaaatggagaggaatcatcttggagcaacactcaacgaatgtaatcagtgttttaaagtcttcagcacaaaatcttcccttacacggcacaggaagattcatactggagaaagaccctatggctgcagtgaatgtgggaaatcctacagcagtagatcttaccttgctgttcataagagaatccacaatggggagaaaccctatgaatgcaatgactgtgggaaaaccttcagcagcagatcttaccttactgttcataagagaatccacaatggggagaaaccctacgaatgcagtgactgtgggaaaaccttcagcaattcctcatacctcagaccgcacttgagaattcacactggagaaaaaccgtacaaatgtaaccagtgttttcgtgagttccgcactcagtcaatcttcacaaggcacaagagagttcatacgggggagggtcattatgtatgtaatcagtgtggaaaggctttcggcacgaggtcatctctttcttcgcactatagcattcatacaggggagtacccttacgaatgccacgattgtgggagaaccttcaggaggaggtcgaatctgacacagcacataagaactcatactggagaaaaaccctacacatgtaatgagtgtgggaaatcctttaccaatagcttttctcttacaattcacaggagaatacataatggagagaaatcctatgagtgcagtgattgtggaaaatcctttaatgttctctcatccgttaagaaacacatgagaactcacactggaaaaaaaccctatgaatgtaattattgcgggaaatccttcacaagtaactcctacctttctgtgcatacgagaatgcataataggcaaatgtgaattcaataactgtgggaaaagcattcattgatctttcatgcctcagataacatgagcaaactctaacaagatgtatgaatcacctgctactgtgtaacaaattaccccccaaattttgtggcttcaaacaaaaacacttgttatctcaaatttttgtaggtcaggaattcagaaacaacatagctctatagttcttcaggatatctcaagaggttacagtccagatgtcagcagaactgtaatcatccagaattgttactggtgaagggaccacttccaaatggcttacaagcccgacaagtgcatgctagctgtttccaagggacctgcgcttccaaatcacgtaggcctctcaacagggctgagtttctttatggaagcaacttctccccaagccagtgatatgttcaggaagttggaatatcaagagtcccagcaggaaacagttgacacacacgtcaactgggatgattcaagcgcagtttatatcaaggggctcctcacagaattgtgaacaggatgtagggaaaccacaaaaggtacttgaaatggaacatcggtcttctccccacctcagatacgggtgcttctggttctccagccttcagactcagactcgcaacttacaccattggccctcctggttctcaagcctttggacttcgcactggggtttacactgttggctccccctgttttcaggtctttgagcttggactggagcaagactaccagctttcttggttctccagcttccaaacaaatggcagatcgtaggacacccataagcctgaagggacaaaggaagagaactgcaagttgttgttcaagcgaggagagggctgcattccaggagctgacaggaggtcagtgaggggcaacatccagcctcgagttctggcatcctgcaaactcaatgagaggtgaagccagctggacttcctgggtcgagtggggacttggagaacttttctgttt

SEQ ID NO: 61 - DNA, 4943 bp, Homo sapiens
  misc_feature: Incyte ID No 2264652CB1
aagtaactagaaatttttggtttcctgtcgcagagtagctgtaaatgatcagtcaccaattatgtaataccagaagaaaggggttttataattctattccaattgcttattcaatataaacacttttacctaaattaattttatttttatttgctttctatttgtatttttagagtaacacatgaggtctcccatatatataaaagagtaacattctccaatttcctataccccagtagtagtcactgttttaagtttgatttgtattcttctagatatacttaaattatttaaaaataaatttaaatatttctgtacagtttgatcgggagcacttcagtgaagtttttgtggacctaaaatggtttgaaagcaaagttggtaacaagtacctcaatgaagcagcaggtgtcgcagcagaagaagccaggaactacaaggaaaagaaaaagttaaagggccaggaaaattctctgtgttggactgctttagacaaaaatgaaggcgaaatgataacttctaaggataatttagaagatgagactgaagatgatgacctatttgaaactgagtttagacaatataaaagaacatattacatgacgaagatgggggttgacgtagtatctgatgactttctggctgatcaagctgcatgttatgttcaggcaatacagtggattttgcactattactatcatggagttcagtcctggagctggtattatccttatcattatgcacctttcctgtctgatatacacaacatcagtacactcaaaatccattttgaactaggaaaaccttttaagccatttgaacagcttcttgctgtacttccagcagccagcaaaaatttacttcctgcatgctaccagcatttgatgaccaatgaagactcaccaattatagaatattacccacctgattttaaaactgacctaaatgggaaacaacaggaatgggaagctgtggtgttaatcccttttattgatgagaagcgattattggaagccatggagacatgtaaccactccctcaaaaaggaagagaggaaaagaaaccaacatagtgagtgcctaatgtgctggtatgatagagacacagagtttatctatccttctccatggccagaaaagttccctgccatagaacgatgttgtacaaggtataaaataatatccttagatgcttggcgtgtagacataaacaaaaacaaaataaccagaattgaccagaaagcattatatttctgtggatttcctactctgaaacacatcagacacaaattttttttgaagaaaagtggtgttcaagtattccagcaaagcagtcgtggagaaaacatgatgttggaaatcttagtggatgcagaatcagatgaacttaccgtagaaaatgtagcttcatcagtgcttggaaaatctgtctttgttaattggcctcaccttgaggaagctagagtcgtggctgtatcagatggagaaactaagttttacttggaagaacctccaggaacacagaagctttattcaggaagaactgccccaccatctaaagtggttcatcttggagataaagaacaatctaactgggcaaaagaagtacaaggaatttcagaacactacctgagaagaaaaggaataataataaatgaaacatctgcagttgtgtatgctcagttactcacaggtcgtaaatatcaaataaatcaaaatggtgaagttcgtctagagaaacagtggtcaaaacaagttgttccttttgtttatcaaactattgtcaaggacatccgagctttcgactcccgtttctccaatatcaaaacattggatgatttgtttcctctgagaagtatggtctttatgctgggaactccctattatggctgcactggagaagttcaggattcaggtgatgtgattacagaaggtaggattcgtgtgattttcagcattccatgtgaacccaatcttgatgctttaatacagaaccagcataaatattctataaagtacaacccaggatatgtgttggccagtcgccttggagtgagtggataccttgtttcaaggtttacaggaagtatttttattggaagaggatctaggagaaaccctcatggagaccataaagcaaatgtgggtttaaatctcaaattcaacaagaaaaatgaggaggtacctggatatactaagaaagttggaagtgaatggatgtattcatctgcagcagaacaacttctggcagagtacttagagagagctccagaactatttagttatatagccaaaaatagccaagaggatgtgttctatgaagatgacatttggcctggagaaaatgagaatggggctgaaaaagttcaagaaattattacttggctaaaaggacatcctgtcagtactttatctcgttcttcttgtgatttacaaattctggatgcagctattgttgagaaaattgaggaagaagtcgaaaagtgcaagcaaagaaagaataataagaaggtgcgagtaacagtgaaaccccatttgctatacagacctttagaacagcaacatggagtcattcctgatcgggatgcagaattttgtctttttgaccgtgttgtaaatgtgagagaaaacttctcagttccagttggccttcgaggcaccatcataggaataaaaggagctaatagagaagccaatgtactatttgaagtattatttgatgaagaatttcctggagggttaacaataagatgctcacctggtagaggttatcgactgccaacaagtgccttggtgaacctttctcatgggagtcgctctgaaactggaaatcagaagttgacagccatcgtaaaaccacaaccagctgtacatcaacatagctcaagttcatcagtttcctctgggcatttgggagccctcaaccattcccctcaatcactttttgttcctactcaagtacctactaaagatgatgatgaattctgcaacatttggcagtccttacagggatctggaaagatgcaatactttcagccaactatacaagagaagggtgcagttctacctcaagaaataagccaagtaaatcaacatcataaatctggctttaatgacaacagtgttaaatatcagcaaagaaaacatgaccctcacagaaaatttaaagaagagtgtaagagtcctaaagctgagtgttggtcccaaaaaatgtccaataagcagcctaactctggaattgagaactttttagcatctttgaatatctccaaagaaaatgaagtacagtcatctcatcatggggagcctccaagtgaagagcatttgtcaccacagtcatttgccatgaagggaacacggatgcttaaagaaattctaaaaattgatggctctaacactgtggaccataagaatgaaatcaaacagattgctaatgaaatccctgtttcctctaacagaagagatgaatatggattaccctctcagcctaaacaaaataagaaattagcatcttatatgaacaagcctcacagtgctaatgagtaccataatgttcagtctatggacaatatgtgttggcctgcccccagccagatccctcctgtatccacaccagtaactgaactttctcgaatttgttcccttgttggaatgccacaacctgatttctcctttcttaggatgccacagacaatgaccgtttgccaagtaaaattatctaatggcttactggtacatgggccacagtgccactctgaaaatgaagccaaagagaaagctgcactttttgctttacaacagttgggctccttaggcatgaatttccctttgccttcacaagtatttgcaaattatccttcagctgtaccacctggaaccattcctccagcctttcccccacctactgctaatataatgccttcgtcgtctcatctctttggctcaatgccatggggaccatcggtgccagttcctgggaagcccttccatcatactttatattctgggaccatgcccatggctgggggaataccagggggtgtgcacaatcagtttatacctctgcaggttactaaaaaaagggttgcaaacaaaaagaactttgagaataaggaagcccagagttctcaagccactccagttcagactagccagccagattcttccaacattgtcaaagtaagtccacgggagagctcatcagcttctttgaagtcctctccgattgctcaacctgcatcttcttttcaagttgaaactgcctctcaaggccatagtatatctcaccataagtcaacaccaatctcttcttcaagaagaaaatcaagaaaactggctgttaattttggtgtttctaaaccttctgagtaaatttggctcttagaattaagttaatttcttctctttccatctacctttttataaatacatatctatgtctcataaaaattagaatgtactattttaaaataatatgtgtaaattgaaatttttttcatttttaagttatcaggcacttttcatgctgtttaaaagactgtgtatcaaattgtgcactttaagtatgtgcagtttgttgtatgtcaattatacctcaataaatctgtaataaaaaactaaattaaaccttgcattaaaataatatcacagtatcagtggactaaacattaaaatgtaccactctaatcattggcctcatgattgaagcatcctgaactatgaattagacatcagttagcaataataagcattttttacactatcattgaggaataattacatggagcatgaaatttgggcctccagtataacttactgaatgtggattttatttctctttttaatgatgtaacgaaaattgtcaggagaatggctcttatttatgtgtgttttattatgcttgttgctctgaaggttttaaacctgtgtgaaaggtacttgttg

SEQ ID NO: 62 - DNA, 2585 bp, Homo sapiens
  misc_feature: Incyte ID No 1806372CB1
atggtgtcggtcaccaaatatgaccttactggctgctctgccttctgcaggtcctgccagagagccaccatgacctctcagcctctcaggctagcagaagagtatggcccaagtcctggggagtctgaactggctgtgaacccctttgatgggcttcccttctcttcccgctactatgagctgctgaagcagcgccaagccttgcccatctgggctgctcgctttaccttcttggagcagttggagagtaaccccactggagtggtgctggtgtctggggagcctggttctggcaagagcacccagatccctcagtggtgtgcagagtttgcgctggccagagggttccagaaaggacaggttactgttactcagccctaccctcttgcagcccggagcctggctctgcgggttgctgatgagatggacctgaccctgggtcatgaggttggatacagcatcccccaggaggactgcacggggcccaacaccctgctcaggttctgctgggacaggctgcttctgcaggaggtggcctcgacccgaggcactggagcctggggcgtgctggtactagatgaggctcaggagcggtcggtggcatcagattcactccaggggctactgcaagatgccaggctggaaaaacttccgggggacctcagagtggttgtggttactgacccagcccttgaacctaagctccgagctttctggggcaatcctcctattgtgcatatacccagagagcctggtgagagaccttcccccatctactgggacaccatcccacctgatcgggtggaagctgcctgccaagcagtgcttgaattgtgtcggaaggagcttccaggagatgtgctagtgttcctgcccagtgaggaggaaatttccctgtgctgtgaatccttgtccagggaggtagagtccttgcttctccaagggcttccaccacgagtactgccccttcacccagactgtggacgagccgttcaggctgtgtatgaggacatggatgcccgaaaggttgtggtcactcactggctggctgacttctccttctccctcccttccatccaacatgtcatcgactcaggactggagctccgaagtgtttacaatcctaggatccgagcagaattccaagtgttgaggccaatcagcaagtgtcaggcagaggcaagacgattgcgagcaagagggttcccaccaggatcctgcctctgcctgtatcctaagtccttcttagaactagaagctccaccattgccacaacccagggtgtgtgaggagaatctgagctccctggtgttactactaaaaaggagacagattgcagagccaggggagtgtcacttcctggaccagcctgctccagaagcactgatgcaagccctggaagatttagactatctggcagccctggatgatgatggggacctgtcagatctgggtgtcatactatcagaattccctctggcccctgagctggccaaagccctgctggcctcatgcgagtttgactgtgtggacgagatgctcaccctggctgccatgctcacagctgcccctgggtttacccgtcctccactcagtgcagaagaagctgccctgcgtcgggccctggaacacacggatggtgaccacagttctctgatccaggtgtatgaagcctttatacaaagtggagcagatgaggcttggtgccaggctcgaggtctgaattgggcagcattgtgccaagcccataaacttcggggagaactcctagaactcatgcaacgaattgaacttcccttgtccctaccagcctttggctctgagcagaatcgcagagaccttcagaaagcactggtgtcaggatactttctcaaggtggccagagacacagacgggactggaaattaccttctcctaacccataagcatgtggcccagctctcctcatactgctgctaccgaagccgcagagctcctgccagacccccaccatgggtgctctaccacaatttcaccatatccaaagacaactgcctttccattgtttctgagattcaaccacagatgctggtggaattggcccctccatacttcctgagtaacttgcctcccagtgagagcagagaccttctgaaccagctaagggaaggaatggcagattctacagcagggagcaaatcatcctcagcccaggagttcagagatccctgtgtcctgcagtgacctgcctgcctatggaatggagctgggttcatctcatcacattagattatccctcagggtgacaccaaagcacccagacagatttagaagcccaaagtttagggtcaaatgtaaaccctggaacctgagtcccaagaaatggtagactgggaatggaaagaatggggtaaaccacagtctacatagggaaggactctttccttagccttctcttattgattggagagggactgacatgctcctcattctcttaactttgccaaacccattcttgtactcccttgtgatctataaaagatttttctatgatgccaaaaaaaaaaaaaaaaataaaaaaaaaaaaaaaaaaaaaaaaaaaaaaa

SEQ ID NO: 63 - DNA, 1888 bp, Homo sapiens
  misc_feature: Incyte ID No 2010564CB1
agcggaggggatttatttttcaatctaaagtttttcacctctctctagtaagacatctgaaactcataagccatctttaagaatttcttacaaaaacatgctggaagacagtggatcttgttgtatcctgaagatttttttctcttcgttattttaaattaattgtcaacagatgtgcaactgttaggttggttcttaagtcactcggagaagagagagatgcatctactcaaggttggcacttggagaaacaacactgcctcttcctggcttatgaagttcagtgttctttggcttgttagtcagaactgttgcagagcaagtgttgtttggatggcttatatgaacatatcatttcatgttgggaatcatgtgttgtcagagttgggagagactggagtctttggaagaagctccactttgaagagagtggcaggagttatagtgccaccagagggaaaaatccaaaatgcatgtaatcccaataccattttcagccgatcaaagtactcagagacctggcttgcacttattgaacggggaggttgtaccttcacacagaaaattaaagtggcaactgagaagggagccagtggagtgatcatctataacgttccaggtactggcaaccaggtgttccccatgtttcatcaggcatttgaagatgtcgttgtggttatgattggtaacttaaaaggcacggaaattttccatttaattaagaagggagttctcattacagccgtggttgaggtggggagaaagcacatcatctggatgaatcactatttggtctcttttgtgattgtcacaactgctaccttagcatatttcatcttttatcacattcatagactttgtttagcaaggattcagaaccggagatggcagcgattaacaacagatcttcagaacacatttggacaactccaacttcgagtagtaaaagagggggatgaagaaataaatccaaatggggatagctgcgtaatttgctttgaacgctataagcctaatgacatagttcgtattctgacttgtaaacattttttccacaagaattgcattgacccctggattttaccccatgggacatgccccatttgcaaatgtgatattcttaaagttttggggattcaagtggttgttgaaaatggaacagaacctttgcaagttctaatgtcaaatgaactgcctgaaaccttatcacctagtgaagaggagacaaataatgaagtttctcctgcaggaacctcagataaagtaatccatgtggaggagaaccctacttctcagaataatgacatccagcctcattcagtagtggaagatgttcatccttcaccttgatggcatgacttttgaggaagtgtattaaacctgtatgtgaaatcaggtcctaatactgacaagcagtttgtctgtttgaagtgtggtttttgtgtccttttttgttacttcagtaattttatacattctatgtccaacctcaaagatagcaaaaaagtcctagtgggattttttttgtgcaattttggacctttgctaagtgtaattttttgtcaatgtatgttactcctgtgagtgtacatatgtatatttatatgtacacattcatgttaaagctaaggacaaacttattttcttaaatatttactgtacatatattgggttctattttggtaagaaaattactgatatgtaatatgttctaatacagaaagtatcaattaagtttgaaaacaataaattatcttttatgtgctaggaagagtaaaaattatctttggataacatatttatagtatgtcttagttgtgaggttattgtgctgttttttcttttgcattcttggcatgaacatcttacaaaatcttattattcttat

SEQ ID NO: 64 - DNA, 2991 bp, Homo sapiens
  misc_feature: Incyte ID No 7364908CB1
gaattttgtaggctgctttggtttaggaaataagggaatacactccttaattttcctcttggtcactcccattcttgtctccagggaacatctgcagttaaatatatatttctccatgatatccatgattattgtcttaagatagttctacaaatagaaattcaaatagaattgctggttcaaaggggaacaattaggtgtttgtaaatccacaggaaacatatctgcatttatctaattaaaaatacaccttgccctatgtgatcaaattatacacctagttctgtatgatcatatcatagtcacaaaaggactaaaggtggatacctccttcacatggtgtttgctataagggtcaaacgaccttctcccatcacgaccttctcccacaggatggcctatcagctacctgacttctggtggtggttgtttagagactggtgaatgttagttctggcccataaaaactccattctttcttccagacctgattcctcagacctctgccctatcggaagggaagcagaaaatgatcaggtctcagggtccagtgtcatttgaggatgtggctgtggatttcacccaggaggagtggcagcaactggactatgctcagaggaccctgtacagggatgtgatgctggagatctatagccacctggtctcaatgggatatccagtttccaaaccagatgtcatctccaagttggaacaaggagaagagccatggatcataaagagacacataccaaattggatctatccagacagagagagtagacttgacacccctcaactggatatatttagagatgttttcttccataaggagacactggaaagtattacagggggtcattcattgtactccattttaaaagtctggcaagacaaatttgtcaggcaagtcgtagtcatcaacaacaaaagaatatctgaagagtcaggtcatccatataatatatttggaaaaatatttcatgactgcacagacctagatacttcaaaacaaagactgtgtaagtgtgattcatttgaaaagaccttgaaaccaaatattaacctagtgagttataataggaattttgcaagaaaaaacattgatgagaattttagatgtgggaaaacacctagctacagttcttgctattctaagcatgaaaaaattcatagtggaatgatacactgtgaagctactcattgtggaaagattcttagccataaacaatctcttattcattatgtgaatgttgaaactggggagaagacctatgtatgtgttgaatgtggaaaatcctttctcaagaagtcacagattattatacatcaaagaattcatactggagagaaaccttatgattgtggtgcatgtggaaaagccttcagtgagaagtcacaccttattgcacatcagagaactcatactggggagaaaccttatgattgttctgaatgtggaaaaggcttttctcagaaatcatccctcattatacatcagagagttcactctggggaaaaaccatatgaatgtagtgaatgtgagaaagccttctcccagaaatcacccctcattatacatcagagaatacatactggggaaaagccctatgaatgtagagtgtgggaaagccttttcccagagtcacagctgattatacatcacagagctcatactggagagaagccatgtaagtgtactgaatgtgggaaagcattctgttttatacattaaagagttcacactggtgagaaaccctacaaatgtgctcaatgtgaggaagccttcagcaggaagtcagaactcattatacatcagataattcatactggggagaaaccgtatgaatgtacagaatgtgggaaaacattctctcgcaagtcacaactcatcatacatcagagaacccacactggagagaaaccctataaatgtaccaaatgtggaaaatccttctgtcagcagtcacatctcattggacatcagagaattcacacgggagaacaaccttatgtatgttctgaatgtgggaaagccttctctcagaagtctcacctcccagggcattggtgaattcatacaggagagaaaccttacatatgtgctgaatgtggaaaggccttttctcagaagtcagaccttgttgtacatcagataattcatactggagagaaacctgatcgatgtactgtatgtgggaaggccttcatccagaagtcccaactcactgtacatcagagaattcatacactaatgaaatcataagaatggtctgaacacagaaaagccttcagggtcagttcaagccttaatagatagtgcaacaaccaatggatttgatgattttggggactacatctttgttgataaaattttacaagtgaagtcatgttcctaatgtatttcattctttatcaaagataatagagaagtcaatacgtaaatgatggacattttcactatggcatataaaagtttttaaattgagaaatgaatgattagcataacagaacgaattgcatgtacatctcttttgaagttatgtgctcctgattatactacataacaatcagatatgtgtaagattgttaatgttagcctagtataattttggttatgcagttcttcactatagaggacatcaagaaagtctgcatttgaaaatgccaatatctagaaattgtatttgagggaagggacttgccatgcactcctaaaggtatatgtaaaatttttcttgtagaaagagacactcatttataaatattccatgccaggtaaggatggcacataagtaatttccagttggtgaaacttcaacagacatgaggaggaaacctatcacaaggttcctatctatgtagaatttagacaaacaatgcggaagggggatgtgctgagtattctagttatctttcaagagaaagtctttcaggagtggatgtctcactgttctggtctacccctgaaaagaacctgttctgagaaataca

SEQ ID NO: 65 - DNA, 3874 bp, Homo sapiens
  misc_feature: Incyte ID No 7489960CB1
gccgaggcaggggcagaggctctatgggaggagaccacccggaggatgaagaggatttctacgaggaagagatggactatggagagagtgaggagccaatgggagacgacgactatgacgagtactccaaggagctgaaccagtaccggcgctccaaggacagccgaggccgagggctaagtcgaggccgtggcaggggctcccgaggtcgagggaaaggaatgggtcggggccgaggccgaggtggcagccgaggagggatgaacaagggcggaatgaacgatgacgaagacttctatgatgaggacatgggcgacggtggtggtggaagctaccggagtcgtgaccatgacaagccccaccagcagtcggacaagaaaggcaaagtcatttgcaagtacttcgtggaagggcgctgcacctggggagaccactgtaattttagccatgacatcgaactgccaaagaagcgagaactgtgcaagttttacatcactggattttgcgccagagctgagaactgcccttatatgcacggtgatttcccgtgtaagctgtaccacaccactgggaactgcatcaatggtgacgactgcatgttttcccacgaccctctgaccgaagagacgagggagctcttggataagatgttggccgatgatgcagaagcaggtgccgaggatgagaaggaggtggaggaactgaagaagcagggcatcaaccccctgcccaaaccgccccctggtgtgggcctcctgcccacccctcctcggccccctggcccgcaggctccaacctctcccaacggcaggcccatgcagggtggccccccgcccccgccccctccccctcccccaccgcccgggccccctcagatgcccatgccggtgcatgagccactgtccccgcagcagctgcagcagcaggacatgtacaacaagaagatcccctccttgtttgagatcgtggtgcggcccacgggacagctggctgagaagctgggtgtgaggttccctggacccggtggacccccagggccaatgggccctgggcccaacatgggacccccagggccaatgggcggtccaatgcatcctgacatgcaccccgacatgcacccggacatgcaccctgacatgcacgcagacatgcacgcagacatgccgatgggccctggcatgaatcctggcccacccatgggccctggcggccctccaatgatgccctacggccctggagactccccacattctggaatgatgccccctatcccgccagcccagaacttctatgaaaacttctaccagcagcaggagggcatggagatggagcccggactcctgggggatgcagaggactacgggcactacgaagagctgccaggggagcctggggagcacctcttccctgagcaccctctggagcccagacagcttctctgagggagggcccccatgccggccgaagccaggcgccggtgtccctgacttcctgccctcagcccagagggccctgtacctgaggatccagcagaagcagcaggaggaggaggagagagcgaggaggctggctgagagcagcaagcaggaccgggagaatgaggaaggtgacaccggaaactggtactcaagtgatgaggatgagggtggaagcagtgtcacctccatcctgaagaccttgaggcagcagacgtccagccgacccccggcttcagttggggagctgagcagcagtgggctgggggacccccgcctccagaagggacaccccacaggaagccggctggctgaccctcgcctcagccgggaccccagactcacccgccatgtggaggcttctggcgggtctggcccaggtgattcgggaccctccgatcctcggctggctcgcgccctgcccacctccaagcccgaaggcagccttcattccagccctgtgggccccagcagttccaaggggtctgggccgcccccaacggaggaggaggaaggggagcgggccctgcgggagaaggccgtgaacattcccctggacccactccccgggcaccctctgcgggacccacggtcacagctgcagcagttcagccacatcaagaaggacgtgaccctgagcaagcccagcttcgcccgcaccgtgctctggaatcccgaggacctgatccccctacccatccccaagcaggacgcagtgccccccgtgcccgcggccctgcaatccatgcccaccctggacccccggctgcaccgcgctgccacggcagggccccccaacgcccggcagcgcccgggcgcctccacggattccagcacacagggcgccaacctccccgactttgaacttctgtctcgcatcctcaagacagtcaatgccaccggctcctcggccgcccccggttccagcgacaaacccagtgacccccgggtgcggaaggcccccaccgaccctcggctgcagaaacccacagactctacggcctcctcccgggctgccaagcccggccctgctgaggcgccctctcccaccgccagcccgagtggggatgcctccccaccagccaccgctccctacgacccccgcgtgctggcggccggtggactgggccagggcggagggggcgggcagagcagtgtgctgagcggtatcagcctctacgacccgaggactcccaacgcggggggcaaagccacagagccggctgctgacacgggtgcccagcccaagggtgctgagggcaatggcaagagctcggcctccaaggctaaggagcccccgttcgtccgcaagtctgccctggaacagccagagacagggaaggccggtgctgatgggggcacccccacggacagatacaacagctacaaccggccccggcccaaggctgctgcagcccccgctgccaccaccgccaccccaccccccgagggtgcccaaccccagcccggggtgcacaacctgcccgtgcccaccctcttcgggacggtgaagcagacacccaagacgggctcaggaagcccatttgctgggaacagtccggcccgcgagggtgagcaggatgcggcatccctgaaggatgtttttaaaggcttcgaccccacggcctcccccttttgccagtagtgtccagccagagctgcggctccagccacccttcctagggtggcattcagggcagcacccagggtagggaacttgggggcaaggggaggcaggctgggtgcttccttttttcttttctttttcttttgctttccgatctcttttattttttttaaaggtcaggttctttcctttggaggatttggttcccttggttttttacccatcttaaggttctgggtccggtggtggccgggctccagggggctctctgtgtgtgagaccaaacacggacatctcatgtgccggagaacctggtgtaacnttntccggccagatggcctgggcgtcctaggactcctggtggggctcctggcggtgtggcgggggccttggctgggagaggcccagagggggggttattactgggggccaaaggcatggggcccctttatccccccagaggacccacagcgggacacccgagttgaggtggtctcggcggacacagggtacttgcgaacacgcgctttccttaaacctggggtgggccacggtgaaagggaaaaacacccggggaaaacccaaaggaaacccaagggggcagagggacctgtgccagtgggcgaaaggccagaggtccccacggaggcgcgttaccttaagagaaccccctagaggggtgtccacccccagttcaaactgggtccttttatccacgactgggccggggctgggattaccaaccccacactccagagggcagagaccacgc

SEQ ID NO: 66 - DNA, 1670 bp, Homo sapiens
  misc_feature: Incyte ID No 8555401CB1
agcggattgtgatggtctagataagtgtacatgcttaggccttctgaagcagcatttgaagctgcagtcctgaaaaccatgcaggccggaagagtagataaagaaatatttatttgagatggcacatgtttcttcagaaactcaagatgtttcccccaaagatgaattaactgcttcagaagcctccactaggtctccattgtgtgaacacaccttccctggggactcagacttacggtcaatgattgaagaacatgcttttcaggttttgtcacaaggatccttgttagaaagtccaagttacacagtttgtgtctctgagccagataaagatgatgattttctttctctgaactttcccaggaaactttggaaaatagtggaaagtgaccaattcaagtctatttcatgggatgagaatggaacttgcatagtgattaatgaagaactcttcaagaaagaaattttggaaacaaaggctccttacagaatatttcaaactgatgctatcaaaagttttgttcgacagctcaacctttatggatttagtaaaattcaacagaattttcaaagatctgcctttctagccacctttctgtcagaagagaaagaatcgtctgtcttaagcaagttaaagttctattataatccaaatttcaagcgtggctatccccaacttttagtaagagtgaagagaagaattggtgttaaaaatgcttcacctatatctactttattcaacgaagatttcaacaagaagcattttagagcaggggctaacatggagaatcataattctgccttagctgctgaagctagtgaagaaagtttattttcagcctctaaaaatttaaatatgcctctaacaagggaatcttctgtcagacagataattgcaaattcatctgtccccattagaagtggtttccctcctccttcaccttcaacctcagttggaccatcagaacaaattgcaacagatcaacatgctattttaaatcagttgaccactattcatatgcactctcatagtacctacatgcaagcaaggggccacattgtgaattttattacaaccacaacttctcaataccacatcatatctcccttacaaaatggttattttgggctgacagtggaaccatctgctgttcccacacgatatcctctggtatcagtcaatgaggctccatatcgtaacatgctaccagcaggcaacccgtggttgcaaatgcctacgatcgctgatagatcagctgcccctcattccaggctagctcttcaaccatcaccactggacaaatatcaccctaattacaactgatctgccattaaaagaggaccagattatgaatgacaacagagactaacatttacattgacaaaaaaccctaaaaatttctgcaattatcttattgaacaataaaattgcatgtttacttctaaaaaaaaagaagaaaaaaaaattgnnnnnnnnnnnnnnnnnnnnnnnnnnnnnnnnnnnnnagcttggcctggcgtcgttttacacgtcgtgactgggaacgcccggcgttacccacttaatcgccttcagcaatcccctttcgcagctgggtaatagcgaaagcccgacgatcgcctcccaaagtgcgcacctgatggcgatggacggcctgtagggccattat